SEQUENCE LISTING (101 sequences)

SEQ ID NO: 1 - DNA, 7426 bp, synthetic construct
  source 1..7426: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataacaacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagtatgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgagtgttttagttgccgtggaaacttttcgcctgtctctggcaggcctgggatcggtggcaagcacatcacgccggatgcgacgcaaatgcgtcttatccggcctacacggtgatgatgtggtaggccggagcaggtgagtcgctctccaacgtgaagtttgtcagctatctgtagcccatctctgcatgggcttttttctccgtcaattctctgatgcttcgcgctttttatccgtaaaaagctataatgcactaaaatggtgcaacctgttcaggagactgctttatggcaacaggcacgcagcccgatgctgggcagatcctcaactcgctgattaacagtattttgttaatcgatgacaacctggcgatccattacgccaaccctgccgcgcaacaactgctcgcccaaagctcccgcaaattgtttggtacaccgttaccggaactgttgagctacttctcattaaatatcgagctgatgcaagaaagtctggaggcggggcaaggttttaccgataacgaagtgacgctggtcatcgacgggcgctcgcatatcctttctgtgacggcccagcgtatgccggacggcatgatcctgctggagatggctccgatggataaccagcgccgcttaagtcaggaacagctacagcacgcccagcaggttgctgcccgtgatttagtgcgcggcctggcacatgagattaaaaatccgcttggcggtttacgtggcgcggcgcagctgctcagcaaagcgttacctgacccatcactactcgaatataccaaagtgattatcgaacaggcggaccggctgcgaaatctggtcgaccgtctgttggggccgcagctgcccggtacgcgcgttaccgaaagtattcacaaagtggctgaacgcgtggtaacgctggtgtcgatggaactgccggacaacgtgcggttgattcgtgattacgatcccagcctaccggaactggcgcacgacccggatcaaattgaacaggtcttgctgaatattgtgcgcaatgcgctacaggcgctggggccggaaggcggtgaaatcattctgcgtacccgcaccgcgtttcaactgaccttacacggcgagcgctaccggctggcggcgcggattgatgtggaagataacgggccgggcattccgcctcatttgcaggatacgctgttttacccgatggtcagcggccgcgaaggtggcaccgggcttggcttatccatcgctcgtaatttgattgatcagcattcaggcaaaattgaatttaccagttggccagggcataccgagttctcggtttacctgcctatcaggaaataaaggtgacgtttatgcaacgagggatagtctgggtagtcgatgacgatagttccatccgttgggtgcttgaacgtgcgctcgctggggcaggtttaacctgtacgacgtttgagaacggcgcagaagtgctggaggcgctggcgagcaaaacgccggatgtgctgctttcagatatccgtatgccgggaatggacgggctggcgctgctcaagcagattaaacagcgccatccaatgcttccggtcatcattatgaccgcacattccgatctggatgctgccgtcagcgcctatcaacaaggggcgtttgattatctgcccaaaccgtttgatatcgacgaagcagtggcgctggttgagcgcgctatcagtcattaccaggaacagcagcagccgcgtaatgttcagcttaacggcccaacgaccgatatcatcggcgaagcgccagccatgcaggacgtgttccgtattatcggtcggctttcgcgttcttctattagcgtgctgattaacggcgaatccggcaccggtaaagaactggtcgctcatgccctgcatcgccacagtccgcgcgccaaagcgccgtttatcgcgctgaatatggcagctatcccaaaagatttgatcgaatcagaactgtttggccacgagaaaggcgcgtttactggcgcgaataccattcgtcaggggcgttttgaacaggccgatggcggtacattattcctcgacgaaattggtgatatgccgctggatgtgcagacgcgtttgctgcgcgtgctggcagacggtcagttttaccgcgttggcggctatgcgccggtgaaagtggatgtgcggattatcgctgccactcaccagaatctcgaacagcgagtgcaggaaggtaagttccgtgaggatctgttccaccgcctgaacgttatccgcgttcatctgccgccgctgcgcgaacgtcgggaagatattccccgtctggcgcgccattttttacaggttgccgcgcgcgaactgggcgtagaagcgaagttactgcatccggaaaccgaagctgctctgacgcgtctggcgtggccaggcaacgtgcgccagctggaaaacacctgccgctggctaacggtgatggccgccgggcaggaagtgttgattcaggatttgcccggcgaactgtttgaatcaacggttgcggagagtacttcgcaaatgcaaccggacagctgggcgacgcttcttgcgcagtgggcagacagagcgctgcgttccggtcatcaaaatctgctttccgaagcgcagccagagctggagcggacgttactgacgaccgcgttgcgacatacgcaggggcataaacaggaagcggcgcggctactcggctggggccgcaacaccctgacgcgtaagttaaaagagctggggatggagtgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 2 - DNA, 4840 bp, synthetic construct
  source 1..4840: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgatagcgcaatatttcatcgttggtgcaaaaatgtaacgcactgtgcactgtcatagtgcgttttcattttcaaacttcttaacttcctgctctctttctcgtttttcatttctggcacaccgcttgcaataccttcttcgtgtagcagaaccattgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 3 - DNA, 5207 bp, synthetic construct
  source 1..5207: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgagcttctatatcgcctgcgtagtgattacctgggcggtatatggtcggcattctaaaaaataaaccgttactcgtcatacttcgggttacatgtgctgcggctgcgttcattcaccccagtcacttactttagtaagctcctgggattcattcacttgccgccttcctgtaaaccgaattatatagagtaaaatatttgattatcctttgcgcggcatgatgtcgcgctttttttatgcgtcatttagttacaacatactaatgttatatggtttatttcgccggatttcattaagagccattaatatgttacccatggggaatactccttaatacccatctgcataaaaatcttaatagtttaaataactacaggtataaaacgtcttaatttacagtctgttatgtggtggctgttaattatcctaaaggggtatcttaggaatttactttatttttcatccccatcactcttgatcgttatcaattcccacgctgtttcagagcgttaccttgcccttagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 4 - DNA, 4834 bp, synthetic construct
  source 1..4834: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgtttttaactggcctgtcaggcagtggtgcgtttttctaccgctattgaggtaggtcaatttgcgaaggcggattattttgtggcaaacagatgttctttttgatttcgcgcaaaaagattcagaattttactgttagtttcctcgcgcagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 5 - DNA, 5917 bp, synthetic construct
  source 1..5917: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaattctggtacgcctggcagatattttgcctgccgggcgaacagtgtgatacattgctgtgtcgggtaagccattacgctatccgacacagtgttaaatcctcgcttttttccttccccgaactgaaataaattagcgacacagcttgtggctggtttatcatcaatataaatgtattttttcccgatttcccttttgaggttgatgtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtgacgaaaatcttcatcagagatgacaacggaggaaccgagaaataaagccctgagtttaaccgctcggggctttttgcgttttcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 6 - DNA, 6305 bp, synthetic construct
  source 1..6305: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaattctggtacgcctggcagatattttgcctgccgggcgaacagtgtgatacattgctgtgtcgggtaagccattacgctatccgacacagtgttaaatcctcgcttttttccttccccgaactgaaataaattagcgacacagcttgtggctggtttatcatcaatataaatgtattttttcccgatttcccttttgaggttgatgtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtgacgaaaatcttcatcagagatgacaacggaggaaccgagaaataaagccctgagtttaaccgctcggggctttttgcgttttcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgacgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 7 - DNA, 5119 bp, synthetic construct
  source 1..5119: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 8 - DNA, 7403 bp, synthetic construct
  source 1..7403: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataactcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgtttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaatccgcctttgtcatcttttattgccataagccagtcgatgctggcttattttctttgcagtcaaaatacgggcgttagattttacaacgattggtgattttttgttcgcatgattagccatgtctttttcacggaaatagtgttttatactggttggtgatttcttatcgctatatacctctggtttttagatccctccttgctttaaaacgttataagcgtttaaattgcgcttcaggtgcttaaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 9 - DNA, 3820 bp, synthetic construct
  source 1..3820: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataacgactgttgtacactaccggggccttttggccccgtttttttatctggaggattaatgacaatcacggacctggtactgattcttttcatcgccgcactcctggccttcgcgatctacgatcagttcatcatgccccgccgtaacggccccaccctgctggcaattcctttgctccggcgtggtcgcatcgatagcgttatcttcgtcggattgattgtcattcttatctataacaacgtcacgaatcatggtgcgttaataacgacatggttattaagcgcactggctctgatgggtttttatatattctggatccgcgttccgaagatcatctttaaacaaaaaggttttttcttcgccaatgtctggattgaatatagccgaatcaaagcgatgaacttgtcggaagatggcgtgttggtgatgcaattagaacagcgtcggctgttaatccgcgttcgaaatatcgacgatctggaaaaaatttataagcttctcgtttcaactcaataagttatgaatacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 10 - DNA, 3363 bp, synthetic construct
  source 1..3363: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataacgtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgtttgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 11 - DNA, 3408 bp, synthetic construct
  source 1..3408: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataacgtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcaggacattgtcatgaatatcactgctactgttcttcttgcgtttggtatgtcgatgatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 12 - DNA, 177 bp, synthetic construct
  source 1..177: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataac

SEQ ID NO: 13 - DNA, 27 bp, synthetic construct
  source 1..27: mol_type = other DNA
gaattcattaaagaggagaaaggtacc

SEQ ID NO: 14 - DNA, 717 bp, synthetic construct
  source 1..717: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 15 - DNA, 102 bp, unidentified
  source 1..102: mol_type = unassigned DNA
cttggactcctgttgatagatccagtaatgacctcagaactccatctggatttgttcagaacgctcggttgccgccgggcgttttttattggtgagaatcca

SEQ ID NO: 16 - DNA, 1128 bp, synthetic construct
  source 1..1128: mol_type = other DNA
cctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcct

SEQ ID NO: 17 - DNA, 796 bp, Escherichia coli
  source 1..796: mol_type = genomic DNA
gatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 18 - DNA, 1723 bp, Escherichia coli
  source 1..1723: mol_type = genomic DNA
aacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagtatgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaa

SEQ ID NO: 19 - DNA, 2756 bp, Escherichia coli
  source 1..2756: mol_type = genomic DNA
gtgttttagttgccgtggaaacttttcgcctgtctctggcaggcctgggatcggtggcaagcacatcacgccggatgcgacgcaaatgcgtcttatccggcctacacggtgatgatgtggtaggccggagcaggtgagtcgctctccaacgtgaagtttgtcagctatctgtagcccatctctgcatgggcttttttctccgtcaattctctgatgcttcgcgctttttatccgtaaaaagctataatgcactaaaatggtgcaacctgttcaggagactgctttatggcaacaggcacgcagcccgatgctgggcagatcctcaactcgctgattaacagtattttgttaatcgatgacaacctggcgatccattacgccaaccctgccgcgcaacaactgctcgcccaaagctcccgcaaattgtttggtacaccgttaccggaactgttgagctacttctcattaaatatcgagctgatgcaagaaagtctggaggcggggcaaggttttaccgataacgaagtgacgctggtcatcgacgggcgctcgcatatcctttctgtgacggcccagcgtatgccggacggcatgatcctgctggagatggctccgatggataaccagcgccgcttaagtcaggaacagctacagcacgcccagcaggttgctgcccgtgatttagtgcgcggcctggcacatgagattaaaaatccgcttggcggtttacgtggcgcggcgcagctgctcagcaaagcgttacctgacccatcactactcgaatataccaaagtgattatcgaacaggcggaccggctgcgaaatctggtcgaccgtctgttggggccgcagctgcccggtacgcgcgttaccgaaagtattcacaaagtggctgaacgcgtggtaacgctggtgtcgatggaactgccggacaacgtgcggttgattcgtgattacgatcccagcctaccggaactggcgcacgacccggatcaaattgaacaggtcttgctgaatattgtgcgcaatgcgctacaggcgctggggccggaaggcggtgaaatcattctgcgtacccgcaccgcgtttcaactgaccttacacggcgagcgctaccggctggcggcgcggattgatgtggaagataacgggccgggcattccgcctcatttgcaggatacgctgttttacccgatggtcagcggccgcgaaggtggcaccgggcttggcttatccatcgctcgtaatttgattgatcagcattcaggcaaaattgaatttaccagttggccagggcataccgagttctcggtttacctgcctatcaggaaataaaggtgacgtttatgcaacgagggatagtctgggtagtcgatgacgatagttccatccgttgggtgcttgaacgtgcgctcgctggggcaggtttaacctgtacgacgtttgagaacggcgcagaagtgctggaggcgctggcgagcaaaacgccggatgtgctgctttcagatatccgtatgccgggaatggacgggctggcgctgctcaagcagattaaacagcgccatccaatgcttccggtcatcattatgaccgcacattccgatctggatgctgccgtcagcgcctatcaacaaggggcgtttgattatctgcccaaaccgtttgatatcgacgaagcagtggcgctggttgagcgcgctatcagtcattaccaggaacagcagcagccgcgtaatgttcagcttaacggcccaacgaccgatatcatcggcgaagcgccagccatgcaggacgtgttccgtattatcggtcggctttcgcgttcttctattagcgtgctgattaacggcgaatccggcaccggtaaagaactggtcgctcatgccctgcatcgccacagtccgcgcgccaaagcgccgtttatcgcgctgaatatggcagctatcccaaaagatttgatcgaatcagaactgtttggccacgagaaaggcgcgtttactggcgcgaataccattcgtcaggggcgttttgaacaggccgatggcggtacattattcctcgacgaaattggtgatatgccgctggatgtgcagacgcgtttgctgcgcgtgctggcagacggtcagttttaccgcgttggcggctatgcgccggtgaaagtggatgtgcggattatcgctgccactcaccagaatctcgaacagcgagtgcaggaaggtaagttccgtgaggatctgttccaccgcctgaacgttatccgcgttcatctgccgccgctgcgcgaacgtcgggaagatattccccgtctggcgcgccattttttacaggttgccgcgcgcgaactgggcgtagaagcgaagttactgcatccggaaaccgaagctgctctgacgcgtctggcgtggccaggcaacgtgcgccagctggaaaacacctgccgctggctaacggtgatggccgccgggcaggaagtgttgattcaggatttgcccggcgaactgtttgaatcaacggttgcggagagtacttcgcaaatgcaaccggacagctgggcgacgcttcttgcgcagtgggcagacagagcgctgcgttccggtcatcaaaatctgctttccgaagcgcagccagagctggagcggacgttactgacgaccgcgttgcgacatacgcaggggcataaacaggaagcggcgcggctactcggctggggccgcaacaccctgacgcgtaagttaaaagagctggggatggagtga

SEQ ID NO: 20 - DNA, 1633 bp, synthetic construct
  source 1..1633: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatc

SEQ ID NO: 21 - DNA, 105 bp, Escherichia coli
  source 1..105: mol_type = genomic DNA
tgctagaggcatcaaataaaacgaaaggctcagtcgaaagactgggcctttcgttttatctgttgtttgtcggtgaacgctctcctgagtaggacaaatccgccg

SEQ ID NO: 22 - DNA, 155 bp, Escherichia coli
  source 1..155: mol_type = genomic DNA
atagcgcaatatttcatcgttggtgcaaaaatgtaacgcactgtgcactgtcatagtgcgttttcattttcaaacttcttaacttcctgctctctttctcgtttttcatttctggcacaccgcttgcaataccttcttcgtgtagcagaaccatt

SEQ ID NO: 23 - DNA, 522 bp, Escherichia coli
  source 1..522: mol_type = genomic DNA
agcttctatatcgcctgcgtagtgattacctgggcggtatatggtcggcattctaaaaaataaaccgttactcgtcatacttcgggttacatgtgctgcggctgcgttcattcaccccagtcacttactttagtaagctcctgggattcattcacttgccgccttcctgtaaaccgaattatatagagtaaaatatttgattatcctttgcgcggcatgatgtcgcgctttttttatgcgtcatttagttacaacatactaatgttatatggtttatttcgccggatttcattaagagccattaatatgttacccatggggaatactccttaatacccatctgcataaaaatcttaatagtttaaataactacaggtataaaacgtcttaatttacagtctgttatgtggtggctgttaattatcctaaaggggtatcttaggaatttactttatttttcatccccatcactcttgatcgttatcaattcccacgctgtttcagagcgttaccttgccctta

SEQ ID NO: 24 - DNA, 149 bp, Escherichia coli
  source 1..149: mol_type = genomic DNA
tttttaactggcctgtcaggcagtggtgcgtttttctaccgctattgaggtaggtcaatttgcgaaggcggattattttgtggcaaacagatgttctttttgatttcgcgcaaaaagattcagaattttactgttagtttcctcgcgca

SEQ ID NO: 25 - DNA, 970 bp, Escherichia coli
  source 1..970: mol_type = genomic DNA
tggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaattctggtacgcctggcagatattttgcctgccgggcgaacagtgtgatacattgctgtgtcgggtaagccattacgctatccgacacagtgttaaatcctcgcttttttccttccccgaactgaaataaattagcgacacagcttgtggctggtttatcatcaatataaatgtattttttcccgatttcccttttgaggttgatgtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtgacgaaaatcttcatcagagatgacaacggaggaaccgag

SEQ ID NO: 26 - DNA, 42 bp, unidentified
  source 1..42: mol_type = unassigned DNA
aaataaagccctgagtttaaccgctcggggctttttgcgttt

SEQ ID NO: 27 - DNA, 220 bp, Escherichia coli
  source 1..220: mol_type = genomic DNA
ttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggc

SEQ ID NO: 28 - DNA, 608 bp, Escherichia coli
  source 1..608: mol_type = genomic DNA
acgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggta

SEQ ID NO: 29 - DNA, 2172 bp, Escherichia coli
  source 1..2172: mol_type = genomic DNA
tttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaa

SEQ ID NO: 30 - DNA, 2415 bp, Escherichia coli
  source 1..2415: mol_type = genomic DNA
tttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaatccgcctttgtcatcttttattgccataagccagtcgatgctggcttattttctttgcagtcaaaatacgggcgttagattttacaacgattggtgattttttgttcgcatgattagccatgtctttttcacggaaatagtgttttatactggttggtgatttcttatcgctatatacctctggtttttagatccctccttgctttaaaacgttataagcgtttaaattgcgcttcaggtgct

SEQ ID NO: 31 - DNA, 303 bp, Escherichia coli
  source 1..303: mol_type = genomic DNA
taaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctccc

SEQ ID NO: 32 - DNA, 522 bp, Escherichia coli
  source 1..522: mol_type = genomic DNA
gactgttgtacactaccggggccttttggccccgtttttttatctggaggattaatgacaatcacggacctggtactgattcttttcatcgccgcactcctggccttcgcgatctacgatcagttcatcatgccccgccgtaacggccccaccctgctggcaattcctttgctccggcgtggtcgcatcgatagcgttatcttcgtcggattgattgtcattcttatctataacaacgtcacgaatcatggtgcgttaataacgacatggttattaagcgcactggctctgatgggtttttatatattctggatccgcgttccgaagatcatctttaaacaaaaaggttttttcttcgccaatgtctggattgaatatagccgaatcaaagcgatgaacttgtcggaagatggcgtgttggtgatgcaattagaacagcgtcggctgttaatccgcgttcgaaatatcgacgatctggaaaaaatttataagcttctcgtttcaactcaataagttatgaat

SEQ ID NO: 33 - DNA, 351 bp, Escherichia coli
  source 1..351: mol_type = genomic DNA
acctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtc

SEQ ID NO: 34 - DNA, 416 bp, Escherichia coli
  source 1..416: mol_type = genomic DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttt

SEQ ID NO: 35 - DNA, 488 bp, Escherichia coli
  source 1..488: mol_type = genomic DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcaggacattgtcatgaatatcactgctactgttcttcttgcgtttggtatgtcgatg

SEQ ID NO: 36 - DNA, 5197 bp, synthetic construct
  source 1..5197: mol_type = other DNA
ggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcgattctgataacaaactagcaacaccagaacagcccgtttgcgggcagcaaaacccgtaccctaggtctagggcggcggatttgtcctactcaggagagcgttcaccgacaaacaacagataaaacgaaaggcccagtctttcgactgagcctttcgttttatttgatgcctctagagcttgcatgcctgcaggtctggacatttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaagaccatacgcgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatatgtatatctccttcttaaatctagaggatcccgtcaatgccaataccccccgctccaggatcgcgtcctctcgagcgggatcccgtcaatgccaatacccctttccgcgccgactgcgggactggcccctggctaaagttgttatcttgcgacacgaaattccccctttttgctgtttgaaacccaaaaaatccgccgcattctattcatctgaagataaaaagcaatcgttttcgtggggaaatatattttttatatgaatgagtgcaaaaaggtgggggaaaaattatcggtgtaaaaacagtgggtaggcctgataagacgcgtcagcgtcgcatcaggcatctgagcatggtgtcggatgcgacgcgagcgccttatccgacctacggttcaggtccgtaggcatgataaaacgcgtcagcgtcgcatcaggcatcggcacacagtgccggatgcaccgcgcgacgcatccgacaataacctactcttccatcgcctggcggatctgatccaacgacgcaggatcatcaatggtcgtcagatctccaggatcgcgtcctctcgaggtgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctga

SEQ ID NO: 37 - DNA, 5141 bp, synthetic construct
  source 1..5141: mol_type = other DNA
ggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcgattctgataacaaactagcaacaccagaacagcccgtttgcgggcagcaaaacccgtaccctaggtctagggcggcggatttgtcctactcaggagagcgttcaccgacaaacaacagataaaacgaaaggcccagtctttcgactgagcctttcgttttatttgatgcctctagagcttgcatgcctgcaggtctggacatttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaagaccatacgcgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatatgtatatctccttcttaaatctagaggatccacgcgctttatcgtcgagtattcttcagaaccttggggatcacgccaccagaaaatgacctcaaacatttcgtcattcagacgctgccattccgggccatgtttcgactgccaccagctctcacttcctacttttaacgccgtcacaccataaccccatgtttactgtgcaatttttcattgattgcagaaatatattgataatattattgataactatttgcatttgcaatagcgtaatggcgcgccgtgggaagcgcggacattaattaaccaactgcactgcgtgtctttcaggatcaaaggttttcgcggtagcgggatgcgtcgtgttgatgacgaccatgcccgacagttgcaattcgtggcaaaaatgcaggaataaaacaatgaacaagaagattcattccctggccttgttggtcaatctggggatttatggggtagcgcaggcacaagagccgaccctcgaggtgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctga

SEQ ID NO: 38 - DNA, 5254 bp, synthetic construct
  source 1..5254: mol_type = other DNA
ggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcgattctgataacaaactagcaacaccagaacagcccgtttgcgggcagcaaaacccgtaccctaggtctagggcggcggatttgtcctactcaggagagcgttcaccgacaaacaacagataaaacgaaaggcccagtctttcgactgagcctttcgttttatttgatgcctctagagcttgcatgcctgcaggtctggacatttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaagaccatacgcgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatatgtatatctccttcttaaatctagaggatctctcgagtcgtcactgcctgtgcattccccattaacgccagtccgagcgccagtgctgaagctgtataatgtaacggtttcatcgtttatctctcttgttgtaccgaatgcgcgaattcacgcgttttatgctcgcggggtaacatgacatgctcgaattacagaaaaataacttttttgttacatttgtaagatagtaaggtgtcagaaagatgacaaggcggtgacggcgtgggtgagggaaaatgggagatggggcacggataagcgggaaaatatagaaggtctgaatcaaactctacagattgctcatcgtttcatgccggatgcggcgtaaacgccttatcaggcctacaagatcgtgcaaattcaacatattgccactcacccagtaggcctgataagcgcagcgcatcaggcaattttacatttgtcacctgtctcaaaggagtcttttgactccctatcaatcaacgtgttattacccgcctaaatacgcacttctcaccgcttcattcgccagcagcgcatcaccagtatcggaaagcactacatggccgttttccagcacgtaggatccatcgaggtgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctga

SEQ ID NO: 39 - DNA, 3268 bp, synthetic construct
  source 1..3268: mol_type = other DNA
ccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcgattctgataacaaactagcaacaccagaacagcccgtttgcgggcagcaaaacccgtaccctaggtctagggcggcggatttgtcctactcaggagagcgttcaccgacaaacaacagataaaacgaaaggcccagtctttcgactgagcctttcgttttatttgatgcctctagagcttgcatgcctgcaggtctggacatttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaagaccatacgcgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatatgtatatctccttcttaaatctagaggatc

SEQ ID NO: 40 - DNA, 231 bp, synthetic construct
  source 1..231: mol_type = other DNA
tcgaggtgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagat

SEQ ID NO: 41 - DNA, 480 bp, Escherichia coli
  source 1..480: mol_type = genomic DNA
aggacgcgatcctggagatctgacgaccattgatgatcctgcgtcgttggatcagatccgccaggcgatggaagagtaggttattgtcggatgcgtcgcgcggtgcatccggcactgtgtgccgatgcctgatgcgacgctgacgcgttttatcatgcctacggacctgaaccgtaggtcggataaggcgctcgcgtcgcatccgacaccatgctcagatgcctgatgcgacgctgacgcgtcttatcaggcctacccactgtttttacaccgataatttttcccccacctttttgcactcattcatataaaaaatatatttccccacgaaaacgattgctttttatcttcagatgaatagaatgcggcggattttttgggtttcaaacagcaaaaagggggaatttcgtgtcgcaagataacaactttagccaggggccagtcccgcagtcggcgcggaaaggggtattggcattgacg

SEQ ID NO: 42 - DNA, 466 bp, Escherichia coli
  source 1..466: mol_type = genomic DNA
ggtcggctcttgtgcctgcgctaccccataaatccccagattgaccaacaaggccagggaatgaatcttcttgttcattgttttattcctgcatttttgccacgaattgcaactgtcgggcatggtcgtcatcaacacgacgcatcccgctaccgcgaaaacctttgatcctgaaagacacgcagtgcagttggttaattaatgtccgcgcttcccacggcgcgccattacgctattgcaaatgcaaatagttatcaataatattatcaatatatttctgcaatcaatgaaaaattgcacagtaaacatggggttatggtgtgacggcgttaaaagtaggaagtgagagctggtggcagtcgaaacatggcccggaatggcagcgtctgaatgacgaaatgtttgaggtcattttctggtggcgtgatccccaaggttctgaagaatactcgacgataaagcgcgt

SEQ ID NO: 43 - DNA, 569 bp, Escherichia coli
  source 1..569: mol_type = genomic DNA
ctacgtgctggaaaacggccatgtagtgctttccgatactggtgatgcgctgctggcgaatgaagcggtgagaagtgcgtatttaggcgggtaataacacgttgattgatagggagtcaaaagactcctttgagacaggtgacaaatgtaaaattgcctgatgcgctgcgcttatcaggcctactgggtgagtggcaatatgttgaatttgcacgatcttgtaggcctgataaggcgtttacgccgcatccggcatgaaacgatgagcaatctgtagagtttgattcagaccttctatattttcccgcttatccgtgccccatctcccattttccctcacccacgccgtcaccgccttgtcatctttctgacaccttactatcttacaaatgtaacaaaaaagttatttttctgtaattcgagcatgtcatgttaccccgcgagcataaaacgcgtgaattcgcgcattcggtacaacaagagagataaacgatgaaaccgttacattatacagcttcagcactggcgctcggactggcgttaatggggaatgcacaggcagtgacgac

SEQ ID NO: 44 - DNA, 85 bp, Escherichia coli
  source 1..85: mol_type = genomic DNA
gcataaaaagggttatccaaaggtcattgcaccaacatggtgcttaatgtttccattgaagcactatattggtgcaacattcaca

SEQ ID NO: 45 - DNA, 85 bp, Escherichia coli
  source 1..85: mol_type = genomic DNA
gcatgataacgccttttaggggcaatttaaaagttggcacagatttcgctttatcttttttacggcgacacggccaaaataattg

SEQ ID NO: 46 - DNA, 313 bp, Escherichia coli
  source 1..313: mol_type = genomic DNA
aacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagt

SEQ ID NO: 47 - DNA, 1750 bp, synthetic construct
  source 1..1750: mol_type = other DNA
aacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagtatgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaagaattcattaaagaggagaaaggtacc

SEQ ID NO: 48 - DNA, 2467 bp, synthetic construct
  source 1..2467: mol_type = other DNA
aacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagtatgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 49 - DNA, 5223 bp, synthetic construct
  source 1..5223: mol_type = other DNA
aacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagtatgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgagtgttttagttgccgtggaaacttttcgcctgtctctggcaggcctgggatcggtggcaagcacatcacgccggatgcgacgcaaatgcgtcttatccggcctacacggtgatgatgtggtaggccggagcaggtgagtcgctctccaacgtgaagtttgtcagctatctgtagcccatctctgcatgggcttttttctccgtcaattctctgatgcttcgcgctttttatccgtaaaaagctataatgcactaaaatggtgcaacctgttcaggagactgctttatggcaacaggcacgcagcccgatgctgggcagatcctcaactcgctgattaacagtattttgttaatcgatgacaacctggcgatccattacgccaaccctgccgcgcaacaactgctcgcccaaagctcccgcaaattgtttggtacaccgttaccggaactgttgagctacttctcattaaatatcgagctgatgcaagaaagtctggaggcggggcaaggttttaccgataacgaagtgacgctggtcatcgacgggcgctcgcatatcctttctgtgacggcccagcgtatgccggacggcatgatcctgctggagatggctccgatggataaccagcgccgcttaagtcaggaacagctacagcacgcccagcaggttgctgcccgtgatttagtgcgcggcctggcacatgagattaaaaatccgcttggcggtttacgtggcgcggcgcagctgctcagcaaagcgttacctgacccatcactactcgaatataccaaagtgattatcgaacaggcggaccggctgcgaaatctggtcgaccgtctgttggggccgcagctgcccggtacgcgcgttaccgaaagtattcacaaagtggctgaacgcgtggtaacgctggtgtcgatggaactgccggacaacgtgcggttgattcgtgattacgatcccagcctaccggaactggcgcacgacccggatcaaattgaacaggtcttgctgaatattgtgcgcaatgcgctacaggcgctggggccggaaggcggtgaaatcattctgcgtacccgcaccgcgtttcaactgaccttacacggcgagcgctaccggctggcggcgcggattgatgtggaagataacgggccgggcattccgcctcatttgcaggatacgctgttttacccgatggtcagcggccgcgaaggtggcaccgggcttggcttatccatcgctcgtaatttgattgatcagcattcaggcaaaattgaatttaccagttggccagggcataccgagttctcggtttacctgcctatcaggaaataaaggtgacgtttatgcaacgagggatagtctgggtagtcgatgacgatagttccatccgttgggtgcttgaacgtgcgctcgctggggcaggtttaacctgtacgacgtttgagaacggcgcagaagtgctggaggcgctggcgagcaaaacgccggatgtgctgctttcagatatccgtatgccgggaatggacgggctggcgctgctcaagcagattaaacagcgccatccaatgcttccggtcatcattatgaccgcacattccgatctggatgctgccgtcagcgcctatcaacaaggggcgtttgattatctgcccaaaccgtttgatatcgacgaagcagtggcgctggttgagcgcgctatcagtcattaccaggaacagcagcagccgcgtaatgttcagcttaacggcccaacgaccgatatcatcggcgaagcgccagccatgcaggacgtgttccgtattatcggtcggctttcgcgttcttctattagcgtgctgattaacggcgaatccggcaccggtaaagaactggtcgctcatgccctgcatcgccacagtccgcgcgccaaagcgccgtttatcgcgctgaatatggcagctatcccaaaagatttgatcgaatcagaactgtttggccacgagaaaggcgcgtttactggcgcgaataccattcgtcaggggcgttttgaacaggccgatggcggtacattattcctcgacgaaattggtgatatgccgctggatgtgcagacgcgtttgctgcgcgtgctggcagacggtcagttttaccgcgttggcggctatgcgccggtgaaagtggatgtgcggattatcgctgccactcaccagaatctcgaacagcgagtgcaggaaggtaagttccgtgaggatctgttccaccgcctgaacgttatccgcgttcatctgccgccgctgcgcgaacgtcgggaagatattccccgtctggcgcgccattttttacaggttgccgcgcgcgaactgggcgtagaagcgaagttactgcatccggaaaccgaagctgctctgacgcgtctggcgtggccaggcaacgtgcgccagctggaaaacacctgccgctggctaacggtgatggccgccgggcaggaagtgttgattcaggatttgcccggcgaactgtttgaatcaacggttgcggagagtacttcgcaaatgcaaccggacagctgggcgacgcttcttgcgcagtgggcagacagagcgctgcgttccggtcatcaaaatctgctttccgaagcgcagccagagctggagcggacgttactgacgaccgcgttgcgacatacgcaggggcataaacaggaagcggcgcggctactcggctggggccgcaacaccctgacgcgtaagttaaaagagctggggatggagtga

SEQ ID NO: 50 - DNA, 5325 bp, synthetic construct
  source 1..5325: mol_type = other DNA
aacaggatcacaaacatcctccgcaaacaagtattgcagagtccctttgtgatcgctttcacggagcataaaaagggttatccaaaggtcattgcaccaacatggtgcttaatgtttccattgaagcactatattggtgcaacattcacatcgtggtgcagcccttttgcacgatggtgcgcatgataacgccttttaggggcaatttaaaagttggcacagatttcgctttatcttttttacggcgacacggccaaaataattgcagatttcgttaccacgacgaccatgaccaatccaggagagttaaagtatgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgagtgttttagttgccgtggaaacttttcgcctgtctctggcaggcctgggatcggtggcaagcacatcacgccggatgcgacgcaaatgcgtcttatccggcctacacggtgatgatgtggtaggccggagcaggtgagtcgctctccaacgtgaagtttgtcagctatctgtagcccatctctgcatgggcttttttctccgtcaattctctgatgcttcgcgctttttatccgtaaaaagctataatgcactaaaatggtgcaacctgttcaggagactgctttatggcaacaggcacgcagcccgatgctgggcagatcctcaactcgctgattaacagtattttgttaatcgatgacaacctggcgatccattacgccaaccctgccgcgcaacaactgctcgcccaaagctcccgcaaattgtttggtacaccgttaccggaactgttgagctacttctcattaaatatcgagctgatgcaagaaagtctggaggcggggcaaggttttaccgataacgaagtgacgctggtcatcgacgggcgctcgcatatcctttctgtgacggcccagcgtatgccggacggcatgatcctgctggagatggctccgatggataaccagcgccgcttaagtcaggaacagctacagcacgcccagcaggttgctgcccgtgatttagtgcgcggcctggcacatgagattaaaaatccgcttggcggtttacgtggcgcggcgcagctgctcagcaaagcgttacctgacccatcactactcgaatataccaaagtgattatcgaacaggcggaccggctgcgaaatctggtcgaccgtctgttggggccgcagctgcccggtacgcgcgttaccgaaagtattcacaaagtggctgaacgcgtggtaacgctggtgtcgatggaactgccggacaacgtgcggttgattcgtgattacgatcccagcctaccggaactggcgcacgacccggatcaaattgaacaggtcttgctgaatattgtgcgcaatgcgctacaggcgctggggccggaaggcggtgaaatcattctgcgtacccgcaccgcgtttcaactgaccttacacggcgagcgctaccggctggcggcgcggattgatgtggaagataacgggccgggcattccgcctcatttgcaggatacgctgttttacccgatggtcagcggccgcgaaggtggcaccgggcttggcttatccatcgctcgtaatttgattgatcagcattcaggcaaaattgaatttaccagttggccagggcataccgagttctcggtttacctgcctatcaggaaataaaggtgacgtttatgcaacgagggatagtctgggtagtcgatgacgatagttccatccgttgggtgcttgaacgtgcgctcgctggggcaggtttaacctgtacgacgtttgagaacggcgcagaagtgctggaggcgctggcgagcaaaacgccggatgtgctgctttcagatatccgtatgccgggaatggacgggctggcgctgctcaagcagattaaacagcgccatccaatgcttccggtcatcattatgaccgcacattccgatctggatgctgccgtcagcgcctatcaacaaggggcgtttgattatctgcccaaaccgtttgatatcgacgaagcagtggcgctggttgagcgcgctatcagtcattaccaggaacagcagcagccgcgtaatgttcagcttaacggcccaacgaccgatatcatcggcgaagcgccagccatgcaggacgtgttccgtattatcggtcggctttcgcgttcttctattagcgtgctgattaacggcgaatccggcaccggtaaagaactggtcgctcatgccctgcatcgccacagtccgcgcgccaaagcgccgtttatcgcgctgaatatggcagctatcccaaaagatttgatcgaatcagaactgtttggccacgagaaaggcgcgtttactggcgcgaataccattcgtcaggggcgttttgaacaggccgatggcggtacattattcctcgacgaaattggtgatatgccgctggatgtgcagacgcgtttgctgcgcgtgctggcagacggtcagttttaccgcgttggcggctatgcgccggtgaaagtggatgtgcggattatcgctgccactcaccagaatctcgaacagcgagtgcaggaaggtaagttccgtgaggatctgttccaccgcctgaacgttatccgcgttcatctgccgccgctgcgcgaacgtcgggaagatattccccgtctggcgcgccattttttacaggttgccgcgcgcgaactgggcgtagaagcgaagttactgcatccggaaaccgaagctgctctgacgcgtctggcgtggccaggcaacgtgcgccagctggaaaacacctgccgctggctaacggtgatggccgccgggcaggaagtgttgattcaggatttgcccggcgaactgtttgaatcaacggttgcggagagtacttcgcaaatgcaaccggacagctgggcgacgcttcttgcgcagtgggcagacagagcgctgcgttccggtcatcaaaatctgctttccgaagcgcagccagagctggagcggacgttactgacgaccgcgttgcgacatacgcaggggcataaacaggaagcggcgcggctactcggctggggccgcaacaccctgacgcgtaagttaaaagagctggggatggagtgacttggactcctgttgatagatccagtaatgacctcagaactccatctggatttgttcagaacgctcggttgccgccgggcgttttttattggtgagaatcca

SEQ ID NO: 51 - DNA, 1738 bp, synthetic construct
  source 1..1738: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccg

SEQ ID NO: 52 - DNA, 182 bp, synthetic construct
  source 1..182: mol_type = other DNA
atagcgcaatatttcatcgttggtgcaaaaatgtaacgcactgtgcactgtcatagtgcgttttcattttcaaacttcttaacttcctgctctctttctcgtttttcatttctggcacaccgcttgcaataccttcttcgtgtagcagaaccattgaattcattaaagaggagaaaggtacc

SEQ ID NO: 53 - DNA, 899 bp, synthetic construct
  source 1..899: mol_type = other DNA
atagcgcaatatttcatcgttggtgcaaaaatgtaacgcactgtgcactgtcatagtgcgttttcattttcaaacttcttaacttcctgctctctttctcgtttttcatttctggcacaccgcttgcaataccttcttcgtgtagcagaaccattgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 54 - DNA, 1001 bp, synthetic construct
  source 1..1001: mol_type = other DNA
atagcgcaatatttcatcgttggtgcaaaaatgtaacgcactgtgcactgtcatagtgcgttttcattttcaaacttcttaacttcctgctctctttctcgtttttcatttctggcacaccgcttgcaataccttcttcgtgtagcagaaccattgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 55 - DNA, 2739 bp, synthetic construct
  source 1..2739: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgatagcgcaatatttcatcgttggtgcaaaaatgtaacgcactgtgcactgtcatagtgcgttttcattttcaaacttcttaacttcctgctctctttctcgtttttcatttctggcacaccgcttgcaataccttcttcgtgtagcagaaccattgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 56 - DNA, 549 bp, synthetic construct
  source 1..549: mol_type = other DNA
agcttctatatcgcctgcgtagtgattacctgggcggtatatggtcggcattctaaaaaataaaccgttactcgtcatacttcgggttacatgtgctgcggctgcgttcattcaccccagtcacttactttagtaagctcctgggattcattcacttgccgccttcctgtaaaccgaattatatagagtaaaatatttgattatcctttgcgcggcatgatgtcgcgctttttttatgcgtcatttagttacaacatactaatgttatatggtttatttcgccggatttcattaagagccattaatatgttacccatggggaatactccttaatacccatctgcataaaaatcttaatagtttaaataactacaggtataaaacgtcttaatttacagtctgttatgtggtggctgttaattatcctaaaggggtatcttaggaatttactttatttttcatccccatcactcttgatcgttatcaattcccacgctgtttcagagcgttaccttgcccttagaattcattaaagaggagaaaggtacc

SEQ ID NO: 57 - DNA, 1266 bp, synthetic construct
  source 1..1266: mol_type = other DNA
agcttctatatcgcctgcgtagtgattacctgggcggtatatggtcggcattctaaaaaataaaccgttactcgtcatacttcgggttacatgtgctgcggctgcgttcattcaccccagtcacttactttagtaagctcctgggattcattcacttgccgccttcctgtaaaccgaattatatagagtaaaatatttgattatcctttgcgcggcatgatgtcgcgctttttttatgcgtcatttagttacaacatactaatgttatatggtttatttcgccggatttcattaagagccattaatatgttacccatggggaatactccttaatacccatctgcataaaaatcttaatagtttaaataactacaggtataaaacgtcttaatttacagtctgttatgtggtggctgttaattatcctaaaggggtatcttaggaatttactttatttttcatccccatcactcttgatcgttatcaattcccacgctgtttcagagcgttaccttgcccttagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 58 - DNA, 1368 bp, synthetic construct
  source 1..1368: mol_type = other DNA
agcttctatatcgcctgcgtagtgattacctgggcggtatatggtcggcattctaaaaaataaaccgttactcgtcatacttcgggttacatgtgctgcggctgcgttcattcaccccagtcacttactttagtaagctcctgggattcattcacttgccgccttcctgtaaaccgaattatatagagtaaaatatttgattatcctttgcgcggcatgatgtcgcgctttttttatgcgtcatttagttacaacatactaatgttatatggtttatttcgccggatttcattaagagccattaatatgttacccatggggaatactccttaatacccatctgcataaaaatcttaatagtttaaataactacaggtataaaacgtcttaatttacagtctgttatgtggtggctgttaattatcctaaaggggtatcttaggaatttactttatttttcatccccatcactcttgatcgttatcaattcccacgctgtttcagagcgttaccttgcccttagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 59 - DNA, 3106 bp, synthetic construct
  source 1..3106: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgagcttctatatcgcctgcgtagtgattacctgggcggtatatggtcggcattctaaaaaataaaccgttactcgtcatacttcgggttacatgtgctgcggctgcgttcattcaccccagtcacttactttagtaagctcctgggattcattcacttgccgccttcctgtaaaccgaattatatagagtaaaatatttgattatcctttgcgcggcatgatgtcgcgctttttttatgcgtcatttagttacaacatactaatgttatatggtttatttcgccggatttcattaagagccattaatatgttacccatggggaatactccttaatacccatctgcataaaaatcttaatagtttaaataactacaggtataaaacgtcttaatttacagtctgttatgtggtggctgttaattatcctaaaggggtatcttaggaatttactttatttttcatccccatcactcttgatcgttatcaattcccacgctgtttcagagcgttaccttgcccttagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 60 - DNA, 176 bp, synthetic construct
  source 1..176: mol_type = other DNA
tttttaactggcctgtcaggcagtggtgcgtttttctaccgctattgaggtaggtcaatttgcgaaggcggattattttgtggcaaacagatgttctttttgatttcgcgcaaaaagattcagaattttactgttagtttcctcgcgcagaattcattaaagaggagaaaggtacc

SEQ ID NO: 61 - DNA, 893 bp, synthetic construct
  source 1..893: mol_type = other DNA
tttttaactggcctgtcaggcagtggtgcgtttttctaccgctattgaggtaggtcaatttgcgaaggcggattattttgtggcaaacagatgttctttttgatttcgcgcaaaaagattcagaattttactgttagtttcctcgcgcagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 62 - DNA, 995 bp, synthetic construct
  source 1..995: mol_type = other DNA
tttttaactggcctgtcaggcagtggtgcgtttttctaccgctattgaggtaggtcaatttgcgaaggcggattattttgtggcaaacagatgttctttttgatttcgcgcaaaaagattcagaattttactgttagtttcctcgcgcagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 63 - DNA, 2733 bp, synthetic construct
  source 1..2733: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgtttttaactggcctgtcaggcagtggtgcgtttttctaccgctattgaggtaggtcaatttgcgaaggcggattattttgtggcaaacagatgttctttttgatttcgcgcaaaaagattcagaattttactgttagtttcctcgcgcagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 64 - DNA, 1012 bp, synthetic construct
  source 1..1012: mol_type = other DNA
tggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaattctggtacgcctggcagatattttgcctgccgggcgaacagtgtgatacattgctgtgtcgggtaagccattacgctatccgacacagtgttaaatcctcgcttttttccttccccgaactgaaataaattagcgacacagcttgtggctggtttatcatcaatataaatgtattttttcccgatttcccttttgaggttgatgtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtgacgaaaatcttcatcagagatgacaacggaggaaccgagaaataaagccctgagtttaaccgctcggggctttttgcgttt

SEQ ID NO: 65 - DNA, 247 bp, synthetic construct
  source 1..247: mol_type = other DNA
ttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgaattcattaaagaggagaaaggtacc

SEQ ID NO: 66 - DNA, 964 bp, synthetic construct
  source 1..964: mol_type = other DNA
ttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 67 - DNA, 1066 bp, synthetic construct
  source 1..1066: mol_type = other DNA
ttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 68 - DNA, 2804 bp, synthetic construct
  source 1..2804: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 69 - DNA, 3816 bp, synthetic construct
  source 1..3816: mol_type = other DNA
tggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaattctggtacgcctggcagatattttgcctgccgggcgaacagtgtgatacattgctgtgtcgggtaagccattacgctatccgacacagtgttaaatcctcgcttttttccttccccgaactgaaataaattagcgacacagcttgtggctggtttatcatcaatataaatgtattttttcccgatttcccttttgaggttgatgtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtgacgaaaatcttcatcagagatgacaacggaggaaccgagaaataaagccctgagtttaaccgctcggggctttttgcgttttcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgttgggcgacagatcacgcaaaagtagaattgtgcaaataaacggcagggtaatttttgaaggtcagcaacaaaagttgattaattctttgaggaacatgcagttatgcatgctgttgaaaaagaggaagatactgactaactctaaagtggtattttacatgcacttacaattgattaaagacaacattcacagtgtggttatttgttacacataggggcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 70 - DNA, 635 bp, synthetic construct
  source 1..635: mol_type = other DNA
acgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtagaattcattaaagaggagaaaggtacc

SEQ ID NO: 71 - DNA, 1352 bp, synthetic construct
  source 1..1352: mol_type = other DNA
acgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 72 - DNA, 1454 bp, synthetic construct
  source 1..1454: mol_type = other DNA
acgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 73 - DNA, 3192 bp, synthetic construct
  source 1..3192: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgacgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 74 - DNA, 4204 bp, synthetic construct
  source 1..4204: mol_type = other DNA
tggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaattctggtacgcctggcagatattttgcctgccgggcgaacagtgtgatacattgctgtgtcgggtaagccattacgctatccgacacagtgttaaatcctcgcttttttccttccccgaactgaaataaattagcgacacagcttgtggctggtttatcatcaatataaatgtattttttcccgatttcccttttgaggttgatgtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtgacgaaaatcttcatcagagatgacaacggaggaaccgagaaataaagccctgagtttaaccgctcggggctttttgcgttttcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgacgcattctgaccgtactggctggtctggctgcgattctgatgtacgtctttaacaccggaacaccagcacatatggcgtggtcaattctcgctctgggcttcttctccagcgcgatctataccaccatcatcaccctgggttcacagcagaccaaagtaccgtcgccaaaactggttaactttgtcctgacctgcggaaccatcggtactatgttgacctttgtggttaccggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 75 - DNA, 85 bp, Escherichia coli
  source 1..85: mol_type = genomic DNA
tgaagatatgtgcgacgagcttttcataaatctgtcataaatctgacgcataatgacgtcgcattaatgatcgcaacctatttat

SEQ ID NO: 76 - DNA, 2199 bp, synthetic construct
  source 1..2199: mol_type = other DNA
tttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaagaattcattaaagaggagaaaggtacc

SEQ ID NO: 77 - DNA, 2916 bp, synthetic construct
  source 1..2916: mol_type = other DNA
tttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 78 - DNA, 3018 bp, synthetic construct
  source 1..3018: mol_type = other DNA
tttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaagaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 79 - DNA, 330 bp, synthetic construct
  source 1..330: mol_type = other DNA
taaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccgaattcattaaagaggagaaaggtacc

SEQ ID NO: 80 - DNA, 1047 bp, synthetic construct
  source 1..1047: mol_type = other DNA
taaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 81 - DNA, 1149 bp, synthetic construct
  source 1..1149: mol_type = other DNA
taaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 82 - DNA, 3564 bp, synthetic construct
  source 1..3564: mol_type = other DNA
tttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaatccgcctttgtcatcttttattgccataagccagtcgatgctggcttattttctttgcagtcaaaatacgggcgttagattttacaacgattggtgattttttgttcgcatgattagccatgtctttttcacggaaatagtgttttatactggttggtgatttcttatcgctatatacctctggtttttagatccctccttgctttaaaacgttataagcgtttaaattgcgcttcaggtgcttaaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 83 - DNA, 5302 bp, synthetic construct
  source 1..5302: mol_type = other DNA
tcgtgaatattttgccggtaatcttagcattattgatacgcttcccactgctacgggggggacgccatttcagcgcgaagtctggaaaacactacgcactatcccctgcgggcaggtaatgcattacggccaactggctgagcaattgggccgtcctggcgcggcgcgtgccgttggtgcggcaaacggatcgaatcccatcagcatcgtcgtaccttgccatcgggttattggccgaaacggcaccatgaccggatatgcaggcggagttcagcgaaaagagtggttattgcgccatgaaggttatcttttgctgtaaacattaaacaatttgtgccagcttgttcacacttttatgtaaagttacccttaacaacttaagggttttcaaatagatagacatatatttacatctaatatcggaattctctgctgttaaggtttgcttagacttacttgctccctaaaaagatgttaaaattgacaaatatcaattacggcttgagcagacctatgatcccggaaaagcgaattatacggcgcattcagtctggcggttgtgctatccattgccaggattgcagcatcagccagcattgcatcccgttcacactcaacgaacatgagcttgatcagcttgataatatcattgagcggaagaagcctattcagaaaggccagacgctgtttaaggctggtgatgaacttaaatcgctttatgccatccgctccggtacgattaaaagttataccatcactgagcaaggcgacgagcaaatcactggtttccatttagcaggcgacctggtgggatttgacgccatcggcagcggccatcacccgagcttcgcgcaggcgctggaaacctcgatggtatgtgaaatcccgttcgaaacgctggacgatttgtccggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggcgatatcggtaactatctgggcctgacggtagaaaccatcagccgtctgctgggtcgcttccagaaaagcggcatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgtatggctatgtatcagaacatgctcgttgttatcgatcctaaccaggacgaccaaccagcattgcggcgagctgtttatttacatcaacggattggtggcaaaattaaagcctttttgccgatctatgacttctcatacgaaatgaccaccctgctctccccggacgaacgtaccgctatgcgtcagggcgtcatcagccagcgtacagcctggatctgctagaggcatcaaataaaacgaaaggctcagtcgaaagactgggcctttcgttttatctgttgtttgtcggtgaacgctctcctgagtaggacaaatccgccgtttttctgcgccacggaaatcaataacctgaagatatgtgcgacgagcttttcataaatctgtcataaatctgacgcataatgacgtcgcattaatgatcgcaacctatttattacaacagggcaaatcatggcgagacgtattctggtcgtagaagatgaagctccaattcgcgaaatggtctgcttcgtgctcgaacaaaatggctttcagccggtcgaagcggaagattatgacagtgctgtgaatcaactgaatgaaccctggccggatttaattctcctcgactggatgttacctggcggctccggtatccagttcatcaaacacctcaagcgcgagtcgatgacccgggatattccagtggtgatgttgaccgccagaggggaagaagaagatcgcgtgcgcggccttgaaaccggcgcggatgactatatcaccaagccgttttcgccgaaggagctggtggcgcgaatcaaagcggtaatgcgccgtatttcgccaatggcggtggaagaggtgattgagatgcagggattaagtctcgacccgacatctcaccgagtgatggcgggcgaagagccgctggagatggggccgacagaatttaaactgctgcacttttttatgacgcatcctgagcgcgtgtacagccgcgagcagctgttaaaccacgtctggggaactaacgtgtatgtggaagaccgcacggtcgatgtccacattcgtcgcctgcgtaaagcactggagcccggcgggcatgaccgcatggtgcagaccgtgcgcggtacaggatatcgtttttcaacccgcttttaacgccttgctcatcggacgcagagcagggcttatgatttcttaactggagtatcttacgtgctggaacggctgtcgtggaaaaggctggtgctggagctgctactttgctgcctcccggctttcatcctgggtgcattttttggttacctgccctggtttttgctggcatcggtaacaggactgcttatctggcatttctggaatttattgcgcctttcatggtggctgtgggtggatcgcagtatgaccccgccaccggggcgtggtagctgggaaccgctactatacggcttacaccagatgcagctgcgaaataaaaaacgccgccgtgaactgggcaatctgattaaacgctttcgtagcggcgcggagtcgctgcccgacgcggtggtgctgaccacggaagagggcggtattttctggtgtaacggtctggcgcaacaaattcttggtttgcgctggccggaagataacgggcagaacatccttaacctactgcgttacccggagtttacgcaatatctgaaaacgcgtgatttttctcgcccgctcaatctggtgctcaacaccgggcggcatctggaaattcgcgtcatgccttatacccacaaacagttgctgatggtggcgcgtgatgtcacgcaaatgcatcaactggaaggggcgcggcgtaacttttttgccaacgtgagccatgagttacgtacgccattgaccgtgttacagggttacctggagatgatgaatgagcagccgctggaaggcgcggtacgcgaaaaagcgttgcacaccatgcgcgagcagacccagcggatggaaggactggtgaagcaattgctgacgctgtcgaaaatagaagccgcaccgacgcatttgctcaatgaaaaggttgatgtgccgatgatgctgcgcgttgttgagcgcgaggctcagactctgagtcagaaaaaacagacatttacctttgagatagataacggcctcaaggtgtctggcaacgaagatcagctacgcagtgcgatttcgaacctggtctataacgccgtgaatcatacgccggaaggcacgcatatcaccgtacgctggcagcgagtgccgcacggtgccgaatttagcgttgaagataacggaccgggcattgcaccggagcatattccgcgcctgaccgagcgtttttatcgcgttgataaagcgcgttcccggcaaaccggcggtagcggattagggttagcgatcgtgaaacatgctgtgaatcatcacgaaagtcgcctgaatattgagagtacagtaggaaaaggaacacgtttcagttttgttatcccggaacgtttaattgccaaaaacagcgattaatccgcctttgtcatcttttattgccataagccagtcgatgctggcttattttctttgcagtcaaaatacgggcgttagattttacaacgattggtgattttttgttcgcatgattagccatgtctttttcacggaaatagtgttttatactggttggtgatttcttatcgctatatacctctggtttttagatccctccttgctttaaaacgttataagcgtttaaattgcgcttcaggtgcttaaatggatgccctgcgtaagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 84 - DNA, 847 bp, synthetic construct
  source 1..847: mol_type = other DNA
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaatgtccagacctgcaggcatgcaagctctagaggcatcaaataaaacgaaaggctcagtcgaaagactgggcctttcgttttatctgttgtttgtcggtgaacgctctcctgagtaggacaaatccgccg

SEQ ID NO: 85 - DNA, 1411 bp, synthetic construct
  source 1..1411: mol_type = other DNA
aggacgcgatcctggagatctgacgaccattgatgatcctgcgtcgttggatcagatccgccaggcgatggaagagtaggttattgtcggatgcgtcgcgcggtgcatccggcactgtgtgccgatgcctgatgcgacgctgacgcgttttatcatgcctacggacctgaaccgtaggtcggataaggcgctcgcgtcgcatccgacaccatgctcagatgcctgatgcgacgctgacgcgtcttatcaggcctacccactgtttttacaccgataatttttcccccacctttttgcactcattcatataaaaaatatatttccccacgaaaacgattgctttttatcttcagatgaatagaatgcggcggattttttgggtttcaaacagcaaaaagggggaatttcgtgtcgcaagataacaactttagccaggggccagtcccgcagtcggcgcggaaaggggtattggcattgacgggatcccgctcgagaggacgcgatcctggagcggggggtattggcattgacgggatcctctagatttaagaaggagatatacatatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaatgtccagacctgcaggcatgcaagctctagaggcatcaaataaaacgaaaggctcagtcgaaagactgggcctttcgttttatctgttgtttgtcggtgaacgctctcctgagtaggacaaatccgccg

SEQ ID NO: 86 - DNA, 1345 bp, synthetic construct
  source 1..1345: mol_type = other DNA
ggtcggctcttgtgcctgcgctaccccataaatccccagattgaccaacaaggccagggaatgaatcttcttgttcattgttttattcctgcatttttgccacgaattgcaactgtcgggcatggtcgtcatcaacacgacgcatcccgctaccgcgaaaacctttgatcctgaaagacacgcagtgcagttggttaattaatgtccgcgcttcccacggcgcgccattacgctattgcaaatgcaaatagttatcaataatattatcaatatatttctgcaatcaatgaaaaattgcacagtaaacatggggttatggtgtgacggcgttaaaagtaggaagtgagagctggtggcagtcgaaacatggcccggaatggcagcgtctgaatgacgaaatgtttgaggtcattttctggtggcgtgatccccaaggttctgaagaatactcgacgataaagcgcgtggatcctctagatttaagaaggagatatacatatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaatgtccagacctgcaggcatgcaagctctagaggcatcaaataaaacgaaaggctcagtcgaaagactgggcctttcgttttatctgttgtttgtcggtgaacgctctcctgagtaggacaaatccgccg

SEQ ID NO: 87 - DNA, 1453 bp, synthetic construct
  source 1..1453: mol_type = other DNA
ctacgtgctggaaaacggccatgtagtgctttccgatactggtgatgcgctgctggcgaatgaagcggtgagaagtgcgtatttaggcgggtaataacacgttgattgatagggagtcaaaagactcctttgagacaggtgacaaatgtaaaattgcctgatgcgctgcgcttatcaggcctactgggtgagtggcaatatgttgaatttgcacgatcttgtaggcctgataaggcgtttacgccgcatccggcatgaaacgatgagcaatctgtagagtttgattcagaccttctatattttcccgcttatccgtgccccatctcccattttccctcacccacgccgtcaccgccttgtcatctttctgacaccttactatcttacaaatgtaacaaaaaagttatttttctgtaattcgagcatgtcatgttaccccgcgagcataaaacgcgtgaattcgcgcattcggtacaacaagagagataaacgatgaaaccgttacattatacagcttcagcactggcgctcggactggcgttaatggggaatgcacaggcagtgacgactcgagagatcctctagatttaagaaggagatatacatatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaatgtccagacctgcaggcatgcaagctctagaggcatcaaataaaacgaaaggctcagtcgaaagactgggcctttcgttttatctgttgtttgtcggtgaacgctctcctgagtaggacaaatccgccg

SEQ ID NO: 88 - DNA, 900 bp, synthetic construct
  source 1..900: mol_type = other DNA
gactgttgtacactaccggggccttttggccccgtttttttatctggaggattaatgacaatcacggacctggtactgattcttttcatcgccgcactcctggccttcgcgatctacgatcagttcatcatgccccgccgtaacggccccaccctgctggcaattcctttgctccggcgtggtcgcatcgatagcgttatcttcgtcggattgattgtcattcttatctataacaacgtcacgaatcatggtgcgttaataacgacatggttattaagcgcactggctctgatgggtttttatatattctggatccgcgttccgaagatcatctttaaacaaaaaggttttttcttcgccaatgtctggattgaatatagccgaatcaaagcgatgaacttgtcggaagatggcgtgttggtgatgcaattagaacagcgtcggctgttaatccgcgttcgaaatatcgacgatctggaaaaaatttataagcttctcgtttcaactcaataagttatgaatacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcgaattcattaaagaggagaaaggtacc

SEQ ID NO: 89 - DNA, 1617 bp, synthetic construct
  source 1..1617: mol_type = other DNA
gactgttgtacactaccggggccttttggccccgtttttttatctggaggattaatgacaatcacggacctggtactgattcttttcatcgccgcactcctggccttcgcgatctacgatcagttcatcatgccccgccgtaacggccccaccctgctggcaattcctttgctccggcgtggtcgcatcgatagcgttatcttcgtcggattgattgtcattcttatctataacaacgtcacgaatcatggtgcgttaataacgacatggttattaagcgcactggctctgatgggtttttatatattctggatccgcgttccgaagatcatctttaaacaaaaaggttttttcttcgccaatgtctggattgaatatagccgaatcaaagcgatgaacttgtcggaagatggcgtgttggtgatgcaattagaacagcgtcggctgttaatccgcgttcgaaatatcgacgatctggaaaaaatttataagcttctcgtttcaactcaataagttatgaatacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 90 - DNA, 1719 bp, synthetic construct
  source 1..1719: mol_type = other DNA
gactgttgtacactaccggggccttttggccccgtttttttatctggaggattaatgacaatcacggacctggtactgattcttttcatcgccgcactcctggccttcgcgatctacgatcagttcatcatgccccgccgtaacggccccaccctgctggcaattcctttgctccggcgtggtcgcatcgatagcgttatcttcgtcggattgattgtcattcttatctataacaacgtcacgaatcatggtgcgttaataacgacatggttattaagcgcactggctctgatgggtttttatatattctggatccgcgttccgaagatcatctttaaacaaaaaggttttttcttcgccaatgtctggattgaatatagccgaatcaaagcgatgaacttgtcggaagatggcgtgttggtgatgcaattagaacagcgtcggctgttaatccgcgttcgaaatatcgacgatctggaaaaaatttataagcttctcgtttcaactcaataagttatgaatacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 91 - DNA, 443 bp, synthetic construct
  source 1..443: mol_type = other DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgtttgaattcattaaagaggagaaaggtacc

SEQ ID NO: 92 - DNA, 1160 bp, synthetic construct
  source 1..1160: mol_type = other DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgtttgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 93 - DNA, 1262 bp, synthetic construct
  source 1..1262: mol_type = other DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgtttgaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 94 - DNA, 1205 bp, synthetic construct
  source 1..1205: mol_type = other DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcaggacattgtcatgaatatcactgctactgttcttcttgcgtttggtatgtcgatgatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 95 - DNA, 1307 bp, synthetic construct
  source 1..1307: mol_type = other DNA
gtttcaactcaataagttatgaatttagccaaagctatgtttagtgtatttttaataatcagacatagcttaggctatattacctcttcccttatttgttatttattttaacgtttcattgatatataaatccaaatgaaaatcgttatcaataaagcaatgaaataatatattccaacagttgttttatattctcaaaatatgttaaggttgcgccctcatttggggagtagccgatttccagattccggaaatgtacgtgtcaacatactcgttgcaaaacgtggcacgtacggactgaatactttcagtcaggcgagaccatatgcacatcaatcgctatgcctgcatgaacgcgaaaaccatcacggcttgttgttcatgtatgcatgtttgctgggggcgatgatgtgttttatggataccccggtcaggacattgtcatgaatatcactgctactgttcttcttgcgtttggtatgtcgatgatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

SEQ ID NO: 96 - DNA, 3634 bp, synthetic construct
  source 1..3634: mol_type = other DNA
gaattcaaaagatcttaagtaagtaagagtatacgtatatcggctaataacgtattaaggcgcttcggcgcctttttttatgggggtattttcatcccaatccacacgtccaacgcacagcaaacaccacgtcgaccctatcagctgcgtgctttctatgagtcgttgctgcataaccggcagttttacaatcgcgttaataaattatttgatatatgaatccagcaccttcagaacgacatccagatcttcttcacgttttagctcatccccctggtgaacgatgtgttccgtcagatgacctttaatcacttcccgcatcagaccgtttaccgcgccacggatagcagcaatctgttgtaaaactgcagcggcttcgtgcggctcgtcgagcattttcttgagcgccacgacctggccctgaatcttactggcacgcgctttcagtttctgtttatcacggattgtatgagacatggcaacacctggttaacaagaatatgaaaaatcatagcactattaatctactggggggtagtatcaggtactgggggggagtagaatcagattgccgaattaatactaagaattattatcatgaccgaatttacaactcttcttcagcaaggaaacgcctggttcttcatccccagcgccatcttacttggtgcgcttcatggcctggaaccagggcactcaaaaacgatgatggcggcgtttatcatcgccatcaaaggcaccattaaacaagcggtgatgctcggactggcagcaactatttcgcataccgcagtggtctggttaattgcctttggcgggatggtgatcagcaagcgctttactgctcaatcagcagaaccgtgggaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatccacctttcgacctgcagcctgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccggatgaggggatcaattctctagagctcgctgatcagaagttcctgatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 97 - DNA, 85 bp, synthetic construct
  source 1..85: mol_type = other DNA
tttctgtttatcacggattgtatgagacatggcaacacctggttaacaagaatatgaaaaatcatagcactattaatctactggg

SEQ ID NO: 98 - DNA, 687 bp, synthetic construct
  source 1..687: mol_type = other DNA
cggcagttttacaatcgcgttaataaattatttgatatatgaatccagcaccttcagaacgacatccagatcttcttcacgttttagctcatccccctggtgaacgatgtgttccgtcagatgacctttaatcacttcccgcatcagaccgtttaccgcgccacggatagcagcaatctgttgtaaaactgcagcggcttcgtgcggctcgtcgagcattttcttgagcgccacgacctggccctgaatcttactggcacgcgctttcagtttctgtttatcacggattgtatgagacatggcaacacctggttaacaagaatatgaaaaatcatagcactattaatctactggggggtagtatcaggtactgggggggagtagaatcagattgccgaattaatactaagaattattatcatgaccgaatttacaactcttcttcagcaaggaaacgcctggttcttcatccccagcgccatcttacttggtgcgcttcatggcctggaaccagggcactcaaaaacgatgatggcggcgtttatcatcgccatcaaaggcaccattaaacaagcggtgatgctcggactggcagcaactatttcgcataccgcagtggtctggttaattgcctttggcgggatggtgatcagcaagcgctttactgctcaatcagcagaaccgtgg

SEQ ID NO: 99 - DNA, 714 bp, synthetic construct
  source 1..714: mol_type = other DNA
cggcagttttacaatcgcgttaataaattatttgatatatgaatccagcaccttcagaacgacatccagatcttcttcacgttttagctcatccccctggtgaacgatgtgttccgtcagatgacctttaatcacttcccgcatcagaccgtttaccgcgccacggatagcagcaatctgttgtaaaactgcagcggcttcgtgcggctcgtcgagcattttcttgagcgccacgacctggccctgaatcttactggcacgcgctttcagtttctgtttatcacggattgtatgagacatggcaacacctggttaacaagaatatgaaaaatcatagcactattaatctactggggggtagtatcaggtactgggggggagtagaatcagattgccgaattaatactaagaattattatcatgaccgaatttacaactcttcttcagcaaggaaacgcctggttcttcatccccagcgccatcttacttggtgcgcttcatggcctggaaccagggcactcaaaaacgatgatggcggcgtttatcatcgccatcaaaggcaccattaaacaagcggtgatgctcggactggcagcaactatttcgcataccgcagtggtctggttaattgcctttggcgggatggtgatcagcaagcgctttactgctcaatcagcagaaccgtgggaattcattaaagaggagaaaggtacc

SEQ ID NO: 100 - DNA, 1431 bp, synthetic construct
  source 1..1431: mol_type = other DNA
cggcagttttacaatcgcgttaataaattatttgatatatgaatccagcaccttcagaacgacatccagatcttcttcacgttttagctcatccccctggtgaacgatgtgttccgtcagatgacctttaatcacttcccgcatcagaccgtttaccgcgccacggatagcagcaatctgttgtaaaactgcagcggcttcgtgcggctcgtcgagcattttcttgagcgccacgacctggccctgaatcttactggcacgcgctttcagtttctgtttatcacggattgtatgagacatggcaacacctggttaacaagaatatgaaaaatcatagcactattaatctactggggggtagtatcaggtactgggggggagtagaatcagattgccgaattaatactaagaattattatcatgaccgaatttacaactcttcttcagcaaggaaacgcctggttcttcatccccagcgccatcttacttggtgcgcttcatggcctggaaccagggcactcaaaaacgatgatggcggcgtttatcatcgccatcaaaggcaccattaaacaagcggtgatgctcggactggcagcaactatttcgcataccgcagtggtctggttaattgcctttggcgggatggtgatcagcaagcgctttactgctcaatcagcagaaccgtgggaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 101 - DNA, 1533 bp, synthetic construct
  source 1..1533: mol_type = other DNA
cggcagttttacaatcgcgttaataaattatttgatatatgaatccagcaccttcagaacgacatccagatcttcttcacgttttagctcatccccctggtgaacgatgtgttccgtcagatgacctttaatcacttcccgcatcagaccgtttaccgcgccacggatagcagcaatctgttgtaaaactgcagcggcttcgtgcggctcgtcgagcattttcttgagcgccacgacctggccctgaatcttactggcacgcgctttcagtttctgtttatcacggattgtatgagacatggcaacacctggttaacaagaatatgaaaaatcatagcactattaatctactggggggtagtatcaggtactgggggggagtagaatcagattgccgaattaatactaagaattattatcatgaccgaatttacaactcttcttcagcaaggaaacgcctggttcttcatccccagcgccatcttacttggtgcgcttcatggcctggaaccagggcactcaaaaacgatgatggcggcgtttatcatcgccatcaaaggcaccattaaacaagcggtgatgctcggactggcagcaactatttcgcataccgcagtggtctggttaattgcctttggcgggatggtgatcagcaagcgctttactgctcaatcagcagaaccgtgggaattcattaaagaggagaaaggtaccatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgacttggactcctgttgatagatccagtaatgacctcagaactccatctggatttgttcagaacgctcggttgccgccgggcgttttttattggtgagaatcca

(19) United States
(12) Patent Application Publication
(10) Pub. No.: US 2026/0265805 A1
(43) Pub. Date: Sep. 10, 2026

(54) ENGINEERED BIOSENSOR STRAINS OF E. COLI FOR CONTINUOUS AEROBIC DETECTION OF ANALYTES

(71) Applicant: QUANTITATIVE BIOSCEINCES, INC., San Diego, CA (US)

(72) Inventors: Scott Warren Cookson, Encinitas, CA (US); Natalie Anne COOKSON, Encinitas, CA (US); Nicholas Sigmund CSICSERY, SanDiego, CA (US); Michael Stephen FERRY, SanDiego, CA (US); William H. MATHER, Encinitas, CA (US); April Heather SAVAGE, San Diego, CA (US); Yi ZHOU, LaJolla, CA (US)

(21) Appl. No.: 19/166,352

(22) PCT Filed: Apr. 22, 2024
(86) PCT No.: PCT/US2024/025712

§ 371 Date: Sep. 17, 2025

Related U.S. Application Data
(60) Provisional application No. 63/461,518, filed on Apr. 24, 2023.

(51) Int. Cl. C12Q 1/02; C12N 15/70

(52) U.S. Cl. CPC C12Q 1/02; C12N 15/70

(57) ABSTRACT

Disclosed is a panel of recombinant Escherichia coli strains for monitoring a plurality of analytes of interest in an aqueous sample, under aerobic physiological conditions, and a method for monitoring a plurality of analytes of interest employing the recombinant E. coli strains. The analytes of interest include ammonium, nitrate, nitrite, phosphate, manganese dication, an iron cation, and nickel dication, or a combination of any one of these analytes in real time, optionally, in a continuous series of aqueous samples, for example of environmental waters, agricultural aqueous run-off, or industrial aqueous inputs and/or outputs, aqueous biological samples, e.g., urine, serum, or cerebrospinal fluid. The disclosed panel of recombinant E. coli strains and the method can also be used to monitor these analytes in food samples that have been macerated and liquified.

### STATEMENT OF GOVERNMENT LICENSE RIGHTS

This invention was made with government support under SBIR STTR Award Number DE-SC0018575, awarded by the United States Department of Energy. The government has certain rights in the invention.

### SEQUENCE LISTING

The instant application contains a Sequence Listing which has been submitted electronically in XML file format and is hereby incorporated by reference in its entirety. Said XML copy, created on Apr. 11, 2024, is named QBI03PCT_SL.xml and is 280,658 bytes in size.

### BACKGROUND OF THE INVENTION

### 1. Field of the Invention

This invention relates to recombinant Escherichia coli strains useful as biosensors for monitoring of analytes in aqueous samples.

### 2. Discussion of the Related Art

Access to clean, reliable water supplies is critical to the quality of life and the economy, yet a vast array of contaminants including heavy metals, nutrients, and emerging contaminants of concern threaten the drinking water of millions of people across the United States and other countries, which can cause critical health problems to those who are, often unknowingly, affected. (See, e.g., Mueller et al., “The widespread and unjust drinking water and clean water crisis in the United States,” Nature Commun. (2021), 12:3544, doi.org/10.1038/s41467-021-23898-z; Allaire et al., “National trends in drinking water quality violations,” PNAS 115(9):2078-2083 (2018), pnas.org/doi/epdf/10.1073/pnas.1719805115).

Human activities have also accelerated the rate and extent of eutrophication of many freshwater and coastal marine ecosystems throughout the world by both point-source discharges and non-point loadings of limiting nutrients, such as nitrogen and phosphorus, into aquatic ecosystems, with dramatic adverse consequences for drinking water sources, fisheries, and recreational water bodies. (See, e.g., Chislock, M. F., Doster, E., Zitomer, R. A. & Wilson, A. E. (2013) “Eutrophication: Causes, Consequences, and Controls in Aquatic Ecosystems,” Nature Education Knowledge 4(4):10, www.nature.com/scitable/knowledge/library/eutrophication-causes-consequences-and-controls-in-aquatic-102364466; Dodds et al., “Eutrophication of U.S. Freshwaters: Analysis of Potential Economic Damages,” Environ. Sci. & Technol. 43(1): (2009) doi: 10.1021/es801217q).

The contaminants at various sites range from common water toxins, such as arsenic and cadmium, to excess nutrients (nitrogenous and phosphorus) and even radionuclides like uranium. Measuring contamination in the environment is critical to human health, but current testing is mostly limited to sporadic sample collection for laboratory analysis. Not only is such assessment costly, but it is inefficient, making it difficult to monitor water with high spatial or temporal resolution. As a result, current methods do not capture the full complexity of how contaminants behave in the environment. (See, e.g., Hasty et al., “Microbial Microfluidic Biosensor,” U.S. Pat. No. 11,209,412B2; Cardemil, C. et al., “Bioluminescent Escherichia coli strains for the quantitative detection of phosphate and ammonia in coastal and suburban watersheds,” DNA Cell Biol, vol. 29, no. 9, pp. 519-31 (2010), doi: 10.1089/dna.2009.0984; DeAngelis et al., “Two novel bacterial biosensors for detection of nitrate availability in the rhizosphere,” Appl. Environ. Microbiol. (2005), 71(12):8537-47, doi: 10.1128/AEM.71.12.8537-8547.2005; Diawara et al., “Arsenic, cadmium, lead, and mercury in surface soils, Pueblo, Colorado: implications for population health risk,” Environ. Geochem. Health (2006), 28(4):297-315, doi: 10.1007/s10653-005-9000-6. Epub 2006 Jun. 4; National Research Council of the National Academies, “Alternatives for Managing the Nation's Complex Contaminated Groundwater Sites” (2013); Bricker et al., “Effects of nutrient enrichment in the nation's estuaries: A decade of change,” Harmful Algae 8(1):21-32 (2008), doi: 10.1016/j.hal.2008.08.028).

The present invention provides a customizable in-line biosensor strain platform that can be used in microfluidic or other devices to house many different “sensor strains” in an aerobic aqueous environment, optionally, on a continuous basis.

### SUMMARY OF THE INVENTION

The present invention relates to a panel of recombinant Escherichia coli strains for monitoring a plurality of analytes of interest in an aqueous sample.

In one aspect, the panel can be used in a customizable in-line biosensor platform that uses a microfluidic device to house many different “sensor strains,” each with the ability to detect a different water contaminant or analyte, optionally on a continuous basis for extended periods, with high sensitivity and selectivity, in particular, for six different analytes of interest—often viewed as environmental contaminants in various settings: ammonium, nitrate, nitrite, phosphate, iron cations, nickel dication, and manganese dication. We used synthetic biology techniques to design and construct novel strains of bacteria that regulate the production of green fluorescent protein (GFP) in response to ambient concentrations of each specific analyte. We calibrated our strains using hundreds of chemical inductions to translate their responses to concentrations in aqueous samples. These engineered recombinant strains can be used as part of a biosensor platform to continuously monitor water for the presence of these six analytes in real time, for example in “source waters,” such as, but not limited to, environmental waters, agricultural aqueous run-off, or industrial aqueous inputs and/or outputs, aqueous biological samples, e.g., urine, serum, or cerebrospinal fluid. The present invention can also be used to monitor these analytes in food samples that have been macerated and liquified into a “source water” for detection purposes within the scope of the inventive method for monitoring a plurality of analytes of interest.

Thus, the present invention relates to a panel of recombinant Escherichia coli strains for monitoring a plurality of analytes of interest in an aqueous sample. The panel includes a set of two or more recombinant Escherichia coli strains that, in a defined aqueous culture medium, constitutively express one or more antibiotic resistance genes providing resistance to one or more antibiotic agents, wherein the one or more antibiotic agents, separately, or in combination, are characterized by both antibacterial and antifungal activity in the aqueous culture medium. Each of the E. coli strains comprises a stable recombinant expression system comprising an expression cassette comprising an analyte-sensitive promoter that specifically responds to at least one of the plurality of analytes of interest, resulting in a modification of expression from the promoter, the promoter being capable of operating under aerobic physiological conditions and being operably linked to a gene encoding a detectable marker. The set of two or more Escherichia coli strains is capable of expressing the detectable marker in the presence of at least one of the plurality of analytes of interest in a continuous series of liquid aqueous samples mixed with fresh defined culture medium, and the set of two or more Escherichia coli strains, and the identity of each Escherichia coli strain in the set can be distinguished from others during marker detection or measurement.

The panel of recombinant Escherichia coli strains for monitoring a plurality of analytes of interest in an aqueous sample is useful for practicing a method for monitoring a plurality of analytes of interest. The method involves mixing a continuous series of liquid aqueous samples with a fresh defined liquid culture medium in a defined dilution ratio to obtain a continuous series of diluted samples; contacting the series of diluted samples with the panel of recombinant E. coli strains comprising a set of two or more of the inventive E. coli strains, under aerobic physiological conditions; and monitoring for the expression of the detectable marker by the set of two or more E. coli strains arrayed in locations, wherein the identity of each E. coli strain can be distinguished; and correlating any expression of the detectable marker by the set of two or more E. coli strains, with the presence of at least one of the plurality of analytes of interest in the aqueous sample with a limit of detection in a concentration range of 1-1000 ppb.

The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows a schematic representation of the general architecture of embodiments of the constructed E. coli biosensor plasmids. A variable sensing promoter region was placed downstream of a constant 5′ insulation unit and upstream of green fluorescent protein (GFP), or another suitable reporter protein. An origin of replication (OOR) and antibiotic resistance cassette were included for plasmid propagation and maintenance purposes, respectively.

FIG. 2 shows schematic representations of variable sensing promoter regions for the engineered E. coli strains. The effect of the sensed analyte on the promoter driving GFP expression is shown for each indicated strain. In FIG. 2, genes are represented as rectangles, and their corresponding upstream promoters as bent arrows. Regulatory elements acting upon the promoters (typically the target analytes) are shown above as either pointed arrows (positive regulation) or blunt arrows (negative regulation). Pointed arrows represent a “lights on” sensor, whereby increased concentrations of the analyte increase levels of GFP, and blunt arrows represent a “lights off” sensor, whereby increased concentrations of the analyte decrease levels of GFP. Dashed lines indicate continuous sections of DNA copied from the E. coli MG1655 genome. Black semi-circles upstream of the gfp gene represent substitution of the Lutz and Bujard strong RBS for the native promoter RBS. (See, R. Lutz and H. Bujard, “Independent and tight regulation of transcriptional units in Escherichia coli via the LacR/O, the TetR/O and AraC/11-12 regulatory elements,” Nucleic Acids Res, vol. 25, no. 6, pp. 1203-1210, 1997, doi: 10.1093/nar/25.6.1203).

FIG. 3 shows a schematic map of an embodiment of an ammonium-sensing plasmid construct based on the glnA promoter, designated plasmid “78_pQBI_P12glnA-glnA-LutzRBS-sfgfp-glnLG_Hygro_p15A,” and having the nucleic acid sequence of SEQ ID NO:1.

FIG. 4 shows a schematic map of an embodiment of an ammonium-sensing plasmid construct based on the glnK promoter, designated plasmid “40_pQBI_Pfnr-fnr(L28H)_PglnK-LutzRBS-sfgfp_Hygro_p15A,” and having the nucleic acid sequence of SEQ ID NO:2.

FIG. 5 shows a schematic map of an embodiment of a nitrate-sensing plasmid construct based on the narG promoter designated plasmid “21_pQBI_Pfnr-fnr(L28H)_PnarGtrim-LutzRBS-sfgfp_Hygro_p15A,” and having the nucleic acid sequence of SEQ ID NO:3.

FIG. 6 shows a schematic map of an embodiment of a nitrate-sensing plasmid construct based on the fdnG promoter designated plasmid “34_pQBI_Pfnr-fnr(L28H)_PfdnGtrim-LutzRBS-sfgfp_Hygro_p15A,” and having the nucleic acid sequence of SEQ ID NO:4.

FIG. 7 shows a schematic map of an embodiment of a nitrite-sensing plasmid construct based on the nrfA promoter designated plasmid “61_pQBI_PnsrR-nsrR_Pfnr-fnr(L28H)_PnrfAtrim-LutzRBS-sfgfp_Hygro_p15A,” and having the nucleic acid sequence of SEQ ID NO:5.

FIG. 8 shows a schematic map of an embodiment of a nitrite-sensing plasmid construct based on the nirB promoter designated plasmid “64_pQBI_PnsrR-nsrR_Pfnr-fnr(L28H)_PnirBtrim-LutzRBS-sfgfp_Hygro_p15A” and having the nucleic acid sequence of SEQ ID NO:6.

FIG. 9 shows a schematic map of an embodiment of a phosphate-sensing plasmid construct based on the phoB promoter designated plasmid “84_pQBI_PphoB-phoBR-LutzRBS-sfgfp_Hygro_p15A” and having the nucleic acid sequence of SEQ ID NO:7.

FIG. 10 shows a schematic map of an embodiment of a phosphate-sensing plasmid construct based on the pstS promoter designated plasmid “85_pQBI_Pfnr-fnr(L28H) PphoB-phoBR_PpstS-LutzRBS-sfgfp_Hygro_p15A and having the nucleic acid sequence of SEQ ID NO:8.

FIG. 11 shows a schematic map of an embodiment of an iron-sensing plasmid construct based on the mntP promoter designated “mntP18_yobD_FurB_PmntP_ribo-LutzRBS-sfgfp_hph_p15A” and having the nucleic acid sequence of SEQ ID NO:9.

FIG. 12 shows a schematic map of an embodiment of a Mn(II)-sensing plasmid construct designated mntP6_MntRB_FurB_PmntP_ribo_trim27-LutzRBS-sfgfp_hph_p15A, having the nucleic acid sequence of SEQ ID NO:10, and based on on modified mntP promoter with the H-NS binding site removed to eliminate Fe(II) regulation of mntP through Fur. With this construct the detection range for Mn(II) was 10-1000 ppb.

FIG. 13 shows a schematic map of an embodiment of a Mn(II)-sensing plasmid designated mntP20_MntRB_FurB_PmntP_ribo_(15AA)-sfgfp_hph_p15A having the nucleic acid sequence of SEQ ID NO:11. With this construct the detection range for Mn(II) was 1000-3000 ppb.

FIG. 14a-d shows representative fluorescence responses for four selected analyte-sensing E. coli strains driving GFP expression near their limits of detection, as described in Example 2 herein: strain narG constructed around the E. coli narG promoter exhibiting induction of fluorescent GFP expression in response to a 25 ppb exposure to nitrate (FIG. 14a); strain glnA constructed around the E. coli glnA promoter exhibiting decreased fluorescence in response to exposure to 500 ppb ammonium (FIG. 14b); strain phoB constructed around the E. coli phoB promoter exhibiting decreased fluorescence in response to a 25 ppb exposure to phosphate (FIG. 14c); and strain nrfA constructed around the E. coli nrfA promoter induction of fluorescent GFP expression in response to exposure to a 250 ppb nitrite (FIG. 14d). Each light gray line represents the median band-pass filtered response of at least six replicate strain banks in the microfluidic array, while the dark gray line represents the median response across multiple inductions. Vertical lines bound 4-h windows of exposure to the analyte shown above each plot.

FIG. 15 shows the results of representative biosensor characterization experiment, in which the glnA strain was subjected to 2-hour pulses of ammonium at increasing concentrations over time. The time series represents the mean of 100 strain banks in the microfluidic device containing the glnA strain. Vertical dark solid lines represent changes in the exposed ammonium concentration; vertical light solid lines represent the start of a 2-hour exposure to the analyte; and vertical light dashed lines represent the start of a 2-hour exposure to pure water. The top panel shows raw fluorescence data extracted from a stack of GFP images, whereas the bottom panel shows the processed fluorescence signal.

FIG. 16a-f shows the results of representative fluorescence amplitude responses for six biosensor strains from a top-performing panel. Strain glnA response increased with increasing ammonium concentration (FIG. 16a). Strain fdnG response increased with increasing nitrate concentration (FIG. 16b). Strain nrfA response increased with increasing nitrite concentration (FIG. 16c). Strain phoB response increased with increasing phosphate concentration (FIG. 16d). Strain ugpB response increased with increasing iron concentration (FIG. 16e). Strain mntP response increased with increasing manganese concentration (FIG. 16f). Error bars represent the standard deviation of multiple inductions at each concentration.

FIG. 17a-b illustrates the amplitude of the fluorescence strain response of strain glnA responding to sequential increasing and decreasing steps in ammonium concentration, from 0 to 7 ppm to 0. FIG. 17a shows the fluorescence strain response during “AC” inductions with 4-h period, where every 2-h exposure was alternated between the analyte calibration stock and pure water. Six 4-h “AC” inductions (lasting 24 h) were performed at each analyte concentration before stepping to the next analyte concentration. FIG. 17b shows the amplitude of the oscillating strain response generated by performing an FFT on the signal shown in FIG. 17a. At each increasing and decreasing step in analyte concentration, the amplitude magnitude correspondingly stepped up and down.

FIG. 18a-b illustrates the amplitude of the fluorescence strain response of strain fdnG responding to sequential increasing and decreasing steps in nitrate concentration, from 0 to 200 ppb to 0. FIG. 18a shows the fluorescence strain response during “AC” inductions with 4-h period, where every 2-h exposure was alternated between the analyte calibration stock and pure water. Three 4-h “AC” inductions (lasting 12 h) were performed at each analyte concentration before stepping to the next analyte concentration. FIG. 18b shows the amplitude of the oscillating strain response generated by performing an FFT on the signal shown in FIG. 18a. At each increasing and decreasing step in analyte concentration, the amplitude magnitude correspondingly stepped up and down.

FIG. 19a-b illustrates the amplitude of the fluorescence strain response of strain phoB responding to sequential increasing and decreasing steps in phosphate concentration, from 0 to 2500 ppb to 0. FIG. 19a shows the fluorescence strain response during “AC” inductions with 4-h period, where every 2-h exposure was alternated between the analyte calibration stock and pure water. Six 4-h “AC” inductions (lasting 24 h) were performed at each analyte concentration before stepping to the next analyte concentration. FIG. 19b shows the amplitude of the oscillating strain response generated by performing an FFT on the signal shown in FIG. 19a. At each increasing and decreasing step in analyte concentration, the amplitude magnitude correspondingly stepped up and down.

FIG. 20a-b illustrates representative results of strain codB responding to sequential increasing and decreasing steps in iron concentration, from 0 to 1000 ppb to 0. FIG. 20a shows the fluorescence strain response during “AC” inductions with 4-h period, where every 2-h exposure was alternated between the analyte calibration stock and pure water. Three 4-h “AC” inductions (lasting 12 h) were performed at each analyte concentration before stepping to the next analyte concentration. FIG. 20b shows the amplitude of the oscillating strain response generated by performing an FFT on the signal shown in FIG. 20a. At each increasing and decreasing step in analyte concentration, the amplitude magnitude correspondingly stepped up and down.

FIG. 21a-b illustrates representative results of strain fes responding to sequential increasing and decreasing steps in iron concentration, from 0 to 1000 ppb to 0. FIG. 21a shows the fluorescence strain response during “AC” inductions with 4-h period, where every 2-h exposure was alternated between the analyte calibration stock and pure water. Three 4-h “AC” inductions (lasting 12 h) were performed at each analyte concentration before stepping to the next analyte concentration. FIG. 21b shows the amplitude of the oscillating strain response generated by performing an FFT on the signal shown in FIG. 21a. At each increasing and decreasing step in analyte concentration, the amplitude magnitude correspondingly stepped up and down.

FIG. 22a-b illustrates representative results of strain ugpB responding to sequential increasing and decreasing steps in iron concentration, from 0 to 1000 ppb to 0. FIG. 22a shows the fluorescence strain response during “AC” inductions with 4-h period, where every 2-h exposure was alternated between the analyte calibration stock and pure water. Three 4-h “AC” inductions (lasting 12 h) were performed at each analyte concentration before stepping to the next analyte concentration. FIG. 22b shows the amplitude of the oscillating strain response generated by performing an FFT on the signal shown in FIG. 22a. At increasing and decreasing steps in analyte concentration above a threshold (around 20-50 ppb), the amplitude magnitude correspondingly stepped up and down.

FIG. 23a-b illustrates ammonium dosing and consumption in an actively growing outdoor algae pond culture throughout two growth experiments spanning 22 days, as described in Example 3 herein. Vertical dashed lines mark pond management events including inoculation with Scenedesmus obliquus (UTEX 393) culture, biomass harvesting, and BG-11 medium dosing. FIG. 23a shows the fluorescence response of the engineered E. coli strain glnA to changing ammonium concentration in the managed pond culture. FIG. 23b shows sensor data from panel FIG. 23a, calibrated to triplicate pond grab samples analyzed by Hach ammonium test kits (black circles), both before (gray line), and after (black line) calibration to account for a diurnal pattern of evaporation in that embodiment of the microfluidic biosensor device hardware. Biosensor measurements agreed well within a target±15% accuracy window indicated by error bars.

FIG. 24 shows a schematic map of an embodiment of an iron (Fe(II))-sensing plasmid designated 99_pQBI_codB_hph having the nucleic acid sequence of SEQ ID NO:36. With this construct the limit of detection for iron was 10 ppb.

FIG. 25 shows a schematic map of an embodiment of an iron (Fe(II))-sensing plasmid designated 100_pQBI_fes_hph having the nucleic acid sequence of SEQ ID NO:37. With this construct the limit of detection for iron was 10 ppb.

FIG. 26 shows a schematic map of an embodiment of an iron (Fe(II))-sensing plasmid designated 101_pQBI_ugpB_hph having the nucleic acid sequence of SEQ ID NO:38. With this construct the limit of detection for iron was 50 ppb.

FIG. 27 illustrates a schematic map of the variable sensing promoter region found in each E. coli strain listed in Table 1, with the nucleotide sequence of each variable sensing promoter region indicated by its SEQ ID NO. As shown in FIG. 27, a variable sensing promoter region designed to be sensitive to ammonium comprises the nucleotide sequence of SEQ ID NO:50 or SEQ ID NO:55; a variable sensing promoter region designed to be sensitive to nitrate comprises the nucleotide sequence of SEQ ID NO:59 or SEQ ID NO:63; a variable sensing promoter region designed to be sensitive to nitrite comprises the nucleotide sequence of SEQ ID NO:69 or SEQ ID NO:74; a variable sensing promoter region designed to be sensitive to phosphate comprises the nucleotide sequence of SEQ ID NO:78 or SEQ ID NO:83; a variable sensing promoter region designed to be sensitive to iron comprises the nucleotide sequence of SEQ ID NO:85, SEQ ID NO:86, SEQ ID NO:87, or SEQ ID NO:90; and a variable sensing promoter region designed to be sensitive to manganese (II) comprises the nucleotide sequence of SEQ ID NO:93 or SEQ ID NO:95. In FIG. 27, “T0” means the terminator sequence (SEQ ID NO:15) from Escherichia phage Lambda, and “T22” means the terminator sequence from Salmonella phage P22 (SEQ ID NO:26), but other terminator sequences can be used instead. Black semi-circles upstream of the gfp gene represent substitution of the Lutz and Bujard strong ribosomal binding site (RBS) for the native promoter RBS. (See, R. Lutz and H. Bujard, “Independent and tight regulation of transcriptional units in Escherichia coli via the LacR/O, the TetR/O and AraC/11-12 regulatory elements,” Nucleic Acids Res, vol. 25, no. 6, pp. 1203-1210, 1997, doi: 10.1093/nar/25.6.1203); however, other suitable reporter genes can be used instead.

FIG. 28a-c illustrates an embodiment of a microfluidic device used to house (here shown in a dual strain array) and control the microenvironment of the engineered recombinant E. coli strains, while monitoring their fluorescence responses to targets in the source water, and representative results. FIG. 28a shows a schematic representation of the imaged region of this split microfluidic device embodiment, where each half of the chip is loaded with a replicate array of sensing strains, and shows a magnified cell reservoir. In the “AC” sensing mode, a source water of unknown composition flows across one strain array, and reference water flows across the other strain array. These flows are redirected to alternate between the strain arrays with the “AC” period of typically, but not necessarily, 4 hours. In this manner, the strains are imaged periodically to monitor their response to a continuous series of steps in water composition. FIG. 28a represents different microfluidic channel heights using different shades of gray. Specifically, light gray indicates cell reservoirs where the engineered strains are spotted and cultured; medium gray indicates cross channels where fresh nutrients and source or reference water flow past the strains and are delivered by a combination of convection and diffusion; and dark gray indicates manifold channels of low resistance that serve to equalize flow rates across the arrays. The cell reservoir is divided into a spotting region where cells are deposited and dehydrated during chip manufacture and an extraction region where fluorescence response data is calculated from the camera images. Each camera image frame (represented by the white rectangular extents) is registered to a reference frame generated from the CAD file of the microfluidic chip design (represented by the black rectangular extents) using an affine transformation available from OpenCV Open Source Computer Vision Library (docs.opencv.org/3.4/d4/d61/tutorial_warp_affine.html), prior to fluorescence data extraction. Registration of the image stack compensates for possible image drift over many days. FIG. 28b shows an overlay of magnified images acquired in the transmitted and GFP fluorescence light spectra; manifold flows (indicated by vertical downward-facing arrows) and cross-flows (indicated by horizontally-facing arrows) are illustrated feeding to 12 representative addressable cell reservoirs. A unique engineered recombinant E. coli strain of the invention is spotted in each spotting region, causing variations in fluorescence response across the extraction regions. FIG. 28c illustrates a magnification the boxed cell reservoir in FIG. 28b, showing the variation in GFP fluorescence between the spotting region and the extraction region. This is likely a consequence of variations in cell packing density within the cell reservoir, as well as limited diffusion rates for different analytes. We generally opt to measure strain response signal from the extraction region adjacent to cross-flow, because cells in this region are not diffusion-limited and therefore more metabolically active, but other regions can be chosen. (See, e.g., G. Graham et al., “Genome-scale transcriptional dynamics and environmental biosensing,” Proc Nat Acad Sci USA 117(6):3301-3306 (2020), doi: 10.1073/pnas.1913003117).

FIG. 29a-b shows the calibration of fluorescence responses of the inventive glnA engineered recombinant E. coli strain to the NH₄⁺ inductions shown in FIG. 17a-b. In FIG. 29a, the raw signal Alternating Conditions (AC) amplitude (dark gray trace) is temporally aligned and scaled to the ammonium (designated “NH₄—N”) inducer concentrations (light gray trace), and mean amplitude is calculated within the shaded time windows away from the induction transitions. FIG. 29b shows the data fit to a model for response amplitude (A) as a function of NH₄—N concentration in ppm (X). Specifically, we used the model A=A0*(X/(C0+X)), where C0=17.69395271 and A0=12.0225801. This strain calibration can be used to map subsequent strain responses to NH₄ concentrations.

FIG. 30 shows a schematic representation of an embodiment of the processing of “raw” aqueous sample obtained from source water to obtain “conditioned” aqueous sample doses in a continuous stream, or series, of diluted (aqueous) samples, flowing in a channel, tubing, or other suitable liquid-conveying conduit, toward a microfluidic device (or “chip”) (upward arrow in center points to a microfluidic device, which is not shown in FIG. 30, but see, e.g., FIG. 28a-c) housing a panel of recombinant E. coli strains of the invention (not shown).

FIG. 31 shows a schematic representation of a variable sensing promoter region for the nickel dication-sensing engineered E. coli strain listed in Table 2, with the nucleotide sequence of each variable sensing promoter region indicated by its SEQ ID NO. The effect of the sensed analyte on the promoter driving GFP expression is shown for the strain. Genes are represented as rectangles, and their corresponding upstream promoters as bent arrows. Regulatory elements acting upon the promoters (typically the target analytes) are shown above as either pointed arrows (positive regulation) or blunt arrows (negative regulation). Pointed arrows represent a “lights on” sensor, whereby increased concentrations of the analyte increase levels of GFP, and blunt arrows represent a “lights off” sensor, whereby increased concentrations of the analyte decrease levels of GFP. Dashed lines indicate continuous sections of DNA copied from the E. coli MG1655 genome. In FIG. 31, “T0” means the terminator sequence (SEQ ID NO:15) from Escherichia phage Lambda, but other terminator sequences can be used instead. Black semi-circles upstream of the gfp gene represent substitution of the Lutz and Bujard strong ribosomal binding site (RBS) for the native promoter RBS. (See, R. Lutz and H. Bujard, “Independent and tight regulation of transcriptional units in Escherichia coli via the LacR/O, the TetR/O and AraC/11-12 regulatory elements,” Nucleic Acids Res, vol. 25, no. 6, pp. 1203-1210, 1997, doi: 10.1093/nar/25.6.1203); however, other suitable reporter genes can be used instead.

FIG. 32 shows a schematic map of an embodiment of a nickel dication-sensing plasmid construct based on the rcnAB promoter, designated plasmid “pQBI_PWTW001_Nickel_prcnA,” and having the nucleic acid sequence of SEQ ID NO:96.

FIG. 33a-c shows the renAB strain response to nickel dication in a microfluidic chip as we probed the strain detection limit. FIG. 33a shows the mean cellular fluorescence response across all strain banks (gray line) over approximately seven days. FIG. 33b shows the signal in FIG. 33a processed by a band pass filter (black line) and then a Fast Fourier Transform (FFT) with 4 h period (one complete “AC” cycle) to calculate the oscillation amplitude (gray line). FIG. 33c shows a time-lapse series of images cropped to one strain bank in the microfluidic chip.

FIG. 34a-c shows data from a calibration procedure for an embodiment of the inventive nickel dication sensing strain, using the rcnAB response amplitude and associated Ni(II) concentration data. FIG. 34a shows calibration data selection, in which time windows of strain responses located between transitions in analyte concentration (gray shading) are associated with known Ni(II) concentrations. FIG. 34b shows the strain response amplitude data fit to the analyte concentration. FIG. 34c shows calibrated rcnAB strain response data (black line) overlaid upon the nickel dication induction sequence (gray dashed line) for comparison.

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

The present invention involves a panel of recombinant Escherichia coli strains for monitoring a plurality of analytes of interest in an aqueous sample. An “analyte” is a chemical substance that is the subject of identification and/or measurement, e.g., ammonium, nitrate, nitrite, phosphate, a manganese dication, an iron cation, a nickel dication, or a combination of any one of these.

In some embodiments, the panel of recombinant Escherichia coli strains for monitoring a plurality of analytes of interest in an aqueous sample includes a set of at least 2 different engineered strains, 3 different engineered strains, 4 different engineered strains, 5 different engineered strains, 6 different engineered strains, or 7 different engineered strains, or more.

In some embodiments, the panel of recombinant Escherichia coli strains includes a set of two or more recombinant Escherichia coli strains, which are lyophilized (freeze-dried), or otherwise dehydrated, e.g., by air-drying. (See, e.g., Hasty et al., “Microbial Microfluidic Biosensor,” U.S. Pat. No. 11,209,412B2). After being in a dehydrated state for at least 30-60 days, the set of two or more Escherichia coli strains is capable of being revived under aqueous physiological conditions, and is capable of expressing the optically detectable marker in the presence of at least one of the plurality of analytes of interest.

The inventive panel of E. coli strains and methods can be employed within microfluidic biosensor devices, or “chips,” suitable for continuously monitoring analyte levels in aqueous samples; in such devices, the strains are distinguishable from each other by being arrayed in separate addressable chambers or colonies within the device, and each strain housed in its preselected chamber or colony having the ability to detect one of the different analytes, and/or different concentration ranges of an analyte. Such microfluidic biosensor devices can typically run freely for 30-60 days without intervention. (See, e.g., Hasty et al., “Microbial Microfluidic Biosensor,” U.S. Pat. No. 11,209,412B2). In some embodiments, the panel and/or the microfluidic biosensor containing the inventive panel can run for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or up to 60 days, or any number of days defined by a range between any two aforementioned values, without intervention.

Alternatively, the identity of each Escherichia coli strain can be distinguished from the other strains in the set of two or more Escherichia coli strains by being placed in addressable preselected locations, such as separate cuvettes, wells in microtiter plates, and the like. In other embodiments, the identity of each Escherichia coli strain can be distinguished from the other strains in the set of two or more Escherichia coli strains by expressing a selectable marker identifiably different from other strains in the set, even if cells of the strain are physically mixed with cells of other strains, e.g., by an identifiable emission wavelength. This method of distinguishing strains is also useful in biosensor hardware embodiments of the invention, involving culturing of the strains in “macrofluidic” chemostats or turbidostats.

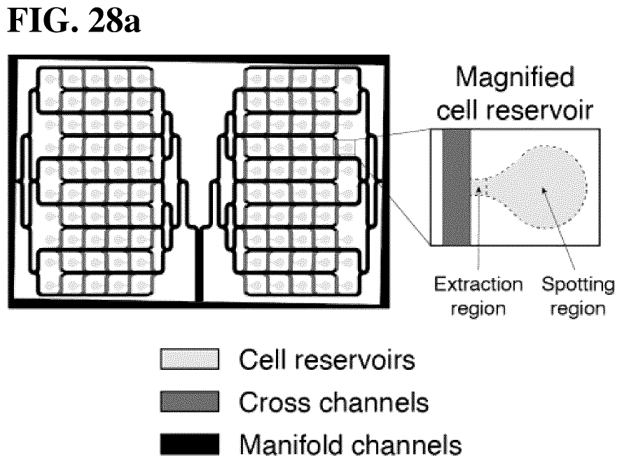

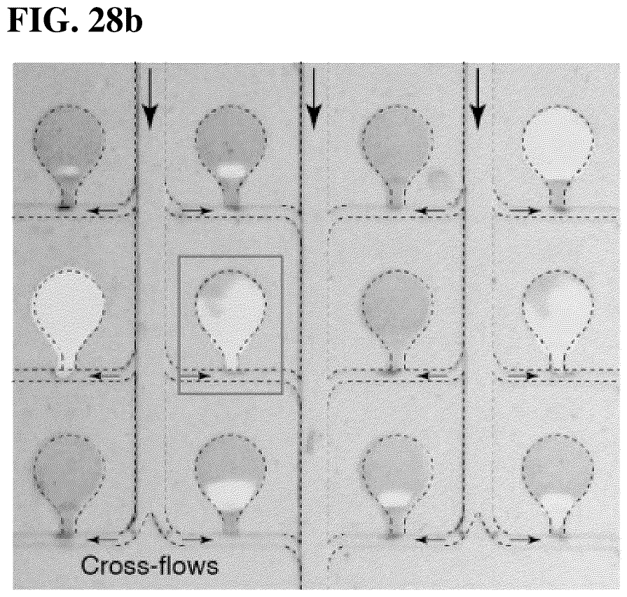

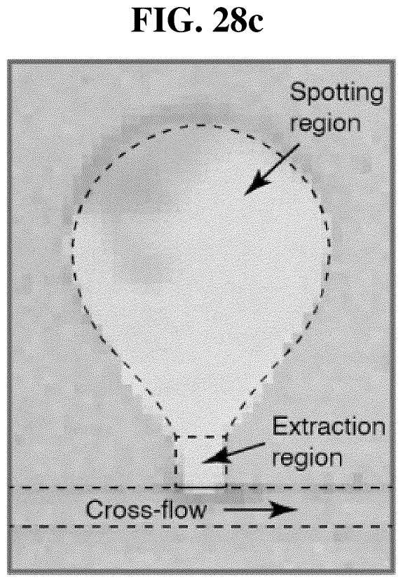

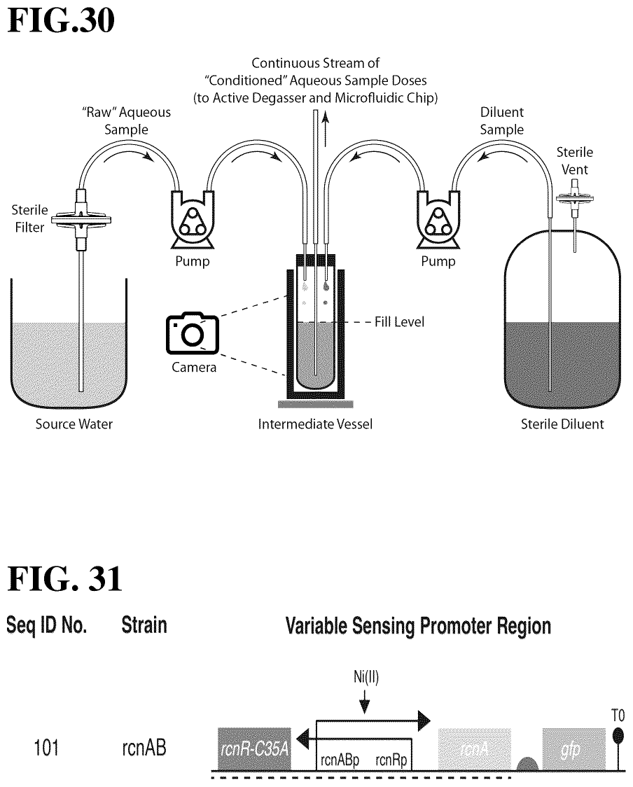

A “continuous series” of aqueous samples means a plurality of chronologically sequential liquid aqueous sample doses for detection or monitoring of the expression of a preselected detectable marker. Optionally, the sample doses are “conditioned” aqueous sample doses. The conditioned aqueous sample doses are generated by serially processing volumes (microliter or larger) of “raw” aqueous sample (i.e., of unprocessed source water), obtained in chronological succession, through an optional sterile filtration step and/or optional dilution before application of the sensing or detection instrumentation. In a merely illustrative example of an embodiment involving the recombinant E. coli strains arrayed in a microfluidic biosensor chip device for housing them, in each raw sample dosing event, the processing of raw aqueous sample (i.e., unprocessed source water) involves the following: 25-100 μL of the raw aqueous sample is sterile-filtered, diluted with 300-375 μL of sterile diluent, and mixed in an intermediate vessel maintained at a 1.0-1.5 mL total volume; a continuous stream of “conditioned” liquid aqueous sample is pumped from this intermediate vessel through an active degasser and thence into the microfluidic chip (see, FIG. 30). Flows of the liquid source water and diluent can be driven, optionally, by pumping, or alternatively, by pressurizing headspaces in closed vessels. Useful pumps, as shown in FIG. 30, include, but are not limited to, peristaltic pumps or centrifugal pumps. Various filters, connecting parts, diplegs, and tubing are shown in FIG. 30, which can be reusable metal or plastic parts and liquid conveying conduits, or can be entirely single-use and disposable, if desired. Optionally, the source water can be sterile-filtered (as shown in FIG. 30) using an inline filter (e.g., for clear source water with relatively few particulates), or by recirculation through the retentate ports of a tangential flow filter (TFF), where sterile filtrate is drawn from the filtrate port of the TFF into the intermediate vessel (for turbid source water). The diluent can be, optionally, ultrapure water, a growth medium, an acid, a base, or a pH buffer, depending on downstream needs. The intermediate vessel can be mixed via magnetic stirrer or bubbling of inert gas. As part of the processing, a camera can be used to continuously image the fill level in the intermediate vessel, as in the embodiment shown in FIG. 30. When the fill level drops below some threshold, a machine learning algorithm analyzing this image stream can be employed to automatically initiate a dosing event, which triggers the flow of both source water and diluent at the appropriate volumetric mixing ratio. Flow meters placed inline in the source and diluent streams can be implemented to ensure an accurate mixing ratio in the intermediate vessel. An electronic computerized “controller” or “microcontroller” or “digital control unit,” terms used interchangeably herein, can be employed to automatically direct the activity of the pump(s), meter(s) or sensor(s), optional valves, and/or data collection. Within the scope of the inventive method for monitoring a plurality of analytes of interest, the continuous series of aqueous samples of source water can be obtained sequentially from the source water over a time period extending from 1 to 5 minutes, or from 5 minutes up to 10 minutes, or from 10 minutes up to 1-4 hours, or from 4 hours up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or up to 60 days, or more. The source water can optionally not be “conditioned” before being directed to the apparatus and/or instrumentation for detection or monitoring of the expression of the preselected detectable marker(s), e.g., a microfluidic device (e.g., see, FIG. 28a-c), if the source water has been pre-processed or does not require pre-processing to be compatible with the apparatus and/or instrumentation.

A protein of interest is used in the practice of the invention, whether a variant or parent protein, is typically produced by recombinant expression technology. The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques.

The term “recombinant protein” or “recombinant polypeptide” as used herein refers to a protein molecule, e.g., an antibody, an enzyme, a transcription factor, a detectable marker (e.g., a fluorescent protein or a luminescent protein), which is expressed using a recombinant DNA molecule. A “recombinant host cell” is a cell that contains and/or expresses a recombinant nucleic acid, e.g., a recombinant Escherichia coli cell of the invention.

The term “naturally occurring,” where it occurs in the specification in connection with biological materials such as polypeptides, nucleic acids, host cells, and the like, refers to materials which are found in nature.

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences may include promoters comprising one or a plurality of recognition sites for transcription factors, activator sequences, transcription enhancer (or enhancer-like) sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). The selection of a particular promoter and enhancer depends on what cell type is to be used to express the protein of interest. Some promoters and enhancers have a broad host range while others are functional in a limited subset of cell types (for review see Voss, et al., Trends Biochem. Sci., 11:287 (1986) and Maniatis, et al., Science 236:1237 (1987)).

A “promoter” is a region of DNA including a site at which RNA polymerase binds to initiate transcription of messenger RNA by one or more downstream structural genes. Promoters are located near the transcription start sites of genes, on the same strand and upstream on the DNA (towards the 5′ region of the sense strand). Promoters are typically about 100-1000 bp in length.

The term “modification of expression” from a promoter means either higher measurable expression (i.e., activation or induction) or lower measurable expression (i.e., repression), compared to the level of expression in the absence of an analyte of interest. For example, the measurable expression difference can be by at least 1.5-fold, or more, or by at least two-fold, or more, or by at least three-fold, or more, or by at least five-fold, or by at least ten-fold, or more, compared to the level of expression in the absence of an analyte of interest.

“Physiological conditions” are conditions of pH, temperature, nutrients, and the like, that allow the E. coli cells to express a recombinant expression cassette. “Aerobic” physiological conditions are those above the Pasteur Point for a particular microbial organism (in this case a strain of E. coli), i.e., the partial pressure of oxygen (in equilibrium with a solution; “PO₂”) is above the PO₂ at which a facultative aerobic organism switches to anaerobic metabolism, which is typically below a value of approximately 0.01 (1%) of the present atmospheric oxygen level (“PAL”). (See, Stolper et al., “Aerobic growth at nanomolar oxygen concentrations,” PNAS 107(44):18755-18760 (2010), pnas.org/cgi/doi/10.1073/pnas.1013435107P). Typically, an oxygen sensor is useful to indicate whether aerobic conditions prevail.

In general, a promoter is capable of operating under “aerobic” physiological conditions, if transcription from the promoter can occur when the redox potential (i.e., oxidation/reduction potential; also known as “ORP” or Eₕ) in the aqueous environment surrounding the cell is Eₕ>50-300 mV. The redox potential is measured in millivolts (mV) relative to a standard hydrogen electrode and is commonly measured using a platinum electrode with a saturated calomel electrode as reference. In well-oxidized water, as long as oxygen concentrations stay above about 1 mg O₂/L, the redox potential will be highly positive (i.e., above 300-500 mV). In reduced environments, such as in the deep water of stratified lakes or the sediment of eutrophic lakes, the redox potential will be low, even a negative value. (See, e.g., M. Søndergaard, In: “Encyclopedia of Inland Waters,” Ed. Gene E. Likens, Academic Press (2009), pp. 852-859; C. Tobias et al., “Coastal Wetlands: An Integrated Ecosystem Approach,” Second Edition, Eds. Gerardo M. E. Perillo et al., Elsevier (2019), pp. 539-596). ORP measurements are quick and easy with a ORP probe, but ORP values can give a distorted proxy value of oxygenation in the presence of certain chemicals, e.g., hypochlorite, often found in source waters from industrial or water processing plants.

An “enhancer” is a short (50-1500 bp) region of DNA that can be bound with one or more activator proteins (transcription factors) to activate transcription of a gene.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, proteins can be derivatized as described herein and by other well-known organic chemistry techniques.

A “variant” of a polypeptide comprises an amino acid sequence wherein one or more amino acid residues are inserted into, deleted from and/or substituted into the amino acid sequence relative to another polypeptide sequence. Variants can include fusion proteins.

The term “fusion protein” indicates that the protein includes polypeptide components derived from more than one parental protein or polypeptide. Typically, a fusion protein is expressed from a “fusion gene” in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

A “secreted” protein refers to those proteins capable of being directed to the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments, the antibody protein of interest can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing an ionic detergent or chaotropic agent, such as sodium dodecyl sulfate (SDS), urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing an ionic detergent or chaotropic agent, such as sodium dodecyl sulfate (SDS), urea, guanidinium hydrochloride, or lithium perchlorate.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoranilidate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express the immunoglobulin (e.g., antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

An “expression cassette” refers to a nucleic acid construct, which when introduced into a host cell, results in transcription and/or translation of a RNA or polypeptide, respectively. The expression cassette includes a gene encoding a protein of interest. It includes a promoter, operable in an E. coli cell, for mRNA transcription, one or more gene(s) encoding protein(s) of interest and a mRNA termination. An expression cassette can usefully include among the coding sequences, a gene useful as a selective marker and/or a detectable marker or reporter. In the expression cassette promoter is operably linked 5′ to an open reading frame encoding an exogenous protein of interest; and a polyadenylation site is operably linked 3′ to the open reading frame. Other suitable control sequences can also be included as long as the expression cassette remains operable. The open reading frame can optionally include a coding sequence for more than one protein of interest.

In some embodiments, the “detectable marker” is a protein, e.g., a fluorescent protein. In some embodiments, the fluorescent protein is selected from the group consisting of green fluorescent protein, a yellow fluorescent protein, a cyan fluorescent protein, a red-shifted green fluorescent protein (rs-GFP), and miniSOG. In some embodiments, the detectable protein is a luminescent protein. In some embodiments, the luminescent protein is bacterial luciferase (Lux).

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Recombinant expression technology typically involves the use of a recombinant expression vector comprising an expression cassette and a host cell comprising the recombinant expression vector with the expression cassette or at least the expression cassette, which may for example, be integrated into the host cell genome.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription and translation of a coding region operably linked thereto. Nucleic acid sequences necessary for expression in prokaryotes include a promoter, optionally an operator sequence, a ribosome binding site and possibly other sequences. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (See, e.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Han et al., “Novel signal peptides improve the secretion of recombinant Staphylococcus aureus Alpha toxinH35L in Escherichia coli,” AMB Express (2017) 7:93 Published online 2017 May 12, doi: 10.1186/s13568-017-0394-1). For expression of multi-subunit proteins of interest, separate expression vectors in suitable numbers and proportions, each containing a coding sequence for each of the different subunit monomers, can be used to transform a host cell. In other embodiments, a single expression vector can be used to express the different subunits of the protein of interest.

The term “host cell” means a cell that has been transformed, or is capable of being transformed, with a nucleic acid and thereby expresses a gene or coding sequence of interest. The term includes the progeny of the parent cell, whether or not the progeny is identical in morphology or in genetic make-up to the original parent cell, so long as the gene of interest is present. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Modifications can be made at the DNA level, as well. The peptide-encoding DNA sequence may be changed to codons more compatible with the chosen host cell. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The host cells can be usefully grown in batch culture, fed-batch culture, intensified fed-batch culture (product retention perfusion), or in continuous culture systems employing liquid aqueous medium. Host cells are generally cultured as suspension cultures. That is to say, the cells are suspended in a liquid cell culture medium, rather than adhering to a solid support. In other embodiments, the host cells can be cultured on solid or semi-solid aqueous culture medium, for example, containing agar or agarose, to form a medium, carrier (or microcarrier) or substrate surface to which the cells adhere and form an adhesion layer. Another useful mode of production is a hollow fiber bioreactor with an adherent cell line. Porous microcarriers can be suitable and are available commercially.

“Cell culture medium” or “culture medium,” used interchangeably, is defined, for purposes of the invention, as a sterile medium suitable for growth of cells, in in vitro cell culture. Any medium capable of supporting growth of the appropriate cells in cell culture can be used. Suitably, the culture medium has an osmolality of between 210 and 650 mOsm, preferably 270 to 450 mOsm, more preferably 300 to 350 mOsm. (See, e.g., Cayley S et al., “Large changes in cytoplasmic biopolymer concentration with osmolality indicate that macromolecular crowding may regulate protein-DNA interactions and growth rate in osmotically stressed Escherichia coli K-12,” J. Mol. Recognit. 17(5):488-96 (2004)). Preferably, the osmolality of the cell culture supernatant is maintained within one or more of these ranges throughout the culturing of host cells. The cell culture medium can be based on any basal medium, such as Luria-Bertani (LB) broth, M9 minimal medium, HM9 minimal medium, generally known to the skilled worker, and/or media further described herein. Commercially available media are suitable for culturing various host cells, or can be modified appropriately to suit the cell line employed. The basal medium can comprise a number of ingredients, including amino acids, vitamins, organic and inorganic salts, and sources of carbohydrate, each ingredient being present in an amount which supports the cultivation of a cell which is generally known to the person skilled in the art. The medium can contain auxiliary substances, such as buffer substances like sodium bicarbonate, antioxidants, stabilizers to counteract mechanical stress, or protease inhibitors. Any one of these media may be supplemented, as necessary, with physiologically acceptable salts, such as sodium chloride, calcium, magnesium, and phosphate salts, including salts of amino acids, such as, but not limited to, a lysine, histidine, or proline salt; with buffers, such as HEPES and/or sodium bicarbonate; nucleotides, such as adenosine and thymidine; antibiotics, such as gentamicin, neomycin, tetracycline, puromycin, or kanamycin; trace elements (defined as inorganic compounds usually present at final concentrations in the micromolar range); and glucose or an equivalent carbon and/or energy source, such that the physiological conditions of the cell in, or on, the medium promote expression of the protein of interest by the host cell; any other necessary supplements may also be included at appropriate concentrations that would be known to those skilled in the art.

In the inventive method for continuously monitoring a plurality of analytes of interest, aqueous samples are mixed with fresh defined culture medium in a defined dilution ratio to obtain a series of diluted samples, which are contacted with the inventive panel of recombinant Escherichia coli strains. The term “defined dilution ratio” means that the aqueous samples are mixed with the fresh defined culture medium in a predetermined dilution ratio of aqueous sample to concentrated culture medium, e.g., a dilution ratio of 20:1, 15:1, 10:1, 9:1, 8:1, 7:1, 6:1, 5:1, 4:1, 3:1, 2:1, 1:1, or any other dilution ratio suitable for the nature of the detectable marker selected, the format or platform housing the panel of strains, the instrumentation used for monitoring the expression of the detectable marker by the panel, and the suspected concentration of the analyte of interest in the aqueous sample; and the skilled person can readily adjust the defined dilution ratio as needed. In a typical example, one can add one (1) part of 5× concentrated defined culture medium to four (4) parts of aqueous sample, resulting in a (⅕)× dilution of concentrated culture medium and a (⅘)× dilution of aqueous sample.

The term “inoculation of the cells into the cell culture medium” refers to the step of contacting the cells with the cell culture medium under conditions which are suitable for growth and proliferation of the cells.

The cell culture contemplated herein may be any cell culture independently of the kind and nature of the cultured cells and the growth phase of the cultured cells, e.g. adherent or non-adherent cells; growing, or growth-arrested cells.

The term “sterile,” as used herein, refers to a substance that is free, or essentially free, of microbial and/or viral contamination. In this respect the “contaminant” means a material that is different from the desired components in a preparation being a cell culture medium or at least a component of a cell culture medium. In the context of “sterile filtration,” the term sterile filtration is a functional description that a preparation is filtered through a sterile filter (with a pore size of 0.2 μm or less) to remove bacterial and/or mycoplasma contaminants.

Filtration can be used to sterilize an aqueous fluid and/or to remove particulates. “Batch filtration,” otherwise known as “batch wise filtration” or filtration done in batch mode, refers herein to a process wherein a specific total amount or volume of a preparation, being a cell culture medium or at least a component of a cell culture medium, is filtered in one batch dependent on the capacity of the filter and wherein the filtration process is finalized before the filtrate is directed or fed to the process in which it is used or consumed. The term “continuous filtration” or “online filtration” or “in line filtration” refers to a filtration process, wherein the specific total amount or volume of a preparation, being a cell culture medium or at least a component of a cell culture medium, is filtered through the virus filter continuously dependent on the capacity of the virus filter and wherein the filtration process is still going on when the filtrate is already directed or fed to the process in which it is used or consumed. For example, a continuous filtration step can be used to filter and sterilize the aqueous sample where it first enters a sensor system. In one embodiment, a tangential flow filter with 500 kDa pore size is used; the continuous relatively fast flow of unfiltered sample tangential to the filter membrane acts to “sweep away” particles in the sample that would otherwise blind the filter membrane.

The “cell culture supernatant” is the extracellular medium in which the cells are cultured. This medium is not to be confused with feed medium that may be added to the culture after inoculation of the cells into the cell culture medium and cell growth has been commenced. A “cell culture” means the cell culture supernatant and the cells cultured therein. Conventionally, E. coli cells are cultured at 37° C.±1° C. or ambient temperature.

By “culturing at” or “maintaining at” a temperature, is meant that the temperature to which the process control systems are set, in other words the intended, target temperature, pH, oxygenation level. The culture conditions, such as temperature (typically, but not necessarily, about 20-37° C.), pH (typically, but not necessarily, a cell culture medium is maintained within the range of about pH 6.5-7.5, as modified consistent with the present invention), oxygenation, and the like, will be apparent to the ordinarily skilled artisan. Clearly, there will be small variations of the temperature of a culture over time, and from location to location through the culture vessel. Digital control units and sensory monitors are available commercially or can be constructed by the skilled artisan. Alternative digital control units (DCU) control and monitor the cell culture process are available commercially, made by companies such as B. Braun, New Brunswick, or Sartorius. For in-flask batch culture with shaker, numerous models of suitable cell culture incubators with built-in environmental controls are commercially available, e.g., by Thermo Fisher Scientific. In a typical useful embodiment, a constant culture temperature of 37° C. is maintained, using a proportional-integral-derivative controller (PID) controller (also known as a “three-term” controller) to control a thermoelectric module with input from a 10K thermistor. Such a system can both heat or cool. Alternatively, a system with a resistive heater can only heat.

“Culturing at” or “maintaining at” a temperature that is set at X±Y° C., means that the set point is at a value of from X+Y° C. to X−Y° C. For example, where X is 37.0±0.9° C., the set-point is set at a value of from 37.9 to 36.1° C. For each of the preferred values of X, e.g., X=31, X=32, X=33, X=34, X=35, X=36, or X=37, the set-point is at a value within the range X±0.9° C., ±0.8° C., ±0.7° C., ±0.6° C., ±0.5° C., ±0.4° C., ±0.3° C., ±0.2° C., or ±0.1° C. (See, e.g., Oguchi et al., pH Condition in temperature shift cultivation enhances cell longevity and specific hMab productivity in CHO culture, Cytotechnology. 52(3):199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub-physiological temperatures, Mol. Biotechnol. 39:69-77 (2008)).

For any given set-point, slight variations in temperature may occur. Typically, such variation may occur because heating and cooling elements are only activated after the temperature has deviated somewhat from the set-point. In that case, the set-point is X±Y and the heating or cooling element is activated when the temperature varies by ±Z° C., as appropriate. Typically, the permissible degree of deviation of the temperature from the set-point before heating or cooling elements are activated may be programmed in the process control system. Temperature may be controlled to the nearest ±0.5° C., ±0.4° C., ±0.3° C., ±0.2° C., or even ±0.1° C. by heating and cooling elements controlled by thermostats. Larger differentials in temperature may also be programmed, such as ±1.0° C., ±0.9° C., ±0.8° C., ±0.7° C., or ±0.6° C. The temperature may also be controlled by immersion of the culture vessel in a heating bath at a particular temperature. Conceivably, there is no variation from the set-point because the heating is applied continually. Another source of variation arises due to measurement error in the temperature of the cell culture supernatant. Typical thermometers used in cell culture equipment may have a variability of ±0.3° C., or ±0.2° C., or even ±0.1° C.

Where the temperature set-point is set at a value within the range X±Y° C., and the tolerance of the temperature is ±Z° C. (i.e. a heater or cooler is activated when the temperature deviates by ±Z° C., as appropriate) this can also be expressed as a set-point of (X−Y to X+Y)±Z° C. For each possible value of X, all combinations of ±Y° C. and ±Z° C., as indicated above, are envisaged.

“Culturing at” or “maintaining at” a set point of a particular desired pH value, means that the process control systems are set to that desired pH value, in other words that the set point of pH is the intended, target, pH. “Culturing at” or “maintaining at” a pH that is set at X±Y, means that the set point is at a value of from X+Y to X−Y pH units. For each of the preferred values of X, the set-point is at a value within the range pH X±0.05, ±0.04, ±0.03, ±0.02 or ±0.01.

Where the pH set-point is set at a value within the range X±Y, and the tolerance is ±Z, this can also be expressed as a set-point of (X−Y to X+Y)±Z. For each possible value of X, all combinations of ±Y and ±Z, as indicated above.

For any given pH set-point, slight variations in pH may occur. Typically, such variation can occur because means which control pH are only activated after the pH has deviated somewhat from the set-point. Typically, the pH is controlled to the nearest ±0.05, ±0.04, ±0.03, ±0.02, or ±0.01. Typically, sparging with CO₂ provides additional acid in cell culture. Liquid acids, e.g., HCl or H₃PO₄, are commonly used in microbial cultures. Sodium carbonate is usually the source of added alkali used to maintain pH for cell culture, and NH₄OH is often selected to add alkali in microbial culture.

The cell culture supernatant typically has a CO₂ concentration of 1 to 10% (v/v), for example, 4.0-9.0% (v/v), 5.5-8.5% (v/v), or about 6-8% (v/v). Conventionally, CO₂ concentration is higher than this due to the CO₂ produced by the cells not being removed from the cell culture supernatant. Maintaining the CO₂ concentration at 10% or lower is reported to increase the yield of recombinant protein expression; it helps the dCO2 (or pCO₂) to be kept low if the feed medium is degassed (for example by bubbling air through it) as well as the cell culture supernatant in the bioreactor being sparged. (See, Giovagnoli et al., Cell Culture Processes, US2009/0176269, US2016/0244506, U.S. Pat. No. 9,359,629, EP2235197, EP2574676). Degassing of natural/environmental aqueous samples before routing them to a microfluidic device (or “chip”) is also important, because outgassing within some sensor systems can generate bubbles that act as high resistance blockages to fluid flow into or within a microfluidic chip, which can divert flows and affect cell growth and sensing. Degassing is especially important when sensing in a background of photosynthetic culture medium, as these can contain levels of dissolved oxygen above 200%.

Ways of monitoring culture parameters of temperature, pH and CO₂ concentration are well known in this art and generally rely on probes that are inserted into the bioreactor, or included in loops through which the culture medium is circulated, or inserted into extracted samples of culture medium. Suitable monitoring equipment and appropriate alternatives are commercially available or can be constructed by the skilled artisan. Alternative gas analyzers are commercially available, such as RapidLab® 248 (Siemens) and others made by Nova® Biomedical, Radiometer America and Roche Diagnostics. Mass flow controllers can also be used to control gas and liquid additions in labs that are properly equipped. A suitable in-line dCO₂ (or pCO₂) sensor and its use are described in Pattison et al (2000) Biotechnol. Prog. 16:769-774. A suitable in-line pH sensor is Mettler Toledo InPro 3100/125/Pt100 (Mettler-Toledo Ingold, Inc., Bedford, Mass.). A suitable off-line system for measuring dCO₂ (or pCO₂), in addition to pH and pO₂ is the BioProfile pHOx (Nova Biomedical Corporation, Waltham Mass.). In this system, or dCO2 (or pCO₂) is measured by potentiometric electrodes within the range 3-200 mmHg with an imprecision resolution of 5%. The pH may be measured in this system at a temperature of 37° C., which is close to the temperature of the cell culture supernatant in the bioreactor. Ways of altering the specified parameter in order to keep it at the predefined level are also well known. For example, keeping the temperature constant usually involves heating or cooling the bioreactor or the feed medium (if it is a fed-batch or continuous process); keeping the pH constant usually involves choosing and supplying enough of an appropriate buffer (typically bicarbonate) and adding acid, such as hydrochloric acid, or alkali, such as sodium hydroxide, sodium carbonate or a mixture thereof, to the feed medium as necessary; and keeping the CO₂ concentration constant usually involves adjusting the sparging rate (see further below), or regulating the flow of CO₂ in the head space. It is possible that the calibration of an in-line pH probe may drift over time, such as over periods of days or weeks, during which the cells are cultured. In that event, it may be beneficial to reset the in-line probe by using measurements obtained from a recently calibrated off-line probe. A suitable off-line probe is the BioProfile pHOx (Nova Biomedical Corporation, Waltham Mass.).

Cell cultures need oxygen for the cells to grow. Normally, this is provided by forcing oxygen into the culture through injection ports. It is also necessary to remove the CO₂ that accumulates due to the respiration of the cells. This is achieved by “sparging,” i.e., passing a gas through the bioreactor in order to entrain and flush out the CO₂. Conventionally, this can also be done using oxygen. However, the inventors have found that it is advantageous to use air instead. It has been found that usually a conventional inert gas such as nitrogen is less effective at sparging CO₂ than using air. Given that air is about 20% (v/v) oxygen, one might have thought that five times as much air would be used. However, this has been found to be inadequate in large scale cultures, particularly in cultures at 2500 L scale. In a 2500 L bioreactor, 7 to 10 times as much air, preferably about 9 times as much air, is used. For example, under standard conditions, the 2500 L bioreactor is sparged with O₂ at a 10-μm bubble size at a rate of 0.02 VVH (volume O₂ per volume of culture per hour). The same 2500 L bioreactor used according to the method of the invention would be sparged with air at a 10-μm bubble size at a rate of 0.18 VVH.

Hence, the use of surprisingly high volumes of air has been found to provide adequate oxygen supply and to remove the unwanted CO₂. Flushing the bioreactor head space with air or pure oxygen is also a useful mechanism for removing excess CO₂.

In accordance with inventive method, the culturing of a plurality of E. coli cells can be any conventional type of culture, such as batch, fed-batch, intensified fed-batch, or continuous. Suitable continuous cultures included repeated batch, chemostat, turbidostat or perfusion culture. For purposes of the present invention, the desired scale of the recombinant expression will be dependent on the type of expression system and the quantity of different theoretical antibody variants to be studied. As noted herein, typically, 100 milligrams of total antibody protein will suffice, requiring only a batch cell culture of 20 mL to 500 mL; while larger scale culture batches or continuous cell culture methods can be employed, larger volumes are typically not cost-effective.

A batch culture starts with all the nutrients and cells that are needed, and the culture proceeds to completion, i.e. until the nutrients are exhausted or the culture is stopped for some reason.

A fed-batch culture is a batch process in the sense that it starts with the cells and nutrients but it is then fed with further nutrients in a controlled way. The fed-batch strategy is typically used in bio-industrial processes to reach a high cell density in the bioreactor. The feed solution is usually highly concentrated to avoid dilution of the bioreactor. The controlled addition of the nutrient directly affects the growth rate of the culture and allows one to avoid overflow metabolism (formation of metabolic by-products) and oxygen limitation (anaerobiosis). In most cases the growth-limiting nutrient is glucose which is fed to the culture as a highly concentrated glucose syrup (for example 500-850 g/L).

Different strategies can be used to control the growth in a fed-batch process. For example, any one of dissolved oxygen tension (DOT, pO2), oxygen uptake rate (OUR), glucose concentration, lactate concentration, pH and ammonia concentration can be used to monitor and control the culture growth by keeping that parameter constant. In a continuous culture, nutrients are added and, typically, medium is extracted in order to remove unwanted by-products and maintain a steady state. Suitable continuous culture methods are repeated batch culture, chemostat, turbidostat and perfusion culture.

In a repeated batch culture, also known as serial subculture, the cells are placed in a culture medium and grown to a desired cell density. To avoid the onset of a decline phase and cell death, the culture is diluted with complete growth medium before the cells reach their maximum concentration. The amount and frequency of dilution varies widely and depends on the growth characteristics of the cell line and convenience of the culture process. The process can be repeated as many times as required and, unless cells and medium are discarded at subculture, the volume of culture will increase stepwise as each dilution is made. The increasing volume may be handled by having a reactor of sufficient size to allow dilutions within the vessel or by dividing the diluted culture into several vessels. The rationale of this type of culture is to maintain the cells in an exponentially growing state. Serial subculture is characterized in that the volume of culture is always increasing stepwise, there can be multiple harvests, the cells continue to grow and the process can continue for as long as desired.

In the chemostat and turbidostat methods, the extracted medium contains cells. Thus, the cells remaining in the cell culture vessel must grow to maintain a steady state. In the chemostat method, the growth rate is typically controlled by controlling the dilution rate i.e. the rate at which fresh medium is added. The cells are cultured at a sub-maximal growth rate, which is achieved by restricting the dilution rate. The growth rate is typically high. In contrast, in the turbidostat method, the dilution rate is set to permit the maximum growth rate that the cells can achieve at the given operating conditions, such as pH and temperature.

In some embodiments the inventive recombinant Escherichia coli strains can be cultured in a bioreactor. The bioreactor can be a stainless steel, glass or plastic vessel of 0.01 (i.e., 10-mL) to 10000 (ten thousand) liters capacity, for example, 0.01, 0.015, 0.10, 0.25, 0.30, 0.35, 1, 2, 5, 10, 15, 20, 25, 30, 50, 75, 100, 500, 1000, 2500, 5000 or 8000 liters. The vessel is usually rigid but flexible plastic bags or bioreactor liners can be used. These flexible plastic bioreactor bags and liners are generally of the “single use” type. In an intensified fed-batch culture, culture vessels, reactors or chambers, of any one of various capacities are used to grow suspensions of cells. Each culture vessel can be connected via inlets to an array of porous tangential flow filters which in turn are connected via outlets back to the culture vessel. After cell growth, the suspensions of cells and growth medium are pumped through the array of porous tangential flow filters to concentrate the cell suspension. The cell suspension is recycled through the filters and culture vessel allowing a portion of the old growth medium to be removed. A supply of fresh sterile medium is added to the concentrated cell suspension to maintain a nominal volume in the culture vessel. (See, e.g., Zijlstra et al., Process for the culturing of cells, U.S. Pat. Nos. 8,119,368, 8,222,001, 8,440,458).

In a perfusion or continuous culture, the extracted medium is depleted of cells, because most of the cells are retained in the culture vessel, for example, by being retained on a membrane through which the extracted medium flows. However, typically such a membrane retains 100% of cells, and so a proportion are removed when the medium is extracted. Alternatively, sonic cell separation technology achieves separation of cells from the media matrix with high-frequency, resonant ultrasonic waves rather than using a physical barrier, unlike tangential-flow filtration (TFF) or alternating tangential flow filtration (ATF); the cells are held back using an acoustic field as the bioprocess fluid flows through an open channel. The use of acoustic waves allows differentiation of particles of equal size, and thus the technology can be used for the separation of particles from the nano- to macro-scales. (See, e.g., Challenger, C. A., An acoustic wave-based technology for cell harvesting applications may help enable continuous manufacturing, BioPharm International 30(9):30 (2017)). Regardless of the technology employed to separate the cells from the extracted medium, it may not be crucial to operate perfusion cultures at very high growth rates, as the majority of the cells are retained in the culture vessel.

Continuous cultures, particularly repeated batch, chemostat and turbidostat cultures, are typically operated at high growth rates. According to common practice, it is typical to seek to maintain growth rates at maximum, or close to maximum, in an effort to obtain maximum volumetric productivity. Volumetric productivity is measured in units of protein quantity or activity per volume of culture per time interval. Higher cell growth equates to a higher volume of culture being produced per day and so is conventionally considered to reflect a higher volumetric productivity. A suitable fully continuous process can have a perfusion bioreactor coupled to recombinant protein harvesting and protein purification steps, for example, a multi-column chromatography capture step, followed by flow-through virus inactivation, multi-column intermediate purification, a flow-through membrane adsorber polishing step, continuous virus filtration and a final ultrafiltration step operated in continuous mode. (See, e.g., Crowley et al., Process for cell culturing by continuous perfusion and alternating tangential flow, U.S. Pat. No. 8,206,981).

The cell density is commonly monitored in cell cultures. In principle, a high cell density would be considered to be desirable since, provided that the productivity per cell is maintained, this should lead to a higher productivity per bioreactor volume. However, increasing the cell density can actually be harmful to the cells, and the productivity per cell is reduced. There is therefore a need to monitor cell density. To date, in cell culture processes, this has been done by extracting samples of the culture and analyzing them under a microscope or using a cell counting device such as the CASY TT device sold by Scharfe System GmbH, Reutlingen, Germany. It can be advantageous to analyze the cell density by means of a suitable probe introduced into the bioreactor itself (or into a loop through which the medium and suspended cells are passed and then returned to the bioreactor). Such probes are available commercially from Aber Instruments, for example the Biomass Monitor 220, 210 220 or 230. The cells in the culture act as tiny capacitors under the influence of an electric field, since the non-conducting cell membrane allows a build-up of charge. The resulting capacitance can be measured; it is dependent upon the cell type and is directly proportional to the concentration of viable cells. A probe of 10 to 25 mm diameter uses two electrodes to apply a radio frequency field to the biomass and a second pair of electrodes to measure the resulting capacitance of the polarized cells. Electronic processing of the resulting signal produces an output which is an accurate measurement of the concentration of viable cells. The system is insensitive to cells with leaky membranes, the medium, gas bubbles and debris. Alternatively, cell viability can be measured by use of a vital dye (or vital stain) to stain small-aliquot samples of culture sampled periodically, and microscopically enumerated to determine viable cell count. For example Trypan blue is a vital dye commonly used for this purpose. Automated cell counters supplied by Beckman (e.g., Vi-Cell™ XR) and other companies are available. Examples include cell counting instruments made by other manufacturers, e.g., Nova Biomedical, Olympus, Thermo Fisher Scientific and Eppendorf. Cells can also be counted using flow cytometry or manually by using a hemocytometer.

Typically, source cell culture of the inventive E. coli sensing strains that will be used to spot a microfluidic device has an OD600 value of approximately 0.1 to 1.0, which equates to approximately 1.0×10⁸ cells/mL to 1.0×10⁹ cells/mL. (See, e.g., bionumbers.hms.harvard.edu/bionumber.aspx?id=100985&ver=14&trm=OD600+coli&org=) This can also be used as a typical high range for cell density in other liquid cell culture biosensors. Cell densities in the microfluidic device sensors presented here tend to be much higher than in liquid culture due to the close packing of cells in the microfluidic cell traps. If a bacterial cell is estimated to occupy approximately 5.0 μm³ of excluded volume on average (approximating a cell as an extruded square shape 5.0 μm long with a cross section 1.0 μm×1.0 μm), then close packing of this shape leads to a maximum cell density of 0.2×10¹² cells/mL. This close packing density estimate is a few orders of magnitude larger than what is typically expected for liquid culture cell density. This increased cell density allows for microfluidic devices to have a higher signal to noise ratio for a given volume relative to liquid cell culture sensors, since the density of sensing units (cells) is higher in microfluidic traps.

The term “buffer” or “buffered solution” refers to solutions which resist changes in pH by the action of its conjugate acid-base range. Examples of useful buffers that control pH at ranges of about pH 4 to about pH 8 include phosphate, bicarbonate, acetate, MES, citrate, Tris, bis-tris, histidine, arginine, succinate, citrate, glutamate, and lactate, or a combination of two or more of these, or other mineral acid or organic acid buffers. Salts containing sodium, ammonium, and potassium cations are often used in making a buffered solution.

A “domain” or “region” (used interchangeably herein) of a polynucleotide is any portion of the entire polynucleotide, up to and including the complete polynucleotide, but typically comprising less than the complete polynucleotide. A domain can, but need not, fold independently (e.g., DNA hairpin folding) of the rest of the polynucleotide chain and/or be correlated with a particular biological, biochemical, or structural function or location, such as a coding region or a regulatory region.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3.times. the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:
Algorithm: Needleman et al., 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with proteins of interest, such as activator or repressor proteins, include, but are not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. By methods known to the skilled artisan, proteins, can be “engineered” or modified for improved target affinity, selectivity, stability, and/or manufacturability before the coding sequence of the “engineered” protein is included in the expression cassette.

### Cloning DNA

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+ mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the polypeptide of interest, e.g., antibody sequences.

Sequencing of DNA is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74:5463-5467, which is incorporated herein by reference), or so-called “Next Generation Sequencing” (NGS) techniques. By comparing the sequence of the cloned nucleic acid with published sequences of genes and cDNAs, one of skill will readily be able to determine, depending on the region sequenced. One source of gene sequence information is the National Center for Biotechnology Information, National Library of Medicine, National Institutes of Health, Bethesda, MD. Embodiments of the present invention can involve NGS sequencing, as a preferred method for confirming the presence of all engineered DNA constructs prior to the transfection step(s). (See, e.g., Buermans, H. P. J., & den Dunnen, J. T., Next generation sequencing technology: Advances and applications, Biochimica et Biophysica Acta-Molecular Basis of Disease 1842(10): 1932-1941 (2014)).

Chemical synthesis of parts or the whole of a coding region containing codons reflecting desires protein changes can be cloned into an expression vector by either restriction digest and ligation of 5′ and 3′ ends of fragments or the entire open reading frame (ORF), containing nucleotide overhangs that are generated by restriction enzyme digestion and which are compatible to the destination vector. The fragments or inserts are typically ligated into the destination vector using a T4 ligase or other common enzyme. Other useful methods are similar to the above except that the cut site for the restriction enzyme is at location different from the recognition sequence. Alternatively, isothermal assembly (i.e., “Gibson Assembly”) can be employed, in which nucleotide overhangs are generated during synthesis of fragments or ORFs; digestion by exonucleases is employed. Alternatively, nucleotide overhangs can be ligated ex vivo by a ligase or polymerase or in vivo by intracellular processes.

Alternatively, homologous recombination can be employed, similar to isothermal assembly, except exonuclease activity of T4 DNA ligase can used on both insert and vector and ligation can be performed in vivo.

Another useful cloning method is the so-called “TOPO” method, in which a complete insert containing a 3′ adenosine overhang (generated by Taq polymerase) is present, and Topoisomerase I ligates the insert into a TOPO vector.

Another useful cloning method is degenerate or error-prone PCR exploiting degenerate primers and/or a thermally stable low-fidelity polymerase caused by the polymerase within certain reaction conditions. Fragments or inserts are then cloned into an expression vector.

The above are merely examples of known cloning techniques, and the skilled practitioner knows how to employ any other suitable cloning techniques.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components can include one or more of the following: a signal sequence (that may, for example, direct secretion of the expressed protein by the recombinant host cells); an origin of replication, one or more selection marker and/or reporter protein encoding genes (that may, for example, encode a fluorescent protein, such as a green fluorescent protein (GFP), an enhanced green fluorescent protein (EGFP), a red-shifted green fluorescent protein (rs-GFP), a yellow fluorescent protein (YFP), a red fluorescent protein (RFP), a cyan fluorescent protein (e.g., CyOFP1), mini Singlet Oxygen Generator (miniSOG), a luminescent protein (e.g., luciferase), or the like, or may confer antibiotic or other drug resistance, or complement auxotrophic deficiencies of the host cells or supply critical nutrients not available in the medium, e.g., dihydrofolate reductase or glutamine synthetase selection markers), an enhancer element, a promoter, and a transcription termination sequence, all of which are well known in the art.

By way of further illustration, the following embodiments of the present invention are enumerated:

By way of further illustration, the following numbered embodiments are encompassed by the present invention:

Embodiment 1: A panel of recombinant Escherichia coli strains for monitoring a plurality of analytes of interest in an aqueous sample, comprising:
a set of two or more recombinant Escherichia coli strains that, in a defined aqueous culture medium, constitutively express one or more antibiotic resistance genes providing resistance to one or more antibiotic agents, wherein the one or more antibiotic agents, separately, or in combination, are characterized by both antibacterial and antifungal activity in the aqueous culture medium, each strain comprising a stable recombinant expression system comprising an expression cassette comprising an analyte-sensitive promoter that specifically responds to at least one of the plurality of analytes of interest, resulting in a modification of expression from the promoter, said promoter being capable of operating under aerobic physiological conditions and being operably linked to a gene encoding a detectable marker; and
wherein the set of two or more Escherichia coli strains is capable of expressing the detectable marker in the presence of at least one of the plurality of analytes of interest in a continuous series of aqueous samples diluted with fresh defined culture medium, and the set of two or more Escherichia coli strains, wherein the identity of each Escherichia coli strain can be distinguished.

Embodiment 2: The panel according to Embodiment 1, wherein the one or more antibiotic resistance genes comprise a hygromycin B resistance gene.

Embodiment 3: The panel according to any one of Embodiments 1-2, wherein the one or more antibiotic resistance genes comprise:
(i) a first antibiotic resistance gene providing resistance to an antibiotic agent characterized by antibacterial activity; and
(ii) a second antibiotic resistance gene providing resistance to an antibiotic agent characterized by antifungal activity.

Embodiment 4: The panel according to Embodiment 3, wherein the first antibiotic resistance gene provides resistance to penicillin, ampicillin, kanamycin, zeocin, neomycin, polymyxin B, colistin, bacitracin, streptomycin, or spectinomycin.

Embodiment 5: The panel according to any one of Embodiments 3-4, wherein the second antibiotic resistance gene provides resistance to clotrimazole, econazole, miconazole, terbinafine, fluconazole, ketoconazole, nystatin, or amphotericin.

Embodiment 6: The panel according to any one of Embodiments 1-5, wherein the detectable marker is an optically detectable marker.

Embodiment 7: The panel according to any one of Embodiments 1-6, wherein the detectable marker is a fluorescent protein or a luminescent protein.

Embodiment 8: The panel according to any one of Embodiments 1-7, wherein the plurality of analytes of interest comprises one or more ionic species selected from the group consisting of ammonium, nitrate, nitrite, phosphate, manganese dication, nickel dication, and an iron cation, or a combination of any one of these members.

Embodiment 9: The panel according to any one of Embodiments 1-8, wherein the plurality of analytes of interest comprises ammonium and the defined medium does not contain added glutamine.

Embodiment 10: The panel according to any one of Embodiments 1-9, wherein each Escherichia coli strain in the panel expresses the detectable marker in the presence of the at least one of the plurality of analytes of interest, with a limit of detection for the analyte in the aqueous sample being in a range between 1 to 1000 ppb.

Embodiment 11: The panel according to any one of Embodiments 1-10, wherein the limit of detection for the analyte in the aqueous sample is in a range between 1 to 500 ppb.

Embodiment 12: The panel according to any one of Embodiments 1-11, wherein the limit of detection for the analyte in the aqueous sample is in a range between 5 to 250 ppb.

Embodiment 13: The panel according to any one of Embodiments 1-12, wherein the stable recombinant expression system comprises an expression cassette that comprises a variable sensing promoter region sensitive to:
ammonium, wherein variable sensing promoter region comprises a nucleotide sequence selected from the group consisting of SEQ ID NO:50 and SEQ ID NO:55; or
nitrate, wherein variable sensing promoter region comprises a nucleotide sequence selected from the group consisting of SEQ ID NO:59 and SEQ ID NO:63; or
nitrite, wherein variable sensing promoter region comprises a nucleotide sequence selected from the group consisting of SEQ ID NO:69 and SEQ ID NO:74; or
phosphate, wherein variable sensing promoter region comprises a nucleotide sequence selected from the group consisting of SEQ ID NO:78 and SEQ ID NO:83; or
iron, wherein variable sensing promoter region comprises a nucleotide sequence selected from the group consisting of SEQ ID NO:85, SEQ ID NO:86, SEQ ID NO:87, and SEQ ID NO:90; or
manganese (II), wherein variable sensing promoter region comprises a nucleotide sequence selected from the group consisting of SEQ ID NO:93 and SEQ ID NO:95; or
nickel (II), wherein variable sensing promoter region comprises the nucleotide sequence of SEQ ID NO:101.

Embodiment 14: The panel according to any one of Embodiments 1-13, wherein the stable recombinant expression system comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:36, SEQ ID NO:37, SEQ ID NO:38, and SEQ ID NO:96.

Embodiment 15: The panel according to any one of Embodiments 1-14, wherein the set of two or more Escherichia coli strains is arrayed in a microfluidic device.

Embodiment 16: The panel according to any one of Embodiments 1-15, wherein the set of two or more Escherichia coli strains is revivable from a dehydrated state.

Embodiment 17: The panel according to any one of Embodiments 1-16, wherein the set of two or more Escherichia coli strains is in a lyophilized or air-dried state.

Embodiment 18: The panel according to any one of Embodiments 1-17, wherein after being in a dehydrated state for at least 30-60 days, the set of two or more Escherichia coli strains is capable of being revived under aqueous physiological conditions and is capable of expressing the optically detectable marker in the presence of at least one of the plurality of analytes of interest.

Embodiment 19: A method for monitoring a plurality of analytes of interest, comprising:
mixing a continuous series of aqueous samples with a fresh defined culture medium in a defined dilution ratio to obtain a series of diluted samples;
contacting the continuous series of diluted samples with the panel comprising a set of two or more recombinant Escherichia coli strains according to any one of Embodiments 1-18, under aerobic physiological conditions;
monitoring for the expression of the detectable marker by the set of two or more Escherichia coli strains arrayed in locations, wherein the identity of each Escherichia coli strain can be distinguished; and
correlating any expression of the detectable marker by the set of two or more Escherichia coli strains in subpart (c), with the presence of at least one of the plurality of analytes of interest in the aqueous sample with a limit of detection in a concentration range of 1-1000 ppb.

Embodiment 20: The method according to Embodiment 19, wherein the limit of detection is in the concentration range of 1-500 ppb.

Embodiment 21: The method according to any one of Embodiments 19-20, wherein the limit of detection is in the concentration range of 5-250 ppb.

Embodiment 22: The method according to any one of Embodiments 19-21, wherein the plurality of analytes of interest comprises one or more ionic species selected from the group consisting of ammonium, nitrate, nitrite, phosphate, manganese dication, nickel dication, and an iron cation, or a combination of any one of these members.

Embodiment 23: The method according to any one of Embodiments 19-22, wherein the plurality of analytes of interest comprises ammonium and the defined culture medium does not contain added glutamine.

Embodiment 24: The method according to any one of Embodiments 19-23, wherein the detectable marker is an optically detectable marker.

Embodiment 25: The method according to any one of Embodiments 19-24, wherein the detectable marker is a fluorescent protein or a luminescent protein.

Embodiment 26: The method according to any one of Embodiments 19-25, wherein the set of two or more Escherichia coli strains is arrayed in a microfluidic device.

Embodiment 27: The method according to any one of Embodiments 19-26, wherein the continuous series of aqueous samples of source water is obtained sequentially from the source water over a time period extending from 1 minute up to 60 days.

The following working examples are illustrative and not to be construed in any way as limiting the scope of the invention.

### EXAMPLES

### Example 1. Generation of E. coli Strains

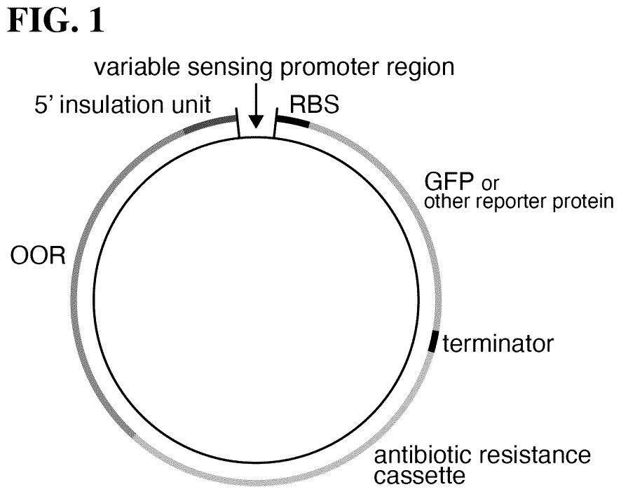

Sensor strain designs were based on promoters native to Escherichia coli and other bacteria that were identified to be sensitive to the analytes of interest. We designed 152 plasmid variants based on 23 promoter elements. Plasmid variants of a single promoter element typically included variations on the promoter sequence, ribosomal binding site (RBS), or other regulatory elements or genes. Of this set, we constructed 87 plasmid variants, either by de novo DNA synthesis, or by using standard cloning methods such as Gibson assembly. (D. G. Gibson, L. Young, R. Y. Chuang, J. C. Venter, C. A. Hutchison, and H. O. Smith, “Enzymatic assembly of DNA molecules up to several hundred kilobases,” Nat Methods, vol. 6, no. 5, pp. 343-345, (2009), doi: 10.1038/nmeth.1318). When native gene sequences were required for amplification, E. coli K-12 MG1655 was used. Plasmid variants share the general architecture shown in FIG. 1.

The core sensing unit of each plasmid was a variable sensing promoter region containing the genetic elements necessary to specifically sense each analyte under aerobic conditions. This unit typically contains an analyte-inducible transcriptional promoter, genes for any necessary regulatory proteins to regulate the analyte-inducible promoter, and the promoter elements necessary to express those transcription factors. The variable sensing promoter region is typically placed upstream of a ribosome binding site (RBS), reporter protein, and transcriptional terminator, such that the analyte-inducible promoter drives reporter expression. For convenience, the following T0 terminator sequence (SEQ ID NO:15) from Escherichia phage Lambda, was employed, but other terminator sequences are also useful: CTTGGACTCCTGTTGATAGATCCAGTAATGACCTCAGAACTCCATCTGGATTTGTT CAGAACGCTCGGTTGCCGCCGGGCGTTTTTTATTGGTGAGAATCCA//SEQ ID NO:15. (See, R. Wu and E. Taylor, “Nucleotide Sequence Analysis of DNA: II. Complete Nucleotide Sequence of the Cohesive Ends of Bacteriophage Lambda DNA,” J. Mol. Biol., vol. 57, no. 3, pp. 491-511 (1971), doi.org/10.1016/0022-2836(71)90105-7).

However, in cases where it is valuable to preserve the gene order presented in the native operon, the RBS-GFP sequence may be inserted within the variable sensing promoter region, as illustrated in FIG. 1. A 5′ insulation unit was placed upstream of the variable sensing promoter region to insulate it from potential regulation by 5′ elements. In the embodiments described herein the following insulator element sequence (SEQ ID NO:12) was employed, but other sequences are also useful: GAATTCAAAAGATCTTAAGTAAGTAAGAGTATACGTATATCGGCTAATAACGTAT TAAGGCGCTTCGGCGCCTTTTTTTATGGGGGTATTTTCATCCCAATCCACACGTCC AACGCACAGCAAACACCACGTCGACCCTATCAGCTGCGTGCTTTCTATGAGTCGT TGCTGCATAAC//SEQ ID NO:12. (See, O. Wright, M. Delmans, G. B. Stan, and T. Ellis, “GeneGuard: A modular plasmid system designed for biosafety,” ACS Synth Biol, vol. 4, no. 3, pp. 307-316 (2015), doi: 10.1021/sb500234s).

All the sensing plasmids contained an origin of replication (OOR) DNA sequence for propagation within bacterial cells. Our engineered sensing strains typically contained the medium copy number OOR, p15A (SEQ ID NO:17): GATATATTCCGCTTCCTCGCTCACTGACTCGCTACGCTCGGTCGTTCGACTGCGGC GAGCGGAAATGGCTTACGAACGGGGCGGAGATTTCCTGGAAGATGCCAGGAAGA TACTTAACAGGGAAGTGAGAGGGCCGCGGCAAAGCCGTTTTTCCATAGGCTCCG CCCCCCTGACAAGCATCACGAAATCTGACGCTCAAATCAGTGGTGGCGAAACCC GACAGGACTATAAAGATACCAGGCGTTTCCCCCTGGCGGCTCCCTCGTGCGCTCT CCTGTTCCTGCCTTTCGGTTTACCGGTGTCATTCCGCTGTTATGGCCGCGTTTGTC TCATTCCACGCCTGACACTCAGTTCCGGGTAGGCAGTTCGCTCCAAGCTGGACTG TATGCACGAACCCCCCGTTCAGTCCGACCGCTGCGCCTTATCCGGTAACTATCGT CTTGAGTCCAACCCGGAAAGACATGCAAAAGCACCACTGGCAGCAGCCACTGGT AATTGATTTAGAGGAGTTAGTCTTGAAGTCATGCGCCGGTTAAGGCTAAACTGAA AGGACAAGTTTTGGTGACTGCGCTCCTCCAAGCCAGTTACCTCGGTTCAAAGAGT TGGTAGCTCAGAGAACCTTCGAAAAACCGCCCTGCAAGGCGGTTTTTTCGTTTTC AGAGCAAGAGATTACGCGCAGACCAAAACGATCTCAAGAAGATCATCTTATTAA TCAGATAAAATATTTCTAGATTTCAGTGCAATTTATCTCTTCAAATGTAGCACCTG AAGTCAGCCCCATACGATATAAGTTGTT//SEQ ID NO:17. (See, G. Selzer, T. Som, T. Itoh, and J.-I. Tomizawa, “The Origin of Replication of Plasmid p15A and Comparative Studies on the Nucleotide Sequences around the Origin of Related Plasmids,” Cell 32(1):P119-129 (1983), doi.org/10.1016/0092-8674(83)90502-0).

However, plasmid copy number can be tuned by swapping in alternative OORs instead of p15A, including (but not limited to) pMB1, pBR322, ColE1, and pSC101. Tuning plasmid copy number typically modifies promoter regulation by altering the ratios of genome- and plasmid-based promoter binding sites and regulatory elements.

All the sensing plasmids described herein also contained a selectable marker for maintaining presence in the host strain by conferring some survival advantage in the cellular environment. For bacteria, this is typically an antibiotic resistance gene. Any number of E. coli expressible antibiotic resistance genes can be used, but we typically employed the hygromycin B phosphotransferase (hph) resistance gene (SEQ ID NO:16) to confer resistance to the hygromycin B antibiotic, which was included in the culture medium because of its dual antibacterial and antifungal activity: CCTTTCGACCTGCAGCCTGTTGACAATTAATCATCGGCATAGTATATCGGCATAG TATAATACGACAAGGTGAGGAACTAAACCATGAAAAAGCCTGAACTCACCGCGA CGTCTGTCGAGAAGTTTCTGATCGAAAAGTTCGACAGCGTCTCCGACCTGATGCA GCTCTCGGAGGGCGAAGAATCTCGTGCTTTCAGCTTCGATGTAGGAGGGCGTGG ATATGTCCTGCGGGTAAATAGCTGCGCCGATGGTTTCTACAAAGATCGTTATGTT TATCGGCACTTTGCATCGGCCGCGCTCCCGATTCCGGAAGTGCTTGACATTGGGG AATTCAGCGAGAGCCTGACCTATTGCATCTCCCGCCGTGCACAGGGTGTCACGTT GCAAGACCTGCCTGAAACCGAACTGCCCGCTGTTCTGCAGCCGGTCGCGGAGGC CATGGATGCGATCGCTGCGGCCGATCTTAGCCAGACGAGCGGGTTCGGCCCATTC GGACCGCAAGGAATCGGTCAATACACTACATGGCGTGATTTCATATGCGCGATTG CTGATCCCCATGTGTATCACTGGCAAACTGTGATGGACGACACCGTCAGTGCGTC CGTCGCGCAGGCTCTCGATGAGCTGATGCTTTGGGCCGAGGACTGCCCCGAAGTC CGGCACCTCGTGCACGCGGATTTCGGCTCCAACAATGTCCTGACGGACAATGGCC GCATAACAGCGGTCATTGACTGGAGCGAGGCGATGTTCGGGGATTCCCAATACG AGGTCGCCAACATCTTCTTCTGGAGGCCGTGGTTGGCTTGTATGGAGCAGCAGAC GCGCTACTTCGAGCGGAGGCATCCGGAGCTTGCAGGATCGCCGCGGCTCCGGGC GTATATGCTCCGCATTGGTCTTGACCAACTCTATCAGAGCTTGGTTGACGGCAAT TTCGATGATGCAGCTTGGGCGCAGGGTCGATGCGACGCAATCGTCCGATCCGGA GCCGGGACTGTCGGGCGTACACAAATCGCCCGCAGAAGCGCGGCCGTCTGGACC GATGGCTGTGTAGAAGTACTCGCCGATAGTGGAAACCGACGCCCCGGATGAGGG GATCAATTCTCTAGAGCTCGCTGATCAGAAGTTCCT//SEQ ID NO:16. (See, H. Tas, C. T. Nguyen, R. Patel, N. H. Kim, and T. E. Kuhlman, “An integrated system for precise genome modification in Escherichia coli,” PLoS One, vol. 10, no. 9 (2015), doi: 10.1371/journal.pone.0136963).

The hygromycin B selection scheme was preferred, because it uses a broad-spectrum antibiotic that acts against both bacteria and fungi, thereby providing greater protection against contaminating species that might infiltrate a microfluidic sensing device in both lab- and field-deployment scenarios.

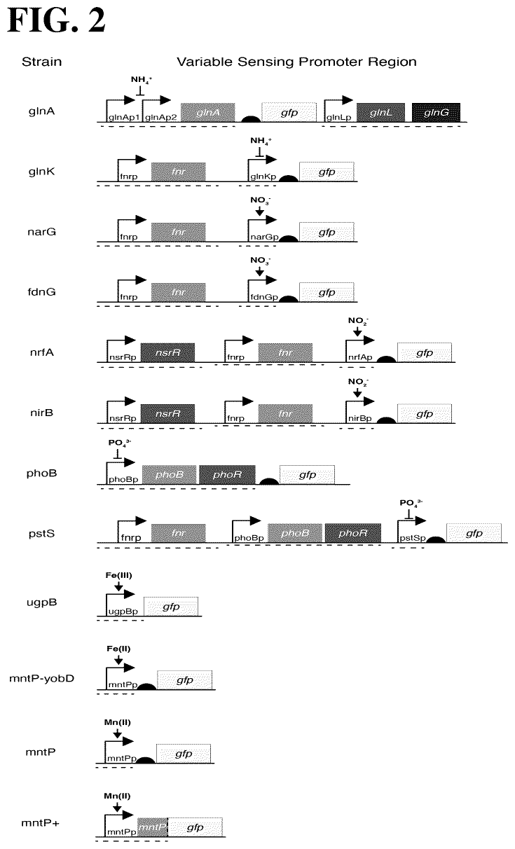

FIG. 2 schematically illustrates some embodiments of the variable promoter region structures for some high-performing sensing strains, wherein the strains are shown in FIG. 2 designated by the promoter that was used. While any RBS that effectively initiates translation can be used, our engineered constructs commonly use the RBS sequence reported by Lutz and Bujard, Ibid. Click or tap here to enter text. (SEQ ID NO:13) or the native RBS found with the inserted promoter in genomic DNA. For convenience, embodiments of the sensing plasmids described herein contained as the reporter protein fast-folding GFP (sfgfp; SEQ ID NO:14): ATGCGTAAAGGCGAAGAGCTGTTCACTGGTGTCGTCCCTATTCTGGTGGAACTGG ATGGTGATGTCAACGGTCATAAGTTTTCCGTGCGTGGCGAGGGTGAAGGTGACG CAACTAATGGTAAACTGACGCTGAAGTTCATCTGTACTACTGGTAAACTGCCGGT ACCTTGGCCGACTCTGGTAACGACGCTGACTTATGGTGTTCAGTGCTTTGCTCGTT ATCCGGACCATATGAAGCAGCATGACTTCTTCAAGTCCGCCATGCCGGAAGGCTA TGTGCAGGAACGCACGATTTCCTTTAAGGATGACGGCACGTACAAAACGCGTGC GGAAGTGAAATTTGAAGGCGATACCCTGGTAAACCGCATTGAGCTGAAAGGCAT TGACTTTAAAGAAGACGGCAATATCCTGGGCCATAAGCTGGAATACAATTTTAAC AGCCACAATGTTTACATCACCGCCGATAAACAAAAAAATGGCATTAAAGCGAAT TTTAAAATTCGCCACAACGTGGAGGATGGCAGCGTGCAGCTGGCTGATCACTACC AGCAAAACACTCCAATCGGTGATGGTCCTGTTCTGCTGCCAGACAATCACTATCT GAGCACGCAAAGCGTTCTGTCTAAAGATCCGAACGAGAAACGCGATCATATGGT TCTGCTGGAGTTCGTAACCGCAGCGGGCATCACGCATGGTATGGATGAACTGTAC AAATGA//SEQ ID NO:14. (See, J. D. Pédelacq, S. Cabantous, T. Tran, T. C. Terwilliger, and G. S. Waldo, “Engineering and characterization of a superfolder green fluorescent protein,” Nat Biotechnol, vol. 24, no. 1, pp. 79-88, January 2006, doi: 10.1038/nbt1172).

However, alternative reporter protein(s) can be used as detectable marker(s), for example, fluorescent proteins (e.g. T-Sapphire, mAmetrine, mAmetrine1.2, YFP, LSSmOrange, LSSmKate2, mKate2, tdKatushka2, E2-Crimson, mCardinal, mCardinal2), luminescent systems (e.g., luxCDABE), pigments, or enzymatic reporters (e.g., horseradish peroxidase).

In E. coli, the fnr(fumarate and nitrate reduction) gene regulates the transcription of hundreds of genes to mediate the transition between anaerobic and aerobic growth conditions, typically activating genes involved in anaerobic metabolism and repressing genes involved in aerobic metabolism. Although the cellular concentration of the FNR transcription factor is similar under both anaerobic and aerobic growth, its activity is directly regulated by oxygen, which inactivates FNR via oxidation of a [4Fe-4S]²⁺ cluster and disassembly of the FNR dimer. (See, Lin, H Y et al., “Activation of yeaR-yoaG operon transcription by the nitrate-responsive regulator NarL is independent of oxygen-responsive regulator Fnr in Escherichia coli K-12,” J Bacteriol, vol. 189, no. 21, pp. 7539-7548 (2007), doi: 10.1128/JB.00953-07; C. Constantinidou, C. et al., “A reassessment of the FNR regulon and transcriptomic analysis of the effects of nitrate, nitrite, NarXL, and NarQP as Escherichia coli K12 adapts from aerobic to anaerobic growth,” Journal of Biological Chemistry, vol. 281, no. 8, pp. 4802-4815 (2006), doi: 10.1074/jbc.M512312200).

Because the sensing strains of the present invention are cultured aerobically, frequently within a gas-permeable microfluidic device, native fnr represses many genes involved in nitrogen regulation, thereby reducing sensitivity to ammonium, nitrate and nitrate. To overcome this challenge, we expressed an fnr mutant in several plasmid variants that activate nitrogen regulation genes in the presence of oxygen. Specifically, we included a plasmid-based copy of fnr containing a substitution of Leucine 28 with Histidine (L28H) (designated herein, the “fnr module,” having the nucleic acid sequence of SEQ ID NO:20) to stabilize its [4Fe-4S]²⁺ cluster and preserve its activity in the presence of oxygen: TCGTGAATATTTTGCCGGTAATCTTAGCATTATTGATACGCTTCCCACTGCTACGG GGGGGACGCCATTTCAGCGCGAAGTCTGGAAAACACTACGCACTATCCCCTGCG GGCAGGTAATGCATTACGGCCAACTGGCTGAGCAATTGGGCCGTCCTGGCGCGG CGCGTGCCGTTGGTGCGGCAAACGGATCGAATCCCATCAGCATCGTCGTACCTTG CCATCGGGTTATTGGCCGAAACGGCACCATGACCGGATATGCAGGCGGAGTTCA GCGAAAAGAGTGGTTATTGCGCCATGAAGGTTATCTTTTGCTGTAAACATTAAAC AATTTGTGCCAGCTTGTTCACACTTTTATGTAAAGTTACCCTTAACAACTTAAGGG TTTTCAAATAGATAGACATATATTTACATCTAATATCGGAATTCTCTGCTGTTAAG GTTTGCTTAGACTTACTTGCTCCCTAAAAAGATGTTAAAATTGACAAATATCAAT TACGGCTTGAGCAGACCTATGATCCCGGAAAAGCGAATTATACGGCGCATTCAG TCTGGCGGTTGTGCTATCCATTGCCAGGATTGCAGCATCAGCCAGCATTGCATCC CGTTCACACTCAACGAACATGAGCTTGATCAGCTTGATAATATCATTGAGCGGAA GAAGCCTATTCAGAAAGGCCAGACGCTGTTTAAGGCTGGTGATGAACTTAAATC GCTTTATGCCATCCGCTCCGGTACGATTAAAAGTTATACCATCACTGAGCAAGGC GACGAGCAAATCACTGGTTTCCATTTAGCAGGCGACCTGGTGGGATTTGACGCCA TCGGCAGCGGCCATCACCCGAGCTTCGCGCAGGCGCTGGAAACCTCGATGGTAT GTGAAATCCCGTTCGAAACGCTGGACGATTTGTCCGGTAAAATGCCGAATCTGCG TCAGCAGATGATGCGTCTGATGAGCGGTGAAATCAAAGGCGATCAGGACATGAT CCTGCTGTTGTCGAAGAAAAATGCCGAGGAACGTCTGGCTGCATTCATCTACAAC CTGTCCCGTCGTTTTGCCCAACGCGGCTTCTCCCCTCGTGAATTCCGCCTGACGAT GACTCGTGGCGATATCGGTAACTATCTGGGCCTGACGGTAGAAACCATCAGCCGT CTGCTGGGTCGCTTCCAGAAAAGCGGCATGCTGGCAGTCAAAGGTAAATACATC ACCATCGAAAATAACGATGCGCTGGCCCAGCTTGCTGGTCATACGCGTAACGTTG CCTGATTTTTCCGCATAACTCACTATCCTTCTGTCATATCATTAAATTTTTCTGATT TATTGATCTGGCAGAAGGTTCATCACTGTTTCATTCACCAGATATGGGTTAATCTT TTAATTACAAACTGCGTTGACAGTTGTTGTAAGGAGACCCTGTATGGCTATGTAT CAGAACATGCTCGTTGTTATCGATCCTAACCAGGACGACCAACCAGCATTGCGGC GAGCTGTTTATTTACATCAACGGATTGGTGGCAAAATTAAAGCCTTTTTGCCGAT CTATGACTTCTCATACGAAATGACCACCCTGCTCTCCCCGGACGAACGTACCGCT ATGCGTCAGGGCGTCATCAGCCAGCGTACAGCCTGGATC//SEQ ID NO:20. (See, P. J. Kiley and W. S. Reznikoff, “Fnr mutants that activate gene expression in the presence of oxygen,” J Bacteriol, vol. 173, no. 1, pp. 16-22 (1991), doi: 10.1128/jb.173.1.16-22.1991; D. M. Bates et al., “Substitution of leucine 28 with histidine in the Escherichia coli transcription factor FNR results in increased stability of the [4Fe-4S]2+ cluster to oxygen,” Journal of Biological Chemistry, vol. 275, no. 9, pp. 6234-6240 (2000), doi: 10.1074/jbc.275.9.6234; H. Corker and R. K. Poole, “Nitric oxide formation by Escherichia coli. Dependence on nitrite reductase, the NO-sensing regulator Fnr, and flavohemoglobin Hmp,” Journal of Biological Chemistry, vol. 278, no. 34, pp. 31584-31592 (2003), doi: 10.1074/jbc.M303282200; K. M. DeAngelis, P. Ji, M. K. Firestone, and S. E. Lindow, “Two Novel Bacterial Biosensors for Detection of Nitrate Availability in the Rhizosphere,” Appl Environ Microbiol, vol. 71, no. 12, pp. 8537-8547 (2005), doi: 10.1128/AEM.71.12.8537).

We terminated fnr transcription with the Tl terminator sequence from E. coli (SEQ ID NO:21; A. Orosz, I. Boros, and P. Venetianer, “Analysis of the complex transcription termination region of the Escherichia coli rrnB gene,” Eur. J. Biochem. 201, 653-659 (1991)): TGCTAGAGGCATCAAATAAAACGAAAGGCTCAGTCGAAAGACTGGGCCTTTCGT TTTATCTGTTGTTTGTCGGTGAACGCTCTCCTGAGTAGGACAAATCCGCCG//SEQ ID NO:21. However, other terminator sequences can be used instead.

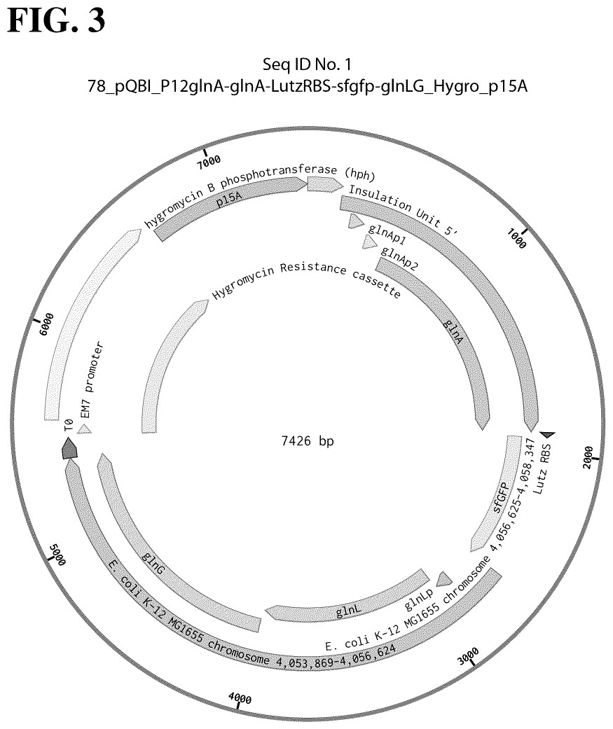

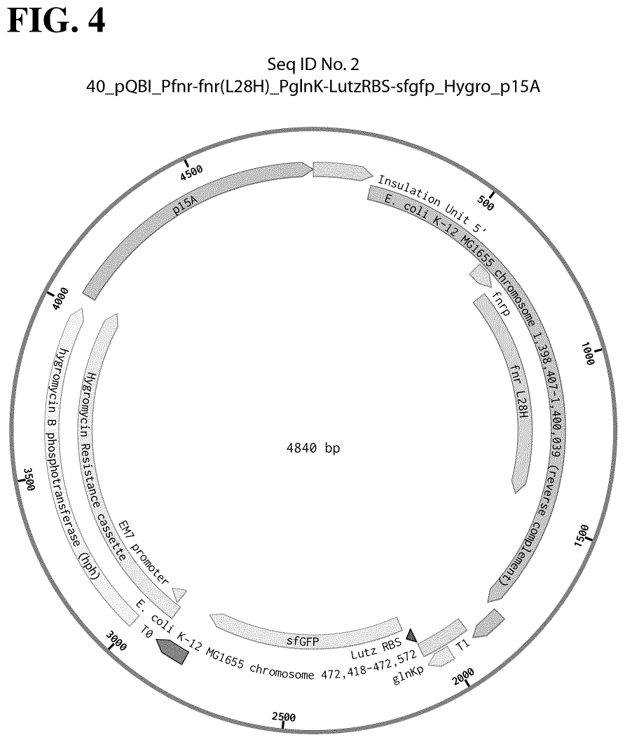

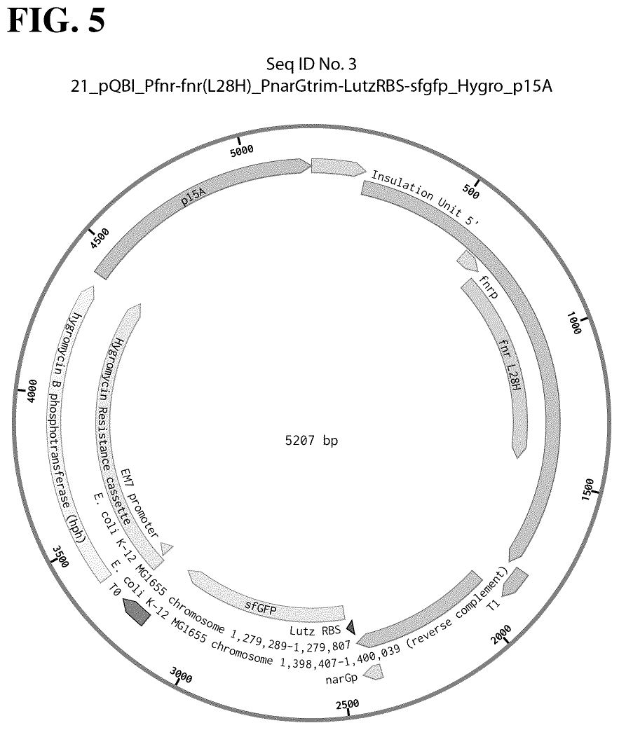

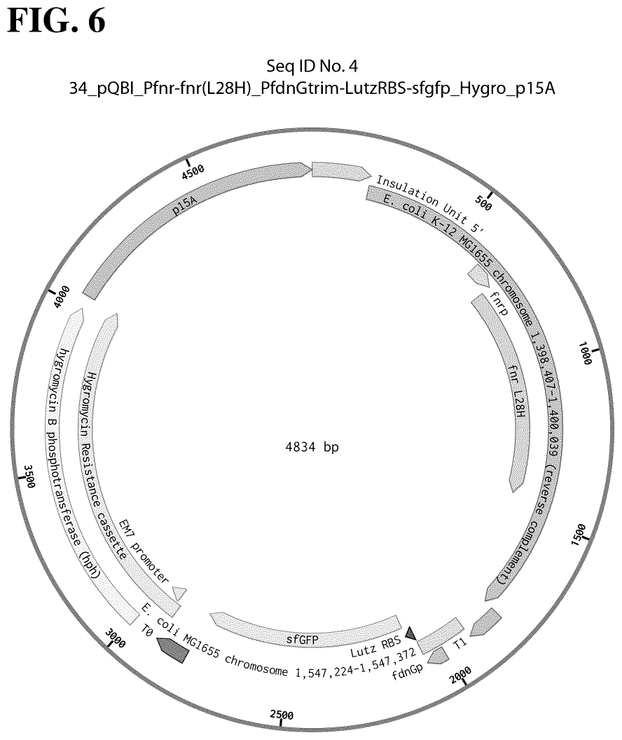

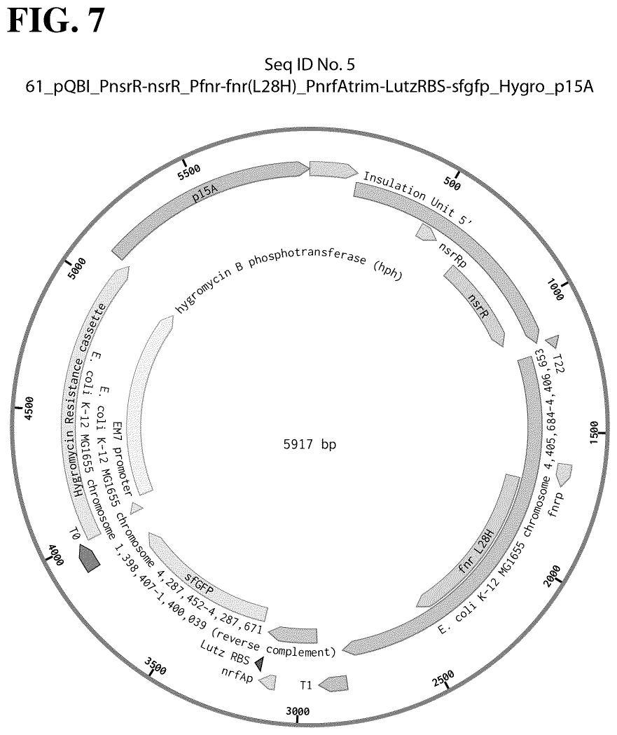

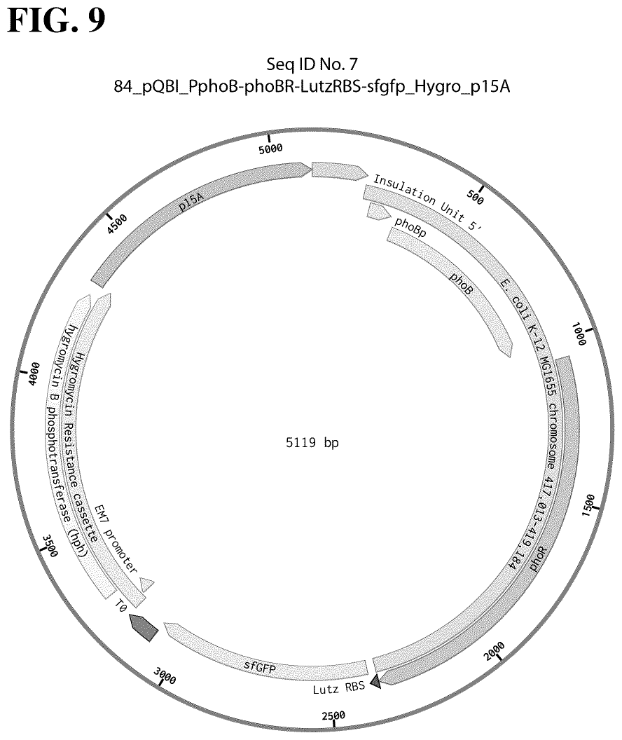

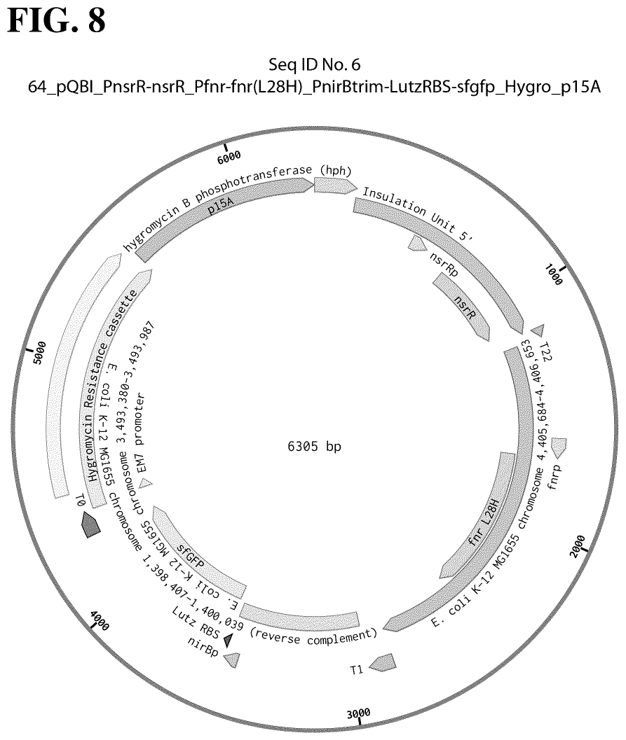

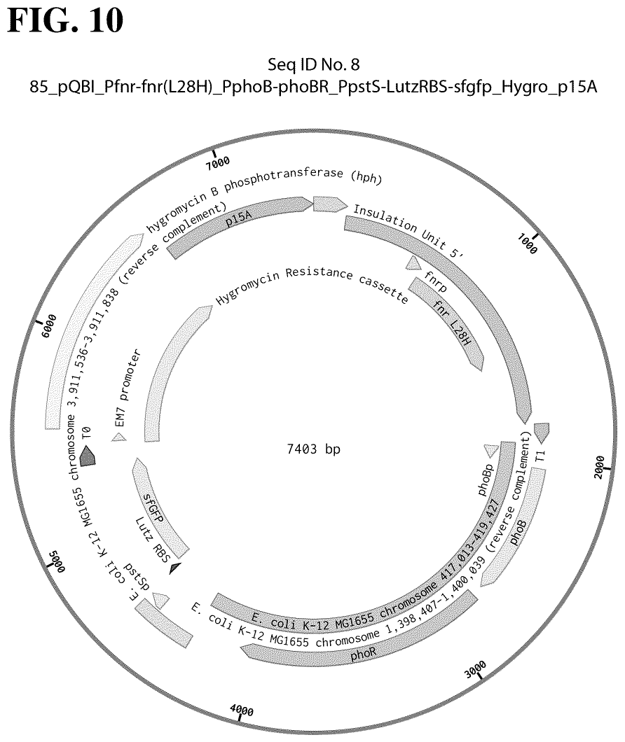

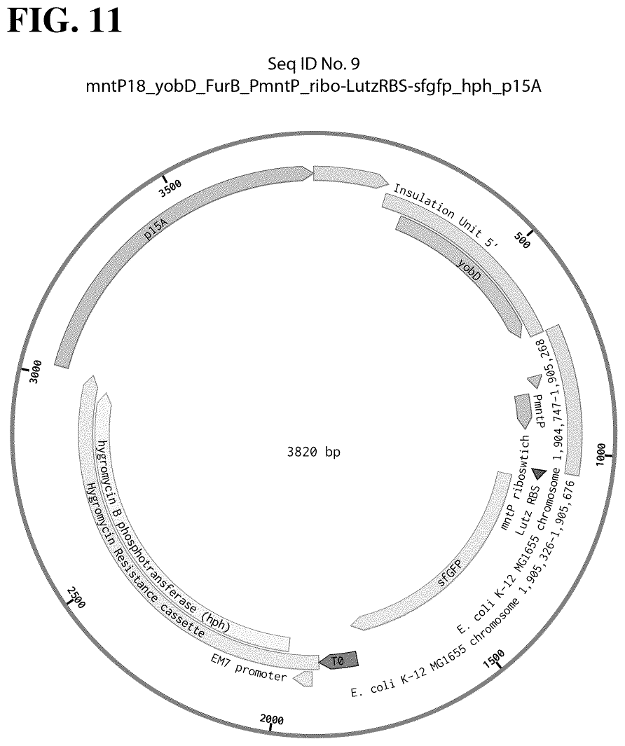

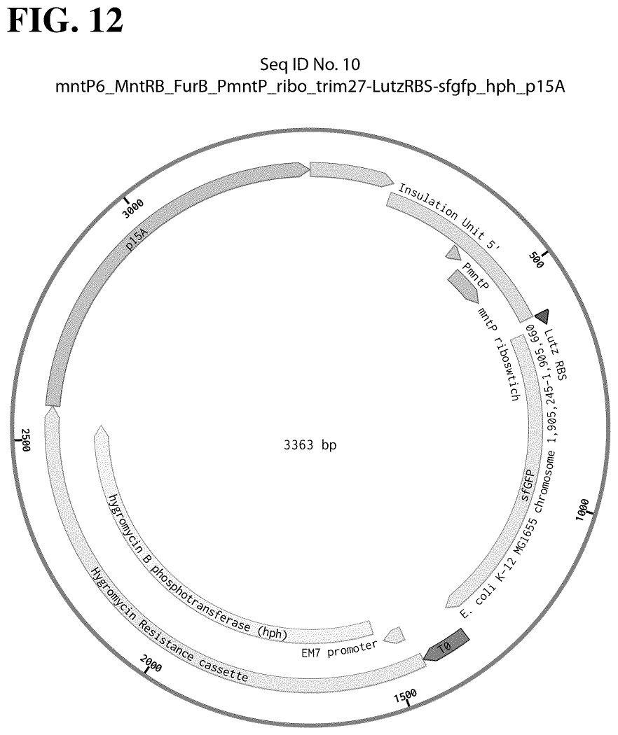

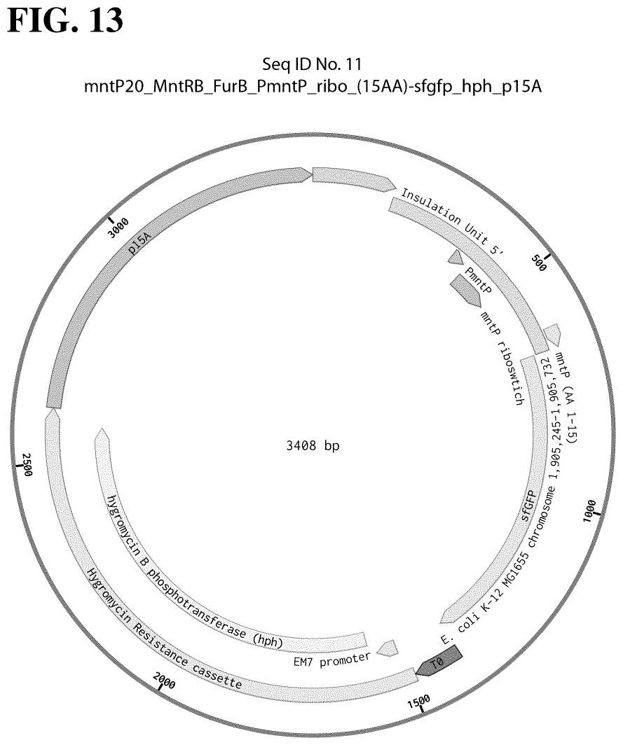

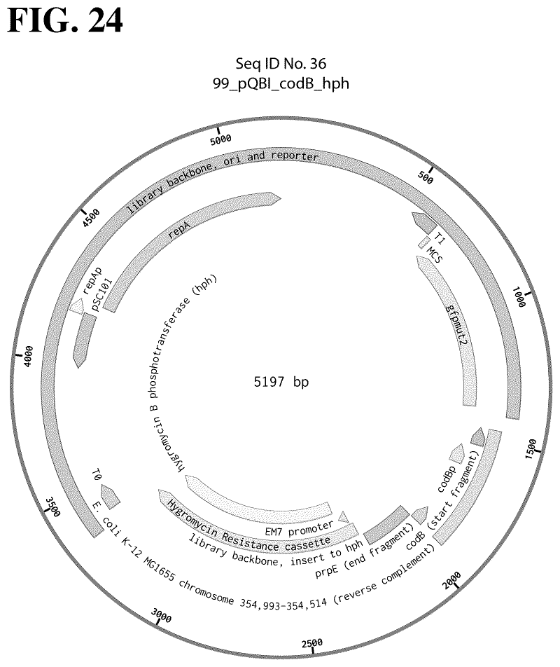

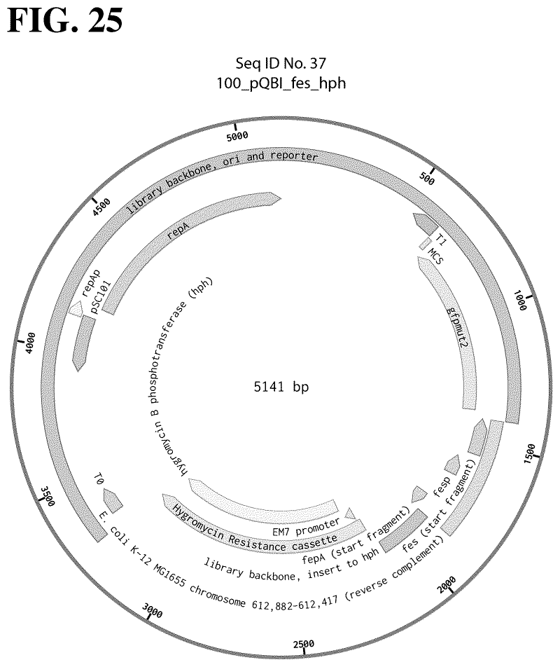

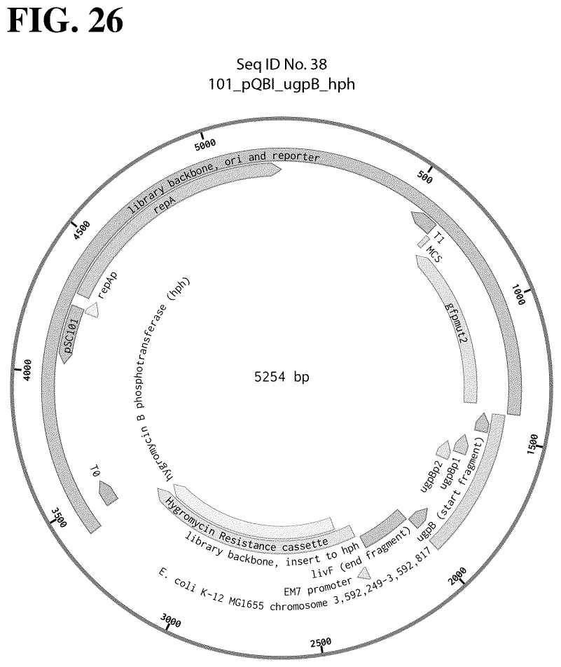

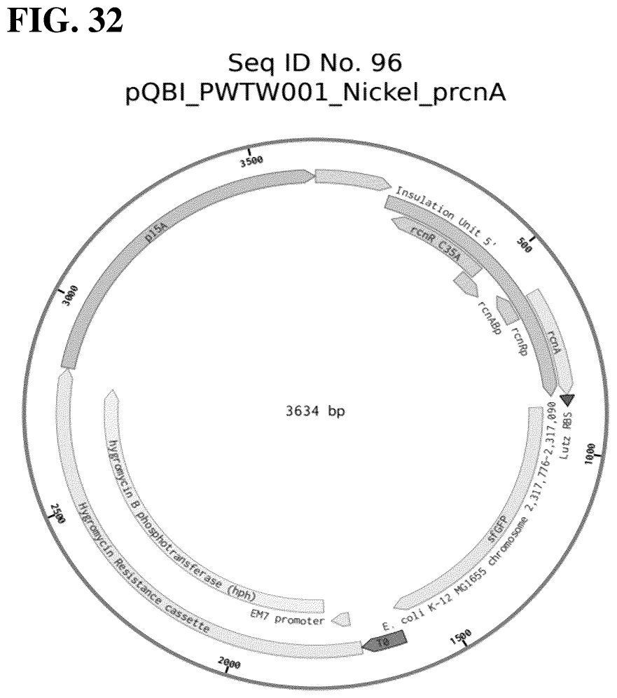

Full DNA sequences for top-performing sensing plasmid constructs are shown, respectively, in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:36, SEQ ID NO:37, SEQ ID NO:38, and SEQ ID NO:96, alongside the full plasmid names and descriptions. Corresponding plasmid maps with main features annotated are shown, respectively, in FIG. 3, FIG. 4, FIG. 5, FIG. 6, FIG. 7, FIG. 8, FIG. 9, FIG. 10, FIG. 11, FIG. 12, FIG. 13, FIG. 24, FIG. 25, FIG. 26, and FIG. 32, which are described below in greater detail.

### Exemplary Strains Sensitive to Ammonium

The E. coli strain that we designated “glnA” contained a plasmid (full plasmid name “78_pQBI_P12glnA-glnA-LutzRBS-sfgfp-glnLG_Hygro_p15A,” schematic map shown in FIG. 3), having the nucleic acid sequence of SEQ ID NO:1:

GAATTCAAAAGATCTTAAGTAAGTAAGAGTATACGTATATCGGCTAATAACGTAT
TAAGGCGCTTCGGCGCCTTTTTTTATGGGGGTATTTTCATCCCAATCCACACGTCC
AACGCACAGCAAACACCACGTCGACCCTATCAGCTGCGTGCTTTCTATGAGTCGT
TGCTGCATAACAACAGGATCACAAACATCCTCCGCAAACAAGTATTGCAGAGTC
CCTTTGTGATCGCTTTCACGGAGCATAAAAAGGGTTATCCAAAGGTCATTGCACC
AACATGGTGCTTAATGTTTCCATTGAAGCACTATATTGGTGCAACATTCACATCG
TGGTGCAGCCCTTTTGCACGATGGTGCGCATGATAACGCCTTTTAGGGGCAATTT
AAAAGTTGGCACAGATTTCGCTTTATCTTTTTTACGGCGACACGGCCAAAATAAT
TGCAGATTTCGTTACCACGACGACCATGACCAATCCAGGAGAGTTAAAGTATGTC
CGCTGAACACGTACTGACGATGCTGAACGAGCACGAAGTGAAGTTTGTTGATTTG
CGCTTCACCGATACTAAAGGTAAAGAACAGCACGTCACTATCCCTGCTCATCAGG
TGAATGCTGAATTCTTCGAAGAAGGCAAAATGTTTGACGGCTCCTCGATTGGCGG
CTGGAAAGGCATTAACGAGTCCGACATGGTGCTGATGCCAGACGCATCCACCGC
AGTGATTGACCCGTTCTTCGCCGACTCCACCCTGATTATCCGTTGCGACATCCTTG
AACCTGGCACCCTGCAAGGCTATGACCGTGACCCGCGCTCCATTGCGAAGCGCG
CCGAAGATTACCTGCGTTCCACTGGCATTGCCGACACCGTACTGTTCGGGCCAGA
ACCTGAATTCTTCCTGTTCGATGACATCCGTTTCGGATCATCTATCTCCGGTTCCC
ACGTTGCTATCGACGATATCGAAGGCGCATGGAACTCCTCCACCCAATACGAAG
GTGGTAACAAAGGTCACCGTCCGGCAGTGAAAGGCGGTTACTTCCCGGTTCCACC
GGTAGACTCGGCTCAGGATATTCGTTCTGAAATGTGTCTGGTGATGGAACAGATG
GGTCTGGTGGTTGAAGCCCATCACCACGAAGTAGCGACTGCTGGTCAGAACGAA
GTGGCTACCCGCTTCAATACCATGACCAAAAAAGCTGACGAAATTCAGATCTAC
AAATATGTTGTGCACAACGTAGCGCACCGCTTCGGTAAAACCGCGACCTTTATGC
CAAAACCGATGTTCGGTGATAACGGCTCCGGTATGCACTGCCACATGTCTCTGTC
TAAAAACGGCGTTAACCTGTTCGCAGGCGACAAATACGCAGGTCTGTCTGAGCA
GGCGCTGTACTACATTGGCGGCGTAATCAAACACGCTAAAGCGATTAACGCCCT
GGCAAACCCGACCACCAACTCTTATAAGCGTCTGGTCCCGGGCTATGAAGCACC
GGTAATGCTGGCTTACTCTGCGCGTAACCGTTCTGCGTCTATCCGTATTCCGGTGG
TTTCTTCTCCGAAAGCACGTCGTATCGAAGTACGTTTCCCGGATCCGGCAGCTAA
CCCGTACCTGTGCTTTGCTGCCCTGCTGATGGCCGGTCTTGATGGTATCAAGAAC
AAGATCCATCCGGGCGAAGCCATGGACAAAAACCTGTATGACCTGCCGCCAGAA
GAAGCGAAAGAGATCCCACAGGTTGCAGGCTCTCTGGAAGAAGCACTGAACGAA
CTGGATCTGGACCGCGAGTTCCTGAAAGCCGGTGGCGTGTTCACTGACGAAGCA
ATTGATGCGTACATCGCTCTGCGTCGCGAAGAAGATGACCGCGTGCGTATGACTC
CGCATCCGGTAGAGTTTGAGCTGTACTACAGCGTCTAAGAATTCATTAAAGAGGA
GAAAGGTACCATGCGTAAAGGCGAAGAGCTGTTCACTGGTGTCGTCCCTATTCTG
GTGGAACTGGATGGTGATGTCAACGGTCATAAGTTTTCCGTGCGTGGCGAGGGTG
AAGGTGACGCAACTAATGGTAAACTGACGCTGAAGTTCATCTGTACTACTGGTAA
ACTGCCGGTACCTTGGCCGACTCTGGTAACGACGCTGACTTATGGTGTTCAGTGC
TTTGCTCGTTATCCGGACCATATGAAGCAGCATGACTTCTTCAAGTCCGCCATGC
CGGAAGGCTATGTGCAGGAACGCACGATTTCCTTTAAGGATGACGGCACGTACA
AAACGCGTGCGGAAGTGAAATTTGAAGGCGATACCCTGGTAAACCGCATTGAGC
TGAAAGGCATTGACTTTAAAGAAGACGGCAATATCCTGGGCCATAAGCTGGAAT
ACAATTTTAACAGCCACAATGTTTACATCACCGCCGATAAACAAAAAAATGGCA
TTAAAGCGAATTTTAAAATTCGCCACAACGTGGAGGATGGCAGCGTGCAGCTGG
CTGATCACTACCAGCAAAACACTCCAATCGGTGATGGTCCTGTTCTGCTGCCAGA
CAATCACTATCTGAGCACGCAAAGCGTTCTGTCTAAAGATCCGAACGAGAAACG
CGATCATATGGTTCTGCTGGAGTTCGTAACCGCAGCGGGCATCACGCATGGTATG
GATGAACTGTACAAATGAGTGTTTTAGTTGCCGTGGAAACTTTTCGCCTGTCTCT
GGCAGGCCTGGGATCGGTGGCAAGCACATCACGCCGGATGCGACGCAAATGCGT
CTTATCCGGCCTACACGGTGATGATGTGGTAGGCCGGAGCAGGTGAGTCGCTCTC
CAACGTGAAGTTTGTCAGCTATCTGTAGCCCATCTCTGCATGGGCTTTTTTCTCCG
TCAATTCTCTGATGCTTCGCGCTTTTTATCCGTAAAAAGCTATAATGCACTAAAAT
GGTGCAACCTGTTCAGGAGACTGCTTTATGGCAACAGGCACGCAGCCCGATGCT
GGGCAGATCCTCAACTCGCTGATTAACAGTATTTTGTTAATCGATGACAACCTGG
CGATCCATTACGCCAACCCTGCCGCGCAACAACTGCTCGCCCAAAGCTCCCGCAA
ATTGTTTGGTACACCGTTACCGGAACTGTTGAGCTACTTCTCATTAAATATCGAG
CTGATGCAAGAAAGTCTGGAGGCGGGGCAAGGTTTTACCGATAACGAAGTGACG
CTGGTCATCGACGGGCGCTCGCATATCCTTTCTGTGACGGCCCAGCGTATGCCGG
ACGGCATGATCCTGCTGGAGATGGCTCCGATGGATAACCAGCGCCGCTTAAGTC
AGGAACAGCTACAGCACGCCCAGCAGGTTGCTGCCCGTGATTTAGTGCGCGGCC
TGGCACATGAGATTAAAAATCCGCTTGGCGGTTTACGTGGCGCGGCGCAGCTGCT
CAGCAAAGCGTTACCTGACCCATCACTACTCGAATATACCAAAGTGATTATCGAA
CAGGCGGACCGGCTGCGAAATCTGGTCGACCGTCTGTTGGGGCCGCAGCTGCCC
GGTACGCGCGTTACCGAAAGTATTCACAAAGTGGCTGAACGCGTGGTAACGCTG
GTGTCGATGGAACTGCCGGACAACGTGCGGTTGATTCGTGATTACGATCCCAGCC
TACCGGAACTGGCGCACGACCCGGATCAAATTGAACAGGTCTTGCTGAATATTGT
GCGCAATGCGCTACAGGCGCTGGGGCCGGAAGGCGGTGAAATCATTCTGCGTAC
CCGCACCGCGTTTCAACTGACCTTACACGGCGAGCGCTACCGGCTGGCGGCGCG
GATTGATGTGGAAGATAACGGGCCGGGCATTCCGCCTCATTTGCAGGATACGCTG
TTTTACCCGATGGTCAGCGGCCGCGAAGGTGGCACCGGGCTTGGCTTATCCATCG
CTCGTAATTTGATTGATCAGCATTCAGGCAAAATTGAATTTACCAGTTGGCCAGG
GCATACCGAGTTCTCGGTTTACCTGCCTATCAGGAAATAAAGGTGACGTTTATGC
AACGAGGGATAGTCTGGGTAGTCGATGACGATAGTTCCATCCGTTGGGTGCTTGA
ACGTGCGCTCGCTGGGGCAGGTTTAACCTGTACGACGTTTGAGAACGGCGCAGA
AGTGCTGGAGGCGCTGGCGAGCAAAACGCCGGATGTGCTGCTTTCAGATATCCG
TATGCCGGGAATGGACGGGCTGGCGCTGCTCAAGCAGATTAAACAGCGCCATCC
AATGCTTCCGGTCATCATTATGACCGCACATTCCGATCTGGATGCTGCCGTCAGC
GCCTATCAACAAGGGGCGTTTGATTATCTGCCCAAACCGTTTGATATCGACGAAG
CAGTGGCGCTGGTTGAGCGCGCTATCAGTCATTACCAGGAACAGCAGCAGCCGC
GTAATGTTCAGCTTAACGGCCCAACGACCGATATCATCGGCGAAGCGCCAGCCA
TGCAGGACGTGTTCCGTATTATCGGTCGGCTTTCGCGTTCTTCTATTAGCGTGCTG
ATTAACGGCGAATCCGGCACCGGTAAAGAACTGGTCGCTCATGCCCTGCATCGC
CACAGTCCGCGCGCCAAAGCGCCGTTTATCGCGCTGAATATGGCAGCTATCCCAA
AAGATTTGATCGAATCAGAACTGTTTGGCCACGAGAAAGGCGCGTTTACTGGCG
CGAATACCATTCGTCAGGGGCGTTTTGAACAGGCCGATGGCGGTACATTATTCCT
CGACGAAATTGGTGATATGCCGCTGGATGTGCAGACGCGTTTGCTGCGCGTGCTG
GCAGACGGTCAGTTTTACCGCGTTGGCGGCTATGCGCCGGTGAAAGTGGATGTGC
GGATTATCGCTGCCACTCACCAGAATCTCGAACAGCGAGTGCAGGAAGGTAAGT
TCCGTGAGGATCTGTTCCACCGCCTGAACGTTATCCGCGTTCATCTGCCGCCGCT
GCGCGAACGTCGGGAAGATATTCCCCGTCTGGCGCGCCATTTTTTACAGGTTGCC
GCGCGCGAACTGGGCGTAGAAGCGAAGTTACTGCATCCGGAAACCGAAGCTGCT
CTGACGCGTCTGGCGTGGCCAGGCAACGTGCGCCAGCTGGAAAACACCTGCCGC
TGGCTAACGGTGATGGCCGCCGGGCAGGAAGTGTTGATTCAGGATTTGCCCGGC
GAACTGTTTGAATCAACGGTTGCGGAGAGTACTTCGCAAATGCAACCGGACAGC
TGGGCGACGCTTCTTGCGCAGTGGGCAGACAGAGCGCTGCGTTCCGGTCATCAA
AATCTGCTTTCCGAAGCGCAGCCAGAGCTGGAGCGGACGTTACTGACGACCGCG
TTGCGACATACGCAGGGGCATAAACAGGAAGCGGCGCGGCTACTCGGCTGGGGC
CGCAACACCCTGACGCGTAAGTTAAAAGAGCTGGGGATGGAGTGACTTGGACTC
CTGTTGATAGATCCAGTAATGACCTCAGAACTCCATCTGGATTTGTTCAGAACGC
TCGGTTGCCGCCGGGCGTTTTTTATTGGTGAGAATCCACCTTTCGACCTGCAGCCT
GTTGACAATTAATCATCGGCATAGTATATCGGCATAGTATAATACGACAAGGTGA
GGAACTAAACCATGAAAAAGCCTGAACTCACCGCGACGTCTGTCGAGAAGTTTC
TGATCGAAAAGTTCGACAGCGTCTCCGACCTGATGCAGCTCTCGGAGGGCGAAG
AATCTCGTGCTTTCAGCTTCGATGTAGGAGGGCGTGGATATGTCCTGCGGGTAAA
TAGCTGCGCCGATGGTTTCTACAAAGATCGTTATGTTTATCGGCACTTTGCATCG
GCCGCGCTCCCGATTCCGGAAGTGCTTGACATTGGGGAATTCAGCGAGAGCCTG
ACCTATTGCATCTCCCGCCGTGCACAGGGTGTCACGTTGCAAGACCTGCCTGAAA
CCGAACTGCCCGCTGTTCTGCAGCCGGTCGCGGAGGCCATGGATGCGATCGCTGC
GGCCGATCTTAGCCAGACGAGCGGGTTCGGCCCATTCGGACCGCAAGGAATCGG
TCAATACACTACATGGCGTGATTTCATATGCGCGATTGCTGATCCCCATGTGTAT
CACTGGCAAACTGTGATGGACGACACCGTCAGTGCGTCCGTCGCGCAGGCTCTC
GATGAGCTGATGCTTTGGGCCGAGGACTGCCCCGAAGTCCGGCACCTCGTGCAC
GCGGATTTCGGCTCCAACAATGTCCTGACGGACAATGGCCGCATAACAGCGGTC
ATTGACTGGAGCGAGGCGATGTTCGGGGATTCCCAATACGAGGTCGCCAACATC
TTCTTCTGGAGGCCGTGGTTGGCTTGTATGGAGCAGCAGACGCGCTACTTCGAGC
GGAGGCATCCGGAGCTTGCAGGATCGCCGCGGCTCCGGGCGTATATGCTCCGCA
TTGGTCTTGACCAACTCTATCAGAGCTTGGTTGACGGCAATTTCGATGATGCAGC
TTGGGCGCAGGGTCGATGCGACGCAATCGTCCGATCCGGAGCCGGGACTGTCGG
GCGTACACAAATCGCCCGCAGAAGCGCGGCCGTCTGGACCGATGGCTGTGTAGA
AGTACTCGCCGATAGTGGAAACCGACGCCCCGGATGAGGGGATCAATTCTCTAG
AGCTCGCTGATCAGAAGTTCCTGATATATTCCGCTTCCTCGCTCACTGACTCGCTA
CGCTCGGTCGTTCGACTGCGGCGAGCGGAAATGGCTTACGAACGGGGCGGAGAT
TTCCTGGAAGATGCCAGGAAGATACTTAACAGGGAAGTGAGAGGGCCGCGGCAA
AGCCGTTTTTCCATAGGCTCCGCCCCCCTGACAAGCATCACGAAATCTGACGCTC
AAATCAGTGGTGGCGAAACCCGACAGGACTATAAAGATACCAGGCGTTTCCCCC
TGGCGGCTCCCTCGTGCGCTCTCCTGTTCCTGCCTTTCGGTTTACCGGTGTCATTC
CGCTGTTATGGCCGCGTTTGTCTCATTCCACGCCTGACACTCAGTTCCGGGTAGG
CAGTTCGCTCCAAGCTGGACTGTATGCACGAACCCCCCGTTCAGTCCGACCGCTG
CGCCTTATCCGGTAACTATCGTCTTGAGTCCAACCCGGAAAGACATGCAAAAGCA
CCACTGGCAGCAGCCACTGGTAATTGATTTAGAGGAGTTAGTCTTGAAGTCATGC
GCCGGTTAAGGCTAAACTGAAAGGACAAGTTTTGGTGACTGCGCTCCTCCAAGCC
AGTTACCTCGGTTCAAAGAGTTGGTAGCTCAGAGAACCTTCGAAAAACCGCCCTG
CAAGGCGGTTTTTTCGTTTTCAGAGCAAGAGATTACGCGCAGACCAAAACGATCT
CAAGAAGATCATCTTATTAATCAGATAAAATATTTCTAGATTTCAGTGCAATTTA
TCTCTTCAAATGTAGCACCTGAAGTCAGCCCCATACGATATAAGTTGTT//SEQ ID NO: 1.

FIG. 3 shows a schematic plasmid map of an ammonium-sensing plasmid based on the native glnA promoter in E. coli. The glnA gene codes for the glutamine synthetase enzyme, which combines L-glutamate and ammonia to synthesize L-glutamine. The glnALG operon in the Ntr regulon is activated by increasing concentration of transcriptional activator NRI~P during ammonium starvation. The glnA promoter is believed to be highly responsive to ammonium due to its two adjacent high-affinity NRI binding sites. (See, M. R. Atkinson, T. A. Blauwkamp, V. Bondarenko, V. Studitsky, and A. J. Ninfa, “Activation of the glnA, glnK, and nac promoters as Escherichia coli undergoes the transition from nitrogen excess growth to nitrogen starvation,” J Bacteriol, vol. 184, no. 19, pp. 5358-5363, (2002)). We constructed plasmid versions with both the upstream glnAp1 and downstream glnAp2 promoter elements combined (SEQ ID NO:18): AACAGGATCACAAACATCCTCCGCAAACAAGTATTGCAGAGTCCCTTTGTGATCG CTTTCACGGAGCATAAAAAGGGTTATCCAAAGGTCATTGCACCAACATGGTGCTT AATGTTTCCATTGAAGCACTATATTGGTGCAACATTCACATCGTGGTGCAGCCCT TTTGCACGATGGTGCGCATGATAACGCCTTTTAGGGGCAATTTAAAAGTTGGCAC AGATTTCGCTTTATCTTTTTTACGGCGACACGGCCAAAATAATTGCAGATTTCGTT ACCACGACGACCATGACCAATCCAGGAGAGTTAAAGTATGTCCGCTGAACACGT ACTGACGATGCTGAACGAGCACGAAGTGAAGTTTGTTGATTTGCGCTTCACCGAT ACTAAAGGTAAAGAACAGCACGTCACTATCCCTGCTCATCAGGTGAATGCTGAA TTCTTCGAAGAAGGCAAAATGTTTGACGGCTCCTCGATTGGCGGCTGGAAAGGC ATTAACGAGTCCGACATGGTGCTGATGCCAGACGCATCCACCGCAGTGATTGACC CGTTCTTCGCCGACTCCACCCTGATTATCCGTTGCGACATCCTTGAACCTGGCACC CTGCAAGGCTATGACCGTGACCCGCGCTCCATTGCGAAGCGCGCCGAAGATTAC CTGCGTTCCACTGGCATTGCCGACACCGTACTGTTCGGGCCAGAACCTGAATTCT TCCTGTTCGATGACATCCGTTTCGGATCATCTATCTCCGGTTCCCACGTTGCTATC GACGATATCGAAGGCGCATGGAACTCCTCCACCCAATACGAAGGTGGTAACAAA GGTCACCGTCCGGCAGTGAAAGGCGGTTACTTCCCGGTTCCACCGGTAGACTCGG CTCAGGATATTCGTTCTGAAATGTGTCTGGTGATGGAACAGATGGGTCTGGTGGT TGAAGCCCATCACCACGAAGTAGCGACTGCTGGTCAGAACGAAGTGGCTACCCG CTTCAATACCATGACCAAAAAAGCTGACGAAATTCAGATCTACAAATATGTTGTG CACAACGTAGCGCACCGCTTCGGTAAAACCGCGACCTTTATGCCAAAACCGATG TTCGGTGATAACGGCTCCGGTATGCACTGCCACATGTCTCTGTCTAAAAACGGCG TTAACCTGTTCGCAGGCGACAAATACGCAGGTCTGTCTGAGCAGGCGCTGTACTA CATTGGCGGCGTAATCAAACACGCTAAAGCGATTAACGCCCTGGCAAACCCGAC CACCAACTCTTATAAGCGTCTGGTCCCGGGCTATGAAGCACCGGTAATGCTGGCT TACTCTGCGCGTAACCGTTCTGCGTCTATCCGTATTCCGGTGGTTTCTTCTCCGAA AGCACGTCGTATCGAAGTACGTTTCCCGGATCCGGCAGCTAACCCGTACCTGTGC TTTGCTGCCCTGCTGATGGCCGGTCTTGATGGTATCAAGAACAAGATCCATCCGG GCGAAGCCATGGACAAAAACCTGTATGACCTGCCGCCAGAAGAAGCGAAAGAG ATCCCACAGGTTGCAGGCTCTCTGGAAGAAGCACTGAACGAACTGGATCTGGAC CGCGAGTTCCTGAAAGCCGGTGGCGTGTTCACTGACGAAGCAATTGATGCGTAC ATCGCTCTGCGTCGCGAAGAAGATGACCGCGTGCGTATGACTCCGCATCCGGTAG AGTTTGAGCTGTACTACAGCGTCTAA//SEQ ID NO:18, as well as only the glnAp2 promoter element, and we found the dual promoter version provided better sensitivity.

Because ammonium-limited induction of the glnA promoter requires the phosphorylated form of dimerized NRI (encoded by the glnG gene; see, Cardemil, C. et al., “Bioluminescent Escherichia coli strains for the quantitative detection of phosphate and ammonia in coastal and suburban watersheds,” DNA Cell Biol, vol. 29, no. 9, pp. 519-31 (2010), doi: 10.1089/dna.2009.0984), and we wished to avoid dilution of cellular NRI with multiple plasmid-based copies of glnA, we included on the plasmid, downstream of the Lutz RBS and GFP reporter coding sequences, the native glnLG promoter-gene sequence (SEQ ID NO:19):

TGTTTTAGTTGCCGTGGAAACTTTTCGCCTGTCTCTGGCAGGCCTGGGATCGGTGGCAAGCACATCACGCCGGATGCGA
CGCAAATGCGTCTTATCCGGCCTACACGGTGATGATGTGGTAGGCCGGAGCAGGTGAGTCGCTCTCCAACGTGAAGTTT
GTCAGCTATCTGTAGCCCATCTCTGCATGGGCTTTTTTCTCCGTCAATTCTCTGATGCTTCGCGCTTTTTATCCGTAAA
AAGCTATAATGCACTAAAATGGTGCAACCTGTTCAGGAGACTGCTTTATGGCAACAGGCACGCAGCCCGATGCTGGGCA
GATCCTCAACTCGCTGATTAACAGTATTTTGTTAATCGATGACAACCTGGCGATCCATTACGCCAACCCTGCCGCGCAA
CAACTGCTCGCCCAAAGCTCCCGCAAATTGTTTGGTACACCGTTACCGGAACTGTTGAGCTACTTCTCATTAAATATCG
AGCTGATGCAAGAAAGTCTGGAGGCGGGGCAAGGTTTTACCGATAACGAAGTGACGCTGGTCATCGACGGGCGCTCGCA
TATCCTTTCTGTGACGGCCCAGCGTATGCCGGACGGCATGATCCTGCTGGAGATGGCTCCGATGGATAACCAGCGCCGC
TTAAGTCAGGAACAGCTACAGCACGCCCAGCAGGTTGCTGCCCGTGATTTAGTGCGCGGCCTGGCACATGAGATTAAAA
ATCCGCTTGGCGGTTTACGTGGCGCGGCGCAGCTGCTCAGCAAAGCGTTACCTGACCCATCACTACTCGAATATACCAA
AGTGATTATCGAACAGGCGGACCGGCTGCGAAATCTGGTCGACCGTCTGTTGGGGCCGCAGCTGCCCGGTACGCGCGTT
ACCGAAAGTATTCACAAAGTGGCTGAACGCGTGGTAACGCTGGTGTCGATGGAACTGCCGGACAACGTGCGGTTGATTC
GTGATTACGATCCCAGCCTACCGGAACTGGCGCACGACCCGGATCAAATTGAACAGGTCTTGCTGAATATTGTGCGCAA
TGCGCTACAGGCGCTGGGGCCGGAAGGCGGTGAAATCATTCTGCGTACCCGCACCGCGTTTCAACTGACCTTACACGGC
GAGCGCTACCGGCTGGCGGCGCGGATTGATGTGGAAGATAACGGGCCGGGCATTCCGCCTCATTTGCAGGATACGCTGT
TTTACCCGATGGTCAGCGGCCGCGAAGGTGGCACCGGGCTTGGCTTATCCATCGCTCGTAATTTGATTGATCAGCATTC
AGGCAAAATTGAATTTACCAGTTGGCCAGGGCATACCGAGTTCTCGGTTTACCTGCCTATCAGGAAAtaaAGGTGACGT
TTATGCAACGAGGGATAGTCTGGGTAGTCGATGACGATAGTTCCATCCGTTGGGTGCTTGAACGTGCGCTCGCTGGGGC
AGGTTTAACCTGTACGACGTTTGAGAACGGCGCAGAAGTGCTGGAGGCGCTGGCGAGCAAAACGCCGGATGTGCTGCTT
TCAGATATCCGTATGCCGGGAATGGACGGGCTGGCGCTGCTCAAGCAGATTAAACAGCGCCATCCAATGCTTCCGGTCA
TCATTATGACCGCACATTCCGATCTGGATGCTGCCGTCAGCGCCTATCAACAAGGGGCGTTTGATTATCTGCCCAAACC
GTTTGATATCGACGAAGCAGTGGCGCTGGTTGAGCGCGCTATCAGTCATTACCAGGAACAGCAGCAGCCGCGTAATGTT
CAGCTTAACGGCCCAACGACCGATATCATCGGCGAAGCGCCAGCCATGCAGGACGTGTTCCGTATTATCGGTCGGCTTT
CGCGTTCTTCTATTAGCGTGCTGATTAACGGCGAATCCGGCACCGGTAAAGAACTGGTCGCTCATGCCCTGCATCGCCA
CAGTCCGCGCGCCAAAGCGCCGTTTATCGCGCTGAATATGGCAGCTATCCCAAAAGATTTGATCGAATCAGAACTGTTT
GGCCACGAGAAAGGCGCGTTTACTGGCGCGAATACCATTCGTCAGGGGCGTTTTGAACAGGCCGATGGCGGTACATTAT
TCCTCGACGAAATTGGTGATATGCCGCTGGATGTGCAGACGCGTTTGCTGCGCGTGCTGGCAGACGGTCAGTTTTACCG
CGTTGGCGGCTATGCGCCGGTGAAAGTGGATGTGCGGATTATCGCTGCCACTCACCAGAATCTCGAACAGCGAGTGCAG
GAAGGTAAGTTCCGTGAGGATCTGTTCCACCGCCTGAACGTTATCCGCGTTCATCTGCCGCCGCTGCGCGAACGTCGGG
AAGATATTCCCCGTCTGGCGCGCCATTTTTTACAGGTTGCCGCGCGCGAACTGGGCGTAGAAGCGAAGTTACTGCATCC
GGAAACCGAAGCTGCTCTGACGCGTCTGGCGTGGCCAGGCAACGTGCGCCAGCTGGAAAACACCTGCCGCTGGCTAACG
GTGATGGCCGCCGGGCAGGAAGTGTTGATTCAGGATTTGCCCGGCGAACTGTTTGAATCAACGGTTGCGGAGAGTACTT
CGCAAATGCAACCGGACAGCTGGGCGACGCTTCTTGCGCAGTGGGCAGACAGAGCGCTGCGTTCCGGTCATCAAAATCT
GCTTTCCGAAGCGCAGCCAGAGCTGGAGCGGACGTTACTGACGACCGCGTTGCGACATACGCAGGGGCATAAACAGGAA
GCGGCGCGGCTACTCGGCTGGGGCCGCAACACCCTGACGCGTAAGTTAAAAGAGCTGGGGATGGAGTGA//SEQ ID
NO: 19.

The E. coli strain that we designated “glnK” contained an ammonium-sensing plasmid (full plasmid name “40_pQBI_Pfnr-fnr(L28H)_PglnK-LutzRBS-sfgfp_Hygro_p15A,” schematic plasmid map shown in FIG. 4), having the nucleic acid sequence of SEQ ID NO:2: GAATTCAAAAGATCTTAAGTAAGTAAGAGTATACGTATATCGGCTAATAACGTAT TAAGGCGCTTCGGCGCCTTTTTTTATGGGGGTATTTTCATCCCAATCCACACGTCC AACGCACAGCAAACACCACGTCGACCCTATCAGCTGCGTGCTTTCTATGAGTCGT TGCTGCATAACTCGTGAATATTTTGCCGGTAATCTTAGCATTATTGATACGCTTCC CACTGCTACGGGGGGGACGCCATTTCAGCGCGAAGTCTGGAAAACACTACGCAC TATCCCCTGCGGGCAGGTAATGCATTACGGCCAACTGGCTGAGCAATTGGGCCGT CCTGGCGCGGCGCGTGCCGTTGGTGCGGCAAACGGATCGAATCCCATCAGCATC GTCGTACCTTGCCATCGGGTTATTGGCCGAAACGGCACCATGACCGGATATGCAG GCGGAGTTCAGCGAAAAGAGTGGTTATTGCGCCATGAAGGTTATCTTTTGCTGTA AACATTAAACAATTTGTGCCAGCTTGTTCACACTTTTATGTAAAGTTACCCTTAAC AACTTAAGGGTTTTCAAATAGATAGACATATATTTACATCTAATATCGGAATTCT CTGCTGTTAAGGTTTGCTTAGACTTACTTGCTCCCTAAAAAGATGTTAAAATTGA CAAATATCAATTACGGCTTGAGCAGACCTATGATCCCGGAAAAGCGAATTATAC GGCGCATTCAGTCTGGCGGTTGTGCTATCCATTGCCAGGATTGCAGCATCAGCCA GCATTGCATCCCGTTCACACTCAACGAACATGAGCTTGATCAGCTTGATAATATC ATTGAGCGGAAGAAGCCTATTCAGAAAGGCCAGACGCTGTTTAAGGCTGGTGAT GAACTTAAATCGCTTTATGCCATCCGCTCCGGTACGATTAAAAGTTATACCATCA CTGAGCAAGGCGACGAGCAAATCACTGGTTTCCATTTAGCAGGCGACCTGGTGG GATTTGACGCCATCGGCAGCGGCCATCACCCGAGCTTCGCGCAGGCGCTGGAAA CCTCGATGGTATGTGAAATCCCGTTCGAAACGCTGGACGATTTGTCCGGTAAAAT GCCGAATCTGCGTCAGCAGATGATGCGTCTGATGAGCGGTGAAATCAAAGGCGA TCAGGACATGATCCTGCTGTTGTCGAAGAAAAATGCCGAGGAACGTCTGGCTGC ATTCATCTACAACCTGTCCCGTCGTTTTGCCCAACGCGGCTTCTCCCCTCGTGAAT TCCGCCTGACGATGACTCGTGGCGATATCGGTAACTATCTGGGCCTGACGGTAGA AACCATCAGCCGTCTGCTGGGTCGCTTCCAGAAAAGCGGCATGCTGGCAGTCAA AGGTAAATACATCACCATCGAAAATAACGATGCGCTGGCCCAGCTTGCTGGTCAT ACGCGTAACGTTGCCTGATTTTTCCGCATAACTCACTATCCTTCTGTCATATCATT AAATTTTTCTGATTTATTGATCTGGCAGAAGGTTCATCACTGTTTCATTCACCAGA TATGGGTTAATCTTTTAATTACAAACTGCGTTGACAGTTGTTGTAAGGAGACCCT GTATGGCTATGTATCAGAACATGCTCGTTGTTATCGATCCTAACCAGGACGACCA ACCAGCATTGCGGCGAGCTGTTTATTTACATCAACGGATTGGTGGCAAAATTAAA GCCTTTTTGCCGATCTATGACTTCTCATACGAAATGACCACCCTGCTCTCCCCGGA CGAACGTACCGCTATGCGTCAGGGCGTCATCAGCCAGCGTACAGCCTGGATCTG CTAGAGGCATCAAATAAAACGAAAGGCTCAGTCGAAAGACTGGGCCTTTCGTTT TATCTGTTGTTTGTCGGTGAACGCTCTCCTGAGTAGGACAAATCCGCCGATAGCG CAATATTTCATCGTTGGTGCAAAAATGTAACGCACTGTGCACTGTCATAGTGCGT TTTCATTTTCAAACTTCTTAACTTCCTGCTCTCTTTCTCGTTTTTCATTTCTGGCAC ACCGCTTGCAATACCTTCTTCGTGTAGCAGAACCATTGAATTCATTAAAGAGGAG AAAGGTACCATGCGTAAAGGCGAAGAGCTGTTCACTGGTGTCGTCCCTATTCTGG TGGAACTGGATGGTGATGTCAACGGTCATAAGTTTTCCGTGCGTGGCGAGGGTGA AGGTGACGCAACTAATGGTAAACTGACGCTGAAGTTCATCTGTACTACTGGTAAA CTGCCGGTACCTTGGCCGACTCTGGTAACGACGCTGACTTATGGTGTTCAGTGCT TTGCTCGTTATCCGGACCATATGAAGCAGCATGACTTCTTCAAGTCCGCCATGCC GGAAGGCTATGTGCAGGAACGCACGATTTCCTTTAAGGATGACGGCACGTACAA AACGCGTGCGGAAGTGAAATTTGAAGGCGATACCCTGGTAAACCGCATTGAGCT GAAAGGCATTGACTTTAAAGAAGACGGCAATATCCTGGGCCATAAGCTGGAATA CAATTTTAACAGCCACAATGTTTACATCACCGCCGATAAACAAAAAAATGGCATT AAAGCGAATTTTAAAATTCGCCACAACGTGGAGGATGGCAGCGTGCAGCTGGCT GATCACTACCAGCAAAACACTCCAATCGGTGATGGTCCTGTTCTGCTGCCAGACA ATCACTATCTGAGCACGCAAAGCGTTCTGTCTAAAGATCCGAACGAGAAACGCG ATCATATGGTTCTGCTGGAGTTCGTAACCGCAGCGGGCATCACGCATGGTATGGA TGAACTGTACAAATGACTTGGACTCCTGTTGATAGATCCAGTAATGACCTCAGAA CTCCATCTGGATTTGTTCAGAACGCTCGGTTGCCGCCGGGCGTTTTTTATTGGTGA GAATCCACCTTTCGACCTGCAGCCTGTTGACAATTAATCATCGGCATAGTATATC GGCATAGTATAATACGACAAGGTGAGGAACTAAACCATGAAAAAGCCTGAACTC ACCGCGACGTCTGTCGAGAAGTTTCTGATCGAAAAGTTCGACAGCGTCTCCGACC TGATGCAGCTCTCGGAGGGCGAAGAATCTCGTGCTTTCAGCTTCGATGTAGGAGG GCGTGGATATGTCCTGCGGGTAAATAGCTGCGCCGATGGTTTCTACAAAGATCGT TATGTTTATCGGCACTTTGCATCGGCCGCGCTCCCGATTCCGGAAGTGCTTGACA TTGGGGAATTCAGCGAGAGCCTGACCTATTGCATCTCCCGCCGTGCACAGGGTGT CACGTTGCAAGACCTGCCTGAAACCGAACTGCCCGCTGTTCTGCAGCCGGTCGCG GAGGCCATGGATGCGATCGCTGCGGCCGATCTTAGCCAGACGAGCGGGTTCGGC CCATTCGGACCGCAAGGAATCGGTCAATACACTACATGGCGTGATTTCATATGCG CGATTGCTGATCCCCATGTGTATCACTGGCAAACTGTGATGGACGACACCGTCAG TGCGTCCGTCGCGCAGGCTCTCGATGAGCTGATGCTTTGGGCCGAGGACTGCCCC GAAGTCCGGCACCTCGTGCACGCGGATTTCGGCTCCAACAATGTCCTGACGGAC AATGGCCGCATAACAGCGGTCATTGACTGGAGCGAGGCGATGTTCGGGGATTCC CAATACGAGGTCGCCAACATCTTCTTCTGGAGGCCGTGGTTGGCTTGTATGGAGC AGCAGACGCGCTACTTCGAGCGGAGGCATCCGGAGCTTGCAGGATCGCCGCGGC TCCGGGCGTATATGCTCCGCATTGGTCTTGACCAACTCTATCAGAGCTTGGTTGA CGGCAATTTCGATGATGCAGCTTGGGCGCAGGGTCGATGCGACGCAATCGTCCG ATCCGGAGCCGGGACTGTCGGGCGTACACAAATCGCCCGCAGAAGCGCGGCCGT CTGGACCGATGGCTGTGTAGAAGTACTCGCCGATAGTGGAAACCGACGCCCCGG ATGAGGGGATCAATTCTCTAGAGCTCGCTGATCAGAAGTTCCTGATATATTCCGC TTCCTCGCTCACTGACTCGCTACGCTCGGTCGTTCGACTGCGGCGAGCGGAAATG GCTTACGAACGGGGCGGAGATTTCCTGGAAGATGCCAGGAAGATACTTAACAGG GAAGTGAGAGGGCCGCGGCAAAGCCGTTTTTCCATAGGCTCCGCCCCCCTGACA AGCATCACGAAATCTGACGCTCAAATCAGTGGTGGCGAAACCCGACAGGACTAT AAAGATACCAGGCGTTTCCCCCTGGCGGCTCCCTCGTGCGCTCTCCTGTTCCTGC CTTTCGGTTTACCGGTGTCATTCCGCTGTTATGGCCGCGTTTGTCTCATTCCACGC CTGACACTCAGTTCCGGGTAGGCAGTTCGCTCCAAGCTGGACTGTATGCACGAAC CCCCCGTTCAGTCCGACCGCTGCGCCTTATCCGGTAACTATCGTCTTGAGTCCAA CCCGGAAAGACATGCAAAAGCACCACTGGCAGCAGCCACTGGTAATTGATTTAG AGGAGTTAGTCTTGAAGTCATGCGCCGGTTAAGGCTAAACTGAAAGGACAAGTT TTGGTGACTGCGCTCCTCCAAGCCAGTTACCTCGGTTCAAAGAGTTGGTAGCTCA GAGAACCTTCGAAAAACCGCCCTGCAAGGCGGTTTTTTCGTTTTCAGAGCAAGAG ATTACGCGCAGACCAAAACGATCTCAAGAAGATCATCTTATTAATCAGATAAAA TATTTCTAGATTTCAGTGCAATTTATCTCTTCAAATGTAGCACCTGAAGTCAGCCC CATACGATATAAGTTGTT//SEQ ID NO:2. The 40_pQBI_Pfnr-fnr(L28H)_PglnK-LutzRBS-sfgfp_Hygro_p15A plasmid in this ammonium-sensing “glnK” strain is based on the native glnK promoter in E. coli, having the nucleic acid of SEQ ID NO:22:

ATAGCGCAATATTTCATCGTTGGTGCAAAAATGTAACGCACTGTGCACT
GTCATAGTGCGTTTTCATTTTCAAACTTCTTAACTTCCTGCTCTCTTTC
TCGTTTTTCATTTCTGGCACACCGCTTGCAATACCTTCTTCGTGTAGCA
GAACCATT//SEQ ID NO: 22.

The glnK gene codes for nitrogen regulatory protein PII-2, which, like the PII-1 protein encoded by glnB, controls the activity of glutamine synthetase. Like glnALG, the glnKamtB operon resides in the Ntr regulon and is activated by increasing concentration of transcriptional activator NRI~P during ammonium starvation. It requires elevated NRI concentrations to be activated. (See, M. R. Atkinson, T. A. Blauwkamp, V. Bondarenko, V. Studitsky, and A. J. Ninfa, “Activation of the glnA, glnK, and nac promoters as Escherichia coli undergoes the transition from nitrogen excess growth to nitrogen starvation,” J Bacteriol, vol. 184, no. 19, pp. 5358-5363, (2002)). The fnr module (SEQ ID NO:20), described hereinabove, was included in this plasmid, as the FNR dual regulator both activates and represses transcription from the glnK promoter. (See, e.g., R. Kumar and K. Shimizu, “Transcriptional regulation of main metabolic pathways of cyoA, cydB, fnr, and fur gene knockout Escherichia coli in C-limited and N-limited aerobic continuous cultures,” Microb Cell Fact, vol. 10, (January 2011), doi: 10.1186/1475-2859-10-3).

### Exemplary Strains Sensitive to Nitrate

The E. coli strain that we designated “narG” contained a nitrate-sensing plasmid (full plasmid name “21_pQBI_Pfnr-fnr(L28H)_PnarGtrim-LutzRBS-sfgfp_Hygro_p15A,” schematic plasmid map shown in FIG. 5) having the nucleic acid sequence of SEQ ID NO:3: GAATTCAAAAGATCTTAAGTAAGTAAGAGTATACGTATATCGGCTAATAACGTAT TAAGGCGCTTCGGCGCCTTTTTTTATGGGGGTATTTTCATCCCAATCCACACGTCC AACGCACAGCAAACACCACGTCGACCCTATCAGCTGCGTGCTTTCTATGAGTCGT TGCTGCATAACTCGTGAATATTTTGCCGGTAATCTTAGCATTATTGATACGCTTCC CACTGCTACGGGGGGGACGCCATTTCAGCGCGAAGTCTGGAAAACACTACGCAC TATCCCCTGCGGGCAGGTAATGCATTACGGCCAACTGGCTGAGCAATTGGGCCGT CCTGGCGCGGCGCGTGCCGTTGGTGCGGCAAACGGATCGAATCCCATCAGCATC GTCGTACCTTGCCATCGGGTTATTGGCCGAAACGGCACCATGACCGGATATGCAG GCGGAGTTCAGCGAAAAGAGTGGTTATTGCGCCATGAAGGTTATCTTTTGCTGTA AACATTAAACAATTTGTGCCAGCTTGTTCACACTTTTATGTAAAGTTACCCTTAAC AACTTAAGGGTTTTCAAATAGATAGACATATATTTACATCTAATATCGGAATTCT CTGCTGTTAAGGTTTGCTTAGACTTACTTGCTCCCTAAAAAGATGTTAAAATTGA CAAATATCAATTACGGCTTGAGCAGACCTATGATCCCGGAAAAGCGAATTATAC GGCGCATTCAGTCTGGCGGTTGTGCTATCCATTGCCAGGATTGCAGCATCAGCCA GCATTGCATCCCGTTCACACTCAACGAACATGAGCTTGATCAGCTTGATAATATC ATTGAGCGGAAGAAGCCTATTCAGAAAGGCCAGACGCTGTTTAAGGCTGGTGAT GAACTTAAATCGCTTTATGCCATCCGCTCCGGTACGATTAAAAGTTATACCATCA CTGAGCAAGGCGACGAGCAAATCACTGGTTTCCATTTAGCAGGCGACCTGGTGG GATTTGACGCCATCGGCAGCGGCCATCACCCGAGCTTCGCGCAGGCGCTGGAAA CCTCGATGGTATGTGAAATCCCGTTCGAAACGCTGGACGATTTGTCCGGTAAAAT GCCGAATCTGCGTCAGCAGATGATGCGTCTGATGAGCGGTGAAATCAAAGGCGA TCAGGACATGATCCTGCTGTTGTCGAAGAAAAATGCCGAGGAACGTCTGGCTGC ATTCATCTACAACCTGTCCCGTCGTTTTGCCCAACGCGGCTTCTCCCCTCGTGAAT TCCGCCTGACGATGACTCGTGGCGATATCGGTAACTATCTGGGCCTGACGGTAGA AACCATCAGCCGTCTGCTGGGTCGCTTCCAGAAAAGCGGCATGCTGGCAGTCAA AGGTAAATACATCACCATCGAAAATAACGATGCGCTGGCCCAGCTTGCTGGTCAT ACGCGTAACGTTGCCTGATTTTTCCGCATAACTCACTATCCTTCTGTCATATCATT AAATTTTTCTGATTTATTGATCTGGCAGAAGGTTCATCACTGTTTCATTCACCAGA TATGGGTTAATCTTTTAATTACAAACTGCGTTGACAGTTGTTGTAAGGAGACCCT GTATGGCTATGTATCAGAACATGCTCGTTGTTATCGATCCTAACCAGGACGACCA ACCAGCATTGCGGCGAGCTGTTTATTTACATCAACGGATTGGTGGCAAAATTAAA GCCTTTTTGCCGATCTATGACTTCTCATACGAAATGACCACCCTGCTCTCCCCGGA CGAACGTACCGCTATGCGTCAGGGCGTCATCAGCCAGCGTACAGCCTGGATCTG CTAGAGGCATCAAATAAAACGAAAGGCTCAGTCGAAAGACTGGGCCTTTCGTTT TATCTGTTGTTTGTCGGTGAACGCTCTCCTGAGTAGGACAAATCCGCCGAGCTTC TATATCGCCTGCGTAGTGATTACCTGGGCGGTATATGGTCGGCATTCTAAAAAAT AAACCGTTACTCGTCATACTTCGGGTTACATGTGCTGCGGCTGCGTTCATTCACCC CAGTCACTTACTTTAGTAAGCTCCTGGGATTCATTCACTTGCCGCCTTCCTGTAAA CCGAATTATATAGAGTAAAATATTTGATTATCCTTTGCGCGGCATGATGTCGCGC TTTTTTTATGCGTCATTTAGTTACAACATACTAATGTTATATGGTTTATTTCGCCG GATTTCATTAAGAGCCATTAATATGTTACCCATGGGGAATACTCCTTAATACCCA TCTGCATAAAAATCTTAATAGTTTAAATAACTACAGGTATAAAACGTCTTAATTT ACAGTCTGTTATGTGGTGGCTGTTAATTATCCTAAAGGGGTATCTTAGGAATTTA CTTTATTTTTCATCCCCATCACTCTTGATCGTTATCAATTCCCACGCTGTTTCAGA GCGTTACCTTGCCCTTAGAATTCATTAAAGAGGAGAAAGGTACCATGCGTAAAG GCGAAGAGCTGTTCACTGGTGTCGTCCCTATTCTGGTGGAACTGGATGGTGATGT CAACGGTCATAAGTTTTCCGTGCGTGGCGAGGGTGAAGGTGACGCAACTAATGG TAAACTGACGCTGAAGTTCATCTGTACTACTGGTAAACTGCCGGTACCTTGGCCG ACTCTGGTAACGACGCTGACTTATGGTGTTCAGTGCTTTGCTCGTTATCCGGACC ATATGAAGCAGCATGACTTCTTCAAGTCCGCCATGCCGGAAGGCTATGTGCAGG AACGCACGATTTCCTTTAAGGATGACGGCACGTACAAAACGCGTGCGGAAGTGA AATTTGAAGGCGATACCCTGGTAAACCGCATTGAGCTGAAAGGCATTGACTTTAA AGAAGACGGCAATATCCTGGGCCATAAGCTGGAATACAATTTTAACAGCCACAA TGTTTACATCACCGCCGATAAACAAAAAAATGGCATTAAAGCGAATTTTAAAATT CGCCACAACGTGGAGGATGGCAGCGTGCAGCTGGCTGATCACTACCAGCAAAAC ACTCCAATCGGTGATGGTCCTGTTCTGCTGCCAGACAATCACTATCTGAGCACGC AAAGCGTTCTGTCTAAAGATCCGAACGAGAAACGCGATCATATGGTTCTGCTGG AGTTCGTAACCGCAGCGGGCATCACGCATGGTATGGATGAACTGTACAAATGAC TTGGACTCCTGTTGATAGATCCAGTAATGACCTCAGAACTCCATCTGGATTTGTTC AGAACGCTCGGTTGCCGCCGGGCGTTTTTTATTGGTGAGAATCCACCTTTCGACC TGCAGCCTGTTGACAATTAATCATCGGCATAGTATATCGGCATAGTATAATACGA CAAGGTGAGGAACTAAACCATGAAAAAGCCTGAACTCACCGCGACGTCTGTCGA GAAGTTTCTGATCGAAAAGTTCGACAGCGTCTCCGACCTGATGCAGCTCTCGGAG GGCGAAGAATCTCGTGCTTTCAGCTTCGATGTAGGAGGGCGTGGATATGTCCTGC GGGTAAATAGCTGCGCCGATGGTTTCTACAAAGATCGTTATGTTTATCGGCACTT TGCATCGGCCGCGCTCCCGATTCCGGAAGTGCTTGACATTGGGGAATTCAGCGAG AGCCTGACCTATTGCATCTCCCGCCGTGCACAGGGTGTCACGTTGCAAGACCTGC CTGAAACCGAACTGCCCGCTGTTCTGCAGCCGGTCGCGGAGGCCATGGATGCGA TCGCTGCGGCCGATCTTAGCCAGACGAGCGGGTTCGGCCCATTCGGACCGCAAG GAATCGGTCAATACACTACATGGCGTGATTTCATATGCGCGATTGCTGATCCCCA TGTGTATCACTGGCAAACTGTGATGGACGACACCGTCAGTGCGTCCGTCGCGCAG GCTCTCGATGAGCTGATGCTTTGGGCCGAGGACTGCCCCGAAGTCCGGCACCTCG TGCACGCGGATTTCGGCTCCAACAATGTCCTGACGGACAATGGCCGCATAACAG CGGTCATTGACTGGAGCGAGGCGATGTTCGGGGATTCCCAATACGAGGTCGCCA ACATCTTCTTCTGGAGGCCGTGGTTGGCTTGTATGGAGCAGCAGACGCGCTACTT CGAGCGGAGGCATCCGGAGCTTGCAGGATCGCCGCGGCTCCGGGCGTATATGCT CCGCATTGGTCTTGACCAACTCTATCAGAGCTTGGTTGACGGCAATTTCGATGAT GCAGCTTGGGCGCAGGGTCGATGCGACGCAATCGTCCGATCCGGAGCCGGGACT GTCGGGCGTACACAAATCGCCCGCAGAAGCGCGGCCGTCTGGACCGATGGCTGT GTAGAAGTACTCGCCGATAGTGGAAACCGACGCCCCGGATGAGGGGATCAATTC TCTAGAGCTCGCTGATCAGAAGTTCCTGATATATTCCGCTTCCTCGCTCACTGACT CGCTACGCTCGGTCGTTCGACTGCGGCGAGCGGAAATGGCTTACGAACGGGGCG GAGATTTCCTGGAAGATGCCAGGAAGATACTTAACAGGGAAGTGAGAGGGCCGC GGCAAAGCCGTTTTTCCATAGGCTCCGCCCCCCTGACAAGCATCACGAAATCTGA CGCTCAAATCAGTGGTGGCGAAACCCGACAGGACTATAAAGATACCAGGCGTTT CCCCCTGGCGGCTCCCTCGTGCGCTCTCCTGTTCCTGCCTTTCGGTTTACCGGTGT CATTCCGCTGTTATGGCCGCGTTTGTCTCATTCCACGCCTGACACTCAGTTCCGGG TAGGCAGTTCGCTCCAAGCTGGACTGTATGCACGAACCCCCCGTTCAGTCCGACC GCTGCGCCTTATCCGGTAACTATCGTCTTGAGTCCAACCCGGAAAGACATGCAAA AGCACCACTGGCAGCAGCCACTGGTAATTGATTTAGAGGAGTTAGTCTTGAAGTC ATGCGCCGGTTAAGGCTAAACTGAAAGGACAAGTTTTGGTGACTGCGCTCCTCCA AGCCAGTTACCTCGGTTCAAAGAGTTGGTAGCTCAGAGAACCTTCGAAAAACCG CCCTGCAAGGCGGTTTTTTCGTTTTCAGAGCAAGAGATTACGCGCAGACCAAAAC GATCTCAAGAAGATCATCTTATTAATCAGATAAAATATTTCTAGATTTCAGTGCA ATTTATCTCTTCAAATGTAGCACCTGAAGTCAGCCCCATACGATATAAGTTGTT/SEQ ID NO:3. The 21_pQBI_Pfnr-fnr(L28H)_PnarGtrim-LutzRBS-sfgfp_Hygro_p15A plasmid in this nitrate-sensing “narG” strain is based on the native narG promoter in E. coli having the nucleic acid sequence of SEQ ID NO:23:

AGCTTCTATATCGCCTGCGTAGTGATTACCTGGGCGGTATATGGTCGGC
ATTCTAAAAAATAAACCGTTACTCGTCATACTTCGGGTTACATGTGCTG
CGGCTGCGTTCATTCACCCCAGTCACTTACTTTAGTAAGCTCCTGGGAT
TCATTCACTTGCCGCCTTCCTGTAAACCGAATTATATAGAGTAAAATAT
TTGATTATCCTTTGCGCGGCATGATGTCGCGCTTTTTTTATGCGTCATT
TAGTTACAACATACTAATGTTATATGGTTTATTTCGCCGGATTTCATTA
AGAGCCATTAATATGTTACCCATGGGGAATACTCCTTAATACCCATCTG
CATAAAAATCTTAATAGTTTAAATAACTACAGGTATAAAACGTCTTAAT
TTACAGTCTGTTATGTGGTGGCTGTTAATTATCCTAAAGGGGTATCTTA
GGAATTTACTTTATTTTTCATCCCCATCACTCTTGATCGTTATCAATTC
CCACGCTGTTTCAGAGCGTTACCTTGCCCTTA//SEQ ID NO: 23.

The narG gene codes for the alpha subunit of nitrate reductase A, which is a membrane-bound enzyme encoded by the narGHJI operon. The expression pattern of narGHJI is complementary to the operon for a second nitrate reductase enzyme, napFDAGHBC. Whereas napFDAGHBC is maximally expressed at relatievly low nitrate concentration below 1 mM, and expression is suppressed at relatively high nitrate concentration above 7 mM, narGHJI is weakly expressed at low nitrate concentration below 4 mM and maximally expressed at high nitrate concentration above 7 mM. Nitrate, the product of both enzymes, has only a minor effect on the expression of both operons. (See, e.g., Constantinidou, C. et al., “A reassessment of the FNR regulon and transcriptomic analysis of the effects of nitrate, nitrite, NarXL, and NarQP as Escherichia coli K12 adapts from aerobic to anaerobic growth,” Journal of Biological Chemistry, vol. 281, no. 8, pp. 4802-4815 (2006), doi: 10.1074/jbc.M512312200; H. Wang, C. P. Tseng, and R. P. Gunsalus, “The napF and narG nitrate reductase operons in Escherichia coli are differentially expressed in response to submicromolar concentrations of nitrate but not nitrite,” J Bacteriol, vol. 181, no. 17, pp. 5303-5308 (1999)). The fnr module (SEQ ID NO:20), described hereinabove, was included in this plasmid, as the FNR dual regulator both activates and represses transcription from the narG promoter. (See, e.g., Constantinidou, C. et al., “A reassessment of the FNR regulon and transcriptomic analysis of the effects of nitrate, nitrite, NarXL, and NarQP as Escherichia coli K12 adapts from aerobic to anaerobic growth,” Journal of Biological Chemistry, vol. 281, no. 8, pp. 4802-4815 (2006), doi: 10.1074/jbc.M512312200; K. M. Deangelis, P. Ji, M. K. Firestone, and S. E. Lindow, “Two Novel Bacterial Biosensors for Detection of Nitrate Availability in the Rhizosphere,” Appl Environ Microbiol, vol. 71, no. 12, pp. 8537-8547, (2005), doi: 10.1128/AEM.71.12.8537; S. Federowicz et al., “Determining the Control Circuitry of Redox Metabolism at the Genome-Scale,” PLoS Genet, vol. 10, no. 4 (2014), doi: 10.1371/journal.pgen. 1004264).

The E. coli strain that we designated “fdnG” contained a nitrate-sensing plasmid (full plasmid name “34_pQBI_Pfnr-fnr(L28H)_PfdnGtrim-LutzRBS-sfgfp_Hygro_p15A,” schematic plasmid map shown in FIG. 6) having the nucleic acid sequence of SEQ ID NO:4: GAATTCAAAAGATCTTAAGTAAGTAAGAGTATACGTATATCGGCTAATAACGTAT TAAGGCGCTTCGGCGCCTTTTTTTATGGGGGTATTTTCATCCCAATCCACACGTCC AACGCACAGCAAACACCACGTCGACCCTATCAGCTGCGTGCTTTCTATGAGTCGT TGCTGCATAACTCGTGAATATTTTGCCGGTAATCTTAGCATTATTGATACGCTTCC CACTGCTACGGGGGGGACGCCATTTCAGCGCGAAGTCTGGAAAACACTACGCAC TATCCCCTGCGGGCAGGTAATGCATTACGGCCAACTGGCTGAGCAATTGGGCCGT CCTGGCGCGGCGCGTGCCGTTGGTGCGGCAAACGGATCGAATCCCATCAGCATC GTCGTACCTTGCCATCGGGTTATTGGCCGAAACGGCACCATGACCGGATATGCAG GCGGAGTTCAGCGAAAAGAGTGGTTATTGCGCCATGAAGGTTATCTTTTGCTGTA AACATTAAACAATTTGTGCCAGCTTGTTCACACTTTTATGTAAAGTTACCCTTAAC AACTTAAGGGTTTTCAAATAGATAGACATATATTTACATCTAATATCGGAATTCT CTGCTGTTAAGGTTTGCTTAGACTTACTTGCTCCCTAAAAAGATGTTAAAATTGA CAAATATCAATTACGGCTTGAGCAGACCTATGATCCCGGAAAAGCGAATTATAC GGCGCATTCAGTCTGGCGGTTGTGCTATCCATTGCCAGGATTGCAGCATCAGCCA GCATTGCATCCCGTTCACACTCAACGAACATGAGCTTGATCAGCTTGATAATATC ATTGAGCGGAAGAAGCCTATTCAGAAAGGCCAGACGCTGTTTAAGGCTGGTGAT GAACTTAAATCGCTTTATGCCATCCGCTCCGGTACGATTAAAAGTTATACCATCA CTGAGCAAGGCGACGAGCAAATCACTGGTTTCCATTTAGCAGGCGACCTGGTGG GATTTGACGCCATCGGCAGCGGCCATCACCCGAGCTTCGCGCAGGCGCTGGAAA CCTCGATGGTATGTGAAATCCCGTTCGAAACGCTGGACGATTTGTCCGGTAAAAT GCCGAATCTGCGTCAGCAGATGATGCGTCTGATGAGCGGTGAAATCAAAGGCGA TCAGGACATGATCCTGCTGTTGTCGAAGAAAAATGCCGAGGAACGTCTGGCTGC ATTCATCTACAACCTGTCCCGTCGTTTTGCCCAACGCGGCTTCTCCCCTCGTGAAT TCCGCCTGACGATGACTCGTGGCGATATCGGTAACTATCTGGGCCTGACGGTAGA AACCATCAGCCGTCTGCTGGGTCGCTTCCAGAAAAGCGGCATGCTGGCAGTCAA AGGTAAATACATCACCATCGAAAATAACGATGCGCTGGCCCAGCTTGCTGGTCAT ACGCGTAACGTTGCCTGATTTTTCCGCATAACTCACTATCCTTCTGTCATATCATT AAATTTTTCTGATTTATTGATCTGGCAGAAGGTTCATCACTGTTTCATTCACCAGA TATGGGTTAATCTTTTAATTACAAACTGCGTTGACAGTTGTTGTAAGGAGACCCT GTATGGCTATGTATCAGAACATGCTCGTTGTTATCGATCCTAACCAGGACGACCA ACCAGCATTGCGGCGAGCTGTTTATTTACATCAACGGATTGGTGGCAAAATTAAA GCCTTTTTGCCGATCTATGACTTCTCATACGAAATGACCACCCTGCTCTCCCCGGA CGAACGTACCGCTATGCGTCAGGGCGTCATCAGCCAGCGTACAGCCTGGATCTG CTAGAGGCATCAAATAAAACGAAAGGCTCAGTCGAAAGACTGGGCCTTTCGTTT TATCTGTTGTTTGTCGGTGAACGCTCTCCTGAGTAGGACAAATCCGCCGTTTTTAA CTGGCCTGTCAGGCAGTGGTGCGTTTTTCTACCGCTATTGAGGTAGGTCAATTTG CGAAGGCGGATTATTTTGTGGCAAACAGATGTTCTTTTTGATTTCGCGCAAAAAG ATTCAGAATTTTACTGTTAGTTTCCTCGCGCAGAATTCATTAAAGAGGAGAAAGG TACCATGCGTAAAGGCGAAGAGCTGTTCACTGGTGTCGTCCCTATTCTGGTGGAA CTGGATGGTGATGTCAACGGTCATAAGTTTTCCGTGCGTGGCGAGGGTGAAGGTG ACGCAACTAATGGTAAACTGACGCTGAAGTTCATCTGTACTACTGGTAAACTGCC GGTACCTTGGCCGACTCTGGTAACGACGCTGACTTATGGTGTTCAGTGCTTTGCT CGTTATCCGGACCATATGAAGCAGCATGACTTCTTCAAGTCCGCCATGCCGGAAG GCTATGTGCAGGAACGCACGATTTCCTTTAAGGATGACGGCACGTACAAAACGC GTGCGGAAGTGAAATTTGAAGGCGATACCCTGGTAAACCGCATTGAGCTGAAAG GCATTGACTTTAAAGAAGACGGCAATATCCTGGGCCATAAGCTGGAATACAATTT TAACAGCCACAATGTTTACATCACCGCCGATAAACAAAAAAATGGCATTAAAGC GAATTTTAAAATTCGCCACAACGTGGAGGATGGCAGCGTGCAGCTGGCTGATCA CTACCAGCAAAACACTCCAATCGGTGATGGTCCTGTTCTGCTGCCAGACAATCAC TATCTGAGCACGCAAAGCGTTCTGTCTAAAGATCCGAACGAGAAACGCGATCAT ATGGTTCTGCTGGAGTTCGTAACCGCAGCGGGCATCACGCATGGTATGGATGAAC TGTACAAATGACTTGGACTCCTGTTGATAGATCCAGTAATGACCTCAGAACTCCA TCTGGATTTGTTCAGAACGCTCGGTTGCCGCCGGGCGTTTTTTATTGGTGAGAATC CACCTTTCGACCTGCAGCCTGTTGACAATTAATCATCGGCATAGTATATCGGCAT AGTATAATACGACAAGGTGAGGAACTAAACCATGAAAAAGCCTGAACTCACCGC GACGTCTGTCGAGAAGTTTCTGATCGAAAAGTTCGACAGCGTCTCCGACCTGATG CAGCTCTCGGAGGGCGAAGAATCTCGTGCTTTCAGCTTCGATGTAGGAGGGCGTG GATATGTCCTGCGGGTAAATAGCTGCGCCGATGGTTTCTACAAAGATCGTTATGT TTATCGGCACTTTGCATCGGCCGCGCTCCCGATTCCGGAAGTGCTTGACATTGGG GAATTCAGCGAGAGCCTGACCTATTGCATCTCCCGCCGTGCACAGGGTGTCACGT TGCAAGACCTGCCTGAAACCGAACTGCCCGCTGTTCTGCAGCCGGTCGCGGAGG CCATGGATGCGATCGCTGCGGCCGATCTTAGCCAGACGAGCGGGTTCGGCCCATT CGGACCGCAAGGAATCGGTCAATACACTACATGGCGTGATTTCATATGCGCGATT GCTGATCCCCATGTGTATCACTGGCAAACTGTGATGGACGACACCGTCAGTGCGT CCGTCGCGCAGGCTCTCGATGAGCTGATGCTTTGGGCCGAGGACTGCCCCGAAGT CCGGCACCTCGTGCACGCGGATTTCGGCTCCAACAATGTCCTGACGGACAATGGC CGCATAACAGCGGTCATTGACTGGAGCGAGGCGATGTTCGGGGATTCCCAATAC GAGGTCGCCAACATCTTCTTCTGGAGGCCGTGGTTGGCTTGTATGGAGCAGCAGA CGCGCTACTTCGAGCGGAGGCATCCGGAGCTTGCAGGATCGCCGCGGCTCCGGG CGTATATGCTCCGCATTGGTCTTGACCAACTCTATCAGAGCTTGGTTGACGGCAA TTTCGATGATGCAGCTTGGGCGCAGGGTCGATGCGACGCAATCGTCCGATCCGGA GCCGGGACTGTCGGGCGTACACAAATCGCCCGCAGAAGCGCGGCCGTCTGGACC GATGGCTGTGTAGAAGTACTCGCCGATAGTGGAAACCGACGCCCCGGATGAGGG GATCAATTCTCTAGAGCTCGCTGATCAGAAGTTCCTGATATATTCCGCTTCCTCGC TCACTGACTCGCTACGCTCGGTCGTTCGACTGCGGCGAGCGGAAATGGCTTACGA ACGGGGCGGAGATTTCCTGGAAGATGCCAGGAAGATACTTAACAGGGAAGTGAG AGGGCCGCGGCAAAGCCGTTTTTCCATAGGCTCCGCCCCCCTGACAAGCATCACG AAATCTGACGCTCAAATCAGTGGTGGCGAAACCCGACAGGACTATAAAGATACC AGGCGTTTCCCCCTGGCGGCTCCCTCGTGCGCTCTCCTGTTCCTGCCTTTCGGTTT ACCGGTGTCATTCCGCTGTTATGGCCGCGTTTGTCTCATTCCACGCCTGACACTCA GTTCCGGGTAGGCAGTTCGCTCCAAGCTGGACTGTATGCACGAACCCCCCGTTCA GTCCGACCGCTGCGCCTTATCCGGTAACTATCGTCTTGAGTCCAACCCGGAAAGA CATGCAAAAGCACCACTGGCAGCAGCCACTGGTAATTGATTTAGAGGAGTTAGT CTTGAAGTCATGCGCCGGTTAAGGCTAAACTGAAAGGACAAGTTTTGGTGACTGC GCTCCTCCAAGCCAGTTACCTCGGTTCAAAGAGTTGGTAGCTCAGAGAACCTTCG AAAAACCGCCCTGCAAGGCGGTTTTTTCGTTTTCAGAGCAAGAGATTACGCGCAG ACCAAAACGATCTCAAGAAGATCATCTTATTAATCAGATAAAATATTTCTAGATT TCAGTGCAATTTATCTCTTCAAATGTAGCACCTGAAGTCAGCCCCATACGATATA AGTTGTT//SEQ ID NO:4. The 34_pQBI_Pfnr-fnr(L28H)_PfdnGtrim-LutzRBS-sfgfp_Hygro_p15A plasmid in this nitrate-sensing “fdnG” strain is based on the native fdnG promoter in E. coli having the nucleic acid sequence of SEQ ID NO:24):

TTTTTAACTGGCCTGTCAGGCAGTGGTGCGTTTTTCTACCGCTATTGAG
GTAGGTCAATTTGCGAAGGCGGATTATTTTGTGGCAAACAGATGTTCTT
TTTGATTTCGCGCAAAAAGATTCAGAATTTTACTGTTAGTTTCCTCGCG
CA//SEQ ID NO: 24.

The fdnG gene codes for the alpha subunit of formate dehydrogenase N, and, like the narGHJI operon, the fdnGHI operon is strongly induced by nitrate. fdnG expression is more strongly activated by nitrate than by nitrite and increases with nitrate concentration. (Constantinidou, C. et al., “A reassessment of the FNR regulon and transcriptomic analysis of the effects of nitrate, nitrite, NarXL, and NarQP as Escherichia coli K12 adapts from aerobic to anaerobic growth,” Journal of Biological Chemistry, vol. 281, no. 8, pp. 4802-4815 (2006), doi: 10.1074/jbc.M512312200). The previously described fnr module is included in this plasmid, as the FNR dual regulator both activates and represses transcription from the fdnG promoter. (Constantinidou, C. et al., “A reassessment of the FNR regulon and transcriptomic analysis of the effects of nitrate, nitrite, NarXL, and NarQP as Escherichia coli K12 adapts from aerobic to anaerobic growth,” Journal of Biological Chemistry, vol. 281, no. 8, pp. 4802-4815 (2006), doi: 10.1074/jbc.M512312200; Federowicz et al., “Determining the Control Circuitry of Redox Metabolism at the Genome-Scale,” PLoS Genet, vol. 10, no. 4 (2014), doi: 10.1371/journal.pgen. 1004264; J. Li and V. Stewart, “Localization of Upstream Sequence Elements Required for Nitrate and Anaerobic Induction of fdn (Formate Dehydrogenase-N) Operon Expression in Escherichia coli K-12,” J. Bacteriol. 174(15):4935-42 (1992), doi: 10.1128/jb.174.15.4935-4942.1992).

### Exemplary Strains Sensitive to Nitrite

Escherichia coli has two nitrite reductase enzymes encoded by the nrfABCDEFG and nirBDC operons that both reduce nitrite to ammonia but are expressed in a complementary manner. NrfA is a periplasmic cytochrome c enzyme that acts as a nitrite reductase in association with the NrfB cytochrome c as redox partner. (H. Corker and R. K. Poole, “Nitric oxide formation by Escherichia coli. Dependence on nitrite reductase, the NO-sensing regulator Fnr, and flavohemoglobin Hmp,” Journal of Biological Chemistry, vol. 278, no. 34, pp. 31584-31592 (2003), doi: 10.1074/jbc.M303282200). NirB is a cytoplasmic siroheme-dependent reductase. (H. Wang and R. P. Gunsalus, “The nrfA and nirB Nitrite Reductase Operons in Escherichia coli Are Expressed Differently in Response to Nitrate than to Nitrite,” J. Bacteriol. 182(20):5813-22 (2000), doi: 10.1128/JB.182.20.5813-5822.2000).

The primary role of NrfA is to scavenge for limited concentrations of nitrite or nitrate. nrfA is expressed when nitrite concentration is low, but repressed when it is high (maximally expressed at about 0.75-2 mM, with 50% expression at about 0.2-0.9 and 2.6-3.5 mM). Similarly, nrfA is expressed when nitrate concentration is low, but repressed when it is high (maximally expressed at about 1 mM, with 50% expression at about 0.6 and 3.4 mM). NirB primarily serves to detoxify excessive nitrite in the cytoplasm. nirB is not expressed at low nitrite or nitrate conditions, but is expressed to reduce nitrite to ammonia almost exclusively when the level of nitrite or nitrate is relatively high. Specifically, nirB is maximally expressed at about 1.5-2.5 mM nitrite, with 50% expression at about 0.6-1.5 mM nitrite. Similarly, nirB is maximally expressed at about 3 mM nitrate, with 50% expression at about 1 mM nitrate. (See, Constantinidou, C. et al., “A reassessment of the FNR regulon and transcriptomic analysis of the effects of nitrate, nitrite, NarXL, and NarQP as Escherichia coli K12 adapts from aerobic to anaerobic growth,” Journal of Biological Chemistry, vol. 281, no. 8, pp. 4802-4815 (2006), doi: 10.1074/jbc.M512312200; H. Wang and R. P. Gunsalus, “The nrfA and nirB Nitrite Reductase Operons in Escherichia coli Are Expressed Differently in Response to Nitrate than to Nitrite,” J. Bacteriol. 182(20):5813-22 (2000), doi: 10.1128/JB.182.20.5813-5822.2000; H. Corker and R. K. Poole, “Nitric oxide formation by Escherichia coli. Dependence on nitrite reductase, the NO-sensing regulator Fnr, and flavohemoglobin Hmp,” Journal of Biological Chemistry, vol. 278, no. 34, pp. 31584-31592, 2003, doi: 10.1074/jbc.M303282200; T. M. Khlebodarova, N. A. Ree, and V. A. Likhoshvai, “On the control mechanisms of the nitrite level in Escherichia coli cells: The mathematical model,” BMC Microbiol, vol. 16, no. 1, pp. 15-30, 2016, doi: 10.1186/s12866-015-0619-x).

Additionally, nrfA expression is indirectly regulated by its product through a complex feedback mechanism involving nitric oxide (NO) and the nitrite-sensitive repressor, NsrR. The NsrR regulon is involved in the cell stress response by regulating nine operons involved in cell protection against reactive NO. (N. Filenko et al., “The NsrR regulon of Escherichia coli K-12 includes genes encoding the hybrid cluster protein and the periplasmic, respiratory nitrite reductase,” J Bacteriol, vol. 189, no. 12, pp. 4410-4417 (2007), doi: 10.1128/JB.00080-07). NsrR is a weak repressor of nrfA, and NO relieves this repression; NrfA is both the primary driver of NO generation from nitrite and a detoxifying reducer of NO. (See, H. Corker and R. K. Poole, “Nitric oxide formation by Escherichia coli. Dependence on nitrite reductase, the NO-sensing regulator Fnr, and flavohemoglobin Hmp,” Journal of Biological Chemistry, vol. 278, no. 34, pp. 31584-31592, 2003, doi: 10.1074/jbc.M303282200; H. J. E. Beaumont, S. I. Lens, W. N. M. Reijnders, H. v. Westerhoff, and R. J. M. van Spanning, “Expression of nitrite reductase in Nitrosomonas europaea involves NsrR, a novel nitrite-sensitive transcription repressor,” Mol Microbiol, vol. 54, no. 1, pp. 148-158 (2004), doi: 10.1111/j.1365-2958.2004.04248.x; J. H. van Wonderen, B. Burlat, D. J. Richardson, M. R. Cheesman, and J. N. Butt, “The nitric oxide reductase activity of cytochrome c nitrite reductase from Escherichia coli,” Journal of Biological Chemistry, vol. 283, no. 15, pp. 9587-9594 (2008), doi: 10.1074/jbc.M709090200; O. Einsle, “Structure and function of formate-dependent cytochrome c nitrite reductase, NrfA,” in Methods in Enzymology, vol. 496, Academic Press Inc. (2011), pp. 399-422. doi: 10.1016/B978-0-12-386489-5.00016-6).

Regulation by NsrR is extremely sensitive to repressor titration by NsrR binding sites provided on a multicopy plasmid. (N. Filenko et al., “The NsrR regulon of Escherichia coli K-12 includes genes encoding the hybrid cluster protein and the periplasmic, respiratory nitrite reductase,” J Bacteriol, vol. 189, no. 12, pp. 4410-4417 (2007), doi: 10.1128/JB.00080-07). Therefore, in plasmid variants containing NsrR binding sites, we included a sequence module containing the native nsrR promoter and gene (SEQ ID NO:25): TGGACGTTCTGGATGGCCTGAAAGAGGTTAAACTCTGCGTGGCTTACCGTATGCC GGATGGTCGCGAAGTGACTACCACTCCGCTGGCAGCTGACGACTGGAAAGGTGT AGAGCCGATTTACGAAACCATGCCGGGCTGGTCTGAATCCACCTTCGGCGTGAA AGATCGTAGCGGCCTGCCGCAGGCGGCGCTGAACTATATCAAGCGTATTGAAGA GCTGACTGGTGTGCCGATCGATATCATCTCTACCGGTCCGGATCGTACTGAAACC ATGATTCTGCGCGACCCGTTCGACGCGTAATTCTGGTACGCCTGGCAGATATTTT GCCTGCCGGGCGAACAGTGTGATACATTGCTGTGTCGGGTAAGCCATTACGCTAT CCGACACAGTGTTAAATCCTCGCTTTTTTCCTTCCCCGAACTGAAATAAATTAGC GACACAGCTTGTGGCTGGTTTATCATCAATATAAATGTATTTTTTCCCGATTTCCC TTTTGAGGTTGATGTGCAGTTAACGAGTTTCACTGATTACGGATTACGTGCGCTG ATCTACATGGCGTCATTGCCAGAAGGGCGGATGACCAGTATTTCTGAAGTGACTG ACGTCTACGGCGTCTCCCGTAATCATATGGTCAAAATAATCAATCAACTTAGTCG TGCCGGCTACGTGACTGCTGTTCGTGGAAAAAATGGCGGCATTCGCCTGGGTAAA CCGGCGAGTGCGATACGTATTGGTGATGTGGTGCGCGAGCTGGAGCCCTTATCGC TGGTGAATTGCAGCAGTGAGTTTTGCCACATTACACCTGCCTGTAGGTTGAAACA GGCACTTTCTAAGGCCGTGCAAAGTTTTCTTACGGAACTGGATAACTACACGCTT GCCGATTTGGTTGAAGAGAATCAACCGCTTTATAAATTATTGCTGGTGGAGTGAC GAAAATCTTCATCAGAGATGACAACGGAGGAACCGAG//SEQ ID NO:25. We terminated nsrR transcription with the T22 terminator sequence from Salmonella phage P22 (AAATAAAGCCCTGAGTTTAACCGCTCGGGGCTTTTTGCGTTT//SEQ ID NO:26), but other terminator sequences can be used instead.

The E. coli strain that we designated “nrfA” contained a nitrite-sensing plasmid (full plasmid name “61_pQBI_PnsrR-nsrR_Pfnr-fnr(L28H)_PnrfAtrim-LutzRBS-sfgfp_Hygro_p15A,” schematic plasmid map shown in FIG. 7) having the nucleic acid sequence of SEQ ID NO:5: GAATTCAAAAGATCTTAAGTAAGTAAGAGTATACGTATATCGGCTAATAACGTAT TAAGGCGCTTCGGCGCCTTTTTTTATGGGGGTATTTTCATCCCAATCCACACGTCC AACGCACAGCAAACACCACGTCGACCCTATCAGCTGCGTGCTTTCTATGAGTCGT TGCTGCATAACTGGACGTTCTGGATGGCCTGAAAGAGGTTAAACTCTGCGTGGCT TACCGTATGCCGGATGGTCGCGAAGTGACTACCACTCCGCTGGCAGCTGACGACT GGAAAGGTGTAGAGCCGATTTACGAAACCATGCCGGGCTGGTCTGAATCCACCT TCGGCGTGAAAGATCGTAGCGGCCTGCCGCAGGCGGCGCTGAACTATATCAAGC GTATTGAAGAGCTGACTGGTGTGCCGATCGATATCATCTCTACCGGTCCGGATCG TACTGAAACCATGATTCTGCGCGACCCGTTCGACGCGTAATTCTGGTACGCCTGG CAGATATTTTGCCTGCCGGGCGAACAGTGTGATACATTGCTGTGTCGGGTAAGCC ATTACGCTATCCGACACAGTGTTAAATCCTCGCTTTTTTCCTTCCCCGAACTGAAA TAAATTAGCGACACAGCTTGTGGCTGGTTTATCATCAATATAAATGTATTTTTTCC CGATTTCCCTTTTGAGGTTGATGTGCAGTTAACGAGTTTCACTGATTACGGATTAC GTGCGCTGATCTACATGGCGTCATTGCCAGAAGGGCGGATGACCAGTATTTCTGA AGTGACTGACGTCTACGGCGTCTCCCGTAATCATATGGTCAAAATAATCAATCAA CTTAGTCGTGCCGGCTACGTGACTGCTGTTCGTGGAAAAAATGGCGGCATTCGCC TGGGTAAACCGGCGAGTGCGATACGTATTGGTGATGTGGTGCGCGAGCTGGAGC CCTTATCGCTGGTGAATTGCAGCAGTGAGTTTTGCCACATTACACCTGCCTGTAG GTTGAAACAGGCACTTTCTAAGGCCGTGCAAAGTTTTCTTACGGAACTGGATAAC TACACGCTTGCCGATTTGGTTGAAGAGAATCAACCGCTTTATAAATTATTGCTGG TGGAGTGACGAAAATCTTCATCAGAGATGACAACGGAGGAACCGAGAAATAAA GCCCTGAGTTTAACCGCTCGGGGCTTTTTGCGTTTTCGTGAATATTTTGCCGGTAA TCTTAGCATTATTGATACGCTTCCCACTGCTACGGGGGGGACGCCATTTCAGCGC GAAGTCTGGAAAACACTACGCACTATCCCCTGCGGGCAGGTAATGCATTACGGC CAACTGGCTGAGCAATTGGGCCGTCCTGGCGCGGCGCGTGCCGTTGGTGCGGCA AACGGATCGAATCCCATCAGCATCGTCGTACCTTGCCATCGGGTTATTGGCCGAA ACGGCACCATGACCGGATATGCAGGCGGAGTTCAGCGAAAAGAGTGGTTATTGC GCCATGAAGGTTATCTTTTGCTGTAAACATTAAACAATTTGTGCCAGCTTGTTCAC ACTTTTATGTAAAGTTACCCTTAACAACTTAAGGGTTTTCAAATAGATAGACATA TATTTACATCTAATATCGGAATTCTCTGCTGTTAAGGTTTGCTTAGACTTACTTGC TCCCTAAAAAGATGTTAAAATTGACAAATATCAATTACGGCTTGAGCAGACCTAT GATCCCGGAAAAGCGAATTATACGGCGCATTCAGTCTGGCGGTTGTGCTATCCAT TGCCAGGATTGCAGCATCAGCCAGCATTGCATCCCGTTCACACTCAACGAACATG AGCTTGATCAGCTTGATAATATCATTGAGCGGAAGAAGCCTATTCAGAAAGGCC AGACGCTGTTTAAGGCTGGTGATGAACTTAAATCGCTTTATGCCATCCGCTCCGG TACGATTAAAAGTTATACCATCACTGAGCAAGGCGACGAGCAAATCACTGGTTTC CATTTAGCAGGCGACCTGGTGGGATTTGACGCCATCGGCAGCGGCCATCACCCG AGCTTCGCGCAGGCGCTGGAAACCTCGATGGTATGTGAAATCCCGTTCGAAACG CTGGACGATTTGTCCGGTAAAATGCCGAATCTGCGTCAGCAGATGATGCGTCTGA TGAGCGGTGAAATCAAAGGCGATCAGGACATGATCCTGCTGTTGTCGAAGAAAA ATGCCGAGGAACGTCTGGCTGCATTCATCTACAACCTGTCCCGTCGTTTTGCCCA ACGCGGCTTCTCCCCTCGTGAATTCCGCCTGACGATGACTCGTGGCGATATCGGT AACTATCTGGGCCTGACGGTAGAAACCATCAGCCGTCTGCTGGGTCGCTTCCAGA AAAGCGGCATGCTGGCAGTCAAAGGTAAATACATCACCATCGAAAATAACGATG CGCTGGCCCAGCTTGCTGGTCATACGCGTAACGTTGCCTGATTTTTCCGCATAACT CACTATCCTTCTGTCATATCATTAAATTTTTCTGATTTATTGATCTGGCAGAAGGT TCATCACTGTTTCATTCACCAGATATGGGTTAATCTTTTAATTACAAACTGCGTTG ACAGTTGTTGTAAGGAGACCCTGTATGGCTATGTATCAGAACATGCTCGTTGTTA TCGATCCTAACCAGGACGACCAACCAGCATTGCGGCGAGCTGTTTATTTACATCA ACGGATTGGTGGCAAAATTAAAGCCTTTTTGCCGATCTATGACTTCTCATACGAA ATGACCACCCTGCTCTCCCCGGACGAACGTACCGCTATGCGTCAGGGCGTCATCA GCCAGCGTACAGCCTGGATCTGCTAGAGGCATCAAATAAAACGAAAGGCTCAGT CGAAAGACTGGGCCTTTCGTTTTATCTGTTGTTTGTCGGTGAACGCTCTCCTGAGT AGGACAAATCCGCCGTTGGGCGACAGATCACGCAAAAGTAGAATTGTGCAAATA AACGGCAGGGTAATTTTTGAAGGTCAGCAACAAAAGTTGATTAATTCTTTGAGGA ACATGCAGTTATGCATGCTGTTGAAAAAGAGGAAGATACTGACTAACTCTAAAG TGGTATTTTACATGCACTTACAATTGATTAAAGACAACATTCACAGTGTGGTTAT TTGTTACACATAGGGGCGAATTCATTAAAGAGGAGAAAGGTACCATGCGTAAAG GCGAAGAGCTGTTCACTGGTGTCGTCCCTATTCTGGTGGAACTGGATGGTGATGT CAACGGTCATAAGTTTTCCGTGCGTGGCGAGGGTGAAGGTGACGCAACTAATGG TAAACTGACGCTGAAGTTCATCTGTACTACTGGTAAACTGCCGGTACCTTGGCCG ACTCTGGTAACGACGCTGACTTATGGTGTTCAGTGCTTTGCTCGTTATCCGGACC ATATGAAGCAGCATGACTTCTTCAAGTCCGCCATGCCGGAAGGCTATGTGCAGG AACGCACGATTTCCTTTAAGGATGACGGCACGTACAAAACGCGTGCGGAAGTGA AATTTGAAGGCGATACCCTGGTAAACCGCATTGAGCTGAAAGGCATTGACTTTAA AGAAGACGGCAATATCCTGGGCCATAAGCTGGAATACAATTTTAACAGCCACAA TGTTTACATCACCGCCGATAAACAAAAAAATGGCATTAAAGCGAATTTTAAAATT CGCCACAACGTGGAGGATGGCAGCGTGCAGCTGGCTGATCACTACCAGCAAAAC ACTCCAATCGGTGATGGTCCTGTTCTGCTGCCAGACAATCACTATCTGAGCACGC AAAGCGTTCTGTCTAAAGATCCGAACGAGAAACGCGATCATATGGTTCTGCTGG AGTTCGTAACCGCAGCGGGCATCACGCATGGTATGGATGAACTGTACAAATGAC TTGGACTCCTGTTGATAGATCCAGTAATGACCTCAGAACTCCATCTGGATTTGTTC AGAACGCTCGGTTGCCGCCGGGCGTTTTTTATTGGTGAGAATCCACCTTTCGACC TGCAGCCTGTTGACAATTAATCATCGGCATAGTATATCGGCATAGTATAATACGA CAAGGTGAGGAACTAAACCATGAAAAAGCCTGAACTCACCGCGACGTCTGTCGA GAAGTTTCTGATCGAAAAGTTCGACAGCGTCTCCGACCTGATGCAGCTCTCGGAG GGCGAAGAATCTCGTGCTTTCAGCTTCGATGTAGGAGGGCGTGGATATGTCCTGC GGGTAAATAGCTGCGCCGATGGTTTCTACAAAGATCGTTATGTTTATCGGCACTT TGCATCGGCCGCGCTCCCGATTCCGGAAGTGCTTGACATTGGGGAATTCAGCGAG AGCCTGACCTATTGCATCTCCCGCCGTGCACAGGGTGTCACGTTGCAAGACCTGC CTGAAACCGAACTGCCCGCTGTTCTGCAGCCGGTCGCGGAGGCCATGGATGCGA TCGCTGCGGCCGATCTTAGCCAGACGAGCGGGTTCGGCCCATTCGGACCGCAAG GAATCGGTCAATACACTACATGGCGTGATTTCATATGCGCGATTGCTGATCCCCA TGTGTATCACTGGCAAACTGTGATGGACGACACCGTCAGTGCGTCCGTCGCGCAG GCTCTCGATGAGCTGATGCTTTGGGCCGAGGACTGCCCCGAAGTCCGGCACCTCG TGCACGCGGATTTCGGCTCCAACAATGTCCTGACGGACAATGGCCGCATAACAG CGGTCATTGACTGGAGCGAGGCGATGTTCGGGGATTCCCAATACGAGGTCGCCA ACATCTTCTTCTGGAGGCCGTGGTTGGCTTGTATGGAGCAGCAGACGCGCTACTT CGAGCGGAGGCATCCGGAGCTTGCAGGATCGCCGCGGCTCCGGGCGTATATGCT CCGCATTGGTCTTGACCAACTCTATCAGAGCTTGGTTGACGGCAATTTCGATGAT GCAGCTTGGGCGCAGGGTCGATGCGACGCAATCGTCCGATCCGGAGCCGGGACT GTCGGGCGTACACAAATCGCCCGCAGAAGCGCGGCCGTCTGGACCGATGGCTGT GTAGAAGTACTCGCCGATAGTGGAAACCGACGCCCCGGATGAGGGGATCAATTC TCTAGAGCTCGCTGATCAGAAGTTCCTGATATATTCCGCTTCCTCGCTCACTGACT CGCTACGCTCGGTCGTTCGACTGCGGCGAGCGGAAATGGCTTACGAACGGGGCG GAGATTTCCTGGAAGATGCCAGGAAGATACTTAACAGGGAAGTGAGAGGGCCGC GGCAAAGCCGTTTTTCCATAGGCTCCGCCCCCCTGACAAGCATCACGAAATCTGA CGCTCAAATCAGTGGTGGCGAAACCCGACAGGACTATAAAGATACCAGGCGTTT CCCCCTGGCGGCTCCCTCGTGCGCTCTCCTGTTCCTGCCTTTCGGTTTACCGGTGT CATTCCGCTGTTATGGCCGCGTTTGTCTCATTCCACGCCTGACACTCAGTTCCGGG TAGGCAGTTCGCTCCAAGCTGGACTGTATGCACGAACCCCCCGTTCAGTCCGACC GCTGCGCCTTATCCGGTAACTATCGTCTTGAGTCCAACCCGGAAAGACATGCAAA AGCACCACTGGCAGCAGCCACTGGTAATTGATTTAGAGGAGTTAGTCTTGAAGTC ATGCGCCGGTTAAGGCTAAACTGAAAGGACAAGTTTTGGTGACTGCGCTCCTCCA AGCCAGTTACCTCGGTTCAAAGAGTTGGTAGCTCAGAGAACCTTCGAAAAACCG CCCTGCAAGGCGGTTTTTTCGTTTTCAGAGCAAGAGATTACGCGCAGACCAAAAC GATCTCAAGAAGATCATCTTATTAATCAGATAAAATATTTCTAGATTTCAGTGCA ATTTATCTCTTCAAATGTAGCACCTGAAGTCAGCCCCATACGATATAAGTTGTT//SEQ ID NO:5. The 61_pQBI_PnsrR-nsrR_Pfnr-fnr(L28H)_PnrfAtrim-LutzRBS-sfgfp_Hygro_p15A plasmid in this nitrite-sensing “nrfA” strain is based on the native nrfA promoter in E. coli having the nucleic acid sequence of SEQ ID NO:27):

TTGGGCGACAGATCACGCAAAAGTAGAATTGTGCAAATAAACGGCAGGG
TAATTTTTGAAGGTCAGCAACAAAAGTTGATTAATTCTTTGAGGAACAT
GCAGTTATGCATGCTGTTGAAAAAGAGGAAGATACTGACTAACTCTAAA
GTGGTATTTTACATGCACTTACAATTGATTAAAGACAACATTCACAGTG
TGGTTATTTGTTACACATAGGGGC//SEQ ID NO: 27.

The E. coli strain that we designated “nirB” contained a nitrite-sensing plasmid (full plasmid name “64_pQBI_PnsrR-nsrR_Pfnr-fnr(L28H)_PnirBtrim-LutzRBS-sfgfp_Hygro_p15A,” schematic plasmid map shown in FIG. 8) having the nucleic acid sequence of SEQ ID NO:6:

GAATTCAAAAGATCTTAAGTAAGTAAGAGTATACGTATATCGGCTAATAACGTATTAAGGCGCTTCGGCGCCTTTTTTTATG
GGGGTATTTTCATCCCAATCCACACGTCCAACGCACAGCAAACACCACGTCGACCCTATCAGCTGCGTGCTTTCTATGAGTC
GTTGCTGCATAACTGGACGTTCTGGATGGCCTGAAAGAGGTTAAACTCTGCGTGGCTTACCGTATGCCGGATGGTCGCGAAG
TGACTACCACTCCGCTGGCAGCTGACGACTGGAAAGGTGTAGAGCCGATTTACGAAACCATGCCGGGCTGGTCTGAATCCAC
CTTCGGCGTGAAAGATCGTAGCGGCCTGCCGCAGGCGGCGCTGAACTATATCAAGCGTATTGAAGAGCTGACTGGTGTGCCG
ATCGATATCATCTCTACCGGTCCGGATCGTACTGAAACCATGATTCTGCGCGACCCGTTCGACGCGTAATTCTGGTACGCCT
GGCAGATATTTTGCCTGCCGGGCGAACAGTGTGATACATTGCTGTGTCGGGTAAGCCATTACGCTATCCGACACAGTGTTAA
ATCCTCGCTTTTTTCCTTCCCCGAACTGAAATAAATTAGCGACACAGCTTGTGGCTGGTTTATCATCAATATAAATGTATTT
TTTCCCGATTTCCCTTTTGAGGTTGATGTGCAGTTAACGAGTTTCACTGATTACGGATTACGTGCGCTGATCTACATGGCGT
CATTGCCAGAAGGGCGGATGACCAGTATTTCTGAAGTGACTGACGTCTACGGCGTCTCCCGTAATCATATGGTCAAAATAAT
CAATCAACTTAGTCGTGCCGGCTACGTGACTGCTGTTCGTGGAAAAAATGGCGGCATTCGCCTGGGTAAACCGGCGAGTGCG
ATACGTATTGGTGATGTGGTGCGCGAGCTGGAGCCCTTATCGCTGGTGAATTGCAGCAGTGAGTTTTGCCACATTACACCTG
CCTGTAGGTTGAAACAGGCACTTTCTAAGGCCGTGCAAAGTTTTCTTACGGAACTGGATAACTACACGCTTGCCGATTTGGT
TGAAGAGAATCAACCGCTTTATAAATTATTGCTGGTGGAGTGACGAAAATCTTCATCAGAGATGACAACGGAGGAACCGAGA
AATAAAGCCCTGAGTTTAACCGCTCGGGGCTTTTTGCGTTTTCGTGAATATTTTGCCGGTAATCTTAGCATTATTGATACGC
TTCCCACTGCTACGGGGGGGACGCCATTTCAGCGCGAAGTCTGGAAAACACTACGCACTATCCCCTGCGGGCAGGTAATGCA
TTACGGCCAACTGGCTGAGCAATTGGGCCGTCCTGGCGCGGCGCGTGCCGTTGGTGCGGCAAACGGATCGAATCCCATCAGC
ATCGTCGTACCTTGCCATCGGGTTATTGGCCGAAACGGCACCATGACCGGATATGCAGGCGGAGTTCAGCGAAAAGAGTGGT
TATTGCGCCATGAAGGTTATCTTTTGCTGTAAACATTAAACAATTTGTGCCAGCTTGTTCACACTTTTATGTAAAGTTACCC
TTAACAACTTAAGGGTTTTCAAATAGATAGACATATATTTACATCTAATATCGGAATTCTCTGCTGTTAAGGTTTGCTTAGA
CTTACTTGCTCCCTAAAAAGATGTTAAAATTGACAAATATCAATTACGGCTTGAGCAGACCTATGATCCCGGAAAAGCGAAT
TATACGGCGCATTCAGTCTGGCGGTTGTGCTATCCATTGCCAGGATTGCAGCATCAGCCAGCATTGCATCCCGTTCACACTC
AACGAACATGAGCTTGATCAGCTTGATAATATCATTGAGCGGAAGAAGCCTATTCAGAAAGGCCAGACGCTGTTTAAGGCTG
GTGATGAACTTAAATCGCTTTATGCCATCCGCTCCGGTACGATTAAAAGTTATACCATCACTGAGCAAGGCGACGAGCAAAT
CACTGGTTTCCATTTAGCAGGCGACCTGGTGGGATTTGACGCCATCGGCAGCGGCCATCACCCGAGCTTCGCGCAGGCGCTG
GAAACCTCGATGGTATGTGAAATCCCGTTCGAAACGCTGGACGATTTGTCCGGTAAAATGCCGAATCTGCGTCAGCAGATGA
TGCGTCTGATGAGCGGTGAAATCAAAGGCGATCAGGACATGATCCTGCTGTTGTCGAAGAAAAATGCCGAGGAACGTCTGGC
TGCATTCATCTACAACCTGTCCCGTCGTTTTGCCCAACGCGGCTTCTCCCCTCGTGAATTCCGCCTGACGATGACTCGTGGC
GATATCGGTAACTATCTGGGCCTGACGGTAGAAACCATCAGCCGTCTGCTGGGTCGCTTCCAGAAAAGCGGCATGCTGGCAG
TCAAAGGTAAATACATCACCATCGAAAATAACGATGCGCTGGCCCAGCTTGCTGGTCATACGCGTAACGTTGCCTGATTTTT
CCGCATAACTCACTATCCTTCTGTCATATCATTAAATTTTTCTGATTTATTGATCTGGCAGAAGGTTCATCACTGTTTCATT
CACCAGATATGGGTTAATCTTTTAATTACAAACTGCGTTGACAGTTGTTGTAAGGAGACCCTGTATGGCTATGTATCAGAAC
ATGCTCGTTGTTATCGATCCTAACCAGGACGACCAACCAGCATTGCGGCGAGCTGTTTATTTACATCAACGGATTGGTGGCA
AAATTAAAGCCTTTTTGCCGATCTATGACTTCTCATACGAAATGACCACCCTGCTCTCCCCGGACGAACGTACCGCTATGCG
TCAGGGCGTCATCAGCCAGCGTACAGCCTGGATCTGCTAGAGGCATCAAATAAAACGAAAGGCTCAGTCGAAAGACTGGGCC
TTTCGTTTTATCTGTTGTTTGTCGGTGAACGCTCTCCTGAGTAGGACAAATCCGCCGACGCATTCTGACCGTACTGGCTGGT
CTGGCTGCGATTCTGATGTACGTCTTTAACACCGGAACACCAGCACATATGGCGTGGTCAATTCTCGCTCTGGGCTTCTTCT
CCAGCGCGATCTATACCACCATCATCACCCTGGGTTCACAGCAGACCAAAGTACCGTCGCCAAAACTGGTTAACTTTGTCCT
GACCTGCGGAACCATCGGTACTATGTTGACCTTTGTGGTTACCGGCCCGATCGTTGAACATAGCGGTCCGCAGGCGGCACTG
CTTACAGCAAACGGTCTGTACGCTGTCGTCTTTGTGATGTGCTTCCTGTTAGGTTTCGTCAGCCGTCACCGTCAGCATAACA
CCCTGACCTCTCATTAATTGCTCATGCCGGACGGCACTATCGTCGTCCGGCCTTTTCCTCTCTTCCCCCGCTACGTGCATCT
ATTTCTATAAACCCGCTCATTTTGTCTATTTTTTGCACAAACATGAAATATCAGACAATTCCGTGACTTAAGAAAATTTATA
CAAATCAGCAATATACCCATTAAGGAGTATATAAAGGTGAATTTGATTTACATCAATAAGCGGGGTTGCTGAATCGTTAAGG
TAGGCGGTAGAATTCATTAAAGAGGAGAAAGGTACCATGCGTAAAGGCGAAGAGCTGTTCACTGGTGTCGTCCCTATTCTGG
TGGAACTGGATGGTGATGTCAACGGTCATAAGTTTTCCGTGCGTGGCGAGGGTGAAGGTGACGCAACTAATGGTAAACTGAC
GCTGAAGTTCATCTGTACTACTGGTAAACTGCCGGTACCTTGGCCGACTCTGGTAACGACGCTGACTTATGGTGTTCAGTGC
TTTGCTCGTTATCCGGACCATATGAAGCAGCATGACTTCTTCAAGTCCGCCATGCCGGAAGGCTATGTGCAGGAACGCACGA
TTTCCTTTAAGGATGACGGCACGTACAAAACGCGTGCGGAAGTGAAATTTGAAGGCGATACCCTGGTAAACCGCATTGAGCT
GAAAGGCATTGACTTTAAAGAAGACGGCAATATCCTGGGCCATAAGCTGGAATACAATTTTAACAGCCACAATGTTTACATC
ACCGCCGATAAACAAAAAAATGGCATTAAAGCGAATTTTAAAATTCGCCACAACGTGGAGGATGGCAGCGTGCAGCTGGCTG
ATCACTACCAGCAAAACACTCCAATCGGTGATGGTCCTGTTCTGCTGCCAGACAATCACTATCTGAGCACGCAATCGTAACC
AGCGTTCTGTCTAAAGATCCGAACGAGAAACGCGATCATATGGTTCTGCTGGAGTGCAGCGGGCATCACGCATGGTATGGAT
GAACTGTACAAATGACTTGGACTCCTGTTGATAGATCCAGTAATGACCTCAGAACTCCATCTGGATTTGTTCAGAACGCTCG
GTTGCCGCCGGGCGTTTTTTATTGGTGAGAATCCACCTTTCGACCTGCAGCCTGTTGACAATTAATCATCGGCATAGTATAT
CGGCATAGTATAATACGACAAGGTGAGGAACTAAACCATGAAAAAGCCTGAACTCACCGCGACGTCTGTCGAGAAGTTTCTG
ATCGAAAAGTTCGACAGCGTCTCCGACCTGATGCAGCTCTCGGAGGGCGAAGAATCTCGTGCTTTCAGCTTCGATGTAGGAG
GGCGTGGATATGTCCTGCGGGTAAATAGCTGCGCCGATGGTTTCTACAAAGATCGTTATGTTTATCGGCACTTTGCATCGGC
CGCGCTCCCGATTCCGGAAGTGCTTGACATTGGGGAATTCAGCGAGAGCCTGACCTATTGCATCTCCCGCCGTGCACAGGGT
GTCACGTTGCAAGACCTGCCTGAAACCGAACTGCCCGCTGTTCTGCAGCCGGTCGCGGAGGCCATGGATGCGATCGCTGCGG
CCGATCTTAGCCAGACGAGCGGGTTCGGCCCATTCGGACCGCAAGGAATCGGTCAATACACTACATGGCGTGATTTCATATG
CGCGATTGCTGATCCCCATGTGTATCACTGGCAAACTGTGATGGACGACACCGTCAGTGCGTCCGTCGCGCAGGCTCTCGAT
GAGCTGATGCTTTGGGCCGAGGACTGCCCCGAAGTCCGGCACCTCGTGCACGCGGATTTCGGCTCCAACAATGTCCTGACGG
ACAATGGCCGCATAACAGCGGTCATTGACTGGAGCGAGGCGATGTTCGGGGATTCCCAATACGAGGTCGCCAACATCTTCTT
CTGGAGGCCGTGGTTGGCTTGTATGGAGCAGCAGACGCGCTACTTCGAGCGGAGGCATCCGGAGCTTGCAGGATCGCCGCGG
CTCCGGGCGTATATGCTCCGCATTGGTCTTGACCAACTCTATCAGAGCTTGGTTGACGGCAATTTCGATGATGCAGCTTGGG
CGCAGGGTCGATGCGACGCAATCGTCCGATCCGGAGCCGGGACTGTCGGGCGTACACAAATCGCCCGCAGAAGCGCGGCCGT
CTGGACCGATGGCTGTGTAGAAGTACTCGCCGATAGTGGAAACCGACGCCCCGGATGAGGGGATCAATTCTCTAGAGCTCGC
TGATCAGAAGTTCCTGATATATTCCGCTTCCTCGCTCACTGACTCGCTACGCTCGGTCGTTCGACTGCGGCGAGCGGAAATG
GCTTACGAACGGGGCGGAGATTTCCTGGAAGATGCCAGGAAGATACTTAACAGGGAAGTGAGAGGGCCGCGGCAAAGCCGTT
TTTCCATAGGCTCCGCCCCCCTGACAAGCATCACGAAATCTGACGCTCAAATCAGTGGTGGCGAAACCCGACAGGACTATAA
AGATACCAGGCGTTTCCCCCTGGCGGCTCCCTCGTGCGCTCTCCTGTTCCTGCCTTTCGGTTTACCGGTGTCATTCCGCTGT
TATGGCCGCGTTTGTCTCATTCCACGCCTGACACTCAGTTCCGGGTAGGCAGTTCGCTCCAAGCTGGACTGTATGCACGAAC
CCCCCGTTCAGTCCGACCGCTGCGCCTTATCCGGTAACTATCGTCTTGAGTCCAACCCGGAAAGACATGCAAAAGCACCACT
GGCAGCAGCCACTGGTAATTGATTTAGAGGAGTTAGTCTTGAAGTCATGCGCCGGTTAAGGCTAAACTGAAAGGACAAGTTT
TGGTGACTGCGCTCCTCCAAGCCAGTTACCTCGGTTCAAAGAGTTGGTAGCTCAGAGAACCTTCGAAAAACCGCCCTGCAAG
GCGGTTTTTTCGTTTTCAGAGCAAGAGATTACGCGCAGACCAAAACGATCTCAAGAAGATCATCTTATTAATCAGATAAAAT
ATTTCTAGATTTCAGTGCAATTTATCTCTTCAAATGTAGCACCTGAAGTCAGCCCCATACGATATAAGTTGTT//
SEQ ID NO: 6.

The 64_pQBI_PnsrR-nsrR_Pfnr-fnr(L28H)_PnirBtrim-LutzRBS-sfgfp_Hygro_p15A plasmid in this nitrite-sensing “nirB” strain is based on the native nirB promoter in E. coli having the nucleic acid sequence of SEQ ID NO:28):

ACGCATTCTGACCGTACTGGCTGGTCTGGCTGCGATTCTGATGTACGTC
TTTAACACCGGAACACCAGCACATATGGCGTGGTCAATTCTCGCTCTGG
GCTTCTTCTCCAGCGCGATCTATACCACCATCATCACCCTGGGTTCACA
GCAGACCAAAGTACCGTCGCCAAAACTGGTTAACTTTGTCCTGACCTGV
GGAACCATCGGTACTATGTTGACCTTTGTGGTTACCGGCCCGATCGTTG
AACATAGCGGTCCGCAGGCGGCACTGCTTACAGCAAACGGTCTGTACGC
TGTCGTCTTTGTGATGTGCTTCCTGTTAGGTTTCGTCAGCCGTCACCGT
CAGCATAACACCCTGACCTCTCATTAATTGCTCATGCCGGACGGCACTA
TCGTCGTCCGGCCTTTTCCTCTCTTCCCCCGCTACGTGCATCTATTTCT
ATAAACCCGCTCATTTTGTCTATTTTTTGCACAAACATGAAATATCAGA
CAATTCCGTGACTTAAGAAAATTTATACAAATCAGCAATATACCCATTA
AGGAGTATATAAAGGTGAATTTGATTTACATCAATAAGCGGGGTTGCTG
AATCGTTAAGGTAGGCGGTA//SEQ ID NO: 28.

Both the nrfA strain plasmid (61_pQBI_PnsrR-nsrR_Pfnr-fnr(L28H)_PnrfAtrim-LutzRBS-sfgfp_Hygro_p15A) and the nirB strain plasmid (64_pQBI_PnsrR-nsrR_Pfnr-fnr(L28H)_PnirBtrim-LutzRBS-sfgfp_Hygro_p15A) contained the nsrR module (SEQ ID NO:25), as well as the fnr module (SEQ ID NO:20), as the FNR dual regulator activates transcription from the nrfA and nirB promoters. (See, H. Corker and R. K. Poole, “Nitric oxide formation by Escherichia coli. Dependence on nitrite reductase, the NO-sensing regulator Fnr, and flavohemoglobin Hmp,” Journal of Biological Chemistry, vol. 278, no. 34, pp. 31584-31592 (2003), doi: 10.1074/jbc.M303282200).

### Exemplary Strains Sensitive to Phosphate

The E. coli strain that we designated “phoB” contained a phosphate-sensing plasmid (full plasmid name “84_pQBI_PphoB-phoBR-LutzRBS-sfgfp_Hygro_p15A,” schematic plasmid map shown in FIG. 9) having the nucleic acid sequence of SEQ ID NO:7:

GTTTTTATCGCGTTGATAAAGCGCGTTCCCGGCAAACCGGCGGTAGCGGATTAGGGTTAGCGATCGTGAAACATGCTGTG
AATCATCACGAAAGTCGCCTGAATATTGAGAGTACAGTAGGAAAAGGAACACGTTTCAGTTTTGTTATCCCGGAACGTTT
AATTGCCAAAAACAGCGATTAAGAATTCATTAAAGAGGAGAAAGGTACCATGCGTAAAGGCGAAGAGCTGTTCACTGGTG
TCGTCCCTATTCTGGTGGAACTGGATGGTGATGTCAACGGTCATAAGTTTTCCGTGCGTGGCGAGGGTGAAGGTGACGCA
ACTAATGGTAAACTGACGCTGAAGTTCATCTGTACTACTGGTAAACTGCCGGTACCTTGGCCGACTCTGGTAACGACGCT
GACTTATGGTGTTCAGTGCTTTGCTCGTTATCCGGACCATATGAAGCAGCATGACTTCTTCAAGTCCGCCATGCCGGAAG
GCTATGTGCAGGAACGCACGATTTCCTTTAAGGATGACGGCACGTACAAAACGCGTGCGGAAGTGAAATTTGAAGGCGAT
ACCCTGGTAAACCGCATTGAGCTGAAAGGCATTGACTTTAAAGAAGACGGCAATATCCTGGGCCATAAGCTGGAATACAA
TTTTAACAGCCACAATGTTTACATCACCGCCGATAAACAAAAAAATGGCATTAAAGCGAATTTTAAAATTCGCCACAACG
TGGAGGATGGCAGCGTGCAGCTGGCTGATCACTACCAGCAAAACACTCCAATCGGTGATGGTCCTGTTCTGCTGCCAGAC
AATCACTATCTGAGCACGCAAAGCGTTCTGTCTAAAGATCCGAACGAGAAACGCGATCATATGGTTCTGCTGGAGTTCGT
AACCGCAGCGGGCATCACGCATGGTATGGATGAACTGTACAAATGACTTGGACTCCTGTTGATAGATCCAGTAATGACCT
CAGAACTCCATCTGGATTTGTTCAGAACGCTCGGTTGCCGCCGGGCGTTTTTTATTGGTGAGAATCCACCTTTCGACCTG
CAGCCTGTTGACAATTAATCATCGGCATAGTATATCGGCATAGTATAATACGACAAGGTGAGGAACTAAACCATGAAAAA
GCCTGAACTCACCGCGACGTCTGTCGAGAAGTTTCTGATCGAAAAGTTCGACAGCGTCTCCGACCTGATGCAGCTCTCGG
AGGGCGAAGAATCTCGTGCTTTCAGCTTCGATGTAGGAGGGCGTGGATATGTCCTGCGGGTAAATAGCTGCGCCGATGGT
TTCTACAAAGATCGTTATGTTTATCGGCACTTTGCATCGGCCGCGCTCCCGATTCCGGAAGTGCTTGACATTGGGGAATT
CAGCGAGAGCCTGACCTATTGCATCTCCCGCCGTGCACAGGGTGTCACGTTGCAAGACCTGCCTGAAACCGAACTGCCCG
CTGTTCTGCAGCCGGTCGCGGAGGCCATGGATGCGATCGCTGCGGCCGATCTTAGCCAGACGAGCGGGTTCGGCCCATTC
GGACCGCAAGGAATCGGTCAATACACTACATGGCGTGATTTCATATGCGCGATTGCTGATCCCCATGTGTATCACTGGCA
AACTGTGATGGACGACACCGTCAGTGCGTCCGTCGCGCAGGCTCTCGATGAGCTGATGCTTTGGGCCGAGGACTGCCCCG
AAGTCCGGCACCTCGTGCACGCGGATTTCGGCTCCAACAATGTCCTGACGGACAATGGCCGCATAACAGCGGTCATTGAC
TGGAGCGAGGCGATGTTCGGGGATTCCCAATACGAGGTCGCCAACATCTTCTTCTGGAGGCCGTGGTTGGCTTGTATGGA
GCAGCAGACGCGCTACTTCGAGCGGAGGCATCCGGAGCTTGCAGGATCGCCGCGGCTCCGGGCGTATATGCTCCGCATTG
GTCTTGACCAACTCTATCAGAGCTTGGTTGACGGCAATTTCGATGATGCAGCTTGGGCGCAGGGTCGATGCGACGCAATC
GTCCGATCCGGAGCCGGGACTGTCGGGCGTACACAAATCGCCCGCAGAAGCGCGGCCGTCTGGACCGATGGCTGTGTAGA
AGTACTCGCCGATAGTGGAAACCGACGCCCCGGATGAGGGGATCAATTCTCTAGAGCTCGCTGATCAGAAGTTCCTGATA
TATTCCGCTTCCTCGCTCACTGACTCGCTACGCTCGGTCGTTCGACTGCGGCGAGCGGAAATGGCTTACGAACGGGGCGG
AGATTTCCTGGAAGATGCCAGGAAGATACTTAACAGGGAAGTGAGAGGGCCGCGGCAAAGCCGTTTTTCCATAGGCTCCG
CCCCCCTGACAAGCATCACGAAATCTGACGCTCAAATCAGTGGTGGCGAAACCCGACAGGACTATAAAGATACCAGGCGT
TTCCCCCTGGCGGCTCCCTCGTGCGCTCTCCTGTTCCTGCCTTTCGGTTTACCGGTGTCATTCCGCTGTTATGGCCGCGT
TTGTCTCATTCCACGCCTGACACTCAGTTCCGGGTAGGCAGTTCGCTCCAAGCTGGACTGTATGCACGAACCCCCCGTTC
AGTCCGACCGCTGCGCCTTATCCGGTAACTATCGTCTTGAGTCCAACCCGGAAAGACATGCAAAAGCACCACTGGCAGCA
GCCACTGGTAATTGATTTAGAGGAGTTAGTCTTGAAGTCATGCGCCGGTTAAGGCTAAACTGAAAGGACAAGTTTTGGTG
ACTGCGCTCCTCCAAGCCAGTTACCTCGGTTCAAAGAGTTGGTAGCTCAGAGAACCTTCGAAAAACCGCCCTGCAAGGCG
GTTTTTTCGTTTTCAGAGCAAGAGATTACGCGCAGACCAAAACGATCTCAAGAAGATCATCTTATTAATCAGATAAAATA
TTTCTAGATTTCAGTGCAATTTATCTCTTCAAATGTAGCACCTGAAGTCAGCCCCATACGATATAAGTTGTT//SEQ ID
NO: 7.

The 84_pQBI_PphoB-phoBR-LutzRBS-sfgfp_Hygro_p15A plasmid in this phosphate-sensing “phoB” strain is based on the native phoB promoter in E. coli having the nucleic acid sequence of SEQ ID NO:29):

TTTTTCTGCGCCACGGAAATCAATAACCTGAAGATATGTGCGACGAGCT
TTTCATAAATCTGTCATAAATCTGACGCATAATGACGTCGCATTAATGA
TCGCAACCTATTTATTACAACAGGGCAAATCATGGCGAGACGTATTCTG
GTCGTAGAAGATGAAGCTCCAATTCGCGAAATGGTCTGCTTCGTGCTCG
AACAAAATGGCTTTCAGCCGGTCGAAGCGGAAGATTATGACAGTGCTGT
GAATCAACTGAATGAACCCTGGCCGGATTTAATTCTCCTCGACTGGATG
TTACCTGGCGGCTCCGGTATCCAGTTCATCAAACACCTCAAGCGCGAGT
CGATGACCCGGGATATTCCAGTGGTGATGTTGACCGCCAGAGGGGAAGA
AGAAGATCGCGTGCGCGGCCTTGAAACCGGCGCGGATGACTATATCACC
AAGCCGTTTTCGCCGAAGGAGCTGGTGGCGCGAATCAAAGCGGTAATGC
GCCGTATTTCGCCAATGGCGGTGGAAGAGGTGATTGAGATGCAGGGATT
AAGTCTCGACCCGACATCTCACCGAGTGATGGCGGGCGAAGAGCCGCTG
GAGATGGGGCCGACAGAATTTAAACTGCTGCACTTTTTTATGACGCATC
CTGAGCGCGTGTACAGCCGCGAGCAGCTGTTAAACCACGTCTGGGGAAC
TAACGTGTATGTGGAAGACCGCACGGTCGATGTCCACATTCGTCGCCTG
CGTAAAGCACTGGAGCCCGGCGGGCATGACCGCATGGTGCAGACCGTGC
GCGGTACAGGATATCGTTTTTCAACCCGCTTTTAACGCCTTGCTCATCG
GACGCAGAGCAGGGCTTATGATTTCTTAACTGGAGTATCTTACGTGCTG
GAACGGCTGTCGTGGAAAAGGCTGGTGCTGGAGCTGCTACTTTGCTGCC
TCCCGGCTTTCATCCTGGGTGCATTTTTTGGTTACCTGCCCTGGTTTTT
GCTGGCATCGGTAACAGGACTGCTTATCTGGCATTTCTGGAATTTATTG
CGCCTTTCATGGTGGCTGTGGGTGGATCGCAGTATGACCCCGCCACCGG
GGCGTGGTAGCTGGGAACCGCTACTATACGGCTTACACCAGATGCAGCT
GCGAAATAAAAAACGCCGCCGTGAACTGGGCAATCTGATTAAACGCTTT
CGTAGCGGCGCGGAGTCGCTGCCCGACGCGGTGGTGCTGACCACGGAAG
AGGGCGGTATTTTCTGGTGTAACGGTCTGGCGCAACAAATTCTTGGTTT
GCGCTGGCCGGAAGATAACGGGCAGAACATCCTTAACCTACTGCGTTAC
CCGGAGTTTACGCAATATCTGAAAACGCGTGATTTTTCTCGCCCGCTCA
ATCTGGTGCTCAACACCGGGCGGCATCTGGAAATTCGCGTCATGCCTTA
TACCCACAAACAGTTGCTGATGGTGGCGCGTGATGTCACGCAAATGCAT
CAACTGGAAGGGGCGCGGCGTAACTTTTTTGCCAACGTGAGCCATGAGT
TACGTACGCCATTGACCGTGTTACAGGGTTACCTGGAGATGATGAATGA
GCAGCCGCTGGAAGGCGCGGTACGCGAAAAAGCGTTGCACACCATGCGC
GAGCAGACCCAGCGGATGGAAGGACTGGTGAAGCAATTGCTGACGCTGT
CGAAAATAGAAGCCGCACCGACGCATTTGCTCAATGAAAAGGTTGATGT
GCCGATGATGCTGCGCGTTGTTGAGCGCGAGGCTCAGACTCTGAGTCAG
AAAAAACAGACATTTACCTTTGAGATAGATAACGGCCTCAAGGTGTCTG
GCAACGAAGATCAGCTACGCAGTGCGATTTCGAACCTGGTCTATAACGC
CGTGAATCATACGCCGGAAGGCACGCATATCACCGTACGCTGGCAGCGA
GTGCCGCACGGTGCCGAATTTAGCGTTGAAGATAACGGACCGGGCATTG
CACCGGAGCATATTCCGCGCCTGACCGAGCGTTTTTATCGCGTTGATAA
AGCGCGTTCCCGGCAAACCGGCGGTAGCGGATTAGGGTTAGCGATCGTG
AAACATGCTGTGAATCATCACGAAAGTCGCCTGAATATTGAGAGTACAG
TAGGAAAAGGAACACGTTTCAGTTTTGTTATCCCGGAACGTTTAATTGC
CAAAAACAGCGATTAA//SEQ ID NO: 29.

The phoB gene codes for the transcriptional dual regulator PhoB, which activates expression of the Pho regulon to uptake environmental inorganic phosphate (Pᵢ). Specifically, PhoB and the integral membrane sensor histidine kinase, PhoR, comprise a two-component-system (TCS) where, under phosphate-limited conditions, PhoR autophosphorylates then transfers the phosphate group to PhoB. This activated PhoB~P autoregulates the phoB promoter until Pᵢ levels rise to excess, at which point PhoR autophosphorylation is inhibited and PhoB~P is dephosphorylated. (Y. J. Hsieh and B. L. Wanner, “Global regulation by the seven-component Pi signaling system,” Curr. Opin. Microbiol. 13(2):198-203 (2010), doi: 10.1016/j.mib.2010.01.014). Because PhoB binds to sites on the phoB promoter for autoregulation, and both PhoB and PhoR are required components of the TCS, we include both genes behind the native phoB promoter in the plasmid to roughly maintain their relative copy numbers.

The E. coli strain that we designated “pstS” contained a phosphate-sensing plasmid (full plasmid name “85_pQBI_Pfnr-fnr(L28H)_PphoB-phoBR_PpstS-LutzRBS-sfgfp_Hygro_p15A,” schematic plasmid map shown in FIG. 10) having the nucleic acid sequence of SEQ ID NO:8:

GAATTCAAAAGATCTTAAGTAAGTAAGAGTATACGTATATCGGCTAATAACGTAT
TAAGGCGCTTCGGCGCCTTTTTTTATGGGGGTATTTTCATCCCAATCCACACGTCC
AACGCACAGCAAACACCACGTCGACCCTATCAGCTGCGTGCTTTCTATGAGTCGT
TGCTGCATAACTCGTGAATATTTTGCCGGTAATCTTAGCATTATTGATACGCTTCC
CACTGCTACGGGGGGGACGCCATTTCAGCGCGAAGTCTGGAAAACACTACGCAC
TATCCCCTGCGGGCAGGTAATGCATTACGGCCAACTGGCTGAGCAATTGGGCCGT
CCTGGCGCGGCGCGTGCCGTTGGTGCGGCAAACGGATCGAATCCCATCAGCATC
GTCGTACCTTGCCATCGGGTTATTGGCCGAAACGGCACCATGACCGGATATGCAG
GCGGAGTTCAGCGAAAAGAGTGGTTATTGCGCCATGAAGGTTATCTTTTGCTGTA
AACATTAAACAATTTGTGCCAGCTTGTTCACACTTTTATGTAAAGTTACCCTTAAC
AACTTAAGGGTTTTCAAATAGATAGACATATATTTACATCTAATATCGGAATTCT
CTGCTGTTAAGGTTTGCTTAGACTTACTTGCTCCCTAAAAAGATGTTAAAATTGA
CAAATATCAATTACGGCTTGAGCAGACCTATGATCCCGGAAAAGCGAATTATAC
GGCGCATTCAGTCTGGCGGTTGTGCTATCCATTGCCAGGATTGCAGCATCAGCCA
GCATTGCATCCCGTTCACACTCAACGAACATGAGCTTGATCAGCTTGATAATATC
ATTGAGCGGAAGAAGCCTATTCAGAAAGGCCAGACGCTGTTTAAGGCTGGTGAT
GAACTTAAATCGCTTTATGCCATCCGCTCCGGTACGATTAAAAGTTATACCATCA
CTGAGCAAGGCGACGAGCAAATCACTGGTTTCCATTTAGCAGGCGACCTGGTGG
GATTTGACGCCATCGGCAGCGGCCATCACCCGAGCTTCGCGCAGGCGCTGGAAA
CCTCGATGGTATGTGAAATCCCGTTCGAAACGCTGGACGATTTGTCCGGTAAAAT
GCCGAATCTGCGTCAGCAGATGATGCGTCTGATGAGCGGTGAAATCAAAGGCGA
TCAGGACATGATCCTGCTGTTGTCGAAGAAAAATGCCGAGGAACGTCTGGCTGC
ATTCATCTACAACCTGTCCCGTCGTTTTGCCCAACGCGGCTTCTCCCCTCGTGAAT
TCCGCCTGACGATGACTCGTGGCGATATCGGTAACTATCTGGGCCTGACGGTAGA
AACCATCAGCCGTCTGCTGGGTCGCTTCCAGAAAAGCGGCATGCTGGCAGTCAA
AGGTAAATACATCACCATCGAAAATAACGATGCGCTGGCCCAGCTTGCTGGTCAT
ACGCGTAACGTTGCCTGATTTTTCCGCATAACTCACTATCCTTCTGTCATATCATT
AAATTTTTCTGATTTATTGATCTGGCAGAAGGTTCATCACTGTTTCATTCACCAGA
TATGGGTTAATCTTTTAATTACAAACTGCGTTGACAGTTGTTGTAAGGAGACCCT
GTATGGCTATGTATCAGAACATGCTCGTTGTTATCGATCCTAACCAGGACGACCA
ACCAGCATTGCGGCGAGCTGTTTATTTACATCAACGGATTGGTGGCAAAATTAAA
GCCTTTTTGCCGATCTATGACTTCTCATACGAAATGACCACCCTGCTCTCCCCGGA
CGAACGTACCGCTATGCGTCAGGGCGTCATCAGCCAGCGTACAGCCTGGATCTG
CTAGAGGCATCAAATAAAACGAAAGGCTCAGTCGAAAGACTGGGCCTTTCGTTT
TATCTGTTGTTTGTCGGTGAACGCTCTCCTGAGTAGGACAAATCCGCCGTTTTTCT
GCGCCACGGAAATCAATAACCTGAAGATATGTGCGACGAGCTTTTCATAAATCTG
TCATAAATCTGACGCATAATGACGTCGCATTAATGATCGCAACCTATTTATTACA
ACAGGGCAAATCATGGCGAGACGTATTCTGGTCGTAGAAGATGAAGCTCCAATT
CGCGAAATGGTCTGCTTCGTGCTCGAACAAAATGGCTTTCAGCCGGTCGAAGCGG
AAGATTATGACAGTGCTGTGAATCAACTGAATGAACCCTGGCCGGATTTAATTCT
CCTCGACTGGATGTTACCTGGCGGCTCCGGTATCCAGTTCATCAAACACCTCAAG
CGCGAGTCGATGACCCGGGATATTCCAGTGGTGATGTTGACCGCCAGAGGGGAA
GAAGAAGATCGCGTGCGCGGCCTTGAAACCGGCGCGGATGACTATATCACCAAG
CCGTTTTCGCCGAAGGAGCTGGTGGCGCGAATCAAAGCGGTAATGCGCCGTATTT
CGCCAATGGCGGTGGAAGAGGTGATTGAGATGCAGGGATTAAGTCTCGACCCGA
CATCTCACCGAGTGATGGCGGGCGAAGAGCCGCTGGAGATGGGGCCGACAGAAT
TTAAACTGCTGCACTTTTTTATGACGCATCCTGAGCGCGTGTACAGCCGCGAGCA
GCTGTTAAACCACGTCTGGGGAACTAACGTGTATGTGGAAGACCGCACGGTCGA
TGTCCACATTCGTCGCCTGCGTAAAGCACTGGAGCCCGGCGGGCATGACCGCAT
GGTGCAGACCGTGCGCGGTACAGGATATCGTTTTTCAACCCGCTTTTAACGCCTT
GCTCATCGGACGCAGAGCAGGGCTTATGATTTCTTAACTGGAGTATCTTACGTGC
TGGAACGGCTGTCGTGGAAAAGGCTGGTGCTGGAGCTGCTACTTTGCTGCCTCCC
GGCTTTCATCCTGGGTGCATTTTTTGGTTACCTGCCCTGGTTTTTGCTGGCATCGG
TAACAGGACTGCTTATCTGGCATTTCTGGAATTTATTGCGCCTTTCATGGTGGCTG
TGGGTGGATCGCAGTATGACCCCGCCACCGGGGCGTGGTAGCTGGGAACCGCTA
CTATACGGCTTACACCAGATGCAGCTGCGAAATAAAAAACGCCGCCGTGAACTG
GGCAATCTGATTAAACGCTTTCGTAGCGGCGCGGAGTCGCTGCCCGACGCGGTG
GTGCTGACCACGGAAGAGGGCGGTATTTTCTGGTGTAACGGTCTGGCGCAACAA
ATTCTTGGTTTGCGCTGGCCGGAAGATAACGGGCAGAACATCCTTAACCTACTGC
GTTACCCGGAGTTTACGCAATATCTGAAAACGCGTGATTTTTCTCGCCCGCTCAA
TCTGGTGCTCAACACCGGGCGGCATCTGGAAATTCGCGTCATGCCTTATACCCAC
AAACAGTTGCTGATGGTGGCGCGTGATGTCACGCAAATGCATCAACTGGAAGGG
GCGCGGCGTAACTTTTTTGCCAACGTGAGCCATGAGTTACGTACGCCATTGACCG
TGTTACAGGGTTACCTGGAGATGATGAATGAGCAGCCGCTGGAAGGCGCGGTAC
GCGAAAAAGCGTTGCACACCATGCGCGAGCAGACCCAGCGGATGGAAGGACTG
GTGAAGCAATTGCTGACGCTGTCGAAAATAGAAGCCGCACCGACGCATTTGCTC
AATGAAAAGGTTGATGTGCCGATGATGCTGCGCGTTGTTGAGCGCGAGGCTCAG
ACTCTGAGTCAGAAAAAACAGACATTTACCTTTGAGATAGATAACGGCCTCAAG
GTGTCTGGCAACGAAGATCAGCTACGCAGTGCGATTTCGAACCTGGTCTATAACG
CCGTGAATCATACGCCGGAAGGCACGCATATCACCGTACGCTGGCAGCGAGTGC
CGCACGGTGCCGAATTTAGCGTTGAAGATAACGGACCGGGCATTGCACCGGAGC
ATATTCCGCGCCTGACCGAGCGTTTTTATCGCGTTGATAAAGCGCGTTCCCGGCA
AACCGGCGGTAGCGGATTAGGGTTAGCGATCGTGAAACATGCTGTGAATCATCA
CGAAAGTCGCCTGAATATTGAGAGTACAGTAGGAAAAGGAACACGTTTCAGTTT
TGTTATCCCGGAACGTTTAATTGCCAAAAACAGCGATTAATCCGCCTTTGTCATC
TTTTATTGCCATAAGCCAGTCGATGCTGGCTTATTTTCTTTGCAGTCAAAATACGG
GCGTTAGATTTTACAACGATTGGTGATTTTTTGTTCGCATGATTAGCCATGTCTTT
TTCACGGAAATAGTGTTTTATACTGGTTGGTGATTTCTTATCGCTATATACCTCTG
GTTTTTAGATCCCTCCTTGCTTTAAAACGTTATAAGCGTTTAAATTGCGCTTCAGG
TGCTTAAATGGATGCCCTGCGTAAGCGGGGCATTTTTCTTCCTGTTATGTTTTTAA
TCAAACATCCTGCCAACTCCATGTGACAAACCGTCATCTTCGGCTACTTTTTCTCT
GTCACAGAATGAAAATTTTTCTGTCATCTCTTCGTTATTAATGTTTGTAATTGACT
GAATATCAACGCTTATTTAAATCAGACTGAAGACTTTATCTCTCTGTCATAAAAC
TGTCATATTCCTTACATATAACTGTCACCTGTTTGTCCTATTTTGCTTCTCGTAGCC
AACAAACAATGCTTTATGAATCCTCCCGAATTCATTAAAGAGGAGAAAGGTACC
ATGCGTAAAGGCGAAGAGCTGTTCACTGGTGTCGTCCCTATTCTGGTGGAACTGG
ATGGTGATGTCAACGGTCATAAGTTTTCCGTGCGTGGCGAGGGTGAAGGTGACG
CAACTAATGGTAAACTGACGCTGAAGTTCATCTGTACTACTGGTAAACTGCCGGT
ACCTTGGCCGACTCTGGTAACGACGCTGACTTATGGTGTTCAGTGCTTTGCTCGTT
ATCCGGACCATATGAAGCAGCATGACTTCTTCAAGTCCGCCATGCCGGAAGGCTA
TGTGCAGGAACGCACGATTTCCTTTAAGGATGACGGCACGTACAAAACGCGTGC
GGAAGTGAAATTTGAAGGCGATACCCTGGTAAACCGCATTGAGCTGAAAGGCAT
TGACTTTAAAGAAGACGGCAATATCCTGGGCCATAAGCTGGAATACAATTTTAAC
AGCCACAATGTTTACATCACCGCCGATAAACAAAAAAATGGCATTAAAGCGAAT
TTTAAAATTCGCCACAACGTGGAGGATGGCAGCGTGCAGCTGGCTGATCACTACC
AGCAAAACACTCCAATCGGTGATGGTCCTGTTCTGCTGCCAGACAATCACTATCT
GAGCACGCAAAGCGTTCTGTCTAAAGATCCGAACGAGAAACGCGATCATATGGT
TCTGCTGGAGTTCGTAACCGCAGCGGGCATCACGCATGGTATGGATGAACTGTAC
AAATGACTTGGACTCCTGTTGATAGATCCAGTAATGACCTCAGAACTCCATCTGG
ATTTGTTCAGAACGCTCGGTTGCCGCCGGGCGTTTTTTATTGGTGAGAATCCACCT
TTCGACCTGCAGCCTGTTGACAATTAATCATCGGCATAGTATATCGGCATAGTAT
AATACGACAAGGTGAGGAACTAAACCATGAAAAAGCCTGAACTCACCGCGACGT
CTGTCGAGAAGTTTCTGATCGAAAAGTTCGACAGCGTCTCCGACCTGATGCAGCT
CTCGGAGGGCGAAGAATCTCGTGCTTTCAGCTTCGATGTAGGAGGGCGTGGATAT
GTCCTGCGGGTAAATAGCTGCGCCGATGGTTTCTACAAAGATCGTTATGTTTATC
GGCACTTTGCATCGGCCGCGCTCCCGATTCCGGAAGTGCTTGACATTGGGGAATT
CAGCGAGAGCCTGACCTATTGCATCTCCCGCCGTGCACAGGGTGTCACGTTGCAA
GACCTGCCTGAAACCGAACTGCCCGCTGTTCTGCAGCCGGTCGCGGAGGCCATG
GATGCGATCGCTGCGGCCGATCTTAGCCAGACGAGCGGGTTCGGCCCATTCGGA
CCGCAAGGAATCGGTCAATACACTACATGGCGTGATTTCATATGCGCGATTGCTG
ATCCCCATGTGTATCACTGGCAAACTGTGATGGACGACACCGTCAGTGCGTCCGT
CGCGCAGGCTCTCGATGAGCTGATGCTTTGGGCCGAGGACTGCCCCGAAGTCCG
GCACCTCGTGCACGCGGATTTCGGCTCCAACAATGTCCTGACGGACAATGGCCGC
ATAACAGCGGTCATTGACTGGAGCGAGGCGATGTTCGGGGATTCCCAATACGAG
GTCGCCAACATCTTCTTCTGGAGGCCGTGGTTGGCTTGTATGGAGCAGCAGACGC
GCTACTTCGAGCGGAGGCATCCGGAGCTTGCAGGATCGCCGCGGCTCCGGGCGT
ATATGCTCCGCATTGGTCTTGACCAACTCTATCAGAGCTTGGTTGACGGCAATTT
CGATGATGCAGCTTGGGCGCAGGGTCGATGCGACGCAATCGTCCGATCCGGAGC
CGGGACTGTCGGGCGTACACAAATCGCCCGCAGAAGCGCGGCCGTCTGGACCGA
TGGCTGTGTAGAAGTACTCGCCGATAGTGGAAACCGACGCCCCGGATGAGGGGA
TCAATTCTCTAGAGCTCGCTGATCAGAAGTTCCTGATATATTCCGCTTCCTCGCTC
ACTGACTCGCTACGCTCGGTCGTTCGACTGCGGCGAGCGGAAATGGCTTACGAAC
GGGGCGGAGATTTCCTGGAAGATGCCAGGAAGATACTTAACAGGGAAGTGAGAG
GGCCGCGGCAAAGCCGTTTTTCCATAGGCTCCGCCCCCCTGACAAGCATCACGAA
ATCTGACGCTCAAATCAGTGGTGGCGAAACCCGACAGGACTATAAAGATACCAG
GCGTTTCCCCCTGGCGGCTCCCTCGTGCGCTCTCCTGTTCCTGCCTTTCGGTTTAC
CGGTGTCATTCCGCTGTTATGGCCGCGTTTGTCTCATTCCACGCCTGACACTCAGT
TCCGGGTAGGCAGTTCGCTCCAAGCTGGACTGTATGCACGAACCCCCCGTTCAGT
CCGACCGCTGCGCCTTATCCGGTAACTATCGTCTTGAGTCCAACCCGGAAAGACA
TGCAAAAGCACCACTGGCAGCAGCCACTGGTAATTGATTTAGAGGAGTTAGTCTT
GAAGTCATGCGCCGGTTAAGGCTAAACTGAAAGGACAAGTTTTGGTGACTGCGC
TCCTCCAAGCCAGTTACCTCGGTTCAAAGAGTTGGTAGCTCAGAGAACCTTCGAA
AAACCGCCCTGCAAGGCGGTTTTTTCGTTTTCAGAGCAAGAGATTACGCGCAGAC
CAAAACGATCTCAAGAAGATCATCTTATTAATCAGATAAAATATTTCTAGATTTC
AGTGCAATTTATCTCTTCAAATGTAGCACCTGAAGTCAGCCCCATACGATATAAGTTGTT//
SEQ ID NO: 8.

The 85_pQBI_Pfnr-fnr(L28H)_PphoB-phoBR_PpstS-LutzRBS-sfgfp_Hygro_p15A plasmid in this phosphate-sensing “pstS” strain is based on the native pstS promoter in E. coli having the nucleic acid sequence of SEQ ID NO:31):

SEQ ID NO: 31
TAAATGGATGCCCTGCGTAAGCGGGGCATTTTTCTTCCTGTTATGTTTT
TAATCAAACATCCTGCCAACTCCATGTGACAAACCGTCATCTTCGGCTA
CTTTTTCTCTGTCACAGAATGAAAATTTTTCTGTCATCTCTTCGTTATT
AATGTTTGTAATTGACTGAATATCAACGCTTATTTAAATCAGACTGAAG
ATATTCCTTACATATAACTGTCACCTGTTTGTCCTATTTTGCTTCTCGT
AACTTTATCTCTCTGTCATAAAACTGTCGCCAACAAACAATGCTTTATG
AATCCTCCC//.

The Pst (phosphate-specific transport) system regulated by the PhoB/PhoR TCS is the predominant mechanism of Pᵢ uptake in E. coli, and the pstS gene codes for the periplasmic phosphate binding protein of the ABC phosphate transport system. Although the pstSCAB-phoU operon has several internal promoters, the pstS promoter is most strongly activated and transcribes all genes, including the transporter membrane subunits (pstC, pstA), transporter ATP binding subunit (pstB), and a chaperone protein (phoU). (B. Spira, M. Aguena, J. V. de Castro Oliveira, and E. Yagil, “Alternative promoters in the pst operon of Escherichia coli,” Molecular Genetics and Genomics, 284(6):489-498 (2010), doi: 10.1007/s00438-010-0584-x).

When Pᵢ is in excess and bound to PstS, a signal propagates through PhoU to PhoR to dephosphorylate PhoB~P, thereby repressing pstSCAB-phoU expression. However, when Pᵢ is limiting and not bound to PstS, PhoR phosphorylates PhoB to activate pstSCAB-phoU expression. (Y. J. Hsieh and B. L. Wanner, “Global regulation by the seven-component Pi signaling system,” Curr. Opin. Microbiol. 13(2):198-203 (2010), doi: 10.1016/j.mib.2010.01.014). Because PhoB binds to sites on both the phoB and pstS promoters, and both PhoB and PhoR are required components of the TCS, we include both genes behind the native phoB promoter (SEQ ID NO:30) in the 85_pQBI_Pfnr-fnr(L28H)_PphoB-phoBR_PpstS-LutzRBS-sfgfp_Hygro_p15A plasmid to roughly maintain the relative copy numbers of PhoB and PhoR. Additionally, the previously described fnr module is included in this plasmid, as the FNR dual regulator activates transcription from the pstS promoter. (K. Salmon, S. pin Hung, K. Mekjian, P. Baldi, G. W. Hatfield, and R. P. Gunsalus, “Global gene expression profiling in Escherichia coli K12: The effects of oxygen availability and FNR,” Journal of Biological Chemistry 278(32):29837-29855 (2003), doi: 10.1074/jbc.M213060200). The nucleotide sequence of the native phoB promoter in the 85_pQBI_Pfnr-fnr(L28H)_PphoB-phoBR_PpstS-LutzRBS-sfgfp_Hygro_p15A plasmid is SEQ ID NO:30:

SEQ ID NO: 30
TTTTTCTGCGCCACGGAAATCAATAACCTGAAGATATGTGCGACGAGCT
TTTCATAAATCTGTCATAAATCTGACGCATAATGACGTCGCATTAATGA
TCGCAACCTATTTATTACAACAGGGCAAATCATGGCGAGACGTATTCTG
GTCGTAGAAGATGAAGCTCCAATTCGCGAAATGGTCTGCTTCGTGCTCG
AACAAAATGGCTTTCAGCCGGTCGAAGCGGAAGATTATGACAGTGCTGT
GAATCAACTGAATGAACCCTGGCCGGATTTAATTCTCCTCGACTGGATG
TTACCTGGCGGCTCCGGTATCCAGTTCATCAAACACCTCAAGCGCGAGT
CGATGACCCGGGATATTCCAGTGGTGATGTTGACCGCCAGAGGGGAAGA
AGAAGATCGCGTGCGCGGCCTTGAAACCGGCGCGGATGACTATATCACC
AAGCCGTTTTCGCCGAAGGAGCTGGTGGCGCGAATCAAAGCGGTAATGC
GCCGTATTTCGCCAATGGCGGTGGAAGAGGTGATTGAGATGCAGGGATT
AAGTCTCGACCCGACATCTCACCGAGTGATGGCGGGCGAAGAGCCGCTG
GAGATGGGGCCGACAGAATTTAAACTGCTGCACTTTTTTATGACGCATC
CTGAGCGCGTGTACAGCCGCGAGCAGCTGTTAAACCACGTCTGGGGAAC
TAACGTGTATGTGGAAGACCGCACGGTCGATGTCCACATTCGTCGCCTG
CGTAAAGCACTGGAGCCCGGCGGGCATGACCGCATGGTGCAGACCGTGC
GCGGTACAGGATATCGTTTTTCAACCCGCTTTTAACGCCTTGCTCATCG
GACGCAGAGCAGGGCTTATGATTTCTTAACTGGAGTATCTTACGTGCTG
GAACGGCTGTCGTGGAAAAGGCTGGTGCTGGAGCTGCTACTTTGCTGCC
TCCCGGCTTTCATCCTGGGTGCATTTTTTGGTTACCTGCCCTGGTTTTT
GCTGGCATCGGTAACAGGACTGCTTATCTGGCATTTCTGGAATTTATTG
CGCCTTTCATGGTGGCTGTGGGTGGATCGCAGTATGACCCCGCCACCGG
GGCGTGGTAGCTGGGAACCGCTACTATACGGCTTACACCAGATGCAGCT
GCGAAATAAAAAACGCCGCCGTGAACTGGGCAATCTGATTAAACGCTTT
CGTAGCGGCGCGGAGTCGCTGCCCGACGCGGTGGTGCTGACCACGGAAG
AGGGCGGTATTTTCTGGTGTAACGGTCTGGCGCAACAAATTCTTGGTTT
GCGCTGGCCGGAAGATAACGGGCAGAACATCCTTAACCTACTGCGTTAC
CCGGAGTTTACGCAATATCTGAAAACGCGTGATTTTTCTCGCCCGCTCA
ATCTGGTGCTCAACACCGGGCGGCATCTGGAAATTCGCGTCATGCCTTA
TACCCACAAACAGTTGCTGATGGTGGCGCGTGATGTCACGCAAATGCAT
CAACTGGAAGGGGCGCGGCGTAACTTTTTTGCCAACGTGAGCCATGAGT
TACGTACGCCATTGACCGTGTTACAGGGTTACCTGGAGATGATGAATGA
GCAGCCGCTGGAAGGCGCGGTACGCGAAAAAGCGTTGCACACCATGCGC
GAGCAGACCCAGCGGATGGAAGGACTGGTGAAGCAATTGCTGACGCTGT
CGAAAATAGAAGCCGCACCGACGCATTTGCTCAATGAAAAGGTTGATGT
GCCGATGATGCTGCGCGTTGTTGAGCGCGAGGCTCAGACTCTGAGTCAG
AAAAAACAGACATTTACCTTTGAGATAGATAACGGCCTCAAGGTGTCTG
GCAACGAAGATCAGCTACGCAGTGCGATTTCGAACCTGGTCTATAACGC
CGTGAATCATACGCCGGAAGGCACGCATATCACCGTACGCTGGCAGCGA
GTGCCGCACGGTGCCGAATTTAGCGTTGAAGATAACGGACCGGGCATTG
CACCGGAGCATATTCCGCGCCTGACCGAGCGTTTTTATCGCGTTGATAA
AGCGCGTTCCCGGCAAACCGGCGGTAGCGGATTAGGGTTAGCGATCGTG
AAACATGCTGTGAATCATCACGAAAGTCGCCTGAATATTGAGAGTACAG
TAGGAAAAGGAACACGTTTCAGTTTTGTTATCCCGGAACGTTTAATTGC
CAAAAACAGCGATTAATCCGCCTTTGTCATCTTTTATTGCCATAAGCCA
GTCGATGCTGGCTTATTTTCTTTGCAGTCAAAATACGGGCGTTAGATTT
TACAACGATTGGTGATTTTTTGTTCGCATGATTAGCCATGTCTTTTTCA
CGGAAATAGTGTTTTATACTGGTTGGTGATTTCTTATCGCTATATACCT
CTGGTTTTTAGATCCCTCCTTGCTTTAAAACGTTATAAGCGTTTAAATT
GCGCTTCAGGTGCT//.

### Exemplary Strains Sensitive to Mn(II) and Iron Cations

Transition metal ions such as manganese (Mn(II)), iron (Fe(II)) and/or Fe(III) are essential trace nutrients in E. coli, as they serve as cofactors for a variety of enzymatic reactions. However, at high levels, the strong reactivity of these metals becomes toxic. As a result, elegant regulatory mechanisms have evolved to tightly control metal homeostasis. Because iron and manganese can often substitute for each other as enzyme cofactors, their regulatory networks are intertwined. The MntH/MntP importer/exporter system plays a key role in manganese and iron homeostasis. MntH is a Mn(II)/Fe(II):H⁺ symporter that imports Mn(II) at high affinity and Fe(II) at lower affinity. Expression of mntH is repressed by both MntR in the presence of Mn(II) and Fur in the presence of Fe(II). As a result, mntH is expressed and both Mn(II) and Fe(II) are imported when their intracellular levels are low. Regulation of the MntP exporter is more complex. A histone-like nucleoid structuring (H-NS) protein binds upstream of two MntR binding sites and a Fur binding site to repress expression; however, Mn(II)-bound MntR and Fe(II)-bound Fur relieve this repression to activate the mntP promoter. In vivo, the mntP promoter is regulated strongly by Mn(II) and weakly by Fe(II). (See, M. Dambach et al., “The ubiquitous yybP-ykoY riboswitch is a manganese-responsive regulatory element,” Mol. Cell 57(6):1099-1109 (2015), doi: 10.1016/j.molcel.2015.01.035).

Additionally, the 5′-untranslated region (UTR) of the mntP gene contains a riboswitch element of the yybP-ykoY family. The Mn(II) sensing region of the riboswitch has two metal binding sites, where one site tolerates the binding of either Mn(II) or Mg(II) but the other strongly prefers Mn(II). (I. R. Price, A. Gaballa, F. Ding, J. D. Helmann, and A. Ke, “Mn2+-Sensing Mechanisms of yybP-ykoY Orphan Riboswitches,” Mol. Cell 57(6):1110-1123 (2015), doi: 10.1016/j.molcel.2015.02.016). Mn(II)-specific binding induces a structural change that allows ribosome binding and mntP translation. (See, M. Dambach et al., “The ubiquitous yybP-ykoY riboswitch is a manganese-responsive regulatory element,” Mol. Cell 57(6):1099-1109 (2015), doi: 10.1016/j.molcel.2015.01.035). Lastly, the MntS small protein plays a direct or indirect role in regulating Mn(II) concentration. Mn(II) may be a chaperone that helps metallize enzymes, or it may inhibit the mntP export system. The mntS promoter itself is repressed by MntR bound to Mn(II) [33]. (J. E. Martin, L. S. Waters, G. Storz, and J. A. Imlay, “The Escherichia coli Small Protein MntS and Exporter MntP Optimize the Intracellular Concentration of Manganese,” PLoS Genet. 11(3):1-31 (2015), doi: 10.1371/journal.pgen.1004977). Considered altogether, when Mn(II) concentration is low, H-NS, the yybP-ykoY riboswitch, and MntS oppose the MntP exporter to conserve intracellular Mn(II) ions. When Mn(II) concentration is high, H-NS repression is relieved, the yybP-ykoY riboswitch allows translation, and mntS expression is repressed to activate MntP-mediated export of Mn(II) ions.

Based on our understanding of this complex regulatory network, we constructed over 20 different promoter versions using various combinations of the mntS promoter, the mntP promoter, the manganese-sensitive riboswitch, the Fur binding box, and various lengths of upstream genomic sequence. Essentially all of these strains responded strongly to concentrations of Mn(II) 100 ppb and higher, with many responding to much lower levels. However, we consistently found that strains exhibited a long memory to Mn(II) exposure (decreasing sensitivity for about 6 h following exposure), which severely limited dynamic sensing approaches. Because these findings suggested that we were fighting metabolic network adaptation extending beyond the behavior of any particular reporting promoter, we generated mntH and mntS knock-out (KO) versions of our E. coli host strain to disable the native manganese regulatory network. Through extensive testing of our set of manganese promoter constructs transformed into multiple KO host strains, we identified three top-performing plasmids based on the mntP promoter.

The E. coli strain that we designated “mntP-yobD-mntS (KO)” contained a Fe(II)-sensing plasmid (full plasmid name “mntP18_yobD_FurB_PmntP_ribo-LutzRBS-sfgfp_hph_p15A,” schematic plasmid map shown in FIG. 11) having the nucleic acid sequence of SEQ ID NO:9:

SEQ ID NO: 9
GAATTCAAAAGATCTTAAGTAAGTAAGAGTATACGTATATCGGCTAATA
ACGTATTAAGGCGCTTCGGCGCCTTTTTTTATGGGGGTATTTTCATCCC
AATCCACACGTCCAACGCACAGCAAACACCACGTCGACCCTATCAGCTG
CGTGCTTTCTATGAGTCGTTGCTGCATAACGACTGTTGTACACTACCGG
GGCCTTTTGGCCCCGTTTTTTTATCTGGAGGATTAATGACAATCACGGA
CCTGGTACTGATTCTTTTCATCGCCGCACTCCTGGCCTTCGCGATCTAC
GATCAGTTCATCATGCCCCGCCGTAACGGCCCCACCCTGCTGGCAATTC
CTTTGCTCCGGCGTGGTCGCATCGATAGCGTTATCTTCGTCGGATTGAT
TGTCATTCTTATCTATAACAACGTCACGAATCATGGTGCGTTAATAACG
ACATGGTTATTAAGCGCACTGGCTCTGATGGGTTTTTATATATTCTGGA
TCCGCGTTCCGAAGATCATCTTTAAACAAAAAGGTTTTTTCTTCGCCAA
TGTCTGGATTGAATATAGCCGAATCAAAGCGATGAACTTGTCGGAAGAT
GGCGTGTTGGTGATGCAATTAGAACAGCGTCGGCTGTTAATCCGCGTTC
GAAATATCGACGATCTGGAAAAAATTTATAAGCTTCTCGTTTCAACTCA
ATAAGTTATGAATACCTCTTCCCTTATTTGTTATTTATTTTAACGTTTC
ATTGATATATAAATCCAAATGAAAATCGTTATCAATAAAGCAATGAAAT
AATATATTCCAACAGTTGTTTTATATTCTCAAAATATGTTAAGGTTGCG
CCCTCATTTGGGGAGTAGCCGATTTCCAGATTCCGGAAATGTACGTGTC
AACATACTCGTTGCAAAACGTGGCACGTACGGACTGAATACTTTCAGTC
AGGCGAGACCATATGCACATCAATCGCTATGCCTGCATGAACGCGAAAA
CCATCACGGCTTGTTGTTCATGTATGCATGTTTGCTGGGGGCGATGATG
TGTTTTATGGATACCCCGGTCGAATTCATTAAAGAGGAGAAAGGTACCA
TGCGTAAAGGCGAAGAGCTGTTCACTGGTGTCGTCCCTATTCTGGTGGA
ACTGGATGGTGATGTCAACGGTCATAAGTTTTCCGTGCGTGGCGAGGGT
GAAGGTGACGCAACTAATGGTAAACTGACGCTGAAGTTCATCTGTACTA
CTGGTAAACTGCCGGTACCTTGGCCGACTCTGGTAACGACGCTGACTTA
TGGTGTTCAGTGCTTTGCTCGTTATCCGGACCATATGAAGCAGCATGAC
TTCTTCAAGTCCGCCATGCCGGAAGGCTATGTGCAGGAACGCACGATTT
CCTTTAAGGATGACGGCACGTACAAAACGCGTGCGGAAGTGAAATTTGA
AGGCGATACCCTGGTAAACCGCATTGAGCTGAAAGGCATTGACTTTAAA
GAAGACGGCAATATCCTGGGCCATAAGCTGGAATACAATTTTAACAGCC
ACAATGTTTACATCACCGCCGATAAACAAAAAAATGGCATTAAAGCGAA
TTTTAAAATTCGCCACAACGTGGAGGATGGCAGCGTGCAGCTGGCTGAT
CACTACCAGCAAAACACTCCAATCGGTGATGGTCCTGTTCTGCTGCCAG
ACAATCACTATCTGAGCACGCAAAGCGTTCTGTCTAAAGATCCGAACGA
GAAACGCGATCATATGGTTCTGCTGGAGTTCGTAACCGCAGCGGGCATC
ACGCATGGTATGGATGAACTGTACAAATGACTTGGACTCCTGTTGATAG
ATCCAGTAATGACCTCAGAACTCCATCTGGATTTGTTCAGAACGCTCGG
TTGCCGCCGGGCGTTTTTTATTGGTGAGAATCCACCTTTCGACCTGCAG
CCTGTTGACAATTAATCATCGGCATAGTATATCGGCATAGTATAATACG
ACAAGGTGAGGAACTAAACCATGAAAAAGCCTGAACTCACCGCGACGTC
TGTCGAGAAGTTTCTGATCGAAAAGTTCGACAGCGTCTCCGACCTGATG
CAGCTCTCGGAGGGCGAAGAATCTCGTGCTTTCAGCTTCGATGTAGGAG
GGCGTGGATATGTCCTGCGGGTAAATAGCTGCGCCGATGGTTTCTACAA
AGATCGTTATGTTTATCGGCACTTTGCATCGGCCGCGCTCCCGATTCCG
GAAGTGCTTGACATTGGGGAATTCAGCGAGAGCCTGACCTATTGCATCT
CCCGCCGTGCACAGGGTGTCACGTTGCAAGACCTGCCTGAAACCGAACT
GCCCGCTGTTCTGCAGCCGGTCGCGGAGGCCATGGATGCGATCGCTGCG
GCCGATCTTAGCCAGACGAGCGGGTTCGGCCCATTCGGACCGCAAGGAA
TCGGTCAATACACTACATGGCGTGATTTCATATGCGCGATTGCTGATCC
CCATGTGTATCACTGGCAAACTGTGATGGACGACACCGTCAGTGCGTCC
GTCGCGCAGGCTCTCGATGAGCTGATGCTTTGGGCCGAGGACTGCCCCG
AAGTCCGGCACCTCGTGCACGCGGATTTCGGCTCCAACAATGTCCTGAC
GGACAATGGCCGCATAACAGCGGTCATTGACTGGAGCGAGGCGATGTTC
GGGGATTCCCAATACGAGGTCGCCAACATCTTCTTCTGGAGGCCGTGGT
TGGCTTGTATGGAGCAGCAGACGCGCTACTTCGAGCGGAGGCATCCGGA
GCTTGCAGGATCGCCGCGGCTCCGGGCGTATATGCTCCGCATTGGTCTT
GACCAACTCTATCAGAGCTTGGTTGACGGCAATTTCGATGATGCAGCTT
GGGCGCAGGGTCGATGCGACGCAATCGTCCGATCCGGAGCCGGGACTGT
CGGGCGTACACAAATCGCCCGCAGAAGCGCGGCCGTCTGGACCGATGGC
TGTGTAGAAGTACTCGCCGATAGTGGAAACCGACGCCCCGGATGAGGGG
ATCAATTCTCTAGAGCTCGCTGATCAGAAGTTCCTGATATATTCCGCTT
CCTCGCTCACTGACTCGCTACGCTCGGTCGTTCGACTGCGGCGAGCGGA
AATGGCTTACGAACGGGGCGGAGATTTCCTGGAAGATGCCAGGAAGATA
CTTAACAGGGAAGTGAGAGGGCCGCGGCAAAGCCGTTTTTCCATAGGCT
CCGCCCCCCTGACAAGCATCACGAAATCTGACGCTCAAATCAGTGGTGG
CGAAACCCGACAGGACTATAAAGATACCAGGCGTTTCCCCCTGGCGGCT
CCCTCGTGCGCTCTCCTGTTCCTGCCTTTCGGTTTACCGGTGTCATTCC
GCTGTTATGGCCGCGTTTGTCTCATTCCACGCCTGACACTCAGTTCCGG
GTAGGCAGTTCGCTCCAAGCTGGACTGTATGCACGAACCCCCCGTTCAG
TCCGACCGCTGCGCCTTATCCGGTAACTATCGTCTTGAGTCCAACCCGG
AAAGACATGCAAAAGCACCACTGGCAGCAGCCACTGGTAATTGATTTAG
AGGAGTTAGTCTTGAAGTCATGCGCCGGTTAAGGCTAAACTGAAAGGAC
AAGTTTTGGTGACTGCGCTCCTCCAAGCCAGTTACCTCGGTTCAAAGAG
TTGGTAGCTCAGAGAACCTTCGAAAAACCGCCCTGCAAGGCGGTTTTTT
CGTTTTCAGAGCAAGAGATTACGCGCAGACCAAAACGATCTCAAGAAGA
TCATCTTATTAATCAGATAAAATATTTCTAGATTTCAGTGCAATTTATC
TCTTCAAATGTAGCACCTGAAGTCAGCCCCATACGATATAAGTTGTT//.

The mntP18_yobD_FurB_PmntP_ribo-LutzRBS-sfgfp_hph_p15A plasmid in this iron cation-sensing “mntP-yobD-mntS (KO)” strain is based on a variant of the mntP promoter with H-NS and Fur binding sites present but mntR binding sites removed such that mntP repression by H-NS is relieved only by Fe(II) binding to Fur. This plasmid was transformed into an E. coli MG1655 host strain with the mntS gene knocked out to eliminate Mn(II) regulation of mntP through mntS. The mutated mntP promoter had the following nucleotide sequences of SEQ ID NO:32 (upstream of the excised mntR binding sites, extending past the yobD gene) and SEQ ID NO:33 (downstream of the excised mntR binding sites):

SEQ ID NO: 32
GACTGTTGTACACTACCGGGGCCTTTTGGCCCCGTTTTTTTATCTGGAG
GATTAATGACAATCACGGACCTGGTACTGATTCTTTTCATCGCCGCACT
CCTGGCCTTCGCGATCTACGATCAGTTCATCATGCCCCGCCGTAACGGC
CCCACCCTGCTGGCAATTCCTTTGCTCCGGCGTGGTCGCATCGATAGCG
TTATCTTCGTCGGATTGATTGTCATTCTTATCTATAACAACGTCACGAA
TCATGGTGCGTTAATAACGACATGGTTATTAAGCGCACTGGCTCTGATG
GGTTTTTATATATTCTGGATCCGCGTTCCGAAGATCATCTTTAAACAAA
AAGGTTTTTTCTTCGCCAATGTCTGGATTGAATATAGCCGAATCAAAGC
GATGAACTTGTCGGAAGATGGCGTGTTGGTGATGCAATTAGAACAGCGT
CGGCTGTTAATCCGCGTTCGAAATATCGACGATCTGGAAAAAATTTATA
AGCTTCTCGTTTCAACTCAATAAGTTATGAAT//;
and
SEQ ID NO: 33
ACCTCTTCCCTTATTTGTTATTTATTTTAACGTTTCATTGATATATAAA
TCCAAATGAAAATCGTTATCAATAAAGCAATGAAATAATATATTCCAAC
AGTTGTTTTATATTCTCAAAATATGTTAAGGTTGCGCCCTCATTTGGGG
AGTAGCCGATTTCCAGATTCCGGAAATGTACGTGTCAACATACTCGTTG
CAAAACGTGGCACGTACGGACTGAATACTTTCAGTCAGGCGAGACCATA
TGCACATCAATCGCTATGCCTGCATGAACGCGAAAACCATCACGGCTTG
TTGTTCATGTATGCATGTTTGCTGGGGGCGATGATGTGTTTTATGGATA
CCCCGGTC//.

The E. coli strains that we designated “mntP” and “mntP-mntH(KO)” contained a Mn(II)-sensing plasmid (designated “mntP6_MntRB_FurB_PmntP_ribo_trim27-LutzRBS-sfgfp_hph_p15A,” schematic plasmid map shown in FIG. 12) having the nucleic acid sequence of SEQ ID NO:10:

SEQ ID NO: 10
GAATTCAAAAGATCTTAAGTAAGTAAGAGTATACGTATATCGGCTAATA
ACGTATTAAGGCGCTTCGGCGCCTTTTTTTATGGGGGTATTTTCATCCC
AATCCACACGTCCAACGCACAGCAAACACCACGTCGACCCTATCAGCTG
CGTGCTTTCTATGAGTCGTTGCTGCATAACGTTTCAACTCAATAAGTTA
TGAATTTAGCCAAAGCTATGTTTAGTGTATTTTTAATAATCAGACATAG
CTTAGGCTATATTACCTCTTCCCTTATTTGTTATTTATTTTAACGTTTC
ATTGATATATAAATCCAAATGAAAATCGTTATCAATAAAGCAATGAAAT
AATATATTCCAACAGTTGTTTTATATTCTCAAAATATGTTAAGGTTGCG
CCCTCATTTGGGGAGTAGCCGATTTCCAGATTCCGGAAATGTACGTGTC
AACATACTCGTTGCAAAACGTGGCACGTACGGACTGAATACTTTCAGTC
AGGCGAGACCATATGCACATCAATCGCTATGCCTGCATGAACGCGAAAA
CCATCACGGCTTGTTGTTCATGTATGCATGTTTGCTGGGGGCGATGATG
TGTTTGAATTCATTAAAGAGGAGAAAGGTACCATGCGTAAAGGCGAAGA
GCTGTTCACTGGTGTCGTCCCTATTCTGGTGGAACTGGATGGTGATGTC
AACGGTCATAAGTTTTCCGTGCGTGGCGAGGGTGAAGGTGACGCAACTA
ATGGTAAACTGACGCTGAAGTTCATCTGTACTACTGGTAAACTGCCGGT
ACCTTGGCCGACTCTGGTAACGACGCTGACTTATGGTGTTCAGTGCTTT
GCTCGTTATCCGGACCATATGAAGCAGCATGACTTCTTCAAGTCCGCCA
TGCCGGAAGGCTATGTGCAGGAACGCACGATTTCCTTTAAGGATGACGG
CACGTACAAAACGCGTGCGGAAGTGAAATTTGAAGGCGATACCCTGGTA
AACCGCATTGAGCTGAAAGGCATTGACTTTAAAGAAGACGGCAATATCC
TGGGCCATAAGCTGGAATACAATTTTAACAGCCACAATGTTTACATCAC
CGCCGATAAACAAAAAAATGGCATTAAAGCGAATTTTAAAATTCGCCAC
AACGTGGAGGATGGCAGCGTGCAGCTGGCTGATCACTACCAGCAAAACA
CTCCAATCGGTGATGGTCCTGTTCTGCTGCCAGACAATCACTATCTGAG
CACGCAAAGCGTTCTGTCTAAAGATCCGAACGAGAAACGCGATCATATG
GTTCTGCTGGAGTTCGTAACCGCAGCGGGCATCACGCATGGTATGGATG
AACTGTACAAATGACTTGGACTCCTGTTGATAGATCCAGTAATGACCTC
AGAACTCCATCTGGATTTGTTCAGAACGCTCGGTTGCCGCCGGGCGTTT
TTTATTGGTGAGAATCCACCTTTCGACCTGCAGCCTGTTGACAATTAAT
CATCGGCATAGTATATCGGCATAGTATAATACGACAAGGTGAGGAACTA
AACCATGAAAAAGCCTGAACTCACCGCGACGTCTGTCGAGAAGTTTCTG
ATCGAAAAGTTCGACAGCGTCTCCGACCTGATGCAGCTCTCGGAGGGCG
AAGAATCTCGTGCTTTCAGCTTCGATGTAGGAGGGCGTGGATATGTCCT
GCGGGTAAATAGCTGCGCCGATGGTTTCTACAAAGATCGTTATGTTTAT
CGGCACTTTGCATCGGCCGCGCTCCCGATTCCGGAAGTGCTTGACATTG
GGGAATTCAGCGAGAGCCTGACCTATTGCATCTCCCGCCGTGCACAGGG
TGTCACGTTGCAAGACCTGCCTGAAACCGAACTGCCCGCTGTTCTGCAG
CCGGTCGCGGAGGCCATGGATGCGATCGCTGCGGCCGATCTTAGCCAGA
CGAGCGGGTTCGGCCCATTCGGACCGCAAGGAATCGGTCAATACACTAC
ATGGCGTGATTTCATATGCGCGATTGCTGATCCCCATGTGTATCACTGG
CAAACTGTGATGGACGACACCGTCAGTGCGTCCGTCGCGCAGGCTCTCG
ATGAGCTGATGCTTTGGGCCGAGGACTGCCCCGAAGTCCGGCACCTCGT
GCACGCGGATTTCGGCTCCAACAATGTCCTGACGGACAATGGCCGCATA
ACAGCGGTCATTGACTGGAGCGAGGCGATGTTCGGGGATTCCCAATACG
AGGTCGCCAACATCTTCTTCTGGAGGCCGTGGTTGGCTTGTATGGAGCA
GCAGACGCGCTACTTCGAGCGGAGGCATCCGGAGCTTGCAGGATCGCCG
CGGCTCCGGGCGTATATGCTCCGCATTGGTCTTGACCAACTCTATCAGA
GCTTGGTTGACGGCAATTTCGATGATGCAGCTTGGGCGCAGGGTCGATG
CGACGCAATCGTCCGATCCGGAGCCGGGACTGTCGGGCGTACACAAATC
GCCCGCAGAAGCGCGGCCGTCTGGACCGATGGCTGTGTAGAAGTACTCG
CCGATAGTGGAAACCGACGCCCCGGATGAGGGGATCAATTCTCTAGAGC
TCGCTGATCAGAAGTTCCTGATATATTCCGCTTCCTCGCTCACTGACTC
GCTACGCTCGGTCGTTCGACTGCGGCGAGCGGAAATGGCTTACGAACGG
GGCGGAGATTTCCTGGAAGATGCCAGGAAGATACTTAACAGGGAAGTGA
GAGGGCCGCGGCAAAGCCGTTTTTCCATAGGCTCCGCCCCCCTGACAAG
CATCACGAAATCTGACGCTCAAATCAGTGGTGGCGAAACCCGACAGGAC
TATAAAGATACCAGGCGTTTCCCCCTGGCGGCTCCCTCGTGCGCTCTCC
TGTTCCTGCCTTTCGGTTTACCGGTGTCATTCCGCTGTTATGGCCGCGT
TTGTCTCATTCCACGCCTGACACTCAGTTCCGGGTAGGCAGTTCGCTCC
AAGCTGGACTGTATGCACGAACCCCCCGTTCAGTCCGACCGCTGCGCCT
TATCCGGTAACTATCGTCTTGAGTCCAACCCGGAAAGACATGCAAAAGC
ACCACTGGCAGCAGCCACTGGTAATTGATTTAGAGGAGTTAGTCTTGAA
GTCATGCGCCGGTTAAGGCTAAACTGAAAGGACAAGTTTTGGTGACTGC
GCTCCTCCAAGCCAGTTACCTCGGTTCAAAGAGTTGGTAGCTCAGAGAA
CCTTCGAAAAACCGCCCTGCAAGGCGGTTTTTTCGTTTTCAGAGCAAGA
GATTACGCGCAGACCAAAACGATCTCAAGAAGATCATCTTATTAATCAG
ATAAAATATTTCTAGATTTCAGTGCAATTTATCTCTTCAAATGTAGCAC
CTGAAGTCAGCCCCATACGATATAAGTTGTT//.

The mntP6_MntRB_FurB_PmntP_ribo_trim27-LutzRBS-sfgfp_hph_p15A plasmid in these Mn(II)-sensing “mntP” and “mntP-mntH(KO)” strains is based on a variant of the mntP promoter with based on a variant of the mntP promoter with the H-NS binding site removed to eliminate Fe(II) regulation of mntP through Fur. This plasmid was transformed into both E. coli MG1655 wildtype and mntH KO host strains for Mn(II) sensing. The mntH KO further insulated Mn(II) sensing from Fe(II) by eliminating the Mn(II)/Fe(II) dual importer. The mutated mntP promoter had the nucleotide sequence of SEQ ID NO:34:

SEQ ID NO: 34
GTTTCAACTCAATAAGTTATGAATTTAGCCAAAGCTATGTTTAGTGTAT
TTTTAATAATCAGACATAGCTTAGGCTATATTACCTCTTCCCTTATTTG
TTATTTATTTTAACGTTTCATTGATATATAAATCCAAATGAAAATCGTT
ATCAATAAAGCAATGAAATAATATATTCCAACAGTTGTTTTATATTCTC
AAAATATGTTAAGGTTGCGCCCTCATTTGGGGAGTAGCCGATTTCCAGA
TTCCGGAAATGTACGTGTCAACATACTCGTTGCAAAACGTGGCACGTAC
GGACTGAATACTTTCAGTCAGGCGAGACCATATGCACATCAATCGCTAT
GCCTGCATGAACGCGAAAACCATCACGGCTTGTTGTTCATGTATGCATG
TTTGCTGGGGGCGATGATGTGTTT//.

The E. coli strain that we designated “mntP+-mntH(KO)” contained a Mn(II)-sensing plasmid (full plasmid name “mntP20_MntRB_FurB_PmntP_ribo_(15AA)-sfgfp_hph_p15A,” schematic plasmid map shown in FIG. 13), a variant of SEQ ID NO:10 having the nucleic acid sequence of SEQ ID NO:11:

SEQ ID NO: 11
GAATTCAAAAGATCTTAAGTAAGTAAGAGTATACGTATATCGGCTAATA
ACGTATTAAGGCGCTTCGGCGCCTTTTTTTATGGGGGTATTTTCATCCC
AATCCACACGTCCAACGCACAGCAAACACCACGTCGACCCTATCAGCTG
CGTGCTTTCTATGAGTCGTTGCTGCATAACGTTTCAACTCAATAAGTTA
TGAATTTAGCCAAAGCTATGTTTAGTGTATTTTTAATAATCAGACATAG
CTTAGGCTATATTACCTCTTCCCTTATTTGTTATTTATTTTAACGTTTC
ATTGATATATAAATCCAAATGAAAATCGTTATCAATAAAGCAATGAAAT
AATATATTCCAACAGTTGTTTTATATTCTCAAAATATGTTAAGGTTGCG
CCCTCATTTGGGGAGTAGCCGATTTCCAGATTCCGGAAATGTACGTGTC
AACATACTCGTTGCAAAACGTGGCACGTACGGACTGAATACTTTCAGTC
AGGCGAGACCATATGCACATCAATCGCTATGCCTGCATGAACGCGAAAA
CCATCACGGCTTGTTGTTCATGTATGCATGTTTGCTGGGGGCGATGATG
TGTTTTATGGATACCCCGGTCAGGACATTGTCATGAATATCACTGCTAC
TGTTCTTCTTGCGTTTGGTATGTCGATGATGCGTAAAGGCGAAGAGCTG
TTCACTGGTGTCGTCCCTATTCTGGTGGAACTGGATGGTGATGTCAACG
GTCATAAGTTTTCCGTGCGTGGCGAGGGTGAAGGTGACGCAACTAATGG
TAAACTGACGCTGAAGTTCATCTGTACTACTGGTAAACTGCCGGTACCT
TGGCCGACTCTGGTAACGACGCTGACTTATGGTGTTCAGTGCTTTGCTC
GTTATCCGGACCATATGAAGCAGCATGACTTCTTCAAGTCCGCCATGCC
GGAAGGCTATGTGCAGGAACGCACGATTTCCTTTAAGGATGACGGCACG
TACAAAACGCGTGCGGAAGTGAAATTTGAAGGCGATACCCTGGTAAACC
GCATTGAGCTGAAAGGCATTGACTTTAAAGAAGACGGCAATATCCTGGG
CCATAAGCTGGAATACAATTTTAACAGCCACAATGTTTACATCACCGCC
GATAAACAAAAAAATGGCATTAAAGCGAATTTTAAAATTCGCCACAACG
TGGAGGATGGCAGCGTGCAGCTGGCTGATCACTACCAGCAAAACACTCC
AATCGGTGATGGTCCTGTTCTGCTGCCAGACAATCACTATCTGAGCACG
CAAAGCGTTCTGTCTAAAGATCCGAACGAGAAACGCGATCATATGGTTC
TGCTGGAGTTCGTAACCGCAGCGGGCATCACGCATGGTATGGATGAACT
GTACAAATGACTTGGACTCCTGTTGATAGATCCAGTAATGACCTCAGAA
CTCCATCTGGATTTGTTCAGAACGCTCGGTTGCCGCCGGGCGTTTTTTA
TTGGTGAGAATCCACCTTTCGACCTGCAGCCTGTTGACAATTAATCATC
GGCATAGTATATCGGCATAGTATAATACGACAAGGTGAGGAACTAAACC
ATGAAAAAGCCTGAACTCACCGCGACGTCTGTCGAGAAGTTTCTGATCG
AAAAGTTCGACAGCGTCTCCGACCTGATGCAGCTCTCGGAGGGCGAAGA
ATCTCGTGCTTTCAGCTTCGATGTAGGAGGGCGTGGATATGTCCTGCGG
GTAAATAGCTGCGCCGATGGTTTCTACAAAGATCGTTATGTTTATCGGC
ACTTTGCATCGGCCGCGCTCCCGATTCCGGAAGTGCTTGACATTGGGGA
ATTCAGCGAGAGCCTGACCTATTGCATCTCCCGCCGTGCACAGGGTGTC
ACGTTGCAAGACCTGCCTGAAACCGAACTGCCCGCTGTTCTGCAGCCGG
TCGCGGAGGCCATGGATGCGATCGCTGCGGCCGATCTTAGCCAGACGAG
CGGGTTCGGCCCATTCGGACCGCAAGGAATCGGTCAATACACTACATGG
CGTGATTTCATATGCGCGATTGCTGATCCCCATGTGTATCACTGGCAAA
CTGTGATGGACGACACCGTCAGTGCGTCCGTCGCGCAGGCTCTCGATGA
GCTGATGCTTTGGGCCGAGGACTGCCCCGAAGTCCGGCACCTCGTGCAC
GCGGATTTCGGCTCCAACAATGTCCTGACGGACAATGGCCGCATAACAG
CGGTCATTGACTGGAGCGAGGCGATGTTCGGGGATTCCCAATACGAGGT
CGCCAACATCTTCTTCTGGAGGCCGTGGTTGGCTTGTATGGAGCAGCAG
ACGCGCTACTTCGAGCGGAGGCATCCGGAGCTTGCAGGATCGCCGCGGC
TCCGGGCGTATATGCTCCGCATTGGTCTTGACCAACTCTATCAGAGCTT
GGTTGACGGCAATTTCGATGATGCAGCTTGGGCGCAGGGTCGATGCGAC
GCAATCGTCCGATCCGGAGCCGGGACTGTCGGGCGTACACAAATCGCCC
GCAGAAGCGCGGCCGTCTGGACCGATGGCTGTGTAGAAGTACTCGCCGA
TAGTGGAAACCGACGCCCCGGATGAGGGGATCAATTCTCTAGAGCTCGC
TGATCAGAAGTTCCTGATATATTCCGCTTCCTCGCTCACTGACTCGCTA
CGCTCGGTCGTTCGACTGCGGCGAGCGGAAATGGCTTACGAACGGGGCG
GAGATTTCCTGGAAGATGCCAGGAAGATACTTAACAGGGAAGTGAGAGG
GCCGCGGCAAAGCCGTTTTTCCATAGGCTCCGCCCCCCTGACAAGCATC
ACGAAATCTGACGCTCAAATCAGTGGTGGCGAAACCCGACAGGACTATA
AAGATACCAGGCGTTTCCCCCTGGCGGCTCCCTCGTGCGCTCTCCTGTT
CCTGCCTTTCGGTTTACCGGTGTCATTCCGCTGTTATGGCCGCGTTTGT
CTCATTCCACGCCTGACACTCAGTTCCGGGTAGGCAGTTCGCTCCAAGC
TGGACTGTATGCACGAACCCCCCGTTCAGTCCGACCGCTGCGCCTTATC
CGGTAACTATCGTCTTGAGTCCAACCCGGAAAGACATGCAAAAGCACCA
CTGGCAGCAGCCACTGGTAATTGATTTAGAGGAGTTAGTCTTGAAGTCA
TGCGCCGGTTAAGGCTAAACTGAAAGGACAAGTTTTGGTGACTGCGCTC
CTCCAAGCCAGTTACCTCGGTTCAAAGAGTTGGTAGCTCAGAGAACCTT
CGAAAAACCGCCCTGCAAGGCGGTTTTTTCGTTTTCAGAGCAAGAGATT
ACGCGCAGACCAAAACGATCTCAAGAAGATCATCTTATTAATCAGATAA
AATATTTCTAGATTTCAGTGCAATTTATCTCTTCAAATGTAGCACCTGA
AGTCAGCCCCATACGATATAAGTTGTT//.

In plasmid mntP20_MntRB_FurB_PmntP_ribo_(15AA)-sfgfp_hph_p15A, we replaced the Lutz RBS with the native mntP promoter's RBS. Because the native RBS was poorly defined, we included the native mntP promoter sequence through the coding sequence for the first 15 amino acids of MntP (SEQ ID NO:35) to create a fusion with GFP. Like SEQ ID NO:10, we transformed SEQ ID NO:11 into an E. coli mntH KO host strain to further insulate Mn(II)-sensing from iron cation. The following is the nucleotide sequence of SEQ ID NO:35:

SEQ ID NO: 35
GTTTCAACTCAATAAGTTATGAATTTAGCCAAAGCTATGTTTAGTGTAT
TTTTAATAATCAGACATAGCTTAGGCTATATTACCTCTTCCCTTATTTG
TTATTTATTTTAACGTTTCATTGATATATAAATCCAAATGAAAATCGTT
ATCAATAAAGCAATGAAATAATATATTCCAACAGTTGTTTTATATTCTC
AAAATATGTTAAGGTTGCGCCCTCATTTGGGGAGTAGCCGATTTCCAGA
TTCCGGAAATGTACGTGTCAACATACTCGTTGCAAAACGTGGCACGTAC
GGACTGAATACTTTCAGTCAGGCGAGACCATATGCACATCAATCGCTAT
GCCTGCATGAACGCGAAAACCATCACGGCTTGTTGTTCATGTATGCATG
TTTGCTGGGGGCGATGATGTGTTTTATGGATACCCCGGTCAGGACATTG
TCATGAATATCACTGCTACTGTTCTTCTTGCGTTTGGTATGTCGATG//.

Employing the combination of SEQ ID NO:10 and SEQ ID NO:11 in wildtype and KO host strains allowed us to expand the detectable range of Mn(II) to between 10 and 3000 ppb, as we were able to combine the narrower dynamic range of each strain by monitoring all strains in parallel in a microfluidic strain array.

In addition to constructing sensing plasmids based on our reviews of scientific literature, we screened an E. coli fluorescent promoter library for responders to our analytes of interest. (See, A. Zaslaver et al., “A comprehensive library of fluorescent transcriptional reporters for Escherichia coli,” Nat Methods 3 (8):623-628 (2006)). This library was constructed by cloning about 2,000 promoter regions in E. coli K-12 upstream of gfpmut2 on a low-copy plasmid and transforming back into E. coli K-12 MG1655. Specifically, primers were designed to amplify all intergenic regions longer than 40 bp in E. coli K-12 MG1655. In order to include binding sites for transcriptional regulators, these intergenic regions extended 50-150 bp into each upstream and downstream coding region. These promoter regions were cloned into one of two reporter plasmid backbones according to their orientation in the chromosome. Both reporter plasmid backbones incorporated a low-copy pSC101 origin, kanamycin resistance cassette, and gfpmut2 fluorescent reporter gene with a strong ribosome binding site. Sequencing was used verify the identities of the promoter region inserts to above 99% accuracy, with the final library including 1,820 unique promoter regions.

We arrayed this promoter library on a large-format microfluidic device containing 2,048 strain banks and serially exposed to analytes to search for responders. (G. Graham et al., “Genome-scale transcriptional dynamics and environmental biosensing,” Proc Nat Acad Sci USA 117(6):3301-3306 (2020), doi: 10.1073/pnas.1913003117). We discovered three library strains exhibiting promoter responses that scaled roughly linearly with iron concentration. Strain “codB,” containing a promoter for a cytosine transporter, increased GFP production as a step response to increasing iron exposure. Strain “fes,” containing a promoter for ferric enterobactin esterase, decreased GFP production as a step response to increasing iron exposure. Finally, strain “ugpB,” containing a promoter for an ABC transporter periplasmic binding protein, increased GFP production as a step response to increasing iron exposure. After identifying these library strains, we modified their plasmids by swapping the kanamycin resistance cassette from the library backbone with a hygromycin B resistance cassette. Thus our entire suite of sensing strains contained the hph resistance gene and could be arrayed on a single microfluidic device (or “chip”) using a common selective medium containing the hygromycin B antibiotic. We performed whole-plasmid sequencing on these final plasmids and characterized corresponding transformed strains in subsequent experiments before including them in our final sensing panel. SEQ ID NO:36 represents the nucleotide sequence for plasmid 99_pQBI_codB_hph (schematic plasmid map shown in FIG. 24) based on the codB promoter:

SEQ ID NO: 36
GGTTCTTATGGCTCTTGTATCTATCAGTGAAGCATCAAGACTAACAAAC
AAAAGTAGAACAACTGTTCACCGTTACATATCAAAGGGAAAACTGTCCA
TATGCACAGATGAAAACGGTGTAAAAAAGATAGATACATCAGAGCTTTT
ACGAGTTTTTGGTGCATTCAAAGCTGTTCACCATGAACAGATCGACAAT
GTAACAGATGAACAGCATGTAACACCTAATAGAACAGGTGAAACCAGTA
AAACAAAGCAACTAGAACATGAAATTGAACACCTGAGACAACTTGTTAC
AGCTCAACAGTCACACATAGACAGCCTGAAACAGGCGATGCTGCTTATC
GAATCAAAGCTGCCGACAACACGGGAGCCAGTGACGCCTCCCGTGGGGA
AAAAATCATGGCAATTCTGGAAGAAATAGCGCTTTCAGCCGGCAAACCG
GCTGAAGCCGGATCTGCGATTCTGATAACAAACTAGCAACACCAGAACA
GCCCGTTTGCGGGCAGCAAAACCCGTACCCTAGGTCTAGGGCGGCGGAT
TTGTCCTACTCAGGAGAGCGTTCACCGACAAACAACAGATAAAACGAAA
GGCCCAGTCTTTCGACTGAGCCTTTCGTTTTATTTGATGCCTCTAGAGC
TTGCATGCCTGCAGGTCTGGACATTTATTTGTATAGTTCATCCATGCCA
TGTGTAATCCCAGCAGCTGTTACAAACTCAAGAAGGACCATGTGGTCTC
TCTTTTCGTTGGGATCTTTCGAAAGGGCAGATTGTGTGGACAGGTAATG
GTTGTCTGGTAAAAGGACAGGGCCATCGCCAATTGGAGTATTTTGTTGA
TAATGGTCTGCTAGTTGAACGCTTCCATCTTCAATGTTGTGTCTAATTT
TGAAGTTAACTTTGATTCCATTCTTTTGTTTGTCTGCCATGATGTATAC
ATTGTGTGAGTTATAGTTGTATTCCAATTTGTGTCCAAGAATGTTTCCA
TCTTCTTTAAAATCAATACCTTTTAACTCGATTCTATTAACAAGGGTAT
CACCTTCAAACTTGACTTCAGCACGTGTCTTGTAGTTCCCGTCATCTTT
GAAAAATATAGTTCTTTCCTGTACATAACCTTCGGGCATGGCACTCTTG
AAAAAGTCATGCTGTTTCATATGATCTGGGTATCTCGCAAAGCATTGAA
GACCATACGCGAAAGTAGTGACAAGTGTTGGCCATGGAACAGGTAGTTT
TCCAGTAGTGCAAATAAATTTAAGGGTAAGTTTTCCGTATGTTGCATCA
CCTTCACCCTCTCCACTGACAGAAAATTTGTGCCCATTAACATCACCAT
CTAATTCAACAAGAATTGGGACAACTCCAGTGAAAAGTTCTTCTCCTTT
ACTCATATGTATATCTCCTTCTTAAATCTAGAGGATCCCGTCAATGCCA
ATACCCCCCGCTCCAGGATCGCGTCCTCTCGAGCGGGATCCCGTCAATG
CCAATACCCCTTTCCGCGCCGACTGCGGGACTGGCCCCTGGCTAAAGTT
GTTATCTTGCGACACGAAATTCCCCCTTTTTGCTGTTTGAAACCCAAAA
AATCCGCCGCATTCTATTCATCTGAAGATAAAAAGCAATCGTTTTCGTG
GGGAAATATATTTTTTATATGAATGAGTGCAAAAAGGTGGGGGAAAAAT
TATCGGTGTAAAAACAGTGGGTAGGCCTGATAAGACGCGTCAGCGTCGC
ATCAGGCATCTGAGCATGGTGTCGGATGCGACGCGAGCGCCTTATCCGA
CCTACGGTTCAGGTCCGTAGGCATGATAAAACGCGTCAGCGTCGCATCA
GGCATCGGCACACAGTGCCGGATGCACCGCGCGACGCATCCGACAATAA
CCTACTCTTCCATCGCCTGGCGGATCTGATCCAACGACGCAGGATCATC
AATGGTCGTCAGATCTCCAGGATCGCGTCCTCTCGAGGTGAAGACGAAA
GGGCCTCGTGATACGCCTATTTTTATAGGTTAATGTCATGATAATAATG
GTTTCTTAGACGTCGGAATTGCCAGCTGGGGCGCCCTCTGGTAAGGTTG
GGAAGCCCTGCAAAGTAAACTGGATGGCTTTCTTGCCGCCAAGGATCTG
ATGGCGCAGGGGATCAAGATCTGATCAAGAGACAGGATGAGGATCGTTT
CGCATGATTGAACAAGATGTTTTTTATTGGTGAGAATCCACCTTTCGAC
CTGCAGCCTGTTGACAATTAATCATCGGCATAGTATATCGGCATAGTAT
AATACGACAAGGTGAGGAACTAAACCATGAAAAAGCCTGAACTCACCGC
GACGTCTGTCGAGAAGTTTCTGATCGAAAAGTTCGACAGCGTCTCCGAC
CTGATGCAGCTCTCGGAGGGCGAAGAATCTCGTGCTTTCAGCTTCGATG
TAGGAGGGCGTGGATATGTCCTGCGGGTAAATAGCTGCGCCGATGGTTT
CTACAAAGATCGTTATGTTTATCGGCACTTTGCATCGGCCGCGCTCCCG
ATTCCGGAAGTGCTTGACATTGGGGAATTCAGCGAGAGCCTGACCTATT
GCATCTCCCGCCGTGCACAGGGTGTCACGTTGCAAGACCTGCCTGAAAC
CGAACTGCCCGCTGTTCTGCAGCCGGTCGCGGAGGCCATGGATGCGATC
GCTGCGGCCGATCTTAGCCAGACGAGCGGGTTCGGCCCATTCGGACCGC
AAGGAATCGGTCAATACACTACATGGCGTGATTTCATATGCGCGATTGC
TGATCCCCATGTGTATCACTGGCAAACTGTGATGGACGACACCGTCAGT
GCGTCCGTCGCGCAGGCTCTCGATGAGCTGATGCTTTGGGCCGAGGACT
GCCCCGAAGTCCGGCACCTCGTGCACGCGGATTTCGGCTCCAACAATGT
CCTGACGGACAATGGCCGCATAACAGCGGTCATTGACTGGAGCGAGGCG
ATGTTCGGGGATTCCCAATACGAGGTCGCCAACATCTTCTTCTGGAGGC
CGTGGTTGGCTTGTATGGAGCAGCAGACGCGCTACTTCGAGCGGAGGCA
TCCGGAGCTTGCAGGATCGCCGCGGCTCCGGGCGTATATGCTCCGCATT
GGTCTTGACCAACTCTATCAGAGCTTGGTTGACGGCAATTTCGATGATG
CAGCTTGGGCGCAGGGTCGATGCGACGCAATCGTCCGATCCGGAGCCGG
GACTGTCGGGCGTACACAAATCGCCCGCAGAAGCGCGGCCGTCTGGACC
GATGGCTGTGTAGAAGTACTCGCCGATAGTGGAAACCGACGCCCCGGAT
GAGGGGATCAATTCTCTAGAGCTCGCTGATCAGAAGTTCCTGATATATT
CCGCTTCCTTCTTGACGAGTTCTTCTGAGCGGGACTCTGGGGTTCGAGA
GCTCGCTTGGACTCCTGTTGATAGATCCAGTAATGACCTCAGAACTCCA
TCTGGATTTGTTCAGAACGCTCGGTTGCCGCCGGGCGTTTTTTATTGGT
GAGAATCCAAGCACTAGGGACAGTAAGACGGGTAAGCCTGTTGATGATA
CCGCTGCCTTACTGGGTGCATTAGCCAGTCTGAATGACCTGTCACGGGA
TAATCCGAAGTGGTCAGACTGGAAAATCAGAGGGCAGGAACTGCTGAAC
AGCAAAAAGTCAGATAGCACCACATAGCAGACCCGCCATAAAACGCCCT
GAGAAGCCCGTGACGGGCTTTTCTTGTATTATGGGTAGTTTCCTTGCAT
GAATCCATAAAAGGCGCCTGTAGTGCCATTTACCCCCATTCACTGCCAG
AGCCGTGAGCGCAGCGAACTGAATGTCACGAAAAAGACAGCGACTCAGG
TGCCTGATGGTCGGAGACAAAAGGAATATTCAGCGATTTGCCCGAGCTT
GCGAGGGTGCTACTTAAGCCTTTAGGGTTTTAAGGTCTGTTTTGTAGAG
GAGCAAACAGCGTTTGCGACATCCTTTTGTAATACTGCGGAACTGACTA
AAGTAGTGAGTTATACACAGGGCTGGGATCTATTCTTTTTATCTTTTTT
TATTCTTTCTTTATTCTATAAATTATAACCACTTGAATATAAACAAAAA
AAACACACAAAGGTCTAGCGGAATTTACAGAGGGTCTAGCAGAATTTAC
AAGTTTTCCAGCAAAGGTCTAGCAGAATTTACAGATACCCACAACTCAA
AGGAAAAGGACTAGTAATTATCATTGACTAGCCCATCTCAATTGGTATA
GTGATTAAAATCACCTAGACCAATTGAGATGTATGTCTGAATTAGTTGT
TTTCAAAGCAAATGAACTAGCGATTAGTCGCTATGACTTAACGGAGCAT
GAAACCAAGCTAATTTTATGCTGTGTGGCACTACTCAACCCCACGATTG
AAAACCCTACAAGGAAAGAACGGACGGTATCGTTCACTTATAACCAATA
CGCTCAGATGATGAACATCAGTAGGGAAAATGCTTATGGTGTATTAGCT
AAAGCAACCAGAGAGCTGATGACGAGAACTGTGGAAATCAGGAATCCTT
TGGTTAAAGGCTTTGAGATTTTCCAGTGGACAAACTATGCCAAGTTCTC
AAGCGAAAAATTAGAATTAGTTTTTAGTGAAGAGATATTGCCTTATCTT
TTCCAGTTAAAAAAATTCATAAAATATAATCTGGAACATGTTAAGTCTT
TTGAAAACAAATACTCTATGAGGATTTATGAGTGGTTATTAAAAGAACT
AACACAAAAGAAAACTCACAAGGCAAATATAGAGATTAGCCTTGATGAA
TTTAAGTTCATGTTAATGCTTGAAAATAACTACCATGAGTTTAAAAGGC
TTAACCAATGGGTTTTGAAACCAATAAGTAAAGATTTAAACACTTACAG
CAATATGAAATTGGTGGTTGATAAGCGAGGCCGCCCGACTGATACGTTG
ATTTTCCAAGTTGAACTAGATAGACAAATGGATCTCGTAACCGAACTTG
AGAACAACCAGATAAAAATGAATGGTGACAAAATACCAACAACCATTAC
ATCAGATTCCTACCTACATAACGGACTAAGAAAAACACTACACGATGCT
TTAACTGCAAAAATTCAGCTCACCAGTTTTGAGGCAAAATTTTTGAGTG
ACATGCAAAGTAAGTATGATCTCAATGGTTCGTTCTCATGGCTCACGCA
AAAACAACGAACCACACTAGAGAACATACTGGCTAAATACGGAAGGATC
TGA//.

SEQ ID NO:37 represents the nucleotide sequence of plasmid 100_pQBI_fes_hph (schematic plasmid map shown in FIG. 25) based on the fes promoter:

SEQ ID NO: 37
GGTTCTTATGGCTCTTGTATCTATCAGTGAAGCATCAAGACTAACAAACAAAAGT
AGAACAACTGTTCACCGTTACATATCAAAGGGAAAACTGTCCATATGCACAGAT
GAAAACGGTGTAAAAAAGATAGATACATCAGAGCTTTTACGAGTTTTTGGTGCAT
TCAAAGCTGTTCACCATGAACAGATCGACAATGTAACAGATGAACAGCATGTAA
CACCTAATAGAACAGGTGAAACCAGTAAAACAAAGCAACTAGAACATGAAATTG
AACACCTGAGACAACTTGTTACAGCTCAACAGTCACACATAGACAGCCTGAAAC
AGGCGATGCTGCTTATCGAATCAAAGCTGCCGACAACACGGGAGCCAGTGACGC
CTCCCGTGGGGAAAAAATCATGGCAATTCTGGAAGAAATAGCGCTTTCAGCCGG
CAAACCGGCTGAAGCCGGATCTGCGATTCTGATAACAAACTAGCAACACCAGAA
CAGCCCGTTTGCGGGCAGCAAAACCCGTACCCTAGGTCTAGGGCGGCGGATTTGT
CCTACTCAGGAGAGCGTTCACCGACAAACAACAGATAAAACGAAAGGCCCAGTC
TTTCGACTGAGCCTTTCGTTTTATTTGATGCCTCTAGAGCTTGCATGCCTGCAGGT
CTGGACATTTATTTGTATAGTTCATCCATGCCATGTGTAATCCCAGCAGCTGTTAC
AAACTCAAGAAGGACCATGTGGTCTCTCTTTTCGTTGGGATCTTTCGAAAGGGCA
GATTGTGTGGACAGGTAATGGTTGTCTGGTAAAAGGACAGGGCCATCGCCAATT
GGAGTATTTTGTTGATAATGGTCTGCTAGTTGAACGCTTCCATCTTCAATGTTGTG
TCTAATTTTGAAGTTAACTTTGATTCCATTCTTTTGTTTGTCTGCCATGATGTATAC
ATTGTGTGAGTTATAGTTGTATTCCAATTTGTGTCCAAGAATGTTTCCATCTTCTT
TAAAATCAATACCTTTTAACTCGATTCTATTAACAAGGGTATCACCTTCAAACTT
GACTTCAGCACGTGTCTTGTAGTTCCCGTCATCTTTGAAAAATATAGTTCTTTCCT
GTACATAACCTTCGGGCATGGCACTCTTGAAAAAGTCATGCTGTTTCATATGATC
TGGGTATCTCGCAAAGCATTGAAGACCATACGCGAAAGTAGTGACAAGTGTTGG
CCATGGAACAGGTAGTTTTCCAGTAGTGCAAATAAATTTAAGGGTAAGTTTTCCG
TATGTTGCATCACCTTCACCCTCTCCACTGACAGAAAATTTGTGCCCATTAACATC
ACCATCTAATTCAACAAGAATTGGGACAACTCCAGTGAAAAGTTCTTCTCCTTTA
CTCATATGTATATCTCCTTCTTAAATCTAGAGGATCCACGCGCTTTATCGTCGAGT
ATTCTTCAGAACCTTGGGGATCACGCCACCAGAAAATGACCTCAAACATTTCGTC
ATTCAGACGCTGCCATTCCGGGCCATGTTTCGACTGCCACCAGCTCTCACTTCCT
ACTTTTAACGCCGTCACACCATAACCCCATGTTTACTGTGCAATTTTTCATTGATT
GCAGAAATATATTGATAATATTATTGATAACTATTTGCATTTGCAATAGCGTAAT
GGCGCGCCGTGGGAAGCGCGGACATTAATTAACCAACTGCACTGCGTGTCTTTCA
GGATCAAAGGTTTTCGCGGTAGCGGGATGCGTCGTGTTGATGACGACCATGCCCG
ACAGTTGCAATTCGTGGCAAAAATGCAGGAATAAAACAATGAACAAGAAGATTC
ATTCCCTGGCCTTGTTGGTCAATCTGGGGATTTATGGGGTAGCGCAGGCACAAGA
GCCGACCCTCGAGGTGAAGACGAAAGGGCCTCGTGATACGCCTATTTTTATAGGT
TAATGTCATGATAATAATGGTTTCTTAGACGTCGGAATTGCCAGCTGGGGCGCCC
TCTGGTAAGGTTGGGAAGCCCTGCAAAGTAAACTGGATGGCTTTCTTGCCGCCAA
GGATCTGATGGCGCAGGGGATCAAGATCTGATCAAGAGACAGGATGAGGATCGT
TTCGCATGATTGAACAAGATGTTTTTTATTGGTGAGAATCCACCTTTCGACCTGCA
GCCTGTTGACAATTAATCATCGGCATAGTATATCGGCATAGTATAATACGACAAG
GTGAGGAACTAAACCATGAAAAAGCCTGAACTCACCGCGACGTCTGTCGAGAAG
TTTCTGATCGAAAAGTTCGACAGCGTCTCCGACCTGATGCAGCTCTCGGAGGGCG
AAGAATCTCGTGCTTTCAGCTTCGATGTAGGAGGGCGTGGATATGTCCTGCGGGT
AAATAGCTGCGCCGATGGTTTCTACAAAGATCGTTATGTTTATCGGCACTTTGCA
TCGGCCGCGCTCCCGATTCCGGAAGTGCTTGACATTGGGGAATTCAGCGAGAGCC
TGACCTATTGCATCTCCCGCCGTGCACAGGGTGTCACGTTGCAAGACCTGCCTGA
AACCGAACTGCCCGCTGTTCTGCAGCCGGTCGCGGAGGCCATGGATGCGATCGC
TGCGGCCGATCTTAGCCAGACGAGCGGGTTCGGCCCATTCGGACCGCAAGGAAT
CGGTCAATACACTACATGGCGTGATTTCATATGCGCGATTGCTGATCCCCATGTG
TATCACTGGCAAACTGTGATGGACGACACCGTCAGTGCGTCCGTCGCGCAGGCTC
TCGATGAGCTGATGCTTTGGGCCGAGGACTGCCCCGAAGTCCGGCACCTCGTGCA
CGCGGATTTCGGCTCCAACAATGTCCTGACGGACAATGGCCGCATAACAGCGGT
CATTGACTGGAGCGAGGCGATGTTCGGGGATTCCCAATACGAGGTCGCCAACAT
CTTCTTCTGGAGGCCGTGGTTGGCTTGTATGGAGCAGCAGACGCGCTACTTCGAG
CGGAGGCATCCGGAGCTTGCAGGATCGCCGCGGCTCCGGGCGTATATGCTCCGC
ATTGGTCTTGACCAACTCTATCAGAGCTTGGTTGACGGCAATTTCGATGATGCAG
CTTGGGCGCAGGGTCGATGCGACGCAATCGTCCGATCCGGAGCCGGGACTGTCG
GGCGTACACAAATCGCCCGCAGAAGCGCGGCCGTCTGGACCGATGGCTGTGTAG
AAGTACTCGCCGATAGTGGAAACCGACGCCCCGGATGAGGGGATCAATTCTCTA
GAGCTCGCTGATCAGAAGTTCCTGATATATTCCGCTTCCTTCTATCGCCTTCTTGA
CGAGTTCTTCTGAGCGGGACTCTGGGGTTCGAGAGCTCGCTTGGACTCCTGTTGA
TAGATCCAGTAATGACCTCAGAACTCCATCTGGATTTGTTCAGAACGCTCGGTTG
CCGCCGGGCGTTTTTTATTGGTGAGAATCCAAGCACTAGGGACAGTAAGACGGG
TAAGCCTGTTGATGATACCGCTGCCTTACTGGGTGCATTAGCCAGTCTGAATGAC
CTGTCACGGGATAATCCGAAGTGGTCAGACTGGAAAATCAGAGGGCAGGAACTG
CTGAACAGCAAAAAGTCAGATAGCACCACATAGCAGACCCGCCATAAAACGCCC
TGAGAAGCCCGTGACGGGCTTTTCTTGTATTATGGGTAGTTTCCTTGCATGAATCC
ATAAAAGGCGCCTGTAGTGCCATTTACCCCCATTCACTGCCAGAGCCGTGAGCGC
AGCGAACTGAATGTCACGAAAAAGACAGCGACTCAGGTGCCTGATGGTCGGAGA
CAAAAGGAATATTCAGCGATTTGCCCGAGCTTGCGAGGGTGCTACTTAAGCCTTT
AGGGTTTTAAGGTCTGTTTTGTAGAGGAGCAAACAGCGTTTGCGACATCCTTTTG
TAATACTGCGGAACTGACTAAAGTAGTGAGTTATACACAGGGCTGGGATCTATTC
TTTTTATCTTTTTTTATTCTTTCTTTATTCTATAAATTATAACCACTTGAATATAAA
CAAAAAAAACACACAAAGGTCTAGCGGAATTTACAGAGGGTCTAGCAGAATTTA
CAAGTTTTCCAGCAAAGGTCTAGCAGAATTTACAGATACCCACAACTCAAAGGA
AAAGGACTAGTAATTATCATTGACTAGCCCATCTCAATTGGTATAGTGATTAAAA
TCACCTAGACCAATTGAGATGTATGTCTGAATTAGTTGTTTTCAAAGCAAATGAA
CTAGCGATTAGTCGCTATGACTTAACGGAGCATGAAACCAAGCTAATTTTATGCT
GTGTGGCACTACTCAACCCCACGATTGAAAACCCTACAAGGAAAGAACGGACGG
TATCGTTCACTTATAACCAATACGCTCAGATGATGAACATCAGTAGGGAAAATGC
TTATGGTGTATTAGCTAAAGCAACCAGAGAGCTGATGACGAGAACTGTGGAAAT
CAGGAATCCTTTGGTTAAAGGCTTTGAGATTTTCCAGTGGACAAACTATGCCAAG
TTCTCAAGCGAAAAATTAGAATTAGTTTTTAGTGAAGAGATATTGCCTTATCTTTT
CCAGTTAAAAAAATTCATAAAATATAATCTGGAACATGTTAAGTCTTTTGAAAAC
AAATACTCTATGAGGATTTATGAGTGGTTATTAAAAGAACTAACACAAAAGAAA
ACTCACAAGGCAAATATAGAGATTAGCCTTGATGAATTTAAGTTCATGTTAATGC
TTGAAAATAACTACCATGAGTTTAAAAGGCTTAACCAATGGGTTTTGAAACCAAT
AAGTAAAGATTTAAACACTTACAGCAATATGAAATTGGTGGTTGATAAGCGAGG
CCGCCCGACTGATACGTTGATTTTCCAAGTTGAACTAGATAGACAAATGGATCTC
GTAACCGAACTTGAGAACAACCAGATAAAAATGAATGGTGACAAAATACCAACA
ACCATTACATCAGATTCCTACCTACATAACGGACTAAGAAAAACACTACACGAT
GCTTTAACTGCAAAAATTCAGCTCACCAGTTTTGAGGCAAAATTTTTGAGTGACA
TGCAAAGTAAGTATGATCTCAATGGTTCGTTCTCATGGCTCACGCAAAAACAACG
AACCACACTAGAGAACATACTGGCTAAATACGGAAGGATCTGA//.

SEQ ID NO:38 represents the nucleotide sequence of plasmid 101_pQBI_ugpB_hph (schematic plasmid map shown in FIG. 26) based on the ugpB promoter:

SEQ ID NO: 38
GGTTCTTATGGCTCTTGTATCTATCAGTGAAGCATCAAGACTAACAAACAAAAGT
AGAACAACTGTTCACCGTTACATATCAAAGGGAAAACTGTCCATATGCACAGAT
GAAAACGGTGTAAAAAAGATAGATACATCAGAGCTTTTACGAGTTTTTGGTGCAT
TCAAAGCTGTTCACCATGAACAGATCGACAATGTAACAGATGAACAGCATGTAA
CACCTAATAGAACAGGTGAAACCAGTAAAACAAAGCAACTAGAACATGAAATTG
AACACCTGAGACAACTTGTTACAGCTCAACAGTCACACATAGACAGCCTGAAAC
AGGCGATGCTGCTTATCGAATCAAAGCTGCCGACAACACGGGAGCCAGTGACGC
CTCCCGTGGGGAAAAAATCATGGCAATTCTGGAAGAAATAGCGCTTTCAGCCGG
CAAACCGGCTGAAGCCGGATCTGCGATTCTGATAACAAACTAGCAACACCAGAA
CAGCCCGTTTGCGGGCAGCAAAACCCGTACCCTAGGTCTAGGGCGGCGGATTTGT
CCTACTCAGGAGAGCGTTCACCGACAAACAACAGATAAAACGAAAGGCCCAGTC
TTTCGACTGAGCCTTTCGTTTTATTTGATGCCTCTAGAGCTTGCATGCCTGCAGGT
CTGGACATTTATTTGTATAGTTCATCCATGCCATGTGTAATCCCAGCAGCTGTTAC
AAACTCAAGAAGGACCATGTGGTCTCTCTTTTCGTTGGGATCTTTCGAAAGGGCA
GATTGTGTGGACAGGTAATGGTTGTCTGGTAAAAGGACAGGGCCATCGCCAATT
GGAGTATTTTGTTGATAATGGTCTGCTAGTTGAACGCTTCCATCTTCAATGTTGTG
TCTAATTTTGAAGTTAACTTTGATTCCATTCTTTTGTTTGTCTGCCATGATGTATAC
ATTGTGTGAGTTATAGTTGTATTCCAATTTGTGTCCAAGAATGTTTCCATCTTCTT
TAAAATCAATACCTTTTAACTCGATTCTATTAACAAGGGTATCACCTTCAAACTT
GACTTCAGCACGTGTCTTGTAGTTCCCGTCATCTTTGAAAAATATAGTTCTTTCCT
GTACATAACCTTCGGGCATGGCACTCTTGAAAAAGTCATGCTGTTTCATATGATC
TGGGTATCTCGCAAAGCATTGAAGACCATACGCGAAAGTAGTGACAAGTGTTGG
CCATGGAACAGGTAGTTTTCCAGTAGTGCAAATAAATTTAAGGGTAAGTTTTCCG
TATGTTGCATCACCTTCACCCTCTCCACTGACAGAAAATTTGTGCCCATTAACATC
ACCATCTAATTCAACAAGAATTGGGACAACTCCAGTGAAAAGTTCTTCTCCTTTA
CTCATATGTATATCTCCTTCTTAAATCTAGAGGATCTCTCGAGTCGTCACTGCCTG
TGCATTCCCCATTAACGCCAGTCCGAGCGCCAGTGCTGAAGCTGTATAATGTAAC
GGTTTCATCGTTTATCTCTCTTGTTGTACCGAATGCGCGAATTCACGCGTTTTATG
CTCGCGGGGTAACATGACATGCTCGAATTACAGAAAAATAACTTTTTTGTTACAT
TTGTAAGATAGTAAGGTGTCAGAAAGATGACAAGGCGGTGACGGCGTGGGTGAG
GGAAAATGGGAGATGGGGCACGGATAAGCGGGAAAATATAGAAGGTCTGAATC
AAACTCTACAGATTGCTCATCGTTTCATGCCGGATGCGGCGTAAACGCCTTATCA
GGCCTACAAGATCGTGCAAATTCAACATATTGCCACTCACCCAGTAGGCCTGATA
AGCGCAGCGCATCAGGCAATTTTACATTTGTCACCTGTCTCAAAGGAGTCTTTTG
ACTCCCTATCAATCAACGTGTTATTACCCGCCTAAATACGCACTTCTCACCGCTTC
ATTCGCCAGCAGCGCATCACCAGTATCGGAAAGCACTACATGGCCGTTTTCCAGC
ACGTAGGATCCATCGAGGTGAAGACGAAAGGGCCTCGTGATACGCCTATTTTTAT
AGGTTAATGTCATGATAATAATGGTTTCTTAGACGTCGGAATTGCCAGCTGGGGC
GCCCTCTGGTAAGGTTGGGAAGCCCTGCAAAGTAAACTGGATGGCTTTCTTGCCG
CCAAGGATCTGATGGCGCAGGGGATCAAGATCTGATCAAGAGACAGGATGAGGA
TCGTTTCGCATGATTGAACAAGATGTTTTTTATTGGTGAGAATCCACCTTTCGACC
TGCAGCCTGTTGACAATTAATCATCGGCATAGTATATCGGCATAGTATAATACGA
CAAGGTGAGGAACTAAACCATGAAAAAGCCTGAACTCACCGCGACGTCTGTCGA
GAAGTTTCTGATCGAAAAGTTCGACAGCGTCTCCGACCTGATGCAGCTCTCGGAG
GGCGAAGAATCTCGTGCTTTCAGCTTCGATGTAGGAGGGCGTGGATATGTCCTGC
GGGTAAATAGCTGCGCCGATGGTTTCTACAAAGATCGTTATGTTTATCGGCACTT
TGCATCGGCCGCGCTCCCGATTCCGGAAGTGCTTGACATTGGGGAATTCAGCGAG
AGCCTGACCTATTGCATCTCCCGCCGTGCACAGGGTGTCACGTTGCAAGACCTGC
CTGAAACCGAACTGCCCGCTGTTCTGCAGCCGGTCGCGGAGGCCATGGATGCGA
TCGCTGCGGCCGATCTTAGCCAGACGAGCGGGTTCGGCCCATTCGGACCGCAAG
GAATCGGTCAATACACTACATGGCGTGATTTCATATGCGCGATTGCTGATCCCCA
TGTGTATCACTGGCAAACTGTGATGGACGACACCGTCAGTGCGTCCGTCGCGCAG
GCTCTCGATGAGCTGATGCTTTGGGCCGAGGACTGCCCCGAAGTCCGGCACCTCG
TGCACGCGGATTTCGGCTCCAACAATGTCCTGACGGACAATGGCCGCATAACAG
CGGTCATTGACTGGAGCGAGGCGATGTTCGGGGATTCCCAATACGAGGTCGCCA
ACATCTTCTTCTGGAGGCCGTGGTTGGCTTGTATGGAGCAGCAGACGCGCTACTT
CGAGCGGAGGCATCCGGAGCTTGCAGGATCGCCGCGGCTCCGGGCGTATATGCT
CCGCATTGGTCTTGACCAACTCTATCAGAGCTTGGTTGACGGCAATTTCGATGAT
GCAGCTTGGGCGCAGGGTCGATGCGACGCAATCGTCCGATCCGGAGCCGGGACT
GTCGGGCGTACACAAATCGCCCGCAGAAGCGCGGCCGTCTGGACCGATGGCTGT
GTAGAAGTACTCGCCGATAGTGGAAACCGACGCCCCGGATGAGGGGATCAATTC
TCTAGAGCTCGCTGATCAGAAGTTCCTGATATATTCCGCTTCCTTCTATCGCCTTC
TTGACGAGTTCTTCTGAGCGGGACTCTGGGGTTCGAGAGCTCGCTTGGACTCCTG
TTGATAGATCCAGTAATGACCTCAGAACTCCATCTGGATTTGTTCAGAACGCTCG
GTTGCCGCCGGGCGTTTTTTATTGGTGAGAATCCAAGCACTAGGGACAGTAAGAC
GGGTAAGCCTGTTGATGATACCGCTGCCTTACTGGGTGCATTAGCCAGTCTGAAT
GACCTGTCACGGGATAATCCGAAGTGGTCAGACTGGAAAATCAGAGGGCAGGAA
CTGCTGAACAGCAAAAAGTCAGATAGCACCACATAGCAGACCCGCCATAAAACG
CCCTGAGAAGCCCGTGACGGGCTTTTCTTGTATTATGGGTAGTTTCCTTGCATGA
ATCCATAAAAGGCGCCTGTAGTGCCATTTACCCCCATTCACTGCCAGAGCCGTGA
GCGCAGCGAACTGAATGTCACGAAAAAGACAGCGACTCAGGTGCCTGATGGTCG
GAGACAAAAGGAATATTCAGCGATTTGCCCGAGCTTGCGAGGGTGCTACTTAAG
CCTTTAGGGTTTTAAGGTCTGTTTTGTAGAGGAGCAAACAGCGTTTGCGACATCC
TTTTGTAATACTGCGGAACTGACTAAAGTAGTGAGTTATACACAGGGCTGGGATC
TATTCTTTTTATCTTTTTTTATTCTTTCTTTATTCTATAAATTATAACCACTTGAAT
ATAAACAAAAAAAACACACAAAGGTCTAGCGGAATTTACAGAGGGTCTAGCAGA
ATTTACAAGTTTTCCAGCAAAGGTCTAGCAGAATTTACAGATACCCACAACTCAA
AGGAAAAGGACTAGTAATTATCATTGACTAGCCCATCTCAATTGGTATAGTGATT
AAAATCACCTAGACCAATTGAGATGTATGTCTGAATTAGTTGTTTTCAAAGCAAA
TGAACTAGCGATTAGTCGCTATGACTTAACGGAGCATGAAACCAAGCTAATTTTA
TGCTGTGTGGCACTACTCAACCCCACGATTGAAAACCCTACAAGGAAAGAACGG
ACGGTATCGTTCACTTATAACCAATACGCTCAGATGATGAACATCAGTAGGGAA
AATGCTTATGGTGTATTAGCTAAAGCAACCAGAGAGCTGATGACGAGAACTGTG
GAAATCAGGAATCCTTTGGTTAAAGGCTTTGAGATTTTCCAGTGGACAAACTATG
CCAAGTTCTCAAGCGAAAAATTAGAATTAGTTTTTAGTGAAGAGATATTGCCTTA
TCTTTTCCAGTTAAAAAAATTCATAAAATATAATCTGGAACATGTTAAGTCTTTTG
AAAACAAATACTCTATGAGGATTTATGAGTGGTTATTAAAAGAACTAACACAAA
AGAAAACTCACAAGGCAAATATAGAGATTAGCCTTGATGAATTTAAGTTCATGTT
AATGCTTGAAAATAACTACCATGAGTTTAAAAGGCTTAACCAATGGGTTTTGAAA
CCAATAAGTAAAGATTTAAACACTTACAGCAATATGAAATTGGTGGTTGATAAG
CGAGGCCGCCCGACTGATACGTTGATTTTCCAAGTTGAACTAGATAGACAAATGG
ATCTCGTAACCGAACTTGAGAACAACCAGATAAAAATGAATGGTGACAAAATAC
CAACAACCATTACATCAGATTCCTACCTACATAACGGACTAAGAAAAACACTAC
ACGATGCTTTAACTGCAAAAATTCAGCTCACCAGTTTTGAGGCAAAATTTTTGAG
TGACATGCAAAGTAAGTATGATCTCAATGGTTCGTTCTCATGGCTCACGCAAAAA
CAACGAACCACACTAGAGAACATACTGGCTAAATACGGAAGGATCTGA//.

### Exemplary Strains Sensitive to the Ni (II) Dication

We designed a nickel (II)-sensing strain around the E. coli resistance to cobalt and nickel regulator protein, RcnR, and its regulated promoter for the rcnAB genes. Briefly, an RcnR dimer binds to a bidirectional promoter region to repress expression from both the rcnAB promoter (SEQ ID NO:97) and its own rcnR promoter in the absence of nickel (II) or cobalt (II), and it releases from the promoter when nickel or cobalt is present. (See, Iwig, J. S., et al., Nickel homeostasis in Escherichia coli—The renR-rcnA efflux pathway and its linkage to NikR function, Mol. Microbiol. 62:252-262 (2006)). Natively, this mechanism is useful for homeostasis in E. coli, as rcnA encodes a metal efflux pump. We constructed the RcnR nickel dication-sensing plasmid pQBI_PWTW001_Nickel_prenA (SEQ ID NO:96), illustrated schematically in FIG. 32, based on this regulatory mechanism, by recombinantly making a series of recombinant constructs (including SEQ ID NO:98 and SEQ ID NO:99); this ultimately resulted in an expression cassette construct (SEQ ID NO:100), from which a mutated rcnR gene is expressed from the rcnR side of an overlapping bidirectional promoter region and the rcnA gene, a RBS (see above, SEQ ID NO:13), and the gene for superfolder GFP (sfgfp) (see above, SEQ ID NO:14) are expressed in a single transcript from the rcnAB side of the same overlapping bidirectional promoter region. Specifically, we introduced a previously described mutation to the RcnR protein (a C35A amino acid substitution that we term “RcnR-C35A”) to eliminate cobalt responsiveness with minimal effect on nickel sensitivity (See, Cayron, J. et al., Pushing the limits of nickel detection to nanomolar range using a set of engineered bioluminescent Escherichia coli, Environ. Sci. Pollut. Res. 24:4-14 (2017)). The variable sensing promoter region for this construct is shown in FIG. 31, and its DNA sequence, including the base pair substitutions used to generate RcnR-C35A and a transcriptional terminator sequence (see above, SEQ ID NO:15), is provided in SEQ ID NO:101. While we found high sensitivity and specificity of this strain to nickel (II), sensitivity likely could be further improved through modification or deletion of the native rcnA gene to reduce nickel efflux and effectively increase intracellular nickel concentration.

The E. coli strain that we designated “rcnAB” contained a nickel-sensing plasmid (full plasmid name “pQBI_PWTW001_Nickel_prenA,” schematic plasmid map shown in FIG. 32) having the nucleic acid sequence of SEQ ID NO:96:

SEQ ID NO: 96
GAATTCAAAAGATCTTAAGTAAGTAAGAGTATACGTATATCGGCTA
ATAACGTATTAAGGCGCTTCGGCGCCTTTTTTTATGGGGGTATTTTCATCCCAATC
CACACGTCCAACGCACAGCAAACACCACGTCGACCCTATCAGCTGCGTGCTTTCT
ATGAGTCGTTGCTGCATAACCGGCAGTTTTACAATCGCGTTAATAAAttaTTTGATA
TATGAATCCAGCACCTTCAGAACGACATCCAGATCTTCTTCACGTTTTAGCTCATC
CCCCTGGTGAACGATGTGTTCCGTCAGATGACCTTTAATCACTTCCCGCATCAGA
CCGTTTACCGCGCCACGGATAGCAGCAATCTGTTGTAAAACTGCAGCGGCTTCGT
GCGGCTCGTCGAGCATTTTCTTGAGCGCCACGACCTGGCCCTGAATCTTACTGGC
ACGCGCTTTCAGTTTCTGTTTATCACGGATTGTATGAGAcatGGCAACACCTGGTTA
ACAAGAATATGAAAAATCATAGCACTATTAATCTACTGGGGGGTAGTATCAGGT
ACTGGGGGGGAGTAGAATCAGATTGCCGAATTAATACTAAGAATTATTATCATG
ACCGAATTTACAACTCTTCTTCAGCAAGGAAACGCCTGGTTCTTCATCCCCAGCG
CCATCTTACTTGGTGCGCTTCATGGCCTGGAACCAGGGCACTCAAAAACGATGAT
GGCGGCGTTTATCATCGCCATCAAAGGCACCATTAAACAAGCGGTGATGCTCGG
ACTGGCAGCAACTATTTCGCATACCGCAGTGGTCTGGTTAATTGCCTTTGGCGGG
ATGGTGATCAGCAAGCGCTTTACTGCTCAATCAGCAGAACCGTGGGAATTCATTA
AAGAGGAGAAAGGTACCATGCGTAAAGGCGAAGAGCTGTTCACTGGTGTCGTCC
CTATTCTGGTGGAACTGGATGGTGATGTCAACGGTCATAAGTTTTCCGTGCGTGG
CGAGGGTGAAGGTGACGCAACTAATGGTAAACTGACGCTGAAGTTCATCTGTAC
TACTGGTAAACTGCCGGTACCTTGGCCGACTCTGGTAACGACGCTGACTTATGGT
GTTCAGTGCTTTGCTCGTTATCCGGACCATATGAAGCAGCATGACTTCTTCAAGT
CCGCCATGCCGGAAGGCTATGTGCAGGAACGCACGATTTCCTTTAAGGATGACG
GCACGTACAAAACGCGTGCGGAAGTGAAATTTGAAGGCGATACCCTGGTAAACC
GCATTGAGCTGAAAGGCATTGACTTTAAAGAAGACGGCAATATCCTGGGCCATA
AGCTGGAATACAATTTTAACAGCCACAATGTTTACATCACCGCCGATAAACAAA
AAAATGGCATTAAAGCGAATTTTAAAATTCGCCACAACGTGGAGGATGGCAGCG
TGCAGCTGGCTGATCACTACCAGCAAAACACTCCAATCGGTGATGGTCCTGTTCT
GCTGCCAGACAATCACTATCTGAGCACGCAAAGCGTTCTGTCTAAAGATCCGAAC
GAGAAACGCGATCATATGGTTCTGCTGGAGTTCGTAACCGCAGCGGGCATCACG
CATGGTATGGATGAACTGTACAAATGACTTGGACTCCTGTTGATAGATCCAGTAA
TGACCTCAGAACTCCATCTGGATTTGTTCAGAACGCTCGGTTGCCGCCGGGCGTT
TTTTATTGGTGAGAATCCAcctttcgacctgcagcctgttgacaattaatcatcggcatagtatatcggcatagtataat
acgacaaggtgaggaactaaaccatgaaaaagcctgaactcaccgcgacgtctgtcgagaagtttctgatcgaaaagttcgacagcg
tctccgacctgatgcagctctcggagggcgaagaatctcgtgctttcagcttcgatgtaggagggcgtggatatgtcctgcgggtaaat
agctgcgccgatggtttctacaaagatcgttatgtttatcggcactttgcatcggccgcgctcccgattccggaagtgcttgacattgggg
aattcagcgagagcctgacctattgcatctcccgccgtgcacagggtgtcacgttgcaagacctgcctgaaaccgaactgcccgctgtt
ctgcagccggtcgcggaggccatggatgcgatcgctgcggccgatcttagccagacgagcgggttcggcccattcggaccgcaag
gaatcggtcaatacactacatggcgtgatttcatatgcgcgattgctgatccccatgtgtatcactggcaaactgtgatggacgacaccg
tcagtgcgtccgtcgcgcaggctctcgatgagctgatgctttgggccgaggactgccccgaagtccggcacctcgtgcacgcggattt
cggctccaacaatgtcctgacggacaatggccgcataacagcggtcattgactggagcgaggcgatgttcggggattcccaatacga
ggtcgccaacatcttcttctggaggccgtggttggcttgtatggagcagcagacgcgctacttcgagcggaggcatccggagcttgca
ggatcgccgcggctccgggcgtatatgctccgcattggtcttgaccaactctatcagagcttggttgacggcaatttcgatgatgcagct
tgggcgcagggtcgatgcgacgcaatcgtccgatccggagccgggactgtcgggcgtacacaaatcgcccgcagaagcgcggcc
gtctggaccgatggctgtgtagaagtactcgccgatagtggaaaccgacgccccggatgaggggatcaattctctagagctcgctgat
cagaagttcctGATATATTCCGCTTCCTCGCTCACTGACTCGCTACGCTCGGTCGTTCGA
CTGCGGCGAGCGGAAATGGCTTACGAACGGGGCGGAGATTTCCTGGAAGATGCC
AGGAAGATACTTAACAGGGAAGTGAGAGGGCCGCGGCAAAGCCGTTTTTCCATA
GGCTCCGCCCCCCTGACAAGCATCACGAAATCTGACGCTCAAATCAGTGGTGGC
GAAACCCGACAGGACTATAAAGATACCAGGCGTTTCCCCCTGGCGGCTCCCTCGT
GCGCTCTCCTGTTCCTGCCTTTCGGTTTACCGGTGTCATTCCGCTGTTATGGCCGC
GTTTGTCTCATTCCACGCCTGACACTCAGTTCCGGGTAGGCAGTTCGCTCCAAGC
TGGACTGTATGCACGAACCCCCCGTTCAGTCCGACCGCTGCGCCTTATCCGGTAA
CTATCGTCTTGAGTCCAACCCGGAAAGACATGCAAAAGCACCACTGGCAGCAGC
CACTGGTAATTGATTTAGAGGAGTTAGTCTTGAAGTCATGCGCCGGTTAAGGCTA
AACTGAAAGGACAAGTTTTGGTGACTGCGCTCCTCCAAGCCAGTTACCTCGGTTC
AAAGAGTTGGTAGCTCAGAGAACCTTCGAAAAACCGCCCTGCAAGGCGGTTTTTT
CGTTTTCAGAGCAAGAGATTACGCGCAGACCAAAACGATCTCAAGAAGATCATC
TTATTAATCAGATAAAATATTTCTAGATTTCAGTGCAATTTATCTCTTCAAATGTA
GCACCTGAAGTCAGCCCCATACGATATAAGTTGTT//.

### Example 2. Evaluation of Analyte-Detecting Strains

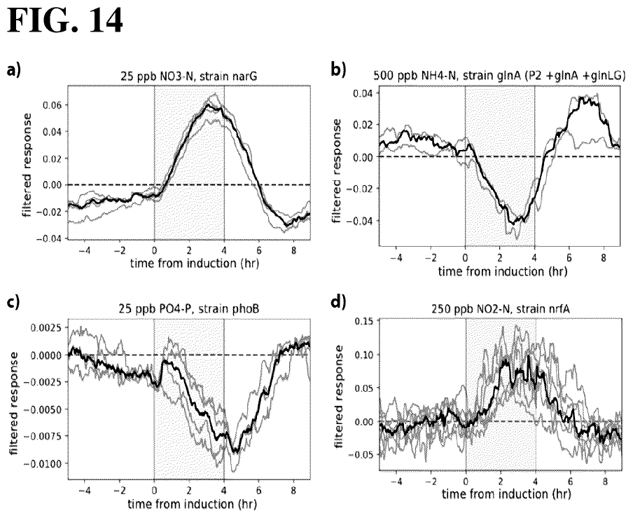

After assembling our set of 87 nutrient sensing plasmid variants, we thoroughly screened members for on- and off-target sensitivity to analytes of interest. We arrayed strains within microfluidic devices and automated parallel exposures to analytes (termed “inductions”) using a computer-controlled multivalve capable of switching between up to ten unique inputs. Furthermore, we automated several sensor prototypes in parallel to perform two inductions per microfluidic device per day for approximately a month. The plots shown in FIG. 14a-d show a total of 140, 4-hour inductions from our response dataset. We induced our strain panel with a minimum of three concentrations of each analyte, with a minimum of three replicate inductions at each concentration. FIG. 14a shows the fluorescence response of sensing strain narG built around the E. coli narG promoter driving GFP in response to a 25 ppb exposure to nitrate. FIG. 14b shows the response of sensing strain glnA built around the E. coli glnA promoter decreasing in fluorescence in response to a 500 ppb exposure to ammonium. FIG. 14c shows the response of sensing strain phoB built around the E. coli phoB promoter decreasing in fluorescence in response to a 25 ppb exposure to phosphate. Finally, FIG. 14d shows the response of sensing strain nrfA built around the E. coli nrfA promoter increasing in fluorescent response to a 250 ppb exposure to nitrite. Note that each of these inductions was performed near the limit of detection (LOD) for the relevant sensing strain, which produces clear responses but with comparatively low signal-to-noise ratio (SNR).

Development of growth media for sensing E. coli strains. In evaluating the performance of each engineered strain, we carefully considered the bacterial growth medium background for potential interference with sensing of the target analyte. It has long been known that cellular response to any particular chemical is intimately linked to the cellular environment, especially growth medium, and so a major task was to develop one or more growth media that would facilitate sensing of nitrogen, phosphate, and heavy metal species. (See, J. Monod, “The Growth of Bacterial Cultures,” Annual Reviews in Microbiology 3(X1):371-394 (1949)).

We started from the growth medium HM9, which is based on M9 medium but is derived from stocks with lower trace metal contamination, among other slight differences. (See, S. J. Beard, R. Hashim, J. Membrillo-Hernández, M. N. Hughes, and R. K. Poole, “Zinc (II) tolerance in Escherichia coli K-12: Evidence that the zntA gene (0732) encodes a cation transport ATPase,” Mol Microbiol 25(5):883-891 (1997), doi: 10.1111/j.1365-2958.1997.mmi518.x; A. I. Graham et al., “Severe zinc depletion of Escherichia coli: Roles for high affinity zinc binding by ZinT, zinc transport and zinc-independent proteins,” Journal of Biological Chemistry 284(27):18377-18389 (2009), doi: 10.1074/jbc.M109.001503). We investigated media based on HM9 but with a nitrogen source different than ammonium, since ammonium in growth medium could affect ammonium sensing and also nitrogen sensing in general. Indeed, ammonium is the preferred nitrogen source for E. coli. (See, W. C. van Heeswijk, H. v. Westerhoff, and F. C. Boogerd, “Nitrogen Assimilation in Escherichia coli: Putting Molecular Data into a Systems Perspective,” Microbiology and Molecular Biology Reviews 77(4):628-695 (2013), doi: 10.1128/mmbr.00025-13).

Other workers have addressed this issue by replacing ammonium with glutamine. (See, e.g., Cardemil, C. et al., “Bioluminescent Escherichia coli strains for the quantitative detection of phosphate and ammonia in coastal and suburban watersheds,” DNA Cell Biol, vol. 29, no. 9, pp. 519-31 (2010), doi: 10.1089/dna.2009.0984). Therefore, our initial investigation replaced ammonium in HM9 with 2 g/L glutamine; a range of glutamine concentrations 50% to 200% relative to this glutamine concentration were also explored. Our early experiments using our own synthetic constructs were based on cells growing in HM9 minus NH₄⁺ plus glutamine media. Glutamine in our growth media proved to be troublesome. Examination of cellular response revealed that different HM9 minus NH₄⁺ plus glutamine media batches became chemically distinct in an age-dependent manner. One of our primary ammonium sensors, the glnA promoter and its variants, exhibited a strong response to different media ages that was consistent with an increasing ammonium concentration with increasing age. We hypothesized that glutamine or some other compound could be degrading. Indeed, glutamine can spontaneously degrade into ammonium and pyroglutamate. (See, e.g., K. Khan and M. Elia, “Factors affecting the stability of L-glutamine in solution,” Clinical Nutrition, 10(4):186-192 (1991), doi: 10.1016/0261-5614(91)90037-D; M. Jagušić et al., “Stability of Minimum Essential Medium functionality despite L-glutamine decomposition,” Cytotechnology 68(4):1171-1183 (2016), doi: 10.1007/s10616-015-9875-8).

We confirmed glutamine degradation using a standard Hach ammonium measurement kit to determine approximate ranges of ammonium for HM9 minus NH₄⁺ plus glutamine media of different ages, and we found a range of 3-8 ppm NH₄⁺ nitrogen for media aged beyond a few days. Because this confounding time-dependent background level of ammonium represented a major risk to our sensing capabilities, we discontinued the use of HM9 minus NH₄⁺ plus glutamine for all experiments. To replace glutamine as a nitrogen source, we instead used a combination of arginine and proline. Cells exhibited healthy growth on this nitrogen background, and we did not detect significant batch-to-batch variability in responses. The strains we ultimately developed were able to detect ammonium and nitrate in this growth medium background.

To detect phosphate, we adapted HM9 medium to have a greatly reduced concentration of phosphate by reducing the concentration of glycerol-2-phosphate. In our “HM9 MES Low Phosphate” E. coli growth medium for sensing phosphate in a source water, we added β-Glycerol phosphate disodium salt pentahydrate (Sigma-Aldrich Cat. No. 50020, C₃H₇Na₂O₆P·5H₂O), such that the final concentration of P at the cells in the microfluidic chip (following dilution with the source water) is 4 μM. As an example, when we employed a microfluidic sensing chip designed to mix 1 part of 5× concentrated growth medium with 4 parts of source water, the 5× concentrated growth medium flowing into the chip contains 20 μM of β-Glycerol phosphate disodium salt pentahydrate. This was diluted to 4 μM after mixing on-chip with source water. This maintained the strains in a low but defined phosphate background in which regulatory networks for phosphate homeostasis were active. HM9 low phosphate growth medium greatly increased our sensitivity to phosphate. Nitrite and heavy metal sensing strains grown in original HM9 growth medium maintained high sensitivity to nitrite in addition to arsenic and cadmium, so we continued to use HM9 for nitrite and heavy metal sensing.

In summary, the specific compositions of the three bacterial growth media described above are as follows:

HM9 (metal and nitrite sensing media): 40 mL 1 M MES (liquid stock), 40 mL 100 mM glycerol-2-phosphate (frozen stock), 20 mL 50 g/L NH₄Cl (liquid stock), 20 mL 185 g/L KCl (liquid stock), 20 mL 20% glucose (liquid stock), 1 mL 10 mM CaCl₂ (liquid stock), 1 mL 200 mM MgSO₄ (liquid stock), 20 μL 50 mM FeCl₃ in 10 mM HCl (liquid stock), 1 mL 75% Tween (liquid stock), remaining 1 L volume filled with Milli-Q water, sterile filtered.

HM9 arg/pro (nitrate and ammonium sensing media): Same as HM9, but NH₄Cl is replaced with 2 g L-Arginine and 2 g L-Proline.

HM9 low phosphate (organic phosphate sensing media): Same as HM9, but 40 mL 100 mM glycerol-2-phosphate is replaced with 40 μL 100 mM glycerol-2-phosphate. This provides a low but defined amount of bioavailable phosphate.

Note that all culture media were supplemented with 100-200 μg/mL hygromycin B as a selection antibiotic to prevent microbial contamination.

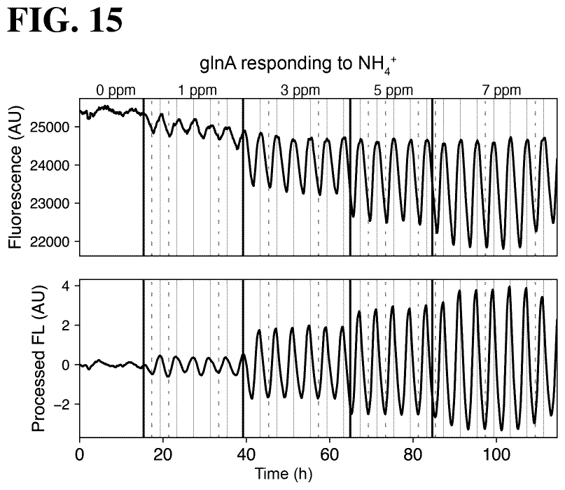

Characterization of strains. To better characterize the top-performing E. coli biosensor strains following screening in the appropriate growth medium background, we performed additional exposures at multiple concentrations of the target analytes. Here, an “AC induction” approach was used, by which cells were alternatingly exposed to 2-hour pulses of water containing the analyte (termed process water), followed by 2-hour pulses of pure water (representing a negative control baseline response). FIG. 15 shows an example of one such experiment, where the ammonium sensing glnA strain was subjected to 2-hour on-off pulses of ammonium spanning 0-7 ppm. The raw fluorescence signal (top panel) is band-pass filtered and normalized relative to the mean to produce a processed fluorescence signal (bottom panel). We subsequently performed a fast Fourier transform (FFT) on the processed signal to translate the amplitude of these AC inductions (i.e., the distance between the minimum and maximum values for each on-off cycle) to a signal that can be calibrated to analyte concentration.

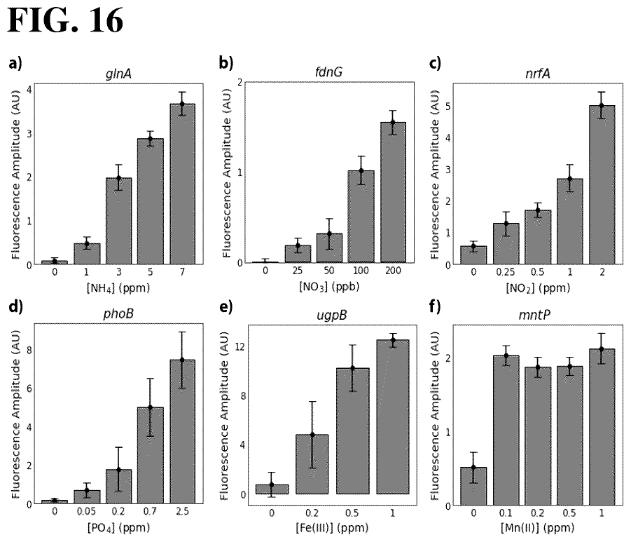

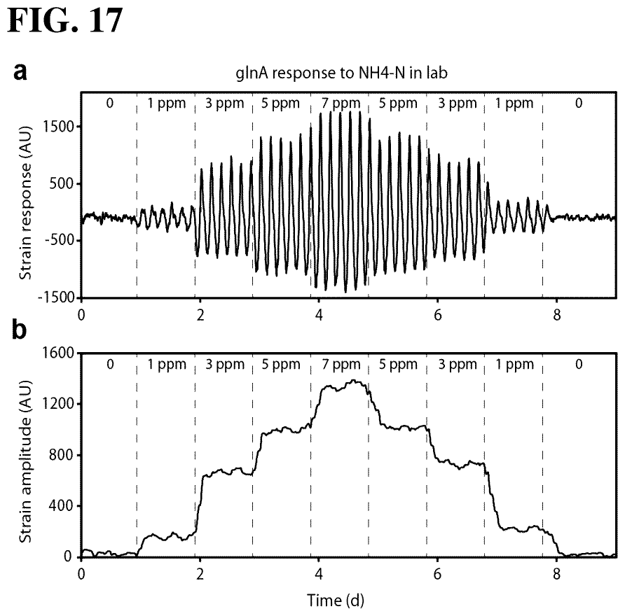

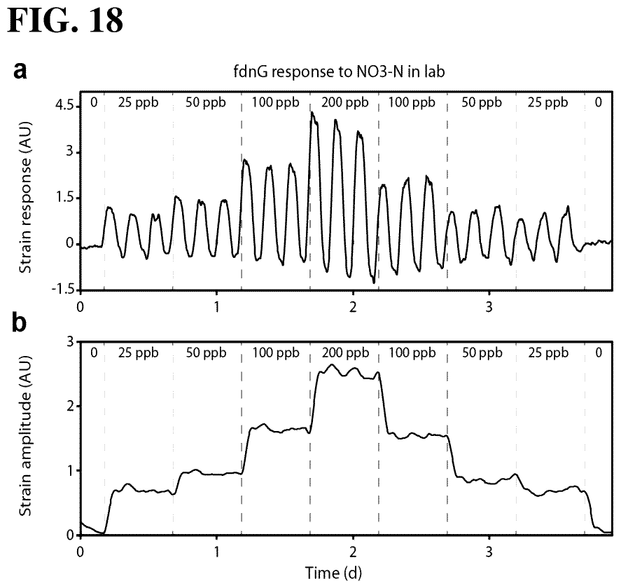

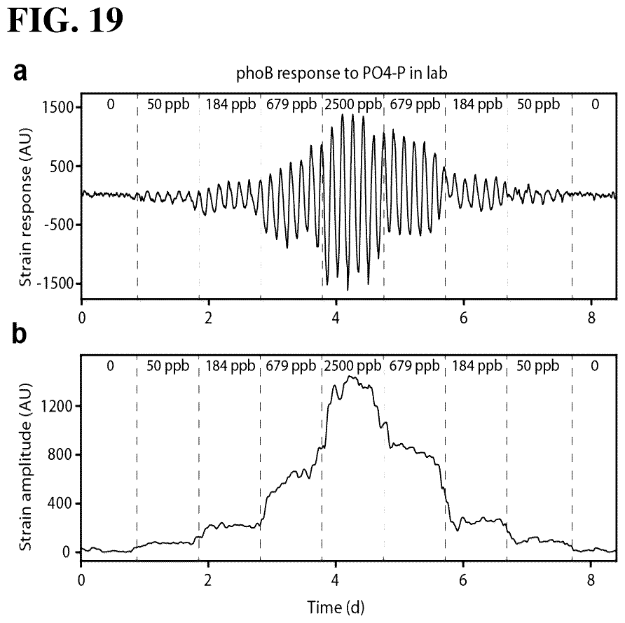

To calibrate our top-performing sensing strain panel, we subjected each strain to at least three “AC” inductions at a minimum of three concentrations of the target analyte. Corresponding fluorescence amplitude responses for six sensing strains are shown in FIG. 16a-f. For all strains, the amplitude of the fluorescence response to “AC” inductions increased with analyte concentration, although not always linearly. Laboratory calibration data can alternatively be presented as sequential “AC” inductions, where analyte concentration steps up and down. The amplitude of the fluorescence strain response correspondingly increases and decreases at each step in analyte concentration, as shown for the glnA strain responding to ammonium in FIG. 17a-b. To calibrate each biosensor strain, we performed a regression analysis between the magnitude of the strain amplitude (shown in FIG. 17b) and the analyte concentration. FIG. 18a-b shows an analogous response for the fdnG strain responding to nitrate during sequential series of “AC” inductions in the laboratory. FIG. 19a-b shows an analogous response for the phoB strain responding to phosphate during sequential series of “AC” inductions in the laboratory.

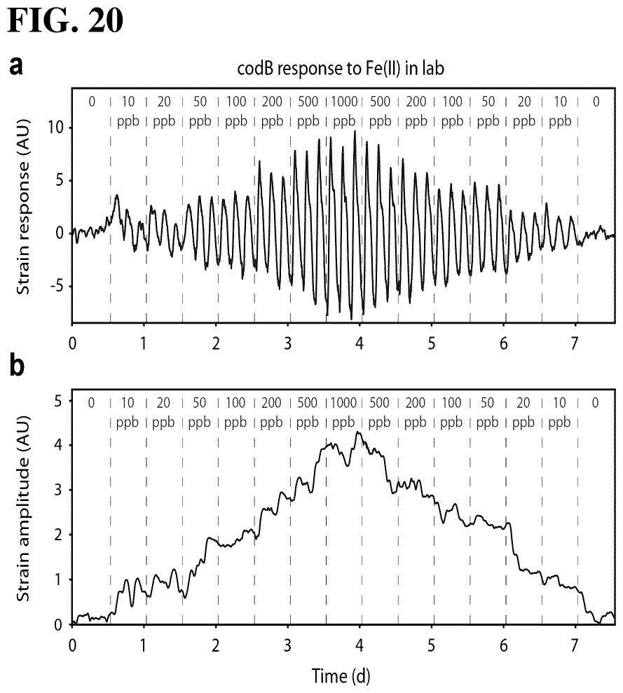

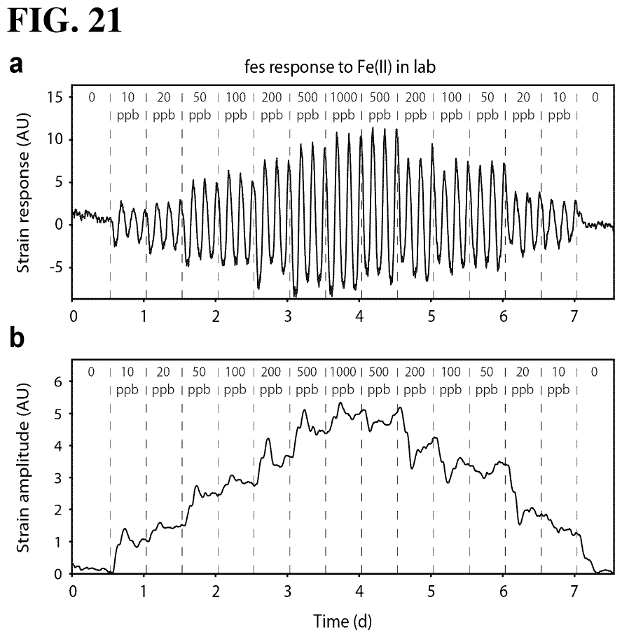

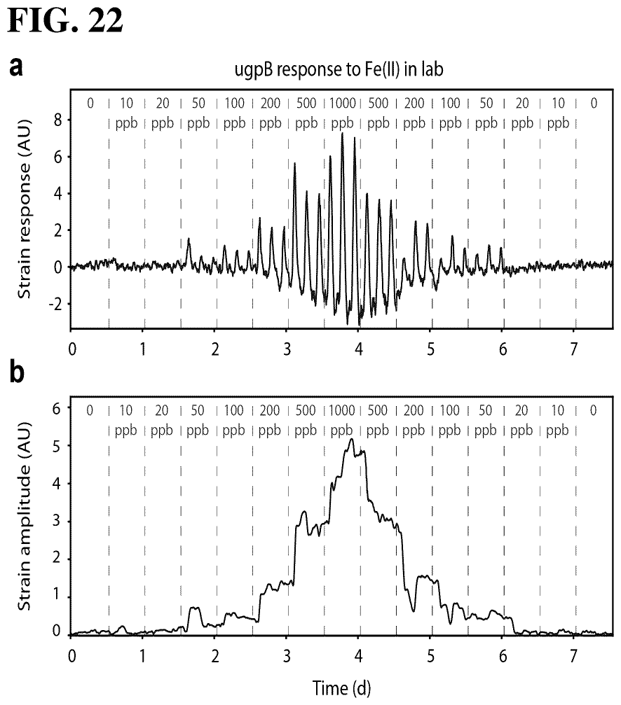

FIG. 20a-b, FIG. 21a-b, and FIG. 22a-b show sequential “AC” step inductions for the three iron-responsive strains identified by screening an E. coli fluorescent promoter library as described hereinabove. E. coli strains codB and fes were sensitive to iron concentrations as low as 10 ppb, whereas the LOD for strain ugpB was around 50 ppb. As described above, arraying multiple strains with various sensitivities within our sensing panel allowed us to expand our sensing range. For example, the responses of strains mntP and mntP-mntH(KO) saturate at 1000 ppb Mn(II); strain mntP+-mntH(KO) extends the useful dynamic range upwards for sensing up to 3000 ppb Mn(II).

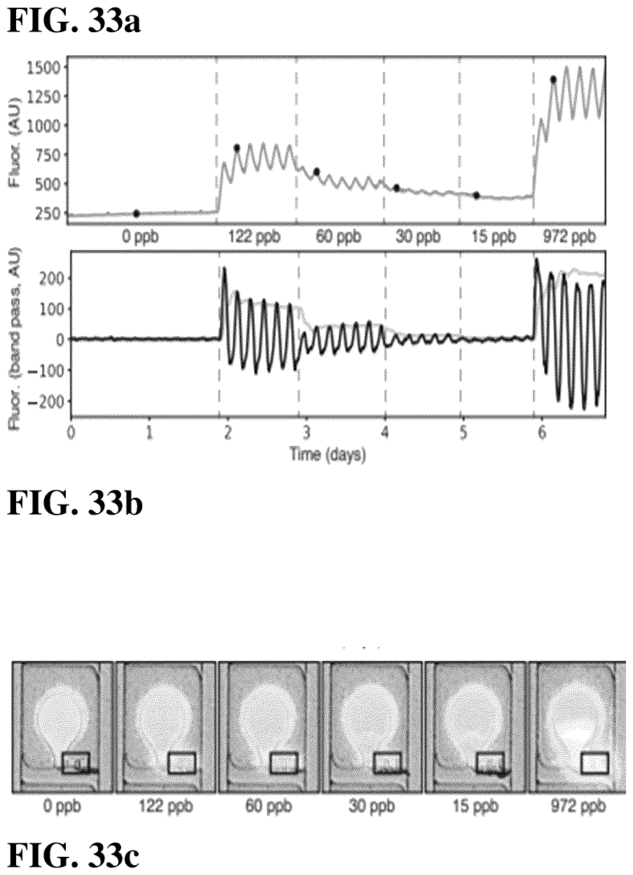

FIG. 33a-c shows the rcnAB strain response to nickel dication in a microfluidic chip as we probed the strain detection limit. We loaded renAB into all elements in our array of strain banks and booted the chip on LB. After transitioning to HM9, we monitored strain fluorescence at all positions while we alternated exposure between pure water (our “reference water” stream) and pure water spiked with various concentrations of Ni(II) (our “process water” stream) every 2 h (see “Alternating Conditions” sensor mode of operation).

FIG. 33a shows the mean cellular fluorescence response across all strain banks (gray line) over approximately seven days. The Ni(II) concentration in the process water was spiked to 122 ppb and then stepped down to 60, 30, and 15 ppb every 24 h. Transitions in the Ni(II) concentration are represented by dashed vertical lines. At 15 ppb the response became indistinguishable from 0; therefore our detection limit is approximately 30 ppb. We subsequently spiked to 972 ppb, likely saturating the response of the genetic regulatory circuit.

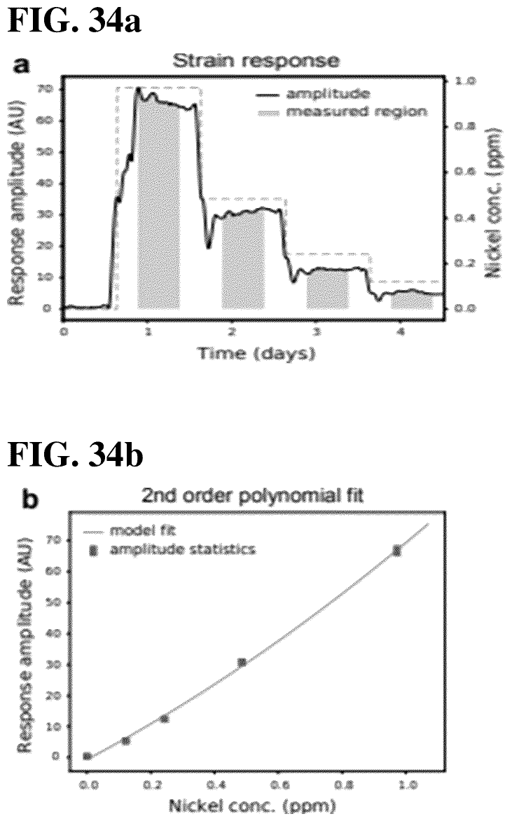

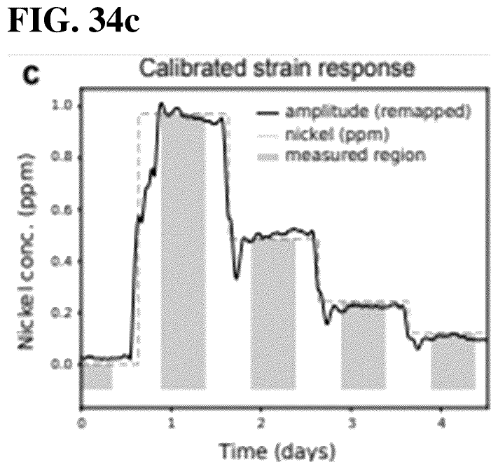

FIG. 33b shows the signal in FIG. 33a processed by a band pass filter (black line) and then a Fast Fourier Transform (FFT) with 4 h period (one complete “AC” cycle) to calculate the oscillation amplitude (gray line). In this manner, oscillation amplitude can be calibrated to Ni(II) concentration (see FIG. 34).

FIG. 33c shows a time-lapse series of images cropped to one strain bank in the microfluidic chip. Each image is a composite where the GFP fluorescence imaging channel intensity is overlaid upon the transmitted light imaging channel. Each image corresponds to a different Ni(II) concentration in the induction sequence and can be mapped to a black circle in FIG. 33a. Strain response signal for each strain bank is calculated as the average pixel intensity in the GFP channel within the two square “biopixel” cell trap regions enveloped by the black rectangles. As evident in the image series, GFP signal intensity in the “biopixel” regions tracks the Ni(II) concentration that the cells are exposed to within the chip.

FIG. 34a-c shows the results of a calibration procedure for our nickel dication sensing strain, using the rcnAB response amplitude and associated Ni(II) concentration data. FIG. 34a shows calibration data selection, in which time windows of strain responses located between transitions in analyte concentration (gray shading) are associated with known Ni(II) concentrations. The strain response amplitude (black line) corresponds to the left y-axis, and the Ni(II) concentration (gray dashed line) corresponds to the right y-axis. FIG. 34b shows the strain response amplitude data fit to the analyte concentration using a second-order polynomial model (C₁*x+C₂*x², where x is Ni(II) concentration). This fit represents a calibration curve, through which strain response can be mapped to analyte concentration.

FIG. 34c shows our calibrated rcnAB strain response data (black line) overlaid upon the nickel dication induction sequence (gray dashed line) for comparison.

A summary of the characteristics of top-performing engineered E. coli biosensor strains of the present invention is provided in Table 1 and Table 2, below. The full plasmid name represents the order of constructs comprising the plasmid. For example, in plasmid “78_pQBI_P12glnA-glnA-LutzRBS-sfgfp-glnLG_Hygro_p15A,” the two parts of the glnA promoter (P1 and P2) drive production of an operon including the glnA gene (transcription coregulator), the sfgfp gene (reporter), and the glnL and glnG genes (also regulators), with the strong Lutz RBS upstream of sfgfp. Lastly, the plasmid name includes the hygromycin resistance cassette “Hygro” and has the p15A origin of replication. The host name specifies what bacterial chassis was used to host the sensing plasmid during sensing experiments, including any knockouts in the host genome. The limit of detection indicates the lowest analyte level that reliably elicited a fluorescence response in our sensing experiments.

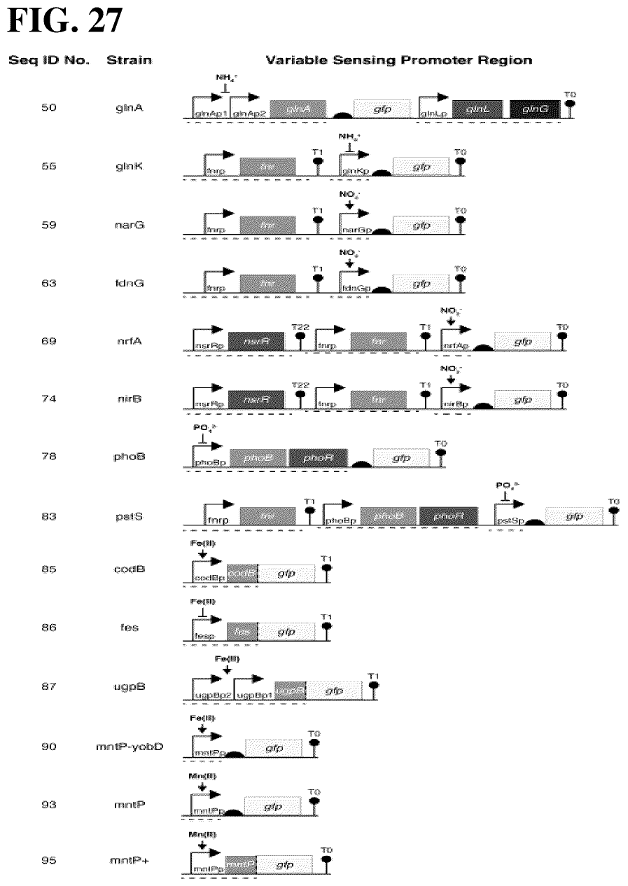

TABLE 1
Top-performing E. coli biosensor strains specific to ammonium, nitrate, nitrite,
phosphate, iron, and manganese. Key promoters and regulatory genes for each
construct are listed in the full plasmid name. Maps of the variable sensing
promoter regions found in each construct are shown schematically in FIG. 27
(with the nucleotide sequence of each construct's variable sensing promoter
region also being indicated). In Table 1, LODs are provided based on experiments
performed with each sensing plasmid transformed into the specified host chassis.
Plasmid sequences are provided, referenced by SEQ ID NO.

| | | | Plasmid | | Limit of |
|---|---|---|---|---|---|
| | Strain | | SEQ ID | | Detection |
| Analyte | Name | Plasmid Name | NO | Host Name | (ppb) |
| NH4⁺ | glnA | 78_pQBI_P12glnA-glnA- | 1 | E. coli | 250 |
| | | LutzRBS-sfgfp- | | MG1655 | |
| | | glnLG_Hygro_p15A | | | |
| | glnK | 40_pQBI_Pfnr- | 2 | E. coli | 100 |
| | | fnr(L28H)_PglnK-LutzRBS- | | MG1655 | |
| | | sfgfp_Hygro_p15A | | | |
| NO₃⁻ | narG | 21_pQBI_Pfnr- | 3 | E. coli | 5 |
| | | fnr(L28H)_PnarGtrim- | | MG1655 | |
| | | LutzRBS-sfgfp_Hygro_p15A | | | |
| | fdnG | 34_pQBI_Pfnr- | 4 | E. coli | 5 |
| | | fnr(L28H)_PfdnGtrim- | | MG1655 | |
| | | LutzRBS-sfgfp_Hygro_p15A | | | |
| NO₂⁻ | nrfA | 61_pQBI_PnsrR-nsrR_Pfnr- | 5 | E. coli | 250 |
| | | fnr(L28H)_PnrfAtrim- | | MG1655 | |
| | | LutzRBS-sfgfp_Hygro_p15A | | | |
| | nirB | 64_pQBI_PnsrR-nsrR_Pfnr- | 6 | E. coli | 100 |
| | | fnr(L28H)_PnirBtrim- | | MG1655 | |
| | | LutzRBS-sfgfp_Hygro_p15A | | | |
| PO₄³⁻ | phoB | 84_pQBI_PphoB-phoBR- | 7 | E. coli | 250 |
| | | LutzRBS-sfgfp_Hygro_p15A | | MG1655 | |
| | pstS | 85_pQBI_Pfnr- | 8 | E. coli | 250 |
| | | fnr(L28H)_PphoB- | | MG1655 | |
| | | phoBR_PpstS-LutzRBS- | | | |
| | | sfgfp_Hygro_p15A | | | |
| Iron | codB | 99_pQBI_codB_hph | 36 | E. coli | 10 |
| (Fe(II)) | | | | MG1655 | |
| | fes | 100_pQBI_fes_hph | 37 | E. coli | 10 |
| | | | | MG1655 | |
| | ugpB | 101_pQBI_ugpB_hph | 38 | E. coli | 50 |
| | | | | MG1655 | |
| | mntP- | mntP18_yobD_FurB_PmntP_{—} | 9 | E. coli | 200 |
| | yobD- | ribo-LutzRBS-sfgfp_hph_p15A | | MG1655 | |
| | mntS(KO) | | | with mntS | |
| | | | | KO | |
| Mn(II) | mntP | mntP6_MntRB_FurB_PmntP_{—} | 10 | E. coli | 10 |
| | | ribo_trim27-LutzRBS- | | MG1655 | |
| | | sfgfp_hph_p15A | | | |
| | mntP- | mntP6_MntRB_FurB_PmntP_{—} | 10 | E. coli | 200 |
| | mntH(KO) | ribo_trim27-LutzRBS- | | MG1655 | |
| | | sfgfp_hph_p15A | | with mntH | |
| | | | | KO | |
| | mntP+- | mntP20_MntRB_FurB_PmntP_{—} | 11 | E. coli | 1000 |
| | mntH(KO) | ribo_(15AA)- | | MG1655 | |
| | | sfgfp_hph_p15A | | with mntH | |
| | | | | KO | |

TABLE 2
Top-performing E. coli biosensor strain specific to nickel (II). A map of the
variable sensing promoter regions found in the construct is shown schematically
in FIG. 31 (with the nucleotide sequence of each construct's variable sensing
promoter region also being indicated). In Table 2, the limit of detection (LOD)
is provided based on experiments performed with each sensing plasmid transformed
into the specified host chassis. Plasmid sequences are provided, referenced by SEQ ID NO.

| | | | Plasmid | | Limit of |
|---|---|---|---|---|---|
| | Strain | | SEQ ID | | Detection |
| Analyte | Name | Plasmid Name | NO | Host Name | (ppb) |
| Nickel | rcnAB | pQBI_PWTW001_Nickel_prcnA | 96 | E. coli | 30 |
| (Ni(II)) | | | | MG1655 | |

### Example 3. “Real-World” Trial of an Inventive Engineered E. coli Strain in a Commercial Algae Pond

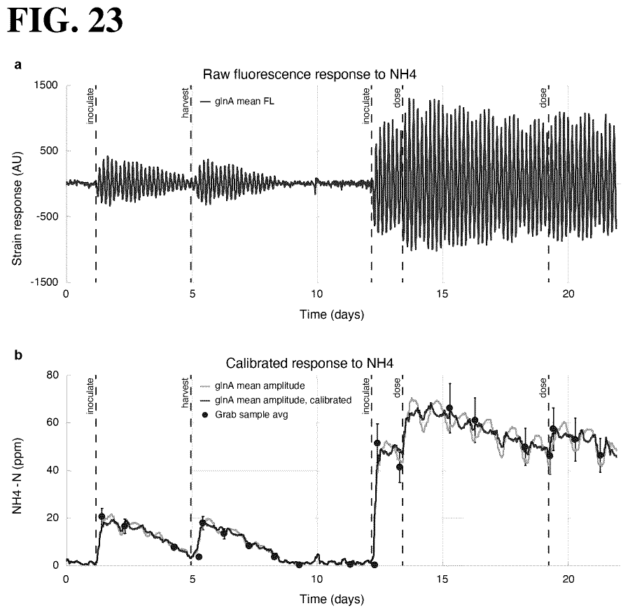

To evaluate the performance of our biosensor strains outside the laboratory, we deployed a hardware prototype to a prominent algae research facility in Mesa, Arizona. Here, the optimal growth conditions for algae biomass production are studied in 1,100-liter outdoor raceway ponds in the challenging desert climate of the southwestern United States of America, where ambient temperature can exceed 115° F. We enclosed a microfluidic device containing our E. coli sensing strain glnA within a robust hardware package designed to orchestrate all sensor operations, including imaging, valving, flow control, temperature control, and remote communications. (See, e.g., Hasty et al., “Microbial Microfluidic Biosensor,” U.S. Pat. No. 11,209,412B2). We constructed a recirculation loop with peristaltic metering pump, mesh strainers, and 500-kDa tangential flow filter (TFF) to rapidly deliver fresh filtered pond culture to our sensor hardware and return the unused majority fraction to the pond, thereby minimizing sensing delay. FIG. 23a-b shows continuous real-time field data for outdoor algae culturing throughout two pond growth experiments each spanning 22 days. The pond was inoculated with Scenedesmus obliquus (UTEX 393) culture in “brackish” (5 parts-per-thousand salinity) BG-11 medium and grew denser as it consumed available nutrients. To maintain sunlight penetration and rapid growth, algae biomass was periodically partially harvested from the pond, and fresh BG-11 medium was added to supply fresh nutrients. Alternatively, the culture was dosed with fresh BG-11 medium without biomass harvesting. These inoculation, harvesting, and dosing events are marked by vertical dashed lines. FIG. 23a shows the raw fluorescence response of our glnA engineered strain to changing ammonium concentration in the managed pond culture. FIG. 23b shows the sensor data from FIG. 23a calibrated to triplicate pond grab samples analyzed by Hach ammonium test kits. Biosensor ammonium measurements agree with Hach measurements well within the ±15% accuracy window deemed acceptable by site management (indicated by error bars). These long-term, continuous, and quantitative analyte data demonstrate the effectiveness of the inventive E. coli biosensor strains in a commercial biomass production application. We noted diurnal oscillations in our NH₄⁺ measurements, which may be explainable in view the particular embodiment of the microfluidic biosensor hardware employed; with this embodiment of the hardware we continuously actively degassed the inlet stream of filtered pond culture before diluting it with a custom additive and pumping it to the microfluidic chip. We found that the sensed NH₄⁺ concentration diurnally dipped at mid-day, when both temperature and photosynthetic productivity peaked. Because NH₄⁺ solubility decreases at higher temperatures, NH₄⁺ outgassing at elevated temperature and vacuum degasser pressure could explain our observations. Additionally, or alternatively, high algae productivity generates high dissolved oxygen (DO) levels that could have “scrubbed” NH₄⁺ from solution during continuous degassing. Whatever the cause, we subsequently improved the microfluidic chip hardware by 1) adding temperature control to stabilize NH₄⁺ solubility, and by 2) only briefly degassing the diluted aqueous culture medium further downstream in transit to the microfluidic chip. These hardware improvements eliminated diurnal oscillations in our NH₄⁺ measurements.

### Example 4. Panel of Recombinant Escherichia coli Strains Incorporated into a Hardware System for Assays

The inventive panel of recombinant Escherichia coli biosensor strains can operate as an assay of analytes when incorporated into a hardware system designed to sample the source water, which is mixed and diluted with concentrated defined culture medium. The plurality of E. coli strains in the platform are useful to detect and quantify a signal coming from the expression of a detectable marker in response to the presence of the analyte of interest. For example, in some embodiments, fluidic, electronic, and optical systems can be designed to continuously draw and mix source water with a concentrated defined culture medium within a microfluidic chip housing the cells and periodically acquiring and processing images of the microfluidic chip to detect and quantify the expression of a detectable marker, such as GFP; then the GFP signal for each strain can be calibrated to a concentration of the on-target analyte of interest, e.g., a contaminant. In this manner, a panel of the inventive E. coli biosensor strains can be employed to continuously monitor a source water for a variety of analytes in the aqueous sample. Optionally, the marker signal data can be gathered and transmitted to an operator or database in real time.

The E. coli biosensor strains can be loaded into predetermined addressable locations arrayed in the hardware device (e.g., a microfluidic device, or “chip”) during its manufacture, or post-manufacture. E. coli culture biomass can be transferred from an agar plate or liquid culture into an array of spotting “reservoirs” in the device, either individually and sequentially by hand or in parallel by pin tool. For example, the pin tool can be automated to robotically select and align the source and target colony arrays, retrieve biomass from the source array, and deposit it upon the target array. Following “loading” of the spotting reservoirs in the hardware (e.g., microfluidic) device, strain viability can be preserved either by air drying or freeze drying. For air drying, the colonies are allowed to dry naturally through evaporation. For freeze drying, the hardware device (e.g., microfluidic chips) are frozen, and vacuum pressure is applied to sublimate water vapor. For example, the microfluidic chip monolith containing the loaded spotting reservoirs can be sealed against a flat surface to form the microfluidic channels using various methods; both chip halves can be treated with oxygen plasma to activate the surfaces before spotting and then brought into contact to form a covalent bond. Alternatively, a pressure-sensitive adhesive can be applied to one chip half and then compressed against the other to rupture adhesive vesicles at the contact points but not along the open microfluidic channels.

The E. coli strains are typically viable in a microfluidic device when shielded from light at refrigerator temperature (4° C.) to room temperature (20° C.) for at least 30 days. When the microfluidic chip is booted in preparation for sensing, rich medium (e.g. Lysogeny Broth (LB)) is flowed into the chip to mix with the source water at a predetermined defined dilution ratio and hydrates the arrayed spotting reservoirs. Strains spotted within each reservoir grow to confluence throughout the following 12-24 hours, with excess culture overflowing each reservoir and washing downstream to an off-chip waste receptacle. After revival of the dehydrated strains, the culture medium can be swapped to a well-defined minimal medium (e.g. M9 or HM9) to ensure the purity of the medium stream and slow the growth rate of the sensing strains. (See, e.g., Hasty et al., “Microbial Microfluidic Biosensor,” U.S. Pat. No. 11,209,412B2).

Culturing conditions within the hardwire device (e.g., a microfluidic chip) can be maintained to ensure that each strain signal has a stable baseline. Flow rates of source water and concentrated defined culture medium can be controlled by continuously measuring them using a flow meter (e.g., in a microfluidic device the sensing range can be 0-80 μl/min) and using a proportional integral derivative (PID) feedback loop to adjust driving pressure (e.g., headspace pressure in a closed fluidic vessel, force applied to a syringe pump, or meniscus elevation generating hydrostatic pressure). Culture temperature can be maintained, preferably at 37° C., by measuring the temperature near the microfluidic chip using a probe (e.g. thermistor or thermocouple) and using a PID feedback loop to adjust output from a temperature control system (e.g. thermoelectric (Peltier) module, resistive heater, or compressor-based cooler). Culture pH can be maintained by including a buffer (e.g. MES, MOPS, HEPES, or PIPES) in the concentrated growth medium. With proper culturing conditions maintained, cellular responses can be continually measured within the microfluidic device for up to a few months.

While the arrayed microfluidic approach described above represents an efficient, miniaturized, and continuous platform embodiment to deploy the panel of sensing strains, numerous alternative deployment strategies are envisaged for practicing the method for continuously monitoring a plurality of analytes of interest. For continuous monitoring applications, the recombinant E. coli strains can be deployed in chemostats spanning the micro- to macro-fluidic scale. Milliliter-scale sensing reactors can be particularly useful to groups without access to precision optics or microfluidic flow control hardware. Beyond continuous applications, many batch culturing approaches can prove valuable for one-time tests. A simple application can involve the culturing of these recombinant biosensing E. coli strains in separate wells of microtiter plate, to which a water sample can be added, followed by incubation and fluorescence measurement. Likewise, the E. coli strains can be deployed in small water collection vials or integrated into paper-based tests. Rather than being identified by spatial address, the inventive E. coli strains can be co-cultured, with each strain expressing a unique fluorescent reporter protein.

In an embodiment where the engineered recombinant E. coli strains produce a fluorescent protein reporter as the detectable marker in response to exposure to on-target water analytes of interest (e.g., contaminants), the fluorescent protein reporter can be a green fluorescent protein (GFP) variant, such as superfolder GFP (sfGFP). Cellular production of sfGFP can be measured by illuminating the cells with blue light (nominally 485 nm wavelength) and measuring the production of green light (nominally 510 nm wavelength). An appropriate sfGFP illumination source can comprise a blue light-emitting diode (LED) (e.g., Cree XLamp XP-E2 Blue, #XPEBBL-L1-0000-00301) and an excitation filter (e.g., Semrock 482/18 nm BrightLine single-band bandpass filter, #FF02-482/18). Additional spectrally-compatible fluorescent protein or dye readouts can be imaged with the use of a multi-band emission filter. For example, cellular production of the mKate2 red fluorescent protein can be imaged by illuminating the cells with an amber LED (e.g., Cree XLamp XP-E2 Amber, #XPEBAM-L1-0000-00901) through an excitation filter (e.g., Semrock 585/40 nm BrightLine single-band bandpass filter, #FF01-585/40). In addition to fluorescent light imaging, a transmitted light image of the cells within the hardware device (e.g., microfluidic device) can be acquired by illuminating with a green LED (e.g., Cree XLamp XP-E2 Green, #XPEBGR-L1-0000-00A01). To distinguish detectable markers or reporters using multiple imaging channels, emitted fluorescence and transmitted green light should pass through a compatible emission filter (e.g., for this configuration, Semrock 527/645 nm BrightLine dual-band bandpass filter, #FF01-527/645). Monochrome images in each spectral channel can be sequentially acquired by a CCD camera (e.g., FLIR Blackfly S Mono 1.6 MP GigE Vision, #BFS-PGE-16S2M-CS).

Experimental noise in image datasets can be minimized by employing tight temporal control of LED switching, LED warm-up, and camera triggering. For long-term continuous sensing, illumination should be stable over multi-day timescales and large temperature swings (>10 C). LED driver circuit design should be constant-current and adjustable, such any variation in LED illumination with time or temperature can be compensated for in hardware, based on known calibration curves.

In alternative embodiments, the basis of cellular readout can be altered from fluorescence to luminance, absorbance, turbidity, or electrochemical. Compatible readout mechanisms include enzyme production (e.g. bioluminescence or chemiluminescence), cellular lysis, or cellular agglutination.

Fluid flow across the microfluidic chip is driven by establishing pressure differentials across the input and output ports. These pressure differentials can be applied using several methods, including pressurizing the headspace of a closed fluidic vessel with dip tube outlet, applying force to a syringe pump, or elevating an open reservoir to generate hydrostatic pressure. All of these methods are preferable to positive-displacement pump styles (e.g. rotary, piston, diaphragm, peristaltic) in that the resulting fluid flow is non-pulsatile. Macro-scale pressure pulses typically generate large flow waves within microfluidic devices that can disrupt colony stability, thereby increasing experimental noise.

In one useful embodiment, a microfluidic pumping system employs compressed gas and electro-pneumatic regulators to pressurize the headspaces of sealed fluidic vessels with dip tubes. Such a system can produce controlled non-pulsatile flows by adjusting headspace pressure based on feedback from a PID control loop and microfluidic flow meter. In some embodiments, electro-pneumatic regulators and peristaltic pumps can be combined to mix media and reference water flow streams from pressurized vessels with a source water flow stream delivered from an open vessel by peristaltic pump. The set points of multiple PID control loops can be configured to achieve proper mixing ratios of the flow streams within the microfluidic device.

For the recombinant E. coli sensing strains expressing fluorescent reporters as the detectable marker in response to the presence of an analyte of interest, a sequence of images can be acquired in all imaging channels (e.g., sfGFP fluorescence, mKate2 fluorescence, transmitted), with a typical period of 5 minutes. These stacks of images can be automatically processed in real time by image analysis software scripts to extract a dynamic response from each biosensor E. coli strain. First, transmitted light images can be registered to a reference mask to correct for drift and potentially larger jumps in image position (see, e.g., FIG. 28a). Next, strain response results can be extracted from predefined locations (i.e., “submasks”) within each microfluidic cell reservoir in the stabilized sfGFP image set. Replicate elements in a microfluidic array of E. coli strains can have their aggregate statistics pooled via a median operation to provide a strain response measure that is robust to outliers and other sources of variability. The final strain response information can then be inserted into a database (e.g., MySQL) for logging or storage.

Using an “Alternating Conditions” (AC) sensor mode of operation, the fluidic input to the sensing E. coli strain panel in a microfluidic device can be alternated between pulses of: 1) source water containing an unknown concentration of the target analyte, and 2) “reference” water with a similar background composition to the source water but not containing the analyte of interest. In practice, a microfluidic chip design can be fluidically split into replicate arrays of sensing recombinant E. coli strains, and these source and reference water streams can be periodically alternated between them using automated valving. When measuring responses from transcriptional reporters with timescales of tens of minutes, the “AC” pulse period can vary from around 1 to 4 hours, with a nominal value of around 2 hours. The “AC” mode is essentially a series of precisely timed inductions, where cells do not return to baseline levels of fluorescence. This leads to periodic modulation of cell fluorescence (see, FIG. 28b), where the oscillation amplitude can be calculated and calibrated to analyte concentration using a number of methods.

The following useful method of calculating the instantaneous oscillation amplitude, G_{AC}, can be easily implemented in both traditional computing environments and web browser environments. For each engineered recombinant E. coli strain, we first calculate either the mean or median fluorescence response across replicate observation regions in the microfluidic device, where response in this case is band pass filtered response. This response leads to signal gₙ at a discrete set of time points tₙ, with n being an integer that labels the time points. We then perform a least-square regression for gₙ using a function ƒₙ with unknown fitting parameters A, B, and C,

ƒₙ=A+B sin(ωtₙ)+C cos(ωtₙ)
where ω=2π/T is the natural frequency for period T (a specified parameter). When calculating the amplitude response at time t, we perform this regression for time points tₙ satisfying t−t_{H}<tₙ≤t for history duration t_{H} (a second specified parameter). The required least-square regression is then a standard calculation that depends on straightforward linear algebra. Application of this process leads to time-dependent fitting parameters A(t), B(t), and C(t) that can be used to quantify the baseline, phase, and amplitude of response. In particular, we define the oscillation amplitude G_{AC} as

$${G}_{A C} ( t ) = [ {B ( t )}^{2} + {C ( t )}^{{2 ]}^{1 / 2}}$$

Calculation of G_{AC} in this manner is rather fast and is readily generalized to other regression methods if desired.

There are two primary parameters that must be specified to calculate G_{AC}. The period parameter T determines the frequency of cellular response that is to be sampled, and T should almost always be set to the experimental drive period used in “AC” mode. The history parameter t_{H} determines how many time points are to be used in the regression. Very short t_{H} integrates over very little data and is susceptible to noise, while very long t_{H} may miss important time-dependent features of analyte concentration. We have found empirically that values t_{H}=T and t_{H}=1.5T produce reasonably time-localized and low-fluctuation amplitude response.

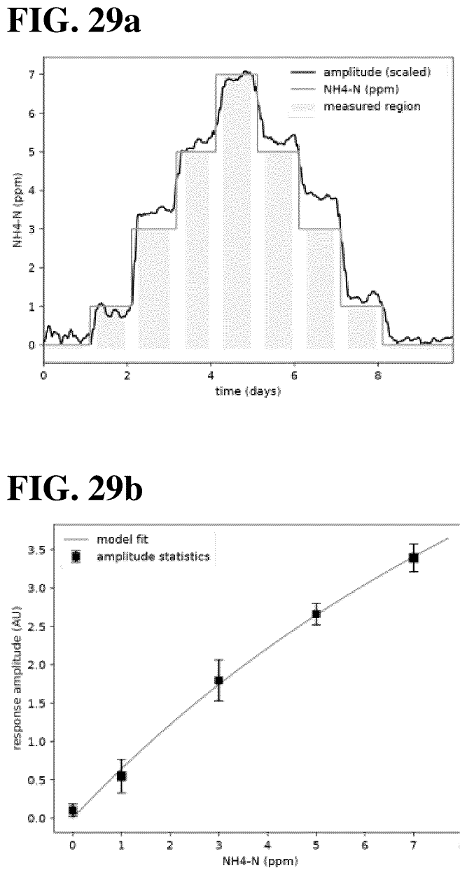

As a final step in quantifying measurements of strain fluorescence response, the strain response amplitude, G_{AC}, can be calibrated to the concentration of the target analyte. For illustration, in FIG. 28c, we overlay plots of 1) the concentrations of analyte in a series of step inductions of an arsenic-responsive engineered E. coli strain in a microfluidic device and 2) the amplitude of the raw “AC” strain response shown in FIG. 28b (calculated as G_{AC} above). The “AC” response amplitude can be fit to the induction concentrations to generate a calibration curve (see, e.g., FIG. 29a-b). Given this calibration curve, the strain response during a future induction with source water containing unknown analyte concentration can be mapped to an analyte concentration.

The previous embodiment of the Alternating Conditions (AC) sensor mode of operation for the sensing recombinant E. coli strain panel is particularly employed to measure analytes in a relatively pure source water stream (e.g., laboratory water containing an unknown concentration of the target analyte of interest). However, most biosensing applications of interest will involve quantitatively measuring analyte concentrations in an environmental source water stream, such as groundwater and surface water. In these scenarios, the composition of the “reference” water stream used in the “AC” analysis technique is matched to the background composition of the source water stream but omits the target analyte to be sensed. This decreases noise in the strain response by reducing biological artifacts due to sudden shifts in extracellular pH, osmolarity, and ionic strength. Ideally, a formulation for the reference water should be optimized such that several key water quality parameters match the source water background. Example key water quality parameters include pH, bicarbonate, carbonate, total organic carbon (TOC), alkalinity, hardness, calcium, chloride, magnesium, manganese, ammonium, nitrate, nitrite, potassium, sodium, and sulfate.

An important application of the inventive panel of recombinant Escherichia coli strains is for sensing nutrient loads in impure water streams. Such source waters can include high nutrient loads in wastewaters or agricultural surface waters as well as nutrients added to culture media in closed or open bioreactors used to produce biomass and other biological products. In the case of algal biomass production in photobioreactors, nutrients are added to algal growth media, and their concentrations are depleted as the culture density increases. The biosensor panel of recombinant E. coli strains can also be used to inform the periodic dosing of nutrients to optimize biomass or bioproduct production.

When using the inventive biosensing panel of E. coli strains to quantify nutrient concentrations in algal cultures, the “reference” water stream used in the “AC” analysis technique should be matched to the algal growth medium, while omitting the sensing target analyte. Common algal growth media used in commercial production include (in order of increasing salinity) Zarrouk's Medium, BG-11 Medium, and F/2 Medium.

1. A panel of recombinant Escherichia coli strains for monitoring a plurality of analytes of interest in an aqueous sample, comprising:
a set of two or more recombinant Escherichia coli strains that, in a defined aqueous culture medium, constitutively express one or more antibiotic resistance genes providing resistance to one or more antibiotic agents, wherein the one or more antibiotic agents, separately, or in combination, are characterized by both antibacterial and antifungal activity in the aqueous culture medium, each strain comprising a stable recombinant expression system comprising an expression cassette comprising an analyte-sensitive promoter that specifically responds to at least one of the plurality of analytes of interest, resulting in a modification of expression from the promoter, said promoter being capable of operating under aerobic physiological conditions and being operably linked to a gene encoding a detectable marker; and
wherein the set of two or more Escherichia coli strains is capable of expressing the detectable marker in the presence of at least one of the plurality of analytes of interest in a continuous series of aqueous samples diluted with fresh defined culture medium, and the set of two or more Escherichia coli strains, wherein the identity of each Escherichia coli strain can be distinguished.

2. The panel of claim 1, wherein the one or more antibiotic resistance genes comprise a hygromycin B resistance gene.

3. The panel according to claim 1, wherein the one or more antibiotic resistance genes comprise:
(i) a first antibiotic resistance gene providing resistance to an antibiotic agent characterized by antibacterial activity; and
(ii) a second antibiotic resistance gene providing resistance to an antibiotic agent characterized by antifungal activity.

4. The panel of claim 3, wherein the first antibiotic resistance gene provides resistance to penicillin, ampicillin, kanamycin, zeocin, neomycin, polymyxin B, colistin, bacitracin, streptomycin, or spectinomycin.

5. The panel according to claim 3, wherein the second antibiotic resistance gene provides resistance to clotrimazole, econazole, miconazole, terbinafine, fluconazole, ketoconazole, nystatin, or amphotericin.

6. The panel according to claim 1, wherein the detectable marker is an optically detectable marker.

7. The panel according to claim 1, wherein the detectable marker is a fluorescent protein or a luminescent protein.

8. The panel according to claim 1, wherein the plurality of analytes of interest comprises one or more ionic species selected from the group consisting of ammonium, nitrate, nitrite, phosphate, manganese dication, nickel dication, and an iron cation, or a combination of any one of these members.

9. The panel according to claim 1, wherein the plurality of analytes of interest comprises ammonium and the defined medium does not contain added glutamine.

10. The panel according to claim 1, wherein each Escherichia coli strain in the panel expresses the detectable marker in the presence of the at least one of the plurality of analytes of interest, with a limit of detection for the analyte in the aqueous sample being in a range between 1 to 1000 ppb.

11. The panel according to claim 10, wherein the limit of detection for the analyte in the aqueous sample is in a range between 1 to 500 ppb.

12. The panel according to claim 11, wherein the limit of detection for the analyte in the aqueous sample is in a range between 5 to 250 ppb.

13. The panel according to claim 1, wherein the stable recombinant expression system comprises an expression cassette that comprises a variable sensing promoter region sensitive to:
(i) ammonium, wherein variable sensing promoter region comprises a nucleotide sequence selected from the group consisting of SEQ ID NO:50 and SEQ ID NO:55;
(ii) nitrate, wherein variable sensing promoter region comprises a nucleotide sequence selected from the group consisting of SEQ ID NO:59 and SEQ ID NO:63;
(iii) nitrite, wherein variable sensing promoter region comprises a nucleotide sequence selected from the group consisting of SEQ ID NO:69 and SEQ ID NO:74;
(iv) phosphate, wherein variable sensing promoter region comprises a nucleotide sequence selected from the group consisting of SEQ ID NO:78 and SEQ ID NO:83;
(v) iron, wherein variable sensing promoter region comprises a nucleotide sequence selected from the group consisting of SEQ ID NO:85, SEQ ID NO:86, SEQ ID NO:87, and SEQ ID NO:90;
(vi) manganese (II), wherein variable sensing promoter region comprises a nucleotide sequence selected from the group consisting of SEQ ID NO:93 and SEQ ID NO:95; or
(vii) nickel (II), wherein variable sensing promoter region comprises the nucleotide sequence of SEQ ID NO:101.

14. The panel according to claim 13, wherein the stable recombinant expression system comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:36, SEQ ID NO:37, SEQ ID NO:38, and SEQ ID NO:96.

15. The panel according to claim 1, wherein the set of two or more Escherichia coli strains is arrayed in a microfluidic device.

16. The panel according to claim 1, wherein the set of two or more Escherichia coli strains is revivable from a dehydrated state.

17. The panel according to claim 1, wherein the set of two or more Escherichia coli strains is in a lyophilized or air-dried state.

18. The panel according to claim 1, wherein after being in a dehydrated state for at least 30-60 days, the set of two or more Escherichia coli strains is capable of being revived under aqueous physiological conditions and is capable of expressing the optically detectable marker in the presence of at least one of the plurality of analytes of interest.

19. A method for monitoring a plurality of analytes of interest, comprising:
(a) mixing a continuous series of aqueous samples with a fresh defined culture medium in a defined dilution ratio to obtain a series of diluted samples;
(b) contacting the continuous series of diluted samples with the panel comprising a set of two or more recombinant Escherichia coli strains according to claim 1, under aerobic physiological conditions;
(c) monitoring for the expression of the detectable marker by the set of two or more Escherichia coli strains arrayed in locations, wherein the identity of each Escherichia coli strain can be distinguished; and
(d) correlating any expression of the detectable marker by the set of two or more Escherichia coli strains in subpart (c), with the presence of at least one of the plurality of analytes of interest in the aqueous sample with a limit of detection in a concentration range of 1-1000 ppb.

20. The method according to claim 19, wherein the limit of detection is in the concentration range of 1-500 ppb.

21. The method according to claim 19, wherein the limit of detection is in the concentration range of 5-250 ppb.

22. The method according to claim 19, wherein the plurality of analytes of interest comprises one or more ionic species selected from the group consisting of ammonium, nitrate, nitrite, phosphate, manganese dication, nickel dication, and an iron cation, or a combination of any one of these members.

23. The method according to claim 19, wherein the plurality of analytes of interest comprises ammonium and the defined culture medium does not contain added glutamine.

24. The method according to claim 19, wherein the detectable marker is an optically detectable marker.

25. The method according to claim 19, wherein the detectable marker is a fluorescent protein or a luminescent protein.

26. The method according to claim 19, wherein the set of two or more Escherichia coli strains is arrayed in a microfluidic device.

27. The method according to claim 19, wherein the continuous series of aqueous samples of source water is obtained sequentially from the source water over a time period extending from 1 minute up to 60 days.
